(19) United States
(12) Patent Application Publication
(10) Pub. No.: US 2026/0053871 A1
(43) Pub. Date: Feb. 26, 2026

(54) A CARBOMER HYDROGEL COMPRISING A PHAGE LYSATE FOR MAINTAINING NATURAL SKIN MICROFLORA AND SUPPRESSING PATHOGENIC BACTERIA

(71) Applicant: FAGOFARMA S.R.O., Praha 2 (CZ)

(72) Inventors: Marek MOSA, Meceriz (CZ); Martin BENESIK, Strani (CZ); Tereza SOPIKOVA, Zlin (CZ)

(73) Assignee: FAGOFARMA S.R.O., Praha 2 (CZ)

(21) Appl. No.: 18/879,967

(22) PCT Filed: Jun. 20, 2023
(86) PCT No.: PCT/CZ2023/050034

§ 371 Date: Dec. 30, 2024

(30) Foreign Application Priority Data
Jul. 1, 2022 (CZ) 2022-297

(51) Int. Cl. A61K 35/76; A61K 9/06; A61K 47/10; A61K 47/18; A61K 47/22; A61K 47/32; A61K 47/44; A61K 47/46

(52) U.S. Cl. CPC A61K 35/76; A61K 9/06; A61K 47/10; A61K 47/18; A61K 47/183; A61K 47/186; A61K 47/22; A61K 47/32; A61K 47/44; A61K 47/46

(57) ABSTRACT

A carbomer hydrogel for maintaining the natural skin microflora and suppressing pathogenic bacteria that contains lytic enzymes and/or highly stable crude/purified Staphylococcus aureus phage lysates with a titer of at least 10⁷ pfu/g of gel and crude/purified Cutibacterium acnes phage lysates with a titer of at least 10⁵ pfu/g of gel.

### CROSS-REFERENCE TO RELATED APPLICATIONS

This application is a National Phase Application of PCT International Application No. PCT/CZ2023/050034, filed on Jun. 20, 2023, claiming the benefit of Czech Patent Application No. PV 2022-297, filed on Jul. 1, 2022, which are all hereby incorporated by reference in their entirety.

### SEQUENCE LISTING INCORPORATION

The “.xml” Sequence Listing filed with this application by EFS and which is entitled P-643504-US_ST26.xml, which is 360 kilobytes in size and which was created on Nov. 4, 2025, is hereby incorporated by reference.

### TECHNICAL FIELD

The invention relates to the composition and preparation of a carbomer hydrogel containing phage lysates effective against Staphylococcus aureus and Cutibacterium acnes (formerly Propionibacterium acnes) bacteria. The invention relates to the composition of a carbomer hydrogel that may, besides the bacteriophages themselves, also contain bacteriophage-encoded lytic enzymes (endolysins). The hydrogel is suitable for maintaining the natural skin microflora and suppressing pathogenic bacteria.

### BACKGROUND ART

Acne vulgaris, one of the most common chronic skin diseases, occurs due to disruption of the natural skin microflora or its partial replacement by pathogenic bacteria. A significant role, though still controversial, is ascribed to the bacteria Cutibacterium acnes (formerly Propionibacterium acnes), Staphylococcus aureus, as well as to a representative of micromycetes of the genus Malassezia (Ramasamy et al., 2019). The bacteria mentioned above frequently show antibiotic resistance. With respect to the resistance as well as to restrict excessive use of antibiotics, there is an effort to find alternative approaches to prevent infections and suppress pathogenic bacteria. The viruses of bacteria, so-called bacteriophages, represent a possible alternative to antibiotics. Unlike antibiotics, they act selectively, so they do not disrupt the natural bacterial microflora and are effective as prevention or an antimicrobial agent against selected pathogens.

The topic of bacteriophages as antimicrobial agents is not new. Their discovery dates back to the 1920s. With the discovery of antibiotics, in the Western counties, especially in the U.S., the research of bacteriophages was put aside in favour of other kinds of prevention and treatment of bacterial infections (phage therapy). However, the interest in bacteriophages and their use for phage therapy continued mainly in countries of the former Eastern Block (Czechoslovakia, Poland, Georgia and countries of the former Soviet Union). With the ever-growing occurrence of bacteria showing antibiotic resistance, bacteriophages started to come to the fore again (Lin et al., 2017). Their advantage is high specificity for the host strain, the capability of multiplication at the site of infection or their natural ability to overcome the host's defence mechanisms.

It is not only bacteriophages but also bacteriophage-encoded lytic enzymes (endolysins) that represent a possible alternative to antibiotics. These enzymes produced by bacteriophages degrade the cellular membrane of bacteria to facilitate releasing of multiplied bacteriophages from the host cells. Endolysins can be mainly used with gram-positive bacteria, even though they are administered from the outside. Moreover, their use together with phages may have a synergistic effect.

Preparations for skin prone to acne mainly comprise washing gels and emulsions, cleansing lotions and serums, peeling agents containing glycolic acid, or sticks containing zinc oxide. After cleaning the skin, hydration after-care is recommended, using various serums, gels and creams containing vitamins C, E, A, B3, urea, collagen, ceramides, aloe vera or herb extracts that may have antibacterial or calming effects, e.g. camomile, calendula officinalis, sage or green tea. A snail extract is famous for its vitamins E, A, C and collagen content, not only in Korea. Treatment preparations for acne-prone skin contain antibiotics, benzoyl peroxide, azelaic acid or ichthamol.

Parabens in the role of preservatives very often appear in preparations designed for irritated skin, even though they may irritate the skin, and using some of them in no-rinse cosmetics is prohibited in the EU. A current trend in cosmetics is to eliminate parabens and use natural or less controversial preservatives.

Only sporadic products containing bacteriophages appear in the cosmetic market. Preparations that are known so far and designed to maintain the natural skin microflora and fight acne vulgaris at the same time often do not contain bacteriophages against Cutibacterium acnes, one of the main causative agents of this disease. Most preparations contain not precisely defined bacteriophages, often at an unknown concentration (titer), or a lytic enzyme only. Preparations that contain crude/not purified phage lysate are also known.

The effect of the preparations mentioned above is chemically based. An advantage compared to them is the natural origin of phages, which are commonly available from natural resources. Phages are a common constituent of every environment and therefore do not represent any load for the organism or environment. Conversely, they are its normal part and provide a natural antimicrobial effect (and help preserve the natural skin microflora). Unlike the above-mentioned available preparations with phages, the present invention contains well-defined phages at a precise concentration effective against C. acnes and S. aureus. In addition, their effect may be synergistically supported by the lytic enzymes. The hydrogel's composition and preservation method ensure phage stability for a specified period. The phage stability is also supported by suitable method of preparation, storage conditions and specific composition. It has been demonstrated that individual components and processes do not negatively impact the gel's biological constituents (phages, enzymes).

### SUMMARY OF INVENTION

The invention consists in the composition and preparation of a hydrogel that contains an aqueous constituent, a gel-forming substance (preferably Carbomerum 980), highly stable crude/purified Staphylococcus aureus phage lysates with a titer of at least 10⁷ pfu/g of gel and crude/purified Cutibacterium acnes phage lysates with a titer of at least 10⁵ pfu/g of gel and/or lytic enzymes (e.g. LysF1, LysSA1).

Accordingly, the invention provides a carbomer hydrogel to maintain the natural skin microflora and to suppress pathogenic bacteria, characterized in that it contains Staphylococcus aureus and Cutibacterium acnes phage lysates with a broad spectrum of activity, both sterilized by filtration through a 0.22 μm filter. The ratio of the stock phage lysates to the gel is max. 1:100 parts by weight of the mixture.

The main active ingredient of the gel is a phage cocktail consisting of crude/purified Staphylococcus aureus phages (deposited under accession numbers DSMZ 33473, DSMZ 33474 and DSMZ 33475) and Cutibacterium acnes phages (deposited under accession numbers DSMZ 34313 and DSMZ 34314) specified in Table 1.

TABLE 1
Bacteriophages used in the carbomer hydrogel.

| Bacteriophage | Family | Genome size (kbp) |
|---|---|---|
| DSMZ 33473 | Myoviridae | 140 |
| DSMZ 33474 | Myoviridae | 142 |
| DSMZ 33475 | Podoviridae | 18 |
| DSMZ 34313 | Siphoviridae | 29 |
| DSMZ 34314 | Siphoviridae | 29 |

An advantage of the gel is that it contains a highly stable cocktail of bacteriophages against S. aureus and C. acnes bacteria, which ensures a broad spectrum of activity. Stability of phages is guaranteed for the minimum shelf-life period on condition of proper storage. Carbomerum 980 is preferably used as the gel forming substance. It is a high molecular-weight polymer of acrylic acid cross-linked by polyalkenyl ethers of saccharides or polyalcohols. In relation to the dried substance, they contain 56.0% to 68.0% of carboxylic groups (—COOH).

Other auxiliary characteristics of the gel are improved by the addition of healing substances such as herb extracts and essential oils, allantoin, chelating substances, e.g. EDTA, antioxidants like α-tocopherol (see Table 4) and preservatives (see Table 2).

According to the invention, another advantage of the gel is its outstanding spreadability and absorbability on the skin and the gradual release of active ingredients throughout the gel absorption period.

The (carbomer) gel containing phages and/or lytic enzymes is applied 1-4 times a day in a layer that is left to be absorbed by the skin.

The hydrogel must be protected from light and stored at a temperature from 2° C. to 8° C. In case of improper storage, the optimum activity may be affected due to bacteriophage degradation.

Protein sequences of LysF1 (SEQ ID NO: 1) and LysSA1 (SEQ ID NO: 2) are included in a separate Sequence Listing. The sequence listing further comprises the sequences of the used bacteriophages, namely SEQ ID NO: 3 for the phage DSMZ33473, SEQ ID NO: 4 for DSMZ33474, SEQ ID NO: 5 for DSMZ33475, SEQ ID NO: 6 for DSMZ34313 and SEQ ID NO: 7 for DSMZ34314.

On Mar. 19, 2020, a deposit of MB Pharma s.r.o Staphylococcus aureus phages with working identification numbers MB402, MB403, and MBSA2 were deposited in the International Patent Organism Depositary Leibniz Institute DSMZ-German Collection of Microorganisms and Cell Cultures, Inhoffenstr. 7 B, D-38124 Braunschweig, Germany under the conditions of the Budapest Treaty and were assigned Accession Numbers DSM 33473, DSM 33474, and DSM 33475, respectively. On Jun. 10, 2022, a deposit of MB Pharma s.r.o Cutibacterium acnes phages with working identification numbers bacteriophage MBPAC2 and bacteriophage MBPAC3 were deposited in the International Patent Organism Depositary Leibniz Institute DSMZ-German Collection of Microorganisms and Cell Cultures, Inhoffenstr. 7 B, D-38124 Braunschweig, Germany under the conditions of the Budapest Treaty and were assigned Accession Numbers DSM 34313 and DSM 34314, respectively. Theses deposits will be maintained in the depositary for a period of 30 years or 5 years after the last request or for the enforceable life of the patent, whichever is longer. Should the deposits from MB Pharma s.r.o. become unviable, the deposits will be replaced by MB Pharma s.r.o. upon request.

### EXAMPLES

The composition of the gel containing bacteriophages and/or lytic proteins is presented in the examples below, which are only illustrative and do not restrict the scope of the subject matter of the invention in any way.

### Example 1

One hundred parts by weight of a tris buffer (composition: 50 mM Tris; 10 mM NaCl; 10 mM CaCl₂; pH=7.5) and 1 part by weight of the carbomer (trade name Carbomerum 980) are dosed into a grinding mortar, left to soak for 15-30 minutes and mixed thoroughly. A 1-10 M sodium hydroxide solution is used to adjust pH to the value of pH 7 (+/−0,3) under continuous stirring. The obtained carbomer gel is sterilized in a steam autoclave at 121° C. for 15 minutes in a suitable vessel. After cooling down to room temperature, preservatives (see Table 2), crude/purified phage lysates with a titer of at least 10⁹ pfu/ml for S. aureus and at least 10⁷ pfu/ml for C. acnes are added to the gel. The phages are added to the gel at the ratio of max. 1:100 parts by weight of the mixture. Finally, the gel is mixed thoroughly.

TABLE 2
describes the preservatives and their
percentages that the gel may contain.

| | Concentration in the |
|---|---|
| Preservative name | final product in wt.-% |
| 2-phenoxyethanol | 0.5-1 |
| Chlorohexidine digluconate solution | 0.002-0.01 |
| Benzalkonium chloride | 0.002-0.01 |
| Potassium sorbate | 0.1-0.2 |
| Euxyl PE9010 | 0.5-1 |

### Example 2

The production method of the carbomer gel in bigger volumes consists in dosing 100 parts by weight of a tris buffer (composition: 50 mM Tris; 10 mM NaCl; 10 mM CaCl₂; PH=7.5) into a vessel and the surface of the fluid is equally covered with 1 part by weight of the carbomer (trade name Carbomerum 980). It is left to soak for 15-30 minutes and then mixed thoroughly using a shaft stirrer. After the addition of the carbomer, the mixture is acidified. The solution must be neutralized with 1-10 M sodium hydroxide under continuous stirring to achieve a gel structure. The pH value of the resulting gel is 7 (+/−0.3). The obtained carbomer gel is sterilized in a suitable vessel in a steam autoclave at 121° C. for 15 minutes. After cooling to room temperature, preservatives (see Table 2), phage crude/purified lysates effective against the bacteria S. aureus and C. acnes are admixed to the gel sterilely, both sterilized by filtration through a 0.22 μm filter. The titer of the stock crude/purified lysate is at least 10⁹ pfu/ml for S. aureus phages and at least 10⁷ pfu/ml for C. acnes phages. The phages are added to the gel at the ratio of max. 1:100 parts by weight of the mixture. The total titer of phages in the final carbomer gel is then at least 10⁷ pfu/g of gel for the phages effective against Staphylococcus aureus and at least 10⁵ pfu/g of gel for the phages effective against Cutibacterium acnes. Finally, the gel is thoroughly mixed.

### Example 3

A lytic enzyme (LysF1, LysSA1) is additionally admixed to the gel containing phages according to Example 1 or 2. In the same way as the bacteriophages, the enzyme is sterilized by filtration through a 0.22 μm filter. The enzyme concentration in the gel is 1-50 μg/g of gel. The amount of the enzyme in the gel results from the concentration of the prepared batch. The enzyme is added in such a way that the gel is not diluted more than 1:100 parts by weight of the mixture. After the addition of the enzyme, the gel is thoroughly mixed.

### Example 4

The preparation procedure of the gel base is equal to Example 1 or 2. In this case, the bacteriophages are not added. After cooling the gel to room temperature, preservatives, see Table 2, and a lytic enzyme (LysF1, LysSA1) are admixed sterilely. Before the addition, the enzyme is sterilized by filtration through a 0.22 μm filter. The enzyme concentration in the gel is 1-50 μg/g of gel. The amount of the enzyme in the gel results from the concentration of the prepared batch and is added in such a way that the gel is not diluted more than 1:100 parts by weight of the mixture. After the addition of the enzyme, the gel is thoroughly mixed.

### Example 5

Table 3 presents a possible composition of the gel. The amounts of ingredients are presented as percentage concentrations.

TABLE 3

| Substance (constituent) name | Content in wt.-% |
|---|---|
| Tris buffer (composition: 50 mM Tris; 10 mM | Up to 100% |
| NaCl; 10 mM CaCl₂; pH = 7.5) | |
| Carbomerum 980 | 1 |
| 2-phenoxyethanol | 0.5-1 |
| Mixture of crude/purified phage lysates | 1 |
| effective against S. aureus bacteria in a | |
| concentration of at least 10⁹ pfu/ml | |
| Mixture of crude/purified phage lysates | 1 |
| effective against C. acnes bacteria in a | |
| concentration of at least 10⁷ pfu/ml | |
| Endolysin LysF1 at a concentration of | 1 |
| 0.1-5 mg/ml | |
| Endolysin LysSA1 at a concentration of | 1 |
| 0.1-5 mg/ml | |

### Example 6

One hundred parts by weight of a tris buffer (composition: 50 mM Tris; 10 mM NaCl; 10 mM CaCl₂; pH=7.5) are dosed into a vessel, and the surface of the fluid is equally covered with 1 part by weight of the carbomer (trade name Carbomerum 980). It is left to soak for 15-30 minutes and then mixed thoroughly using a shaft stirrer. After the addition of the carbomer, the mixture is acidified. The solution must be neutralized with 1-10 M sodium hydroxide under continuous stirring to achieve a gel structure. The pH value of the resulting gel is 7 (+/−0.3). A preservative, see Table 2, is admixed into the obtained gel, and the gel is sterilized in a suitable vessel in a steam autoclave at 121° C. for 15 minutes. After cooling to room temperature, the crude/purified phage lysates and/or lytic enzymes are admixed to the gel sterilely, as specified in Examples 2, 3 or 4.

Besides the biologically active ingredients, the carbomer gel may contain EDTA and antioxidants as required, e.g. α-tocopherol, allantoin, herb extracts, essential oils etc., as specified in Table 4.

TABLE 4
Other possible constituents of the carbomer
hydrogel and their percentage concentrations

| Substance (constituent) name | Content in wt.-% |
|---|---|
| EDTA | 0.005-0.1 |
| α-tocopherol | 0.05-2 |
| Allantoin | 0.2-0.5 |
| Arnica Montana extract | 0.5-1 |
| Matricaria chamomilla extract | 0.1-2 |
| Calendula officinalis extract | 0.1-0.3 |
| Humulus lupul extract | 0.1-2 |
| Rosmarinus Officinalis Leaf extract | 0.05-0.1 |
| Citrus Grandis Seed extract | 0.1-2 |
| Lemongrass oil | 0.2-1.5 |
| Hydrolysed collagen | 0.2-2 |

### Industrial Applicability

The carbomer gel containing highly stable bacteriophages with a broad spectrum of activity and/or lytic enzymes is applicable in the cosmetic or pharmaceutical industry. The preparation is suitable for maintaining the natural skin microflora and suppressing pathogenic bacteria S. aureus and C. acnes.

### References

LIN, Derek M.; KOSKELLA, Britt; LIN, Henry C. Phage therapy: An alternative to antibiotics in the age of multi-drug resistance. World journal of gastrointestinal pharmacology and therapeutics, 2017, 8.3: 162.
RAMASAMY, S., et al. The role of the skin microbiota in acne pathophysiology. British Journal of Dermatology, 2019, 181.4: 691-699.

1.-8. (canceled)

9. A carbomer hydrogel for maintaining natural skin microflora and suppressing pathogenic bacteria comprising crude/purified phage lysates effective against Staphylococcus aureus bacteria with a titer of at least 10⁷ pfu/g of gel and/or phage lysates effective against Cutibacterium acnes bacteria with a titer of at least 10⁵ pfu/g of gel, with a broad spectrum of activity, both sterilized by filtration through a 0.22 μm filter, wherein a ratio of the stock phage lysates and the gel is at most 1:100 parts by weight of the mixture, and a lytic enzyme of SEQ ID NO: 1 and/or SEQ ID NO: 2 at a concentration of 1-50 μg/g of gel.

10. The carbomer hydrogel of claim 9 further comprising phages and preservatives.

11. The carbomer hydrogel of claim 10 further comprising 2-phenoxyethanol at a concentration of 0.5-1 wt.-%.

12. The carbomer hydrogel of claim 9 further comprising antioxidants.

13. The carbomer hydrogel of claim 10 further comprising antioxidants.

14. The carbomer hydrogel of claim 12 further comprising 2-phenoxyethanol at a concentration of 0.5-1 wt.-%, α-tocopherol at a concentration of 0.05-2 wt.-% and EDTA at a concentration of 0.005-0.1 wt.-%.

15. The carbomer hydrogel of claim 13 further comprising 2-phenoxyethanol at a concentration of 0.5-1 wt.-%, α-tocopherol at a concentration of 0.05-2 wt.-% and EDTA at a concentration of 0.005-0.1 wt.-%.

16. The carbomer hydrogel of claim 9 further comprising phages deposited in the patent collection of microorganisms in Leibniz Institute DSMZ, Munich under accession numbers DSM 33473, DSM 33474, DSM 33475, DSM 34313, and DSM 34314.

17. The carbomer hydrogel of claim 10 further comprising phages deposited in the patent collection of microorganisms in Leibniz Institute DSMZ, Munich under accession numbers DSM 33473, DSM 33474, DSM 33475, DSM 34313, and DSM 34314.

18. The carbomer hydrogel of claim 11 further comprising phages deposited in the patent collection of microorganisms in Leibniz Institute DSMZ, Munich under accession numbers DSM 33473, DSM 33474, DSM 33475, DSM 34313, and DSM 34314.

19. The carbomer hydrogel of claim 12 further comprising phages deposited in the patent collection of microorganisms in Leibniz Institute DSMZ, Munich under accession numbers DSM 33473, DSM 33474, DSM 33475, DSM 34313, and DSM 34314.

20. The carbomer hydrogel of claim 13 further comprising phages deposited in the patent collection of microorganisms in Leibniz Institute DSMZ, Munich under accession numbers DSM 33473, DSM 33474, DSM 33475, DSM 34313, and DSM 34314.

21. The carbomer hydrogel of claim 14 further comprising phages deposited in the patent collection of microorganisms in Leibniz Institute DSMZ, Munich under accession numbers DSM 33473, DSM 33474, DSM 33475, DSM 34313, and DSM 34314.

22. The carbomer hydrogel of claim 15 further comprising phages deposited in the patent collection of microorganisms in Leibniz Institute DSMZ, Munich under accession numbers DSM 33473, DSM 33474, DSM 33475, DSM 34313, and DSM 34314.


SEQUENCE LISTING (7 sequences)

SEQ ID NO: 1 - protein, 516 aa, synthetic construct
  REGION 1..516: note = LysSA1
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTIPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFKDPNSSSVDKLAAALEHHHHHH

SEQ ID NO: 2 - protein, 305 aa, synthetic construct
  REGION 1..305: note = LysF1
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFKDPNSSSVDKLAAALEHHHHHH

SEQ ID NO: 3 - DNA, 140175 bp, Staphylococcus aureus
  source 1..140175: mol_type = genomic DNA
  misc_feature 1..140175: note = bacteriophage MB402
ggtaattatgaaaatacaatacctagcacaataaatatagtagctaaagatacagataataaccctatagaatctaatgaattagatgtatataatacagtagagaatagaaatttattacaatcttataaaggtgtaaatacgatagctagagaaataacttctacaaaagagtttactgtatcaggatgggctaaagagatatactcaactaattatctttctaaagtattaaaaccaggtaaagtgtatacgttatcttttgatatggaaataacaggtaatgacccaactcttaaatcttattttgatagtcatggtatatatttatacagtaatactaagggaattgttgttaatggtgttaaatctatggaacgtactataggtaacaaagtatccgtaactcaaacttttacagcccctactattactgaccatagattactaatatatactggaagatatacatctgatggtaaagcatcaactcctccagtgttctttaatacagttaaaattacggaattaaaattgactgagggttcttctaagctagagtactcacctgctccggaagataaacctaacgtaatagaaaaaggaattaaatttaataatatcctaactaatatacagactttaagtattaattcggatactatcttaaaaaatgtaactttatattattcttactatggtgatagttgggtagaactaaagactctaggaaatattagtactggagaaacaacagaaaccaataacttaatagatttatatggattacagacagtagattattctaatataaatccaatgtctaaagtatcattacgttccatttggaatgttaagctaggtgaattgaacaatcaagaaggttctttatataatatgcctaatgattactttaatgctgtatggcaggatatagataaattatcagatattgagctaggttctatgagaatggttaaagacactgagggcggagtattcgatggagctacaggtgaaattattaaggctactctatttaatgtcggtgcttatactgatttagatatgttagcctatactttgactaattatactgaaccgttaacgttaggctctagtcgattaataattgagctaaaagaagaactactaacatcagaatcatttaatgtcgataatagaattaaagtaattgactcaatatatgaggagttaccaaatacaagcattattaaaaatggatttgttgaaagagaagttacaggttctaaatatttagattacggtttatatgagcctatagaagatggtactagatataaacttattgtcgaaggagaatttaaagataatatagaatttatatctttatacaattctaaccctaactttaatgaaacatttatatatccatcagagataattaatggagttgctgaaaaagaatttattgcaaaaccatctactgaagacaaaccaaggttaaatacagatgttagaatatatatacgaccttatgattcaactatctctaaagtaagaagagtagaattaaggaaagtttaataaataagttgacagaaagttaataatatggtatacttataaagtaatatttagtgggtataccatgttatattaataaagaaaacaacagatgaaaggaattaaaaaatatggcaattgcaacgtataattctcatgttgagttagcaaaatatctagttagtaaagctgattcagtttacttaacaattggaaagagcacaccgtggtctaatgaaacaaacccaccgcaacctgatgaaaatgcaacagtattacaggaggttattggatataaaaaagctactaaagttactttagttagaccttctaaatcacctgaagatgataataagaatttaatttcttatggtaataaatcgtgggtagaagtaacacctgaaaatgctaaagctgaaggagctaaatgggtttacttagaaagtagtattgttggtgacgaactacctcttggaacatatagacaagtaggatttgttatggacttagtagcaaaaagtggtattagtaaatttaacttagtacctagtgaagtagaatcaactggaacattattattctttgataataaacaattccaaaatagaagtgagcagacaactgctaaagaaagatttattgtagaagtttaaagaaagggagataattctaaatggcaattaattttaaaggttcaccttatttagatagatttgacccgtctaaagatagaacaaaagtattatttaatcctgatagacctctacaacaggcagaattaaatgaaatgcagtctatagaccaatattatttaaaaaatctaggagacgctatttttaaagacggagataaacaatcagggcttggattcacattgtctgaagataatgtattgacagtaaatcctggttatgtatatatcaatggtaaaataagatattacgataatgacgattcagttaaaataactggcgtaggtaaagaaactattggtattaaattaacagaacgtattgttacacctgatgaagatgctagcctattagaccaaactagtggagtaccaagttacttctctaaaggtgcagatagattagaagaaaagatgtcattaacagttaatgacccgacatcagcaactatttatactttcatggatggggatttatatattcaatcaactaatgctgagatggataaaatcaacaaagtattagctgaacgtacttatgatgagtcaggttcatataaagtaaatggttttgaactattttcagaaggtaatgctgaagatgatgaccacgtttctgtagttgtagatgcaggtaaagcctatgtaaaaggttttaaagtagacaaacccgtatcaacaagaattagtgtacctaaatcttatgacttaggaacagcagaaaatgaaagtactatctttaataagtctaataactctattagtttagctaatagccctgtaaaagaaattagacgtgttacaggtcaagtacttattgaaaaagaacgagttacaagaggagctcaaggtgatggtcaagattttctttcaaataatacagcatttgaaattgtaaaagtttggactgaaacaagccctggagttactacaaaagagtataaacaaggagaagacttcagattaacagatggtcaaacaattgattggtcacctcaaggtcaagaaccttcaggaggtacttcatactacgtttcttataaatataacaaacgtatggaagccggtaaggattatgaagtaacaactcaaggtgaagggttaagtaagaaatggtacattaactttacaccttcaaatggtgctaaacctattgaccaaacagtagtattagtagactatacttactacttggctcgtaaagattcagtgtttattaataagtatggtgatattgcaatattacctggtgaacctaatattatgagattagttacaccaccattaaacacagaccctgagaatttacaattaggtacagttacagtattacctgattcagatgaagccgtatgtatttcatttgcaatcactagattgtctatggaagacttacagaaagttaaaacaagagtagataacttagagtataaccaagcagtaaatgctctagatgatggtgctatggaaggacagaaccctctaacattacgttcagtattcagtgaaggtttcattagtcttgacaaagcagacattacacatcctgacttcggaattgtatttagttttgaagatgcagaagctactctagcttatacagaagcagttaaccaacctaagattattccaggagatacaacagctcatatttggggtagattaatttcagcaccatttactgaggaacgtacaatctaccaaggtcaagcatcagaaacattaaatgttaacccttataatattcctaacaaacaaggtgtgttaaaattaacacctagtgaggataactggattgatactgaaaatgttacaatcactgaacaaaaaactaaaaaagtaactatgaaacgattttggagacataatgaaagttactatggtgagactgagcattacttgtattctaacttacagttagatgcaggacaaaagtggaaaggtgaaacttacgcttatgatagagagcatggtcgtaccggtactttattggaatcaggaggacaacgtactctagaagaaatgattgaattcattagaatcagagatgtatccttcgaagttaaaggactaaaccctaatgataataatttatatttattatttgatggagtaagatgtgctataacacctgcaactggctatagaaaaggctctgaagatggtacgataatgacagatgctaaaggaacagctaaaggtaagtttactattcctgcaggtattcgttgtggtaaccgagaagttacacttaagaatgctaactctacaagtgctacaacttacacagcccaaggacgtaaaaaaaccgctcaagatattattatcagaactcgtgtaacagtaaacttagtagacccgttagcacaatcattccaatatgatgagaatagaactatatcatcattaggattatactttgcttctaaaggtgataaacaatctaatgttgttatccaaattagaggtatgggtgaccaaggttatcctaataaaacaatctatgcagaaacagttatgaatgctgatgatattaaagtatctaataatgctagtgctgaaactagagtatactttgatgaccctatgatggctgaaggcggtaaggagtacgctattgttattattactgagaacagtgattatacaatgtgggtaggtactagaactaagcctaaaattgataaacctaatgaggttatttcaggtaatccatacctacaaggtgtattattcagttcatcaaacgcatcaacatggactcctcaccaaaactctgaccttaaatttggtatttatacttctaaatttaatgagacagcaacgattgaattcgaaccaattaaagatgtatcagcggatagaatagttcttatgtctacgtacttaactcctgagagaacaggatgtacgtgggaaatgaaattaattctagatgatatggcatcttctacaacattcgaccaattgaaatgggagcctatcggtaactatcaagacttagatgttttaggtctagcaagacaagttaagttaagagcaactttcgaatctaatagatatatctcaccattaatgagctctagtgatttaacattcactacattcttaacagagttaacaggttcatatgttggtagagctattgatatgacagaggctccttacaatacagtaagatttagttatgaagctttcttacctaaaggtactaaagttgttcctaagtattctgcggatgatggaaaaacttggaaaacatttactaaatcccctacaactactagagccaataatgagtttacacgctatgtcattgacgagaaagtaaaatcatcaggaacaaatactaaactacaagttagattagatttatcaactgaaaatagctttttacgtcctcgtgttcgtagacttatggttactactagggatgaataaactagaggggttgattgacccctctttatttaataaggagagatttatatgcctagagaagttagagacccttattctcaagctaaattatttatacctacagttgaggaaaaatcaattaaggaattagaaaaaacatacaaagaaaaaattgatgaagctactaagttaatcaatgaattaaagaaagagagaggagaaaaatagatggcatttaactacacgcctcttactgaaacacagaagttaaaagatatgtatcctaaagttaatgatataggtaactttttaaaaacagaagttaaccttagtgatgtaaaacagatatcacaacccgactttaataatattttagcatctatacctgatagtggtaactattatgtaactaattcaaaaggtgctcctagtggagaagctacagcaggatttgtaagattggataaaagaaatgtaaattattataaaatttactattcaccatatagcagtaacaaaatgtatatcaagacttatgctaatggtactgtatatgattggattagttttaaattagatgaaggtagcttatacaatgaaggtaatactttgaatgtaaaggaacttactgaatccacaactcaatatgcaacactagttaatcctccaaaagagaacttaaatacaggttgggttaattacaaagaaagtaaaaatggtgtttcttctttagtagaatttaacccggttaactccacttcaacttttaagatgataagaaagttaccagtacaagaacaaaagcctaacttattgaaagatagtttatttgtttatcctgaaactagctattctaatattaaaacagataactgggatacgcctccattttggggatattcttctaatagtggtcgttcaggagttagatttagaggagagaatacagtacagatagatgatgggtctgatacgtacccttcagtagtttctaataggtttaaaatgggtaaagaactttctgtaggtgatactgtaacggtatcagtatatgctaaaattaatgaccctgctttacttaaagataacttagtttactttgaattagcaggatacgatactgtagatgatactagtaaaaatccttatacaggaggacgtagagaaataacagcaagtgagataacaactgagtggaaaaaatactctttcacattcactatacctgaaaatacaatcggagcatcaggcgttaaagttaattacgtatctttactactaagaatgaattgttcatctagtaaaggtaatggtgctgtagtatactatgccttacctaaattagaaaaatcatctaaagttacaccatttattacacatgaaaatgatgttcgtaaatatgatgagatttggtctaattggcaagaatttattagtaaagatgaattaaaaggtcactcccctgtagatattgaatataatgattattttaaatatcagtggtggaaatctgaagttaatgaaaagagtttaaaagatttagctatgacagtacctcaaggatatcatacattttattgtcaaggctctattgccgggacgcctaagggacgttctattagaggaaccattcaggtagattatgacaaaggtgacccatatagagctaataagtttgttaaattattgtttactgacacagagggtattccttacacattatattatggtggttataaccagggttggaaacccttaaagcaatcagaaacttctactttactatggaaaggtactttagattttgggtctacggaagctgttaacttaaatgactcattagataattacgatttaattgaggtaacttattggactcgttcagcaggacatttttctacaaaaagattagatataaaaaatacatcaaatttactgtatattagagattttaatatttcaaatgatagtaaaggttctagtgtagacttttttgaagggtattgcacttttcctactagaacatcagtacaacctggtatggtaaaatctataactttagacgggtctacaaatacaacaaaagtagcatcatggaatgaaaaggaacgtatacaggtatacaatattatgggaattaatagaggataaagaaaggtggaataaaaaaactatggctgttaaatatgatataggtaataatgagatagtattacatttaagagaaggtaaatatataacagggtttacaacagtaggagggtatgataaggagttaggacaagtaaaagttaatagagaaatcttacctgcttacttctttgataattttgcctatgaaagatatttgtattatagtaaacctgaagaggttatagaaaataaaaactatgtaccaccacaaatcaatgatgatgatgaggaatcccaacaaattactgtacctaaagaacaatatgatagtttaaaagaagaactagagcttatgagaaaacaacaagaagctatgatggaaatgcttcaaaagctcttaggtcaaaaggggtaattataaatggcattaaattttactacaataacggaaaacaatgttattagagacctgactactcaggtcaataacattggagaagaattaacaaaagaaagaaatatatttgacattaccgatgatttagtttataattttaataaatcacagaaaattaaactaactgatgataaaggattaactaaatcttatggaaacataacagcccttagagatataaaagaacctggttattactatataggtgctagaacattagcaacattattagatagacctgatatggaatctcttgatgttgttttacatgtagtacctcttgatacttctagtaaggtagttcaacatttatatacactatctactaacaataaccaaattaaaatgttatatagatttgtctcaggaaactctagttcagaatggcaatttattcaaggattacctagtaataaaaatgctgttatatcaggaactaatattttagatatagcttcaccaggtgtttactttgttatgggaatgacaggaggaatgcctagtggagtaagctccggatttttagacttaagtgtagatgctaatgataatagattagctagactaactgatgctgaaaccggtaaagaatatactagcattaagaaacctacaggaacatacacagcctggaaaaaagaatttgagccaaaagatatggagaaatatctactaagtagtattagagacgatgatagtgcatcattcccactcctagtttatactagtgatagtaaaacatttcaacaagctattatagaccatatagatagaacaggtcaaacaacctttactttctatgttcaaggcggtgtatccggttcccctatgtcgaatagttgtcgagggttattcatgtcagacacacctaatacttctagtttacatggtgtttacaatgctataggtacagatggtagaaatgtaacaggttcagtggtaggtagtaattggacttcaccaaaaacatccccttctcataaagaattatggacaggagcacaatcattcttatctacaggaactactaagaatttatcagatgatattagtaactactcttatgtagaagtttatactacacataagacaacagagaagactaaaggtaatgacaatacaggaactatatgtcataagttttatttagatggtagtggaacttacgtttgttcaggtacatttgtttccggggatagaaccgatacaaaaccccctatcacggagttttatagagtaggtgtatcttttaaaggttctacatggactcttgtagatagtgcagtacaaaatagtaaaactcaatacgttacaagaattataggtattaatatgccatagactaggagaaatttcctagtctttttttttcttgacttgaaaaggattctgtggtatactataactcgtgtaaggatataaggagattaaaatgagattaagaattaagaacttatatacctatgtagaatttgaggaggatgataaatacttaaaagatatatttttaaagagagtccatacgactataggagcaagacaagaaggatttcagtacagccctgcgtacaaaagaggtagttgggatggttatgtagatttttatgtttatgaggaagataaattccccactggacttttatttaaaattgagttattattaggtgagctacaatcaaggtataatttccagtttgaaacaattgatgagcgtgatgaaagtttcttatctgaagaagatattgatgatgagataacattgcttgataataatgtcggtcaaattaccttaagagattaccaatatgaagcagtgtacaatagcttaacattttacaatggtattgctcacttagctactaatggtggtaaaactgaggttgctagtggtattatagaccaattattacctcaattagaaaaaggtgagagagtagcattcttcacaggctctacggagatattccatcagtctgcagataggctccaagagcgtttaaatattcctattggtaaagtaggtgcaggtaagtttgatgttaagcaggttacagttgtaatgatacctactttaaatgcaaaccttaaagacccaacacaaggggtaaaggttacgcctaaacaaaatattagtaaaaagattgctcaagagatattacctaaatttgaaggtggaacaaatcaaaagaaattactaaaagtattacttgataacacaacacctaaaacaaaagtagaacaaaatgtattaagtgccttagagataatttaccaaaatagtaagacagatgcagaagttttattaaacttaagaaatcataatgcacattttcaaaaaattgttagagaaaagaacgaaaagaaatatgataaatatcaagatatgagagattttttagactcagttacagttatgatagttgatgaggcacaccattctaaatctgattcttggtacaataatttaatgacatgtgaaaaagctttatatcgaattgcattaacagggtctatagataaaaaagatgaattactttggatgagattgcaggcgctattcggtaatgttattgcacgaactactaataagtttttaattgatgaaggtcattctgctagaccaacaataaatattatacctgtagctaatcctaatgacatagatagaattgatgattatagggaagcttacgataaaggtataacaaataatgattttaggaataaacttattgcaaaactaacagaaaagtggtataatcaagataaaggtacattgattattgtaaacttcattgaacatggagacacaatatcagaaatgttaaatgatttagatgtagagcattacttcttacatggagaaatagactctgaaactaggagagaaaaattaaacgatatgagaagtggtaagcttaaagtaatgatagctacatcacttattgatgagggtgtagatatatcaggtattaatgcactaatattaggtgcaggaggtaagtcattaagacaaacattgcaacgtattggtcgtgctttacgtaagaaaaaagacgataatacaacacaaatatttgattttaatgatatgacaaatagatttttatatactcatgctaatgagcgtaggaaaatttatgaagaggaagattttgaaataaaagacttaggaaaataggagggtaagagatggcaacaaaaacacaaagaaagctataccaatatctagaggaaaatgctacagaaaataaatttcatatttctactaagaaagagctagcagattctctaggtgtttccatctctgctttatccaataaccttaaaaagttagaagaagaaaataaagtcgttactgtttctaaaagaggaaaaaacggcggggtaataataactttagttagagagtatgacacagaagaattgaaagaattcaataattctacagataatattattacttccgatttacagtatgctaaggcattaagagaaaagcacttcccttcttatagatatgagagaaaagaacaacgtagacgtactaagatagaaatggcacaatacaatgccattaaggatgagaagagaagaattatagcagatatgaatttctattcagaaggtcttccttatccttctaaagatatttttaatatgtcctatgacccggaagggttttataaagcgtacatcttatgtaagttatacgaccaatatgctatttctcatatggatgctaaacatacaagtcatcttaaagcaatgagtaaggcaacaactaaagatgaatacgactaccatcaacatatgtctgaatactatagaaataaaatgattcaaaatttacctagaaatagcgttagtgataatttctttggtagtaaaatgtttaatactttttataatttttatttaaaaataaaagataaaaatattaatgtatttaagtatatgcaaaatgtatttaaaaatgtaacattttattacgagaacggtatgcaacctaatccaataccttctcctaacttctttagctcagataagtattttaaaaactataataattatattaaaggaataaaaaaacggtgttaacagtactaatagacacctaggtgatacagacagcatcattaattcatcagactatgtgaaaaaccctgctgtattacatctacaccaactatatactacaggattaaattctactttacatgatattgatactatgtttgaacaagccttagaccttgaaaatgcctcctatggattatttggagatatgaaacatattattttactacagtataattctatgattgaagaagaaattaagaatttacctagagaagaaaaggatattattaataaatatgtaaaacaatgcataattaatgattattcaccaacaagtatttcaccttctgcaaggttatcaatgtttactatgcagaaagagcatatagtttacaataagcagttaaataaaggaatcaagagagaggatttattaccattaagtctaggaggtatagtgaataaagatttattgagtggtatggatatacaaaacttagaacagaatggtaatgaatacctatatatgagacaacatacttcaacttattatatattaagaatgtttggtgactatttagggtatgaggtaaacttaagagaagtaaaatatattgtagagaaatataatttaattgataaaataccattgacaaaagagggtatgttggattataataaacttatacatttagtagaggaagaggttaataactatgagtaagaagataaaggagcttatccttcataaatcaatgaaggatatacattttgcaagagaagtattagataacttacctaagaatctattttcagcagagtctgaggacatgggttacttatttacagctataaagagaacagcacatatttccgataagatgtcaaatgaagcattagcaattaaagtagaacagcttatgggtaataataaggaagatgaagagaaagtaaccaagacattaacttacttagaagatttatataaagtagacgttaatgaaaaagatgaatctgttaattatgaaatagagaagtatattaaaacagaaatgtcaaaagaagttttagttaaatttattgcagaaaataaacaagaagactctgataatctacatgaacttgtagacaaactaaagcaaatagaagtaagtgacatctcaggaggtaatggggagtttattgacttcttcgaagatacagaaaagaaacaagaactattgagtaatttagctacaaataaattctctactggatttacttctattgacaaccatattgaaggtggtatagcaagaggagaggttggattaatcatagctcctaccggtagaggtaaatcattaatggcttcaaacttagctaagaattatgttaaaagtggattaagtgttttatatattgccttagaggaaaaaatggatagaatggttttgcgtgctgagcaacaaatggcaggagcagaaaagagtcaaattgtaaatcaggatatgtctttaaataataaagtttatgatgcaatacaaaatcattatcagaagaatagaaagttattaggtgacttttatatttctaaacatatgccaggtgaagttacaccaaaccaattagaacaaattattgtcaatacaacaattaagaaggataaaaatattgatgttgttattattgactatcctcacttaatgagaaatccttatgctaaatatcattcagaatcagatgcaggagggaaattgtttgaagatattcgtagattatcacagcaatatggatttgtttgttggacgttagctcaaactaaccgtggtgcttatggttcagatgttattacaagtgagcatgtagaaggttctcgtaagattgtcaatgctgttgaggtgtctttagcagtaaaccaaaaagatgaagaattcaagagcggtttcttaagattgtatttagataaaattcgtaatagctctaacacaggagaacgatttgttaatcttaaagtagaaccaactaagatgattgtaagagatgaaacacctgaagaaaaacaagagcatatacaattgctatcagataatggaaaagaagacacaagtaaatttcaaaataaagataataaaatagaagctataaataacacattcggaggattaccgggagtttaattttttaaaatataccacttgacattttatatgttaggtggtataattattttataaagaataaaggagagattaataatgaaatttgtattctttacagatagccactttcacttatttactaactatgctaaacctgatgagcagtatgtgaatgatagatttagagaacagatacaagctttacagaaaatgtttgatattgcaagagaagaggatgcaacagttatatttggtggggatttattccacaaacgtaacgcagtagatactagagtatataataaggtatttgaaacattccaacttaatagagatatagaagtactaatgttaagaggtaatcatgattcagttacaaatagtttatatacagattctagtatagaacctttcggttacttacctaatgtagaggtttgtaaaaaccttgatactttagggtttttaggagaagaacaggatattaatattgttatggctccttatggagacgagactgaagaaattaaagagtttattaaaaataaatatgtagaagatagagtaaatatcttagtaggtcatttaggtgtagaaggctctttgactggaaaagggtctcatagattagaaggggcatttggataccaggatttattacctgataaatatgatttcattttactaggtcattatcaccgtagacaatatttccaaaatccgaatcatttttatggtggttcattaatgcaacaatcattttctgatgagcaagaagctaatggtgttcatttaatagatacagaaaaaatgactacagaattcatcccaatccatacacgtagatttattactattcaaggagaagatattcctgagaactttgaacagctaatcgaggaagataattttattagggttatcggtacagcaaatcatgctaaggttttagaaatggatgacagtatgaaagataagaatgttgaagttcaaattaaaaaagagtatactgtagagaaacgtattgatagtgatgtgtctgatgaccctttaacaattgctagtacctatgctaaacaatactcacctgaatcagaacaagaaatacttgagtgtttgaaggaggttttataatgaaaaaatatagagaatatctaaataagacagatgcagaaaatttagcagaggattgggagaaagtaaccgaagatttatggaaagtgtttaaagatatgaaacctaaaattaatacattagatatcagtaatgtagtaagtaaagacttagataaaagtaaacctattttacaattccaagattcagatggagtaatagagaatatttgtaatgttgaaggtttagaagatggtctaagtaaaatgaaaaagatttttgatgatagtaattttgaaaagcattattacaatagagtagtagaccatgatgagtattactggattgattatggctctcatcattgtttctttagagttacgaaaggggataagtaatggttgtatttaaacaagtagaagttaataattttttagcaattaaagaagctacgctagagttagacaatagaggattaattctaattgaaggtgagaataaatctaatgagtcatttcattcaaacggctcaggaaaatcaactttaatatctgccattacttacgctttatatggtaaaactgaaaaaggactaaaagcagatgatgtagtaaataatattgagaagaaaaatacatctgttaaacttaagtttgatattggggaagatagttatttaattgaacgttatcgtaaagataaagagaataagaataaagtaaaattattcgttaatgaaaaagagattacaggttcaacaaatgacgttaccgataaacaaatacaagatttatttggtattgagtttaatacttacgttaatgccatcatgtatggtcaaggagatatccctatgttctctcaagcaacagataaaggtaagaaagaaattcttgaatctattactaagacagacgtatataaacaagcacaagatgtagcaaaagagaaagttaaagaagtggaagaacaacaaaataacataagacaggaaatctataaactaggttatcagttatcgacaaaagatgagtactttcaaagagaaatagagcagtacaatcaatataaagaacaattggttcagatagaaaacagtaataaggaaaaagatagattaagagaacaagaggagaagcaaatagaagctcaaatagagcaactagcttcacagataccaacaatacctgaagatgaatttaagcactcagaggagtataataaagcctctcaaagcctagatttactttctaataaattaacggagttaaatcaagtttactcagagtataataccaaagaacaagtactaaaatctgaaatagctacattaagcaatagtctaaatcagttagatacaaatgaccattgtcctgtttgtggctcccctatagataattctcataaattaaaagaacaggaaaatatcaataatcagattgagaataagaaacaagagattactagtgtattagaaatgaaagatacgtataaagaagctattgataaagtaaaagataaatcacaagaaattaaagataaaatgtcacaggaagaccaacaagaacgagagcacaataataagattaacagcataattcaagaggcttctaggattaaatcagacattagttcattagagaataataaaacgtatttaaaagttaaatatcaacatcaatctgttcaaggattagagagagaagaaccaagtaaagaaaaacatgaggaagataagaaagaattacaagaatctattgacaaacatgaagagaatatagtacaattagaaactaagaaaggtaaatatcagcaagctgtagatgcttttagtaataaaggtatacgttcagtagtgttagactttattacaccattcttaaatgaaaaagcaaatgagtaccttcaaactttatcaggttcagatattgaaatagagttccaaactcaagtgaagaatgctaaaggagaactaaaagataagtttgatgttattgttaagaatagcaagggcggaggttcgtacaaatccaattcagcaggagaacaaaaacgtattgatttagcaattagttttgcaattcaggatttaattatgagtaaagatgagatatctacgaatattgcactttacgatgagtgttttgatggattagatactatcggttgtgaaaacgtgattaaattattaaaagatagacttaatacagtaggaacaatatttgtaattactcataataccgagcttaaaccactgtttgaacaaacaattaaaatcgtaaaagaaaatggagtatcaaaactagaggaaaaataatgaaattaaagattttagataaagataatgcaacacttaatgtgtttcatcgtaataaggagcacaaaacaatagataatgtaccaactgctaacttagttgattggtaccctctaagtaatgcttatgagtacaagttaagtagaaacggggaatacttagaattaaaaagattacgttctactttaccttcatcttatggtttagatgataataaccaagatattattagagataataaccatagatgtaaaataggttattggtacaaccctgcagtacgcaaagataatttaaagattatagagaaagctaaacaatatggattacctattataacagaagaatatgatgctaatactgtagagcaaggatttagagatattggagttatattccaaagtcttaaaactattgttgttactagatacctagaaggtaaaacagaagaagaattaagaatatttaacatgaaatcagaagagtcacaactgaatgaagcacttaaagagagtgatttttctgtagatttaacttatagtgacttaggacaaatttataatatgttgttattaatgaaaaaaattagtaaatagtaaggaaggatattatgaggtttgaagactttttaacccaagaattaggagaaccaaaagaaaatactataggtgagctaagatactgttgtccgttttgtggagaaaaaagttataagttctatgttaagcaagccctagactctagtaatggtcagtatcattgtaaaaaatgtgatgaatcaggtaaccctattacatttatgaagacttattataacattacaggtaaacaagcttttgatttattagagtctaagaatatagatatagagagagcccctttacttacgaccaataataaggatttgacagaatcagagaaacttatattaatgcttagaggtgtgcaccaagataaaggaaatactagtattaaacctcctagattacctgaagggtataaattattaaaagataacttaaataataaagagattataccctttttaaaatacttaaaaggcagaggtataactttagaacaaattattaataataatataggttatgttattaatgggagcttttataaagttgacggggaatccaaagtatcattaaggaatagtattatattttttacttatgataatgatggaaattaccagtattggaatacacgaagtatagaaaagaacccttatattaaatctattaatgctcctgctaaacaagatgaagtagggagaaaagatgtcatatttaatttgaatatagcaagaaagaaaaagttcttagttataactgagggtgtatttgatgctttaaccttccatgagtatggcgtagcaacattaggtaaacaagtaactgagaatcaaataaaaaaaataattgattatgttagtatagatacatcaatatatattatgttagacactgatgcattagataataatatagacttagcttataagttaaaaacacattttaataaagtttactttgtacctcatggtgatgaagatgcaaatgatatgggaacaaggaaagcttttgagttattaaaacagaaccgggtgctagtaacacctgaaagtatacagagttacaaaatacaacaaaaacttaaactttaggcttgaccttagagaagttttatgttatactagtaattaagtaattaataaaggagaaaaaaaataatgtcaaataataaaaaagatattttagaatttgtagatgaatacattacagctttaagagttggtaatgagcaacgacaacatcaattagaagaaatgggtaaagaagaaacagcaacattaacagatgtagctaaagctattactaaccttatgttaggtgttaatgagcagatgacagacttagaatataataacgagttaaacttaaatattttaattgatgctttatataaagcagagcttattaatgaagatgtattagactacattcaagaatcaattgataaatcacaagaagaacctaaaaatgaagaagaaaaaggagaacaagaataatggaaaaaaatattagcacacacacaaaaggtattagtcaagcagacatggagaaatggattgaagctgtagtacaaggaactgttgatggtaaacaagttgatgagaaaacagctaaacaattagatagaattggttcacgaagtgtttctttagaagaagcaactcgtattgctaaagttcttaatgctgtaacagctcaagaggttacaggagactttaatgatgcatttaatgcaattgacttaatgatgattatcatggaagatgagttaggagtaactcaagaaaaagtaggtaaagctaaagataaactaaatgaaaaacgagaagcttacctaaaagagaaacaagaagaattacgtcaaaaacaacaagaagaggcacagaaaaaaactgaatctgacagcaatgaaaaagtaattcagttgaagaaaaatgacgaacagtaagaaaaaaggggatacattcgaacgtaaaatagctaaagaattaactgcttggtggggataccaattcaataggtctcctcaatcaggtggtgcttcatggggtaaagataataatgctgtcggagatatagtagtacctcaggaagctaattttcctttagtagtagaatgtaaacatagagaagaatggactatagataacgttcttttaaacaacagagagccacacacatggtgggagcaagtcattaatgatagtagtaaggtgaataagacaccttgcttaatatttactagaaatagagctcagagttatgttgctttaccttatgatgagaaagtatatgaagatttaagaaataatgaataccctgtcatgagaacagactttattattgataatattagaaaagataaatttttttatgatgtccttataactaccatgaatgggttgacctcatttacaccttcttatattatatcttgctacgacaaaaaagatataaaaccatacaagaaggtcgagtctaatttatctgaggtaagtaagcatgaagatgaattgattaatgaccttcttagtgatatataaggaaggtaagataagtatgacaagcaaagaaagaccattaatcgtatatttttcaggtacaggacaaacagaaagattagtaaacaaaattaatattaataattcatttgaaacatttagggttaagagtggaaaagaaaaagtaaataaaccttttatactaataacacctacttataagaaaggtgcaatacctaaacaaatagaaagattcctagaaattaatgggagccctaaagaagttattggtacaggaaataaacaatggggctctaatttctgtggagcaagtaaaaagatttcagagatgtttaagattcctttaattgctaaagtagagcaatcaggacactttaacgagatacaaccaatattagaacactttagtaataaatataaagtagcgtaaaggatgagagatatatggcaacatatggaaaatggattgagttaaataatgaaataactcaattagatgacaatggaaaaaataaactctataaagaccaagaagctttagatgagtatttaaaatatattgaagacaatacaagaaagtttaatagtgaagtagaaagaattagagtattgacaaaagaaggaacatatgataaaatatttgacaacgttcctgacactattattgatgaaatgactaagttagcttacagttttaattttaaattccctagtttcatggcagggcaaaagttttatgaatcttacgcatcaaaacagtatgatgaaaacaaaaaacctatttttgttgaagactatgaacaacataatgttcgagtagctttatatttatttcaaaatgactatgtaaaggctagagaattactagtacaacttatggagcaaacattccaaccatctacacctacgtataacaactcaggacaagctaatagaggtgaactaagctcatgttatctatttgtagtagatgattcaattgagtctttaaactttgttgaagatagtgtagctaatgctagttctaatggtggtggagttgcaattgatttaactagaattagacctaaaggagctccagtacgtaatagacctaattcaagtaaaggtgttattgcttttgctaaagctattgaacataaagttagtatttatgaccagggtggtgtaagacagggtagtggtgctgtttatctaaatatattccataatgatatcctggatttattaagttctaagaaaatcaatgctagtgagtctgttagactcgataaattatctattggtgttacaatccctaacaaatttatggagttagttaaagaaggtaaacctttctatacttttgatacttacgacattaataaagtgtacggtaagtatttagatgagctaaacattgatgaatggtatgataagttactaaataatgatagtatcggtaaagtaaaacatgatgctagagaagttatgacagatattgctaaaacgcaattagaatcaggctacccttatgtattctatattgataatgctaatgataatcacccattgaaaaacctaggtaaagttaaaatgagtaacttatgtacagaaatttcacaattacaagaggtatcagaaatttatccgtactcttacagtaatcagaatgttattaatagagatgttgtctgtacattaggttctcttaacttggttaatgtggttgaaaaaggtttattgaatgaatctgtagatattggtacaagagcattaacaaaagttactgatattatggatttaccttacttacctagtgttcaaaaagcaaatgatgatattagagctatcggtttaggttcaatgaatttacatggacttttagctaagaatatgattagttatggttctagggaagcattagacctagtaaacagtttatatagtgctattaacttccagtctattaagacatctatgttaatggctaaagaaacaggaaaaccatttaaaggctttgagaagtccgattacgctacaggtgaatactttgtaagatacattagagaatccaatcaacctaagacagataaagctaagaaagtcttagataaggtttatattccaacacaagatgattgggatgaattagctaaagcagtgaaagtacatggtttgtataatggttaccgaaaagcagaagcacctactcaatctatatcttatgtacagaatgctacaagttctattatgccagttcctagtgctatagagaatagacaatatggagatatggagacatattacccaatgccttacctaagtcctataactcagttcttctatgaaggagaaacagcttataagattgacaataaacgtattattaatacaagcgcagttgttcagaaacatacagaccaagcagtgtctacaatactttatgtagaatcagaaatccctactaataaactagtatcattatactattatgcttgggaacaaggattaaaatcattatactatacacgttcacgtaaactttctgttattgaatgtgaaacatgttcggtttagaaaggaaatagatatggatattacacaaaaagtaaaacaacataataaaaatgctgtattaaaagcaacaaactggaatattgaagatgacgggatgtctgatatttattgggagcaaggaatctcccaattttggactcctgaagagtttgatgtatcaagagatttaagttcttggaatagtttaactgaaagtgaaaagaacacttataagaaagtccttgcagggctcacagggctcgatacaaagcaaggaggagaaggtatgaacttagtatcctaccacgaaccaagacctaaataccaagctgtatttgcgtttatgggtggtatggaagagatacatgctaaatcttatagtcatatttttacaacattactaagtaataaagaaacaagttatttattagatacttgggtagaagaaaacgactttttaaaagtaaaagctcagtttatcggatattactacgaccaactattaaaacctaatcctactatatttgatagatacatggctaaagtagctagtgcctttttagaaagtgcattattctactcaggattttattatcctttacttcttgcaggaagaggtcagatgacacaatcaggagctattatttataaaattactcaagatgaagcttaccatggttcggcagtaggattaacagctcaatatgattataatcttctaacagaagaagagaaaaaacaagcagataaagaaacttatgaattattagatattctttacactaatgaagtagcgtatacacatagtctatatgacccactagaattaagtgaagacgtaattaactatgttcagtataattttaatagagctcttcaaaaccttggaagagaggactattttaatcctgaaccttataaccctattgtagaaaatcaaactaatgtagacagattacgaaatgttgatttctttagtggtaaagcagactatgaaaaatctacaaatatcaaagatattaaagatgaagatttctcattcttagatagtaaagaatacagtactgccaaggaattcctataaaaaggagaaaagatattatggatagaaaagaagcaatggatttactaagtaaagcagaaatattatttaaaaaacatgatgagttttcatgtgtaagtgatatcaatgaccctatgaagttattcagtaactctaaggatgctaaagctgatgatacgtctaattcttttcagctagagtttatgcatgatatgaccatgtatactttatcttatggctcaggacagctaaaacttattgatttagcagaaggttatgaagcacaaaaagctacaatagttaactcatttcccgaaattattaaaacattagaaaaggatgattcagaagatggaaaaaatgaatagtttagtagatttaaatacagcaattagacaaaagaaagatgttattgtcatgattacacaagataattgtggtaagtgtgagattttaaaaagtgtaatccctatgtttcaagagtcaggtgacattaaaaaacctatcttaacattaaatctagatgctgaagatgtagatagagaaaaagctgttaagttattcgatatcatgagtacaccagtattaattgggtataaagatggtcagttagttaaaaagtatgaagaccaagttacacctatgcaattacaagaattagagtcactttaatttggaatttcctactatctgtgctatactataatagtacaaggtagtaggattttttaatggaaggaagatgacatatcgcaaagaataaaacattaacgatatataatagtgatagatattttaatatacacacaaaagataaagataaaattaatgaggctattaaagtaacacacggtaatgaagaagaaattgaaaagaatatggatgaattaatatctaagtctagaagatatatcatgagggatgaaaagcattacatgctatttaatgagaagtacaataatgataggcttatagaaaaagtatgtaaacacggtggtaaagttacatactatactgattcagtattaccttactatgttttaaaagacttatctagtcaccctgactcagaagttgtttatcgtatgcgcaacggttttactgcaaaagaagtagataatatagctttatcattcatgggtacaaaagttattattgatatttctgtagtatttccttatgtaaacccttatgatattattagaagtttacatgatattaaaacaaatgtagatgaagttcatttatcatttccacgaatattaggggtagatgaaaaacaagaaaagttttatttctttgatggtgaagcttatgatttaaaacccgaatataaagtcgattttgcagataaaattagagtatctttatcagtatggaaaatgtatatctatatcttaacaagtagtcgtgattttgaggatgtagacaatgtaattacgaaattaaaacaacaacgaaagattaagatataaggtgattatatgagtacagcaaatagaagagatatagcaagaaagatatcagagaatacaggttactatatccaagatgtagaggaaatactaagtgcagagacagatgctatttctgacttgctagaagaagggtatactaaagtaaagaatcataaatttatgcaaatagaagttattgaaagaaaaggtaaaaaagcgtgggatggtctgaataaagaatacttccatttacctaatagaaaagctataaaattcaaaccactaaaagaactagaagaggttattgatagacttaatgaagaagagaaataattctcttctttttttattgacaaggtttaaaatatatggtatagtattattaagttaaaaaaggagaggaattaaatgaaagtattaatcttatttgaccacattagagaagagcatttttctgtaagtaaagatgggagtgtgaaatctaatgtactaaatacacctaacggaaaaacacttaagaaattacttgagaagtgttctaacttaaagagagataaaacaaacagagattatgatattgattttctctacaatgcagtacctacacctattagaaatgactacggtaaaatcattaaataccaagatgttaaacaagcagaagtaaagccatactatgagagaatgaataatattattattgataattcttatgatatggtaattcctgtaggtaaactaggtgttaaatacctattaaatgttacagctattggtaaagtaagaggtgtaccaagtaaagtaactattgaaaatggaacatcttctcatgatgtgtgggtattacctacttatagcattgaatatactaatgtaaataaaaatagtgaacgtcatgtagtatcagatttacaaacagttggtaagtttgtagagcaaggagaagaggcatttaaacctaaggaagtatcttacgagttggtagataacattgaaagagtaagagaaatattcaataaggaagtaaagaatgataattatgatggggtagatattaccgcatgggacttagagactaactcattaaaacctgataaagaaggaagtaaacctttagtactatctctatcatggagaaatggtcaaggtgtaactatacccttatacaaatcagactttaactgggaaaacggtcaagatgatattgatgaagtcttagaattgcttaagaattggttagctagtaaagaagatattaaagtagcacataacggtaaatgatttgctgttgtaaaatccctctcatatcgggcatagctttaagtagctgataagagaacctaagtcctgtaataaggatagtggtaatcccgagcttacattattggtgacaatagatggggtgtagagactgagccgaggttttgtagaccaaggtgagacatagtgtatcgacttaatagaggtggtacagtgaaaaaagattatatgacatcagttaaaaataacaaaaaagtatgtagaagatgcaacgaagaattagatttatctaactttaaaacatataagaagaatgataaaacttattatcaaagtatgtgtataccttgtcggaaggaatataataagttagataaaactaaaaatactattaaaaaatgttatgagaaaaacggagataaatatagaagacaaagtaatgagtataatacttctgacagaggtagagagcttaataaaaataggtctaggaaatacagagaaaacaattctttaaaatcgaaagctagaagctctgtaagaaccgcattaagaaatggttctctcataagacctgataagtgttcagagtgtaataaagattgcatacctgaagctcaccatcctgattatactaaacctttagaaataaaatggttatgtaaatcctgtcatgaagatactcatcataaaaaataatcacactatgtaaatgagggacatcaagcccatttaggtaactacaaacaaacctaatggtaagggcttatgaaggtatagtccgttctatatagaaatatataggctaaaacgaaatatgatattaagttcttaatgagtactgaaaactttaaagattttgagagtattcaggatactaaagtaggttggtacctagctgttacccaagaagttaaagaatctttaagattatctgatttagcttatgaggttacagatgtcggaggctatgataaaccattagaagactttaaattatggtttgttactaagttattaagattcttctcagataaaattaaagagatacagaaagaaaataaaaagattgctaagaaagagtatgatgttaaagctcctgaatataaagaatggttagagaataaattaaatgaaacagtagtagaactagatgatactgagaaaaaatttagagttagtgaattagagaaaaagtatattcaactaggtctttcacctgaaattgtaaatatgaatttagttatggataatgatgaattcataaatattgcagaacaatcacctgagtacatggggttatctgactacgctaagtcttacacgttaaatactgcaattaatttaattaatgagtatagagatgtaaaagatgtagttaatgatattgacggaggtaactttaattatgattggttccctattgagttaatgcatccatacgcatcaggagatactgatgtatgtagaagaatttattgtgatgtaattaagaaacttaaagaacaagatagacctaagtcaatgcatttattagaagttaattacccaagacttactaagtctttagctagaattgaatcaaatggtttatattgtgacttagattatatgaaagaaaatgatgagtcatacgagtctgagatggctaagaaccatgctacaatgagagagcactgggctgttaaagaatttgaagaataccaatacaatctttaccaaatggcgttagaagaacatgagaaaaagccaaaagatagagataaagatatccatcagtacagagataaatttaaagatggtaaatggatgttttccccaagttccggagaccataaaggtagagtaatttatgatattctaggaattcaattaccttatgataaagaatatgtcaaggaaaaaccatttaatgctaatgttaaagaagcagaccttacttggcaggactataaaacagacaagaaagctattggttatgcgttagataatttagaattaaaagatgatgttaaagagcttcttgaattacttaaatatcatgctagtatgcagacaaaacgtaattcatttactaagaaattacttaatatgattaataaacaaaaacgaacattacatggttctttttctgagacaggcacagagacatcaagactaagtagtagtaacccttaaattggggttgtaaaactttgttaactgcgggaagagactcgttaggtcttaactactaacttataatggaaacatatataagggcaaacagtaacgtgtttgatatagtaaaaaggttaagaatagagagaatccgcatccaagaccctgaaagtatataaaagtatgggtaaggttcaacgactaggtgttgagacaatacaatcaatacacacccacgaaagcaaaggtattatttctgtggtagggaataataaggagagttatatgaaagagatttggaagaaagtagtaggatttgaaaactacgaggtaagtaataaaggaaaagtaaggaatataaaaactaactatattttaaagccgtggataataaattccggatatgagcaagtatctataggtattgctaatgtattagtacatagattagtggctatgacatttatacctaccgacagctatagtatagttaaccatattgataataataaattaaataactgtgttgaaaatttagaatgggtaagttacaaaggtaatagtgctcacgctaataagcaaggaagattgaatacttatagtgcaagagaaaaacttagttctgtatctaagaaagccatttatcaaaaagatatggaaggtaacatcattaagttatgggattcaccaagtgaagctgaaaaagaatctaatgggtactttaaaagtactaagattagttccgttgctcacggtaaacgtaagcatcatagaagttatacttgggaatacgtatataaggattcaaagagaagtttaaataagtctattaatatgtatgatttaaataataatttattatatgaagatttgacaatgaataaaattatgggtatactagaaatgaataatcataaaacattaagagataaactaagaaatacagatgactttgttgaatacagaggatataaatttaaaaataataattaaaacctaccacagaaatgatatatgatatagtctactcaatagtgagagctattgtgttacctaaacagtaacagattgtaaactaaaaagcttacaaattatagaatttacaaaacttacctgcacacacatcagatgtaaataagtttgattacaaacatccaattaaacgttcatttgtttctagatttgaaaatggagtactgctagggtccgactatagcgccctagagatgcgtattatcggattatttactaaagaccctgatatgctacaatcattcttaaatggggaagatatccataaggctactgcaagtattgtttacaataaaccagtagaagaagtaactaaagaagaacgacaagcaactaaagcagttaacttcggattagcctttggtgaatcacccttctcatttgcaggtaaaaataatatggaagtaagtgaagcagaagaaatatttgaaaagtatttccaaacaaaaccaagtgtaaaaacttctattgacaatgtacatgagtttgtgcaacaatatggttatgttgatacaatgcacggacatagaagatttatccgttcagcccaatcaacagataaaaagataaaaaatgaaggtctaagacagtcatttaacactatcatccaaggttcaggtagtttcttaacaaacatgtctttaacttacttagatgattttattcaatctcgtaatttaaaatcaaaagttattgccacagtacatgatagtatcttaattgattgtcctcctgaagaagctaaaattatggctaaagtgacaattcatattatggaaaacttaccatttgatttcttaaaagcagaaattgatggaaaagaagtacaatatcctattgaagccgatatggaaattgggttaaactataatgatatggttgaatatgatgaggaagaaatagatacatttaattcttaccaaggttatattaagtatatgatgaatttacagaccttagaagattataaagagtcaggtaaactaacagatgaacaatttgaaaaggctactaatgttgttaaaagtgaaaaacatatttatcaagaaatttaataaaagtattgacaatatagttaacttatgttatactatataagtaataaatataaggaggaaaaagagtgaatacaggggagattagatttaatcgttctatggatgaatggattataacaagcatgtaccaggatgagctaggtgggatgaatattgttgttacattctataatagagaagaaaataaacatggttctacagttttaccaacagagtcatctactggagaagtaacagaggaattggcaagtcttgaagaagaatatcctttagctttacctttaagtagtatctcagttaatatttaaaaggaggaactgataaatggaaatacacattgattccctagattttacaaactttactattaaagatagaaatgggaactcacaagagtttgatattacagatgagttaagaattacagagtatacaatacaagaggattttatgcaacaatcagctaaatatgctttttgggcttctatattagagaaggtaagagcatattctgaaatggaacaaagaaacctagaaacaattggtagtaagctaaaccttacaattagacaagagtacgaacaacaaggtaaaaagcctactaaagatatgattgaatctagtgtttatattcacgattcttatcaacaacaacttaaagttgttgaggcttggaattataaagttaaacaacttcaatatgttgtaaaagcttttgagacaagaagagatatgatgattcaattaggtgcagaattacgacaaacaaataaaaatggtggaattactaatccattttcacattaaaaaataaagtaaagaatataattgacaaatataaaaaactatgttataataaataagtaaattaattaaaaggagaaaagataattatggatttcaatcaatttattaacaatgaggcaagcaaattagaaagcaataacagttcttttaacaataatgtagagagctacaaacctaaaaaccctgtactacgtttaggtaatattaaagatgcaaacggaaataaggttgttaaagaaaatgcttttgtacgagtattacctcctgcacaaggaacaaatgttttctttaaagaatttagaacaacaggtattaactattctaagaaagatggttctcagggattcacagggttaacattacctgcagaagagggttcatctgtccttgacccatacattcaggattggataacaaatggtgttcagttcagtagattccctaataaaccaggagtacgctattacattcatgttattgaatactttaataacaatggtcaaattcaaccaaaaacggatgctcaaggaaatgtaatgattcaacctatggaattatctaatacaggatataaagaattattagctaacttaaaagacactatgttaaaaccatcacctaatgcacctcatagctttatctcagcaactgaagcattcctagttaatattgttaaagctaagaaaggtgaaatgtcatggaaagtaagtgtttatcctaatgcccctttaggtgcgttacctcaaggttgggaacaacaattatctgacttagaccaattagcaaaaccaacagaagaacaaaatcctaattttgttaacttcttaatcaataacgttaataacacagagttaagtcatgataactttaaatttaaccgtgaaacaaatgtcttaggtgaagaaccttcagagcctaaacaagcacccacacaacaagatgtagatagtcaaatgccaagtaatatgggaggacaacctaatcagcctcagcaaggtcaagtaggtcagtatgcacaacaaggtcaaagtaatggtcaaggacagcagttacaaggtacacaacaacctatcaataacactcaatttggtcaaggaactccttcaggacaacaaccaagtaacacaggttctgttgattgggataacttagcgcaacaacaatcacaacctgattcaaacccattcaatgattttgatgttagcagtgttgatgattcacaggtaccttttgagacacaacctcaaaatacacaacaagcacctgaaccacaacaaactacacaagagcctccaaaacaaaaacaaacgcaaagtattgacgatgtattaggtggtctagacttagataacctataagatatagagtgccttagagcactcttttatttgagatataattactaggaggatattaaatggcaagagcaaaaaaaggtaaagaagtcgatttaacagatttaaatacaattgatttaggtaaagaattaggattaacattgctatcagatacaaacagagcagatattaaaaacgttatacctacaatggttcctcagtatgactatattttaggtggaggtattccattaggtcggttaacagaagtttacggtttaactggcagtggttgccttaaatagccacagtgacttgtaaagagttattacaaaaccatgtgaattgctgggaagccttaaagacttattaactacaacgtaactggaaacagtaggcgtgaatgtttaaaaataataagtatatgagttatatggtataacgaaaaatctttgttatattatatagtgtaggtaatcagcatccaagcttctaagttgaaaaataagaagaaggttcaacgactatcgaaaagatatctagtaccgagaggtattagagagtaactgagtagagtaaaagatattaatctttgaagtgcatgggaattgcctacgaaatctattagaaaaatggagagtagtataatatggtaaaacgagtttggactaatgaagagaaacaagatatagtaaaatcatttcaagaaggtaaaacgtttaaagaattacaagataagtataatgcacattatttcactatcaagaaaattttagatgagtttaatattgacacaaataaaaaacgaagatggactaataagcaaaaacaagatatactaagaatgtatactaaagagtctatgactatagcagagataaaaaaggtatatacaacacacgcaagagaaataggtagaatactaaaagactttggagtagatacttcttactatcaaactagaagcgttaatagaaatataaatagaaatttctttgaagtaattgatactgaagagaaagcttatatactaggtttattaatggctgatgggtgtgttagatatagacgagaagggcaatgttacttaacactagaattgattgataaagaaatagtaaaaagagttcaaaaagaattgaattcagacagtaaaatatatgaatctcatagaaaaagagactatataaaaaatgaaaaacaaacttatacttttagtgttactgataataaattatgtaatgatttagctaagtacggtatagtacctatgaaaactaaaaaaacggattggttaacacaagatataccttataatttaagaaaacattacttaagaggattgtttgacggtgatggttctatcgggtattataataatagatggtttataactttaattaataatcatcctgaatttttaaaagatgtaggtacttggattgatgatttattaggtttaaaatgtcctaaagtatctaaaactagtacctcttatagaataggttatacaggtaaaaaagctaaagaattaatgaaattactatatcaagataataatattcatataatccgaaaacaaaaacttgcagaccaggcaattcaagatatagtctaagcccactattaaattagtgttaaagtactgggaaactaggggtagtaactgaaatctacttttgcagttcacttatctcgaattgcaacacaattaggtgttatcactatttggattgatattgaaggaacagcagataacaatcgtatggagcaacttggtgtagatgtttcaaaactattctctattcaatcaggagaaggtagacttaaaaatacagtagaattatctgtagagcaagtaggtaaagaattagagtactggattgacactttcaatgaaaagattccgggagtacctattgtatttatttgggactcattaggggctacaagaactcagaaagagattgatggcggtattgatgagaagcaaatgggtcttaaggcatcagctacccaaaaagtaattaatgcagtaacacctaaactaaatgatacaaacacagggttaattgttattaaccaagcccgtgatgatatgaatgcaggtatgtatggtgaccctattaaatctacaggtggtagagcttttgaacatagtgctagtttacgtattaaggttcataaagcatctcagttaaaacagaaaagtgagttaactggtaaagatgaataccatggtcacattatgcgtattgaaactaagaaatctaaactatcacgaccagggcaaaaagctgaagcagacttactatctgattatatggtaggtaaagaagatgaccctatcttattaaatggtatcgacttagaacatactgtatataaagaagcagttgaaagaggtttaattaccaaaggagcatggagaaactatgttacattgaatggtgaagaaattaaacttagagatgctgaatgggttcctgtacttaaagataataaagagttatatctagaattgtttagtagagtttatggagaacacttccctaatggttactcaccattacttaataacaaagtaatcgtaactcaattagaagagtatcaagctcttgaaaactactataaagaatgggctacagataataaacaagaggaacaagaggaagaactaaaaggagaatctcaagaaaaggattctgaataatagatggataatttaatagataaaaacatgaatcaggtaaaagaatctttggggaatgcaaattcctcagatgttcttcctttaccttataaagatatagcaaagaaatttgaagaagtaaaagaaaaaggtgaatcaattatcattgaagaaggtggattcccttatacagattctacagtgatgtatatagaacatgtaacagatagatgggcaggaggatattccttaattagacatgaaggtgaagaagttaaagtacctaagactatccatttctctgatatatatgttaaagataaatcacacaaagtaagaataatcttcgagggggctaatccttatgaagaaagctaataatggtaatagatatgtaatagatatagatggtatacctgttgattttgaaagggatttagatagtttacttaataggtataaaaaccttagatggtcgttatatcataggtacgcagggattttatctaatgattttgaaagacaagaactaagagaatatattgatgagcaatttattaaattagttaaagaatataatattagaagtaaagtggattttcctggatatattaaagctaaactaactttaagagttcaaaatagttatgttaagaagaatgaaaaatataaacgtactgaaattatcggtaaaaaagattatacagtagagtctttaacagaagatttaaatgaagacttcgaggataatcaaattatgagttatgtatttgatgatatagaatttacagaggttcaaagtgagttacttaaagaattacttattaaccctgaaagagaagatgatgcctttatcgtttctcaagtagcggaaaagtttgatatgaaaagaaaagaagtagcaagtgagttgacagaactcagagactatgttagatttaaaataaatgcataccatgagtactatgctaagaaagaattaaataaccatagagttaatactgaaaatcatatttgggaaaactagttacagtgccttccttgtgttatattattatcgagaattcaataataaagcatagggaaggcttttttctatgtcttatagaatgctttaaaatagattactaaaataaagattggagattaagcttatggctaaaaagaatgttaatgatgtattacaacaagaatctgttacagtagcagataagtatttacaagttaaagttaaccgtgacggttatactcgtacacatgaaggacaatatgcgtacaaagtagtttcagagggagaagaattattcttataccctgtacaaacagatggtaaaggtacattaaatgtaatgaagaaatcacctattgcttacactgatggagacaatatccatttcgtagtaaacacagtagtagacccttataatcactcatttatccgtactgaagatattaaaggattagataaaggtaaacaacttattcaagctttcttagctttcgttgaagaccgtttcaaatttggtgtttataacgtatttgttgcaaacaacaaagaggatgtattatctattgtagaccctacagataatgatgcagatgaagttaaagatagtttagagcacgcacatgaagatgtaattgcggatttccctgctagccctgctcgtaaggacgttaaaggcgtagattcaggagaaggtcaaggagacacttcagaaccatcagcacctaagaacgttcaagttactcctaaggaagacggagcagacgtatcagcagaataatatatagataaggatggtaaatttggctaagttaaatttatacaaaggtaatgagttactaaacagcgtagaaaaaacagaaggaaaatcaacaatcacgattgagaatttagatgctaatacggattaccctaaaggtacttttaaagtatcattctcaaatgattcaggagagtcagagaaggtcgatgttcctcagtttaagacaaaagcaattaaagttatttcagttacccttgacgttgatagtttagaccttacagttggagatactcaccaactatcaacaactatcacgcctagtgaagcatctaacaaaaatgtgtcatttgaatcagacaaatcaggtgttgctagcgtaacatcagaaggcttaattgaagcagttagtgcaggaacagctaatgttactgtaactactgaagatggtagtcacactgatattgttgttgtaacagttaaggaacctattcctgaagcacctgcagacgtaacagttgaacctggtgaaaatagcgcagatattactgtataggaggacaataaagaatggaaaagacattaaaagtttatagtaatggtgaagttgtgggctctcaagtagctaataacgatggagctactacagtatctattacaggcttagaagccggaaaaacttatgctaaaggagattttaaagtagcatttgctaatgattcaggtgaatcagaaaaagtagatgttcctgaatttacaactaaaactcctactgaagaaccttcaggagacgcataataattaagaccaactaaaaagttggtctttttttattgacaatttataatatctatgatacactatataagaattaagaaaaggaggggaaagtaatggatattccaacaatattatttagaaatccatatgattatacgaaagtaaaaaaattaatggaaaacaaagagcagtatattgtagtaaagtttgattctgtttctgttcataatttaaatgttcaaggtatgatgaatgtcatccaagattacctacacatctatggttacagagttaaagagtacggacaagaaaattcttctaaagatgatgaaagagacgttaaaggctacttatatgaaagagtaggtgagtagggtatgggaattatagtaaactccaaccatattcaatcagacactttatatgagtatgatagcttttttgatattgagaaagtagatacatttgaagaaggattgctttcaatacaggatgagccaactgttttagcaggattcatctatgatgatatcacatttaataaggtcattaattctaattcagatattgatgattatattaagaataatgatatttattatgtctctgatataggattacttcctgatacttttatcactgttgattctgatagaaaatattattcattattacaacagataactgagttaagtaaagacccttttcctaaatgggtagaggatgatgcaaaaggtttaactaagtattataactttcaagattttgaagatgtatttgatttaaatagtttttacaaaaagaagttgacatggtaagagaaaagtgctataataatggtaatgtatatttattatatgaggttctgcctgattataaattacctctagcttatagtttactttcaaacaaggagcatggtattgttattatcggttcacagacacgttctaataatgatatactgactttttatgttaaaggtatggatgctaaggcaatagctagtatgttcaatgtagaacatgattatgattctaatattttccatacatttgtaaacagtcacattaatattttaggaaatcaaataactaagtttataagagagaaaggaagcagttatgagtaactataaaacaatagaagaagtacaagcagttattattggggtattatttaaagatgaaggtaaaattgtaacatctaagtttaataaaattactaaagagtttggtttagatagaatcggtaaagatgaccttaaagaaattgtagaggatattagacaagacgcttatctaaatgaacttaaaaacaaagcaattaaaggtaaagtaacgttaggtgatttaaaagatgttgcagataaccaagtattcgaaggtaataactaccatgaagaagtatctacttatgtagtagctaaagaaaaagaattgtctcacttaagagaacagcgtaagcacaataggcatactgcataccctcaaattatgtttgatgaacttaaagaacatatggttaaggaattacaaggggaaacattagtagaacatcacggaagtaaagctaatattaatgatacagagctaattgtgttactatcagatttccatattggaagtattgtatctgatatgactaatggtaaatatgattttgaagttcttaaatcaagattaaatcattttattaatacaacagttaaagaaattgaagatagggaaatttctaatgtaactgtttactttgttggggacttagtagaacatattaatatgagagatgttaaccaagcatttgaaacagagtttactttagcagaacaaatctctaaaggtactcgattacttattgatatcctaaatgtactatctaatgtagtttcaggagaactaagatttggtattattggtggtaaccatgaccgtatgcaaggtaacaagaatcagaagatttataatgataacattgcttatgtagtgttagattctttattgttattccaagaacaagggctattaaatggtgtagatattattgataatcgtgaagatatttatactattagagatacctttggcggtaaatctattatcattaaccacggagatgggttaaaaggtaaaggtaatcatatcaataaatttatcttagatagtcatattgacttattaattacaggtcatgtacatcatttctcagtaaaacaagaagattttaatagaatgcacatcgtagcttcatctccgatgggatataataactatgctaaagagttacatttatcaaaaactaaaccttcacagcagttattatttgtaaataaggaaaataaagatattgatattaaaacagtatttttagattaaggatggttaataaatggatacaatttttattataggtgtagcgtttataacttttgcaacatttaacatagtctttagattatttgatttatggactacagagaaaaaaatggtaagtcaaggacaacctccactaagtaactttgagtactatcatgtgatagtaccttacttagtaggtgttattgttattatactgagtattatttttagggattccttgtattccgcacaatcagggttcggtgttattattacaagctttatttacatgctagtttatgttataattggtcttgtagggtcatttgtacttacaatattccaagctagaaaagctagacagtatcaaacacaggaggataataatgaagttcaatgatatttatgagcaattaattaaaaatgatacagtacaaaacattcatgagtctcaagatgacaaaggaaatatttatacaatacagtttgataaaggtaatgataagtatttatttaatgttattaatgatggattcttgaaagaaatgacaaatggtatggtagaccatcctgaaggtcagccatattcagtaagtttaatcaataaagaaacacctagtatgtcagtgaaacaatatttaacagatgtagaagatattgtacctactattagaaaaatggaaaaggatttcttatagagtcaagtctttacttgactctttttactatatatggtatattaatatagaggtgacttaaaaatggattttaattttagtgcttttgataatagctcattagcaatgagaattagtgagggtgtatactatttcaatgatacgccttattactttattgagcatgtagaagaagaaatgtctgagtatgttattgtatatgacatacatgacagagaggaaaaagaaaatcctcagaagaaatatagaatagaaccttaccaacgtacaataccgggaggaacacctcttagtaatttaattaagagtatgatgcctcaacgtaagtatcctaagaaggttacagaagaccctatatttgtagctaatgttattcctttaggaacagatacagtaacaggtaaaaccggtaaaggattttttgaaagagataaggatagaactatctattctcaaaaggaaccaactaaagtcgttcatggtcaatacacaggtgtttttataggtctaacaagtgttaagtggaatagaacatatacccccttagaaagtgttgttgagtactacaaaagggttaaaggagataggttaaatgtctaatgatgtagttaagttctatgaaaaagatattaaagaccttatcagaactaaaaaacacatgttcaaagacgatgaaataactagtgatataaacgatatacgaatcttcaatgagaaagtcatttgtcaaggtaaatgtagaacagattgtttagtgttagaccgtaatggtacagtaatgggtatagagataaaaacagaacgagactctacacaaagattaaataaccaattaaaatattatagtctagtatgtaagtatgtatatgtaatgtgccatgacaaacatgtacctaaagtagaacaaatacttaaaaggtacaaacataatcatgtaggtataatgagttacattagttttaaaggcaaacctgttgtaggtaaatacaaagatgctacaccatcaccacatagaagcccttatcatacaatgaatatattatggaagacaaacttaatgacaatacttagattgattagagaccctcatacgtatagaacagggtatagctataatgttagtggtagatatagtggaggggaaggtaatttctcccaaacaactcaaagtaaaagaatgaaaaaacctgctattattaaccaaataattcattatgtaggggtagataatacttataaactctttacaagaggtgttatctatggttataataataggtgggaagttatagaagaagatttctttaatactatgaagaatggggtaagagtaattaatgagcaaagacaaaccaaatagacgtaaagagatacagcatcaacctgttaactttgcccctacgaatactttaacaggagctaataatagtttctttgctaaaaagccttcagagcctaaagatgcaacatctgttattgaatatcgtatactatttattaaaagatttgataacgtaacaagtacagatgtgaaattacagaaaaagtatgcactaaatcttattagtgaagcacttgatgttaaagaaacttacttgtctcttaagcaaaaaggaaaaaaaacagaatctattttgcatacagatagagtttattatgttcatagaggtaaaaaacttattggaaagtgtagtatcagagaacaaagaacatttaagggtaaacatttgatatttatattcaaaacaagacatagagttaaagcagaaaggaaagataaataatgttaaaaggattttcagaacatgtagacaaacctacaactattaagaccttatacaagaccttaacaagtggtaaagtagaattactaggtgtatcttacgatagtgattacttcccttcaggtgttacagtacaatcttacattgaggatataggtaatgaagatgagggtctacagtttgttaataaggtaaatgtagtagaatcaatgaaacaggctgtagtaggtatgaataatcaattaggttcttcaggtcttggctatgtgagaactgaacaacttaaaaaagagttggaagagactggactaatgacagatttacttgctagaggtactaacttaacctctactaagaaagtagatattgtaagtacttttattgagcctgaggtaacataccaaaatattactatagctaaagatattaaactacgtttgtataaagtagaagaagaatcaccattaaatggttacactcatattgtatacttacttactacagaaaaactatatgatggtcaaacactcttcggtatgctctctaaaaaagataagttatctaaaggagatactgataaattattagcattcttcagaaacaatagtttaataagtaaaagtgtattttgtgttaagttattaagtaaagactactactttaatttatataatacacatgagacagggatattctttttagaagacacagatgttattactattgcttgtggtcagtcatatgttaaagttaacactaaagatattaagtctagttatgttaaaattgaagataagactcataaattaactgagctagtaattaacctaaagggtgacgacacattaactattttattctaggaaaatgttataaatatgtgataattaagtataaatatacgttatataagaagttttcataatgtttttaatacagaaactagttaagttttttctacttgctctagtttctgtgaaattatatttatgaaaagttaaaatatcttttaggtaaaggctttgtaaatagttaaaaaatatattaaaattttatacaaagtagttaataaaattatattacatttatatattatgaaataataacagaaattgtgatatattatatagtgtaaccttgaaacagttgatgttgtagggtttgtttatgttcgttaaactggtttcagaacatcagttaccataaataaatgacagttaaggagagctatataatggctagaaaaaagaatttaagaaataaaaacagtgatataaaagttgttcctgataaagaaaaagaaagtatattatctaagttataccataataagttactacgttcaaaggtagataatgcattagatgaagatatgagttatgatgatattatagaattatgtaaagaatatgatttagaattgtctaaatcagctattacaagatataaaagtaaaagaaaagaagctattgaaaatggttgggatttaggagaattaattgataaacgtaaaaaaacaagtgtaaaagatattaaggaaaaagaaactcctatattagaagaggagcaactttctccattcgaacaatcaaaacatcacacacaaacaatttatgatgatattcaagtactagatatgattatttctaaaggtgcaaaaggattagagtttgtggaaactttagaccctgctttaatgatacgtgcaatggaaactaaagataagattaccggaaatcaattaaaaggtatgtcatttattggacttagagaattacaattaaaacaaacagctcaagatacagctatgagtgaagtattattagaatttatacctgaagagaaacatgaagaggtattacaacgattagaagaactacaaaatgaattctacaaaaatctagatttagatgaggaaagtagaaaattaaaagaagctcttgatagagtaggctatacaatttagatagtgaggttagagtaatggcagatgagattagtttaaatccaatacaagatgctaagccaattgacgatatagtagatatcatgacatacttaaaaaacgggaaagtactgagagttaaacaagacaaccaaggagatatccttgttagaatgagtccagggaaacacaaatttactgaagtatctagagacttagataaagaatcattctactataaaaggcattgggttctctataatgtatctgttaactctcttataacatttgatgtttatctagatgaagaatattcagaaacaactaaggttaagtatcctaaagatactattgtagaatatacaagagaagaccaagaaaaagatgttgctatgattaaagaaatacttacagataataatggtaattatttctatgcacttacaggggaaacaatactctttgatgaaaataaattaaataaagttaaagattagggttgacagctcctatagtttatgatatagtatatgtatactaaaagtaaaggagctaacaattatgtttatttcattaaatcaagaagagaaagaattattaactaaagaggaaagtaaatacacaccattagaaacatcaagagagtttaacacacctaaagaagaattcattgtaacaagctataatgaaggtaaacctttagattacattgcaaaagaagctaaggtaagtatgggattaatttacacagttctaaactactataaagtaggtaagcgtaataagaaatcacctgtagaagaaagaattgcacatatcttaaaagataaaaacttagtcaaagagattattaaggattaccaatatatgaatttacaggacatttatagtaaatataatcttcataagaatggtttatattacatcttagatttataccatgtggagagaaaatctgaacttaaggacaaagcattagaagaggataatattgtcgttgagtaagtaaagaggttataatatgagaaataaaaaatcatttcaagagcagttaaatgacatgcgtaataaagagaaatgggtatctgaagaggagttcactgaagaagtggctcctcctgaagaacctgaagtagaagaagaaaaactatatactttaaatgagttaaaagagagcttactagatgctcaaggattaaaagatgttgtagctgattttcctgcatctaaagatttatatgaacctaataagttatatatctgtacaatacctaaaggatatcagtctaccgaagtacaaccaggacaatatattggtattagtactggattattatcagagtcagaagacttcagccatttaagaggtcaaatgcctagaaacttatatgaaacttctcatgttttaaaacctttgatacgtattaataatacaaatattgaataccaacaacatgagttacttgaagacattaaggatgacaaaaagatatatgatgtagagttagaagacttaagattagcaacaggagaagaagtttctcatttagaaattgttgataataagttttttgaaagtcgtattaatgaagttcttgaccgatacactgaactaacggattccaatgatttacttaagtactatagtaaattacgagaattagtaggtagtgacaaaatgatttattgttcactcctcgataaatgtgttaaaattatagattaatagtagtctcctcttatattataattgtaagaggggacatttttgtatagaggtgttaattatgtcaagaaaagcaagtatattctatatactagtggttattgttttggctttctctatctcatcttattatatatcttctttcatgtatcacgacaaagcaaaaaatgaagtctctactgagttatcgaacacaggaaagattaaagaagaaaagaacgtagaatttgtcggtgactacacattgaaaaaagtggaagataataaagcttattttatggaaacattacctacttacctaccaggtagaacaggagataacagcatagatatgaggtactacaaaacaagtagatttaaggaaggggtaaatttcaagcttattagggtatatactgaagatggagaagataatccaattcataagtataggtttgaagcagtaccaaccaaaaagtaataaggaggtgacttaaatgacaacattaattgtcgtcatctttattgctatcatttattacttatggaacagtgattgagtcaagttaattcttgactctctttttgttttatggtatattaatatatagaaaggagagattaactatggaaatggcagatttagaaagatttgatgcatttgtaagactaatttcagatgatgagctttcggaggaaagaatactggagttaagcgtagacttactaaacccgatactagaaggaggtacagcttacaaggctaaaaaacgtattaagagtaaatttggtaagttagaagcaaaaaattttaaacgaaactataaattcttacttaagtcgatagctcaaatagaccaaaggagataggacaatgacagaaagggaaaaattaattaaagatattgaagaggctaatagagacatacagttacagttaaaagaagtagataattataaggacagcatacgttctaaaggaacaagaaattatatttctacaaaggtattagattctattatggttggtttcatatgatgaagtagatattgtatcaaaacataacttaagacaaataacttttagtgagattaatatacttcatcttaaatatatgagatataataaggcagtagaacaacatactaaattatctaaagaactttttaaaaaagataaagaaactgttgacaagaacaaataagtgtagtatagtattactaaaggaggagagatattatggttatacctagtattaaagcacaaaacaaattcaagaatgagttagagtattataaacaaggtcacattagtgaaagtaaaatgttagaattagcttttgattacatccaagaattagaacaaaataacgaatacgttactaacttgctagaagaggagagatatggtgagtaaatttatcggagtgtacttatttaatttactaatagctattattttaactttaaccttaataggtactattactgactcaattgagagtactttagcccaaataatcgtagggatgttcataatcattactatatatggaatcctatcagcgttaatacctattctagttcataaagctgtatcaccgggatggagctatactgaatggaatgaatcctattacatcagattacctggagaagagaactacaagtactatagtaaatggtatttagatttattaggagttaaagaattttactataagagagacaatggagaagaagtaaaagaaaaaaatatatcatgggcttttcaagctgaagtaaaaagacctgaagatgttaaccactggaaaaaccaattgcttactaatagacctttaacaattttagaatataaaaaattaaagaaattagataaggaaagtgaaattaggaaacaagaagatttagaagaatataaacaatacaatagtaattaaagaggtggaaagcaatgataagctcatttgatagtatactacttgtcatatacattattatagcttttgcagtagctatggcaattatctacttagtatttaaaggtatgactattctactagataagctaatgatgttattattaagtaaaactacattagatgtagaagcttgctctatgataatggcagtcatcagtacaattgtgtttggaattattgtacttttaatatggctagcagtaaataatattttactataaggagatttactatggattttaatgactttataaacagtgaatcggatagggtaggtaagcctaaacaaaagaagaaggtagagaataagctaccttcttctactcctattgaagataaggaaaagaaattaaaagagataagaaagaaatcattatatattgatttaaggagaaaaagaaatgactaaagaaacaaatgtactttacaaagataagtatagagattatactatagttgtaagattagcagggaatattattgttactgaagtagataagaaacataaaacagcatttacacctattatatttgacaatggtgtagaaggcgtagagcttgtaatgcgtataggttctgtagagcttaacatgacagatttacgtgagttcacaaaagaagtatctacggctcagaaagctttagaatattttaataaaaaactttacattaaaggcttgacagatgaagcattttaatatatactaaaagtataaataaaataaagaaaagaggaatgattattatgttattaggaattttatggtttatatggggatttgtatcatactttgtattgatgtttggaattgagttttggaaagatagatggatgccaggtgttatcggagcaggaaccttactactattcttattttggattatgaaatctatccataatgctatgacagtagtatacttgtattaggaggttgtatagatggatatactaattattcattataaagaaacaaataaacgggttttaaaagaaacaatacaaacaatacaaaatcatttaaatgatgaacatggtttggttaagatgacagcaacaaaacttagcagagagaatatagagaaaagatttaataactataatatagtcattgcagaagatgaccctgataattcttatcattacggtgaagctgtagaagacgcagattttattatagacataccaatttcatatttagatatacatgcaggaatagaatgggatgttgataatcctgtagatatgctagataggaatcctgattttatagaagctgtaaataaactaaatgaagacttaatgttataaggaggaaatagaatgctaaatgaaaaactaaaaaacctggaagatacaaaagtatacatgattaatagtattgcaagtttactaagcgcaagtacaggaaaatcaagtaaagtattttttgatgaagggactattaaaattgtaagtggtgaaacaaaagcagtagaagtcattgataacttagttcaccctcactcaggacgtttacctattaaaacaacagaacgtattgcgctaggtagattaacagattctttacagtttgttatttcagaaatagaagtagttaaagaccaaattatagatgaagaaaatgaagcttacattgattttgtgatggaagactggaactgggattaatgcctatggacttattaactattgcttctgttgcttttatagctgtagtcattgttgatttgattaatgatgatatgagctatatgcttactggtactgcaatcttaataaatatttgggcaggattttatggatggtttttcttactgcaagcaggaatgttacttttcttactattagctaggaaagttaaagatgataaggagtcaatactatattccagtgcttcattaatatgtgcactaggaatgataataaatcttctttcattttcttaaaaataagtattgacacctttgtacttttgtattatacttagtatataacaagtacaggagatgattactatgagtaaagaaacaattagaagacaattttcaaatgcaattgagattatggcaacaactaaagaatggtggaacttccctaaaagttttgatacgaataaagaatttaaaattaaaacttttaaaaatgatacacttgtatttgaagtcagagaaggcagtagaaatttaggaagctttgtagtttttacaaacattgattttgattatgataaactagaaggaacttcaacacaatatatgattaattactttgctaagaaattaactaaagatatgtttaactatcataaattacaattatagtaggaggtggaaagatgagagaagagttaaaaccttttaataggaaacaagttaatgttaagggttacttagatgatgttaagtattcaaagcgtagaagacataaaggtaatcaacatgggtgtgttaaaatcacagttactgatgtaaagattaatggtatacctattgaccacgttaacattgaagttggtatctctttctatgaaaaactaaaggagcttcaaggaaagagaattcaatttgtaggtactgtttataagtatgttaaacatgctagggggcgcaaaggtagaattaaaggattttataaagaggattatagcgtaactttagataagaagttacaaaaggaggaaaaataatgattaaaagaagaaaacatttagaccactcattacagcctgagaaaggatggagaacagtaccttttaatgggtattatgaagcgcatcctacgggtttaattagaaataaagtaacgaaaaagttaattaaaggtacacagacaagaaagaaccatcctaagtggactgctcatgagattgtatacctaattaaccctaagaaaacaagttattctaggggagtagttattgcacatacattccctgaaatgattagtcaatcacgaggagaccttaagaacggtcatgtgtgttttaaagatggtgaccgaagtaattgtcatgtagacaatatgtttattggtaaaggtaatgttaacaaaaatatctataaattaaatgattcttatttaactagaaaagatattgaagaggatgttaataatttagttaatgaaagattattctctcaattagaattattgattaagaaaaatgaaccggaaagaattacacctagtaatcactttattaaaagagataataatgtgttcagtatcacagatttatctaaaaactcactagtagagtttgagttagaaatcaagaatattaagtaaggtggttatataaatgaatgagtggtatgctttatgttattataacaaaataggtaaaaagaaaatacctagacaaattaaagctcacagggatgtatctgtattagaggatttaaaagatagattagaagaacaaaatcctaaagaagaatacaagattaaaacaacaaaagaatttgataaggaaagataattaatgttaacacctcaacaaaaggattcattaaaagagcaacaaaaaaaattaagtaaaaagaagaaataagtcttgacaattgagtatacataggttatacttaagttaacaaataaagaggaggtatgacctatgttattcgtaatttttatattggcagtactgtttgtacttggatttatgaatggatggaactcagaagactagataaggagtggttgtaatgaagttagaagataaagtgttagagagaattgattctcttggaaataaagcaggtaacttaagtaatcaagtaatggagtcattagtaaagtatcaaattacgtacggtattatagatattgttgtaagtattttagttattgcactaacaatatttttaggtaaggtttaccttaaagaatataagaaggttaaaatggatttaaaagaaagcttattgtatgatgattacgatgacttaagtggtatcggatggtgttacacaattctattaatactattaacgttattctctctttacgcaatagttgcaggtatcccaactgatattatgagattaattaatccggaagtttatgcagtaaaagatttaattgagcaagttaaaggaggaaattaaaatgaaacaaagagactttgaatttgaagaggattttgtattaacttatgagtgtgaggattgtaagcatttcgaagactggggtcatgatgaagagcctgaagaatgtagtgaatgtggaagtagtgatttaatctcaataatacaagtcatgaagatactgagtgtgatatgtgtcgagggtatattgatatgtggcaagatggatatagatatatgggagataataaagagtatattgaaaaagaggaatcaggtttgatttgtgaagattgttatgagaaattagatatttaataaggaggaaattaatatgaataaagcagtagaacaagcaagtaatgcattaggtcaaggattttcagctatggtatggcatcaagtattagtagggttagggtttattttattaggattggtattatctttactggtttgggtattagtaaaaaaattccatgtaccttttaatcacccgacagcttttgtagtgtactcaattatgttagtgagtattgttgctagttttatttggggcggtttacatgtaattaaccctgagtattatgctattttagaacttaaaggttttataaagtaggaggaattctatgactaaagaagagttagagcaaaaagtaaaagaacttgaagcagagaataaagagcttaaaaaacaaatagaacgttttgaagacgaaggaggaaaaacaaaagatgaacagtagagaaaagaaaattttaacactaacagtaaataattttttaatgttagctttagatattgtagcacttgttaggtacaagaaaggtaaaattaagcaagagaattataacacaggtcaaatttcaagaactatagttacaacagccaactcattaggtattctttacctagaagagcaagaacgtaaagaaaaaaaatctgttaaaataggtactcttgaaagtggtactctaagagggtttaaaaataaataaaaaagtttaagaaacctattgacattaggtttcttttattatatactaagagtataagaaataaggaggttaatttatgaagcattttattttaattttaggaattgtaatactagttattgcattaggtattgttttaccggcatggattttacagttagtactatctgcattcggagttaaagtaagtatttgggtatgtatcggaatatttattttaatcagtgcaataggaagtatgtttagcagaaattaaaggaggaactataaatggcaaaatatgaatcaaatattaatggagagaattatattgcaacaccgtcacaagctttaagagaggcactagcaaaattaataactgaagaaaagagctttgcggagtaccaaactaaaggtgaggagcagtatgaatcacagttacaactaagacactttgatacaatgatatctcagtatgaggaagctattagagtactagaagataaatatagacctcagatttttattccgaaagataataaggaggaaaattaattatgaaagcagaatcaatagcaagattttttaatgacaaagtactacaaatagagggttataaagtaagattcttacaggctagttcatcgtatattttagatatagatactatagatgaatcagtattgtttttagaagctcaagtatctacactttcaggtaaacatttattagatacagctattacaattgagagacctgaaacattaagtgctaaagagctatatacagaaattagtaataaactacaagctattgtaggagaccaaactaaaacaactatagaactatcaagatattttaaggaggaaaaataagtgtctaataaaaccattacaaattatttattaaatttagaaggaataaaaggagaaacgtatagtattattgctcatatcaataaacaaactggttgaggtgataaaggggattattttgaaataagcataagttataaagctgataaagaccctagaacaacgagatatattacaactgaaatttttgttgattatggtagtaataatccaaaagaaattttattacaattaagagataagattttttctattgtagaagaacaggtagagactgacaatgattttattgaatctattaaagaaattaattcaactaaagaattagaaaaactaaagccttatatcaataatgaatattattcaatgtttaaatcttctattgaaaaggaaatacctgtagctttatcttctgaagtactcaatagatgtacaggtaaaacaagcacattagcttatttagcactagaaaaggatttacccttagtagtatcaaatgaacctatgagaaaaatgcttaaaaataaattccctcatcttagagtagcttctgctgaagattattcaaattatgatattaaaggtgaaattgttctaatagatgaagtagatattgaccagttatatagtgctgataaagtatctgttgatgcacttttagtgggtatcattaaaaattaaataaatttataaatacctgttgacagcctgttgacagcaggtattttttatagtatactttagatataaagaaaaaggaggtaatataatgatacccgtaatagttatacttattggactcatattatttttatctagcggttataagttggtattgggtaagtattatgatgatgtagatttaaaaatactatttaccatatttggtgttgggattgcattactacttggaggatttatattataaagcaggagctattttattttaaggagaggtaaatatgaattatagagattttattacagattgtattagcggtggttacaacgtacacatcagtgttacagaaaaacgagtacacattatttctgagatgacatcagcatcttaccctaaaaaggaaattaacttagatgaactacaagcttatgtgtactatatgaataattttggaagtcaaattacaacggaggggttataaatggaattggttattaatattgtagcagtattggttggtatgtatgctatttatttctatgttacaaagtttagtactggcttatcaggtattttaattgttttagggatggctattggtctttacttctacttagactatttaaatgtcagagaaaatgttattcgattagtttcagtaatgttcggagctttcttatttagtattgaaatgatttataataaaattatgttcgaaattaaaaaaagcaatgttcagaagactgttagagtgtatgataaagagcagtaatgattttaccataagagtacctaaattactttaagtgctctctatggtaccttaaagtagcttagaattgaaattaaggagatgaacaattatgtatcctgaaatagatgtggaagaattagcgtataagctaaaaagtacaagagagtatttagagagcattacaacaaaagaagtagaaatttatgaaatctatcatcttaaaacaggtaagttagtttttaaaggtgaatacattgaggtaaaagaattactgaggaaaatgtataaagaaaatttaacacttgtagatgtagatacaatgttaagcattggtaaaggatttattgatgtaattaagaatatatcggcagaaaatgtattccaaataacatataaaaaggagctatcaacaaaatgattaaaatattttcagaagtagataaagaatacaaacctattattactgaaaagtttcctaatggtgagattaattttaaatatgatgatttaaagtatttagtagaagaggacttaagatttgatgttttctttaaatgggaaaatgacgcagacttaatgcatttgtatatgtttactaagtatttagagcaactaggtattaaagataaagctgaatttttagagattgcatatctaccttatagcagaatggatagagtagaagaaggacataataatatgttcagtcttaaatacattacagaatttattaataaccttaattataaatcggtatgggtagcagaacctcatagccctgtaacagaagaattacttactaattcttttgctattgatgttacacttaaattattaaatcagtatattgaaatgtccgaagagcctgtaacaatagtactacctgataaaggggcatacgatagatatctatttgatgtagaacgtatcttaatggaatctaatattgaatcatattcaattgtatatggtgagaagaaacgagattttgaaacaggtaagattaaaggtattaaaataattaaagataaaaatactttatatgataattgtattatactagatgacttaacaagttacggtgggacatttgtcggttgtaaaaaagcccttgacaaacttaaggtaagtagtgtatcattaatattgactcatgcagaacgagcttttgcagaaggagcattacttagctcaggatttaaagatattattgtaacagactctatgttccctaaaaataattgggaaaaagctattgctaaacatagagctagaatcaacggaactgaattacaaataaaagatatcgaaagatatttataaaaggagaaaaataaattatgctaaatccaactttaatgtgtgacttctataaactaagtcacagagaacaataccctgaaggtacagaaattgtatatagtacactagtacctagaagtaataaatattatgaacacagtgataatattgtagtatttggtattcaatcacttgttaaaaaatattttattgatatgtttaataaagagttctttaacagacctaaagaggaagttattaatgaatacaaacgtacagttaaatttacactaggacaagaaaatcctgatgctaaacacttagaacaattacatgacttaggttatttacctattgatgtaagagctttaaaagaaggtactgttgttcatcctaacacacctgttatgacaattgaaaatactcactcagatttcttttggttaactaattacctagaaactattattagtactcaaacatggcaagcaatgactagtgctacactagcatatgatatgcgtaaaatgctagataaatatgcaatggaaacagtaggtaatattgaagcagtagatttccagggtcatgactttagtatgcgtggtatgagttctttagaaacagctcaattaagttcagcaggtcatgcaattagttttaaaggtagtgatacagtacctgtagtggatttcttagaatcatattacaatgcagacgtagagaaggaaatggttgttgcttctatccctgctactgagcactcagtaatgtgtgcaaatggtaattatgaaaccatggatgagtatgaaacatataaacgtatgttaacagaaatatatccaacaggcattttctctattgtgtctgatacttgggacttttggggtaatatgactaaaactttacctagattaaaggatattattatggaacgtaatggtaaagtagtaatcagacctgatagtggagaccctgttaaaattatttgcggagaccctgatgcagacactgaatatgaacgtaaaggtgcagtagaagtgctttgggatacatttggaggtactgaaactgaaaaagggtacaaagtattagatgaacatgtaggattaatttatggagactctattaactatgaacgtgctcaacaaatttgtgaaggattaaaagaaaaaggttttgcaagtattaatgttgtattaggtgtaggtagtttctcttaccaatttaatactcgtgatacccacgggtttgcaatcaaagcaacgtatgctaagattaaaaatgaagaaaaacttatctataaaaatcctaaaacagatagtggtaaacgttcacataaaggtcgagtagctgtatataaagacggttcatgggaagataacttaaccttacatcaatggctaaacaaacaaaatgttaatcaattagaaagagtatttgaagatggtaaactttatagagaccagtcgttaagtgaaattagagaaataattaaaaataattaataaatatttaaactccctattgacaaagggagttttttattatatagtagggctatagtaaataaaggagtgaaagaaatgatttataaaatatcaaaacataattactatagtagatttgagcattccacttatcctcctgatgaggggtttgcgtatgtagattatgtagatgtgattcttattggtgtagataatcctaggaaaagaaagattattaccttaaaagtaaatgagttcaacccggatgactacagagtaggtcataagtacaatattataaaaatactatggtttgaaaaatgggaatggttaaagccataagtaaaaggagagaaataaaatgattatagataaattaaatggagttaaattagaaataggtgggcatgtcgtatcatttagtgtaagaaagtttaatacaattaatggtgagagacaattaatagactaccatcatattaaaagaaatagacaacagtactttagaactactgaagaattttataatgaatataaagaaattaagcctgacaaaaatgaaatagatgaaatgtttgaatctctaggttatgtagatactgagttagatgatgtagtaagaaaccaggaaaaggttactgaaatattaggagttagtgaacaatatttaaatcagttatcttataaagctatagaggagtatgtagataaagtagttacacttgaaattaaagagttgaaaggagagaaatagcatgaataataactgggaaaaagaaggagttaactattgggaaaaagaaggagttaactattgggaaaacgaagactgtcctagggaatacttagagaaagcattcattgacctggtagaatatgttgaaggagttacagtaccacctaaagatgttaagcagttaagagaagataaacttagagaagatattgggttttatgagtacgtagctgataaataaattagtatctacctattgacttaggtaggtatctattatataatagtatacaaggagatgaaaatatgaaaaagttaatagtattacttacaattactatttctctattactagggggttgctctcctgataaccatgaaggtaaagtagtaggagtaggtgaatacagagaaccaactacttatataaaatcaggtagcgttactgtaccagtcattggtgaaatgaaatactatgtagatttagagacagataaaggagaagaccgtgtatatcttaataaagaggtctatcataagtttgataaaggtgatgatttctctaatgtaggtgagaaagtgtataagaatgatgaattaatatataaaggagactaactatgtatttaaatgattatgtaggtaaatttataaaggaagataactattatggatatcaatctacagacttagtatctaattatgttcaacgattaactctaggtaggtacaaaactaagttaaatgctaataaaatgaaatacgaaagattacctagttcttggaaaataattaaagccaaagatttgttaagaacagatgattatagagaaggagatatatttgtatcagaaagaatctccgtattcggttttaatggtattattgtatataaccatgattttaacaatgtaactgttattactcaaaatagagatggtaaagctactaatcctgtagaggagcatttatatccaaagaaagatattgattatattattagacctatcgagagggactacagggaatactttaaaaaatcagattcaaaagaaaaagttactctttcgaagcaagaatataaaaaattattagaggcttataataaaatgaaggaagtgtttaagtaatatgaatagtacaaaattagtagagtactttacaaataaacaaggtaaatctctaatattacctgatgaaaataaagttgagttatatagagttgatgtaacaccttatactatgagacttaatttcacttacaatacagaagttgtagctatagatattgataagttacactcagattctatagaaatgcatataccacaaggtctttatataacaactgttgttaaaattactagtacgcagagtattagttcagttcttcataaggtattagaggaatgggtaagacaagtacaaaatgatggtatattcggattcgtatgggagtaattataatgataagtatagaacatgattatacaataagaactgtagataatagaaaatatacttattatagtaaatacgaatcactagttactttgtatgaaaatattatgagtaaagattgtattgaagtaactaaatatgggaaagataaaaaagttattattgatactagacatattgtatctattgaacgatggtaaataataaggaggagtaaactattatgataaatgcagggcatgctaagtacctatcagaaatttatgaagatgatgtacattatgaaactatagatagtattgtagaagatatactagataatattaatgatggtattattgaagaagctatgaaaggtaatacaagttatcaatatgttcttagagacttaagagtagataatgaagtagaatatagagttatagaagaacttactaaccaaggatatagtgtaaaccacattagtaatgatatagagtacccttctatatctacaaataatttagcagggttagattacttaaatattaaatggtaagggagggaattaatatgataaataaatataaaaagttatgggatgaaataactcaacaaattgttaatgtagaaattattaactttaaaaatgaaacagtaacaatagaatctacagatgattcaggattatcagagataagaggttttgaagaagtagagtttatagattactatggataagatgtttaaagtatataatttataaggaggaaacatatggacttgtttgcaaaaataattattatgtctataggagttgttcccttgttaactattattgttgcacagctaattacagattaccatgataatcattaagtattatagtaataggaaggacaatatttagagtgagagtatgttgactaatgaggaagatatagaatgagaacctaaccaagtaaaactaagtacctttgttatgtactactattactactactattactactactattactactactattactactactaagtacctttgttatgtactactactaagtacctttgttatgtactactaagtacctttgttatgtactactaagtacctttgttatgtactactattactactactattactactactattactattactattactaagtacctgggaattcttttacctctctcactcagcctattacttattaccgacttccctaactacttattctatagttataatattcatttattatacaatacttaaactatagtattctactgttaatctatgctgaagcggtcttaatctatggttattatataataatcttatataatggtacattaatctagtatattacattagaatcattctaatctaggattttaatctttagaccctaggaaaagtggtactaaaatataaaaccctataggtatgggattcttatttttaaaattactaaaaagtattaggttttccctagggcaaagttttaatgtacttaaaatagtaagtagctacttatcatttagggttctataattgagaatattgagagataatccgcttcaattgtaattaattgttgacaactatgaagcgggtatgctataattaggtatagtcaaatttaggagatgaaatagatgattgatatatacttaggagaaggttataataaagaatacttgtctaaagcactcagattaatcaatgaccatgctcctagggagttaagttatgattttaataatgtagaagcggatgttaatattcacacaatgttatatgttaaacctgaagatagatttatatataaggatatatcctattacttcccgggtgatttaattatttgtatagttgatgatgatgctattgtataccaccaaggtgagcagatttcaggtattagtattttaagaatactagaagagatattttaaggaggataagtaatcatgataggaataacaatattaattacgataatgagtatatcaactatctctatgtatatttattttttagtagacttgattcagtcaatcagatataatagttttgataaggtaattaacgtcataacatttgtacttatgacagttataatagcatcaggtattttagctatacttggaatatagagctcatttaagaagcggttaagtagttagaggggatttgtcctaaaatagtataccgcttctatatggaaggctgagaggtcttagaattgaaaggagagatataatgattcatatatttgtaaaagaggattataataaagaaacattaaggagtttacttgagtatattaatgatactgtaggtagggaattaacttatggtattaatacagactatgataaggatgtcgtgattgaaaccgatgaccctatagatgaggaggatacaattgagttatcaggtacaaacatgttcaaggatgacttatgtattcttatagaagagctatactgtaaggcatttgttaatggtgaacctgttattatacgtaagtatgtagaggagatgttataatgattataatatttttaactgaaaaatatgatgccaaggctttaaagaaagtattagaacatattgataattgtagtagtagaggtcttagctatttaatgggaaaaggagaagcggatgtatgtatagagaaaaatgtatttagagaaagagatgatgtaaggattaactcaaacattattgatgaaggtaaactttgtatactaataaatagacatggtttagaatgtagctactatagaggtatatcatgtaatattggttccttcgtaaaggagagattataatgatagagatataccttagtgaaaattatgataagaatttactaaaagcagaattaaaatggattaaagagaccgcttcaagagaactaacttatgatgttaatagaagtccaggattggatgtttatgttaatccctataggtgtactaaagacgaagttgaagaatggagtacacttcctccatttgaagatgatatacttgtatttatagcggagacgtggatacatgaatatcttaagggtgaatcaataggtgtagatagtatggaagagtatgtaaaggagatgtaactaatgtttaaggtatattatacagtctaccatagaggtagtatgaaaactattaaggataagctagatagaagtagtttaatatacttcttgtatgatacttggtataaagatattagtaacgtattccctaatcactataataaagagtttgggagtaagagtgatgatatagatatagataaacttattgaagcggttaatgaggaaggtatattacttatcaatagaggtaattatgttacaataagagaatggtaggataggataaacttaggatagaaaataatttaggatgagttacaataggataggataggatagggggttaagttaggatggatactttaacatacactattattcataaagaatctgatagggtaatagctagcggtttaaatgagacagaaactatgaacttagttcaaaggatgataaatactaatctagttactgatatatcattagatgattataaacgcagaccacatggaaagatagatgtagtcaatttactagtagatattagaagacaaggcgtatttgatttcaatcacatttggcacgtaggataggagggataggatgatagttatatatacagatgtttctaaggattatttaaaagacgagttcttaccttggcttaatgaaagggatagatacttagaatactataaagatgaattacctgaggatatagattcctcttatattgtatcagttgtatactgtaaggatatggaaggtctattagaaagaaaagacattgttcttgataatagttataatgaacctgtagctttattaggtgttcctgagttttttggtaattatagtaattatttctattatagaggagaaagtattagtaaacatgacctaggagaaattgttaggttaaaagcttggcaacgtatgggtggggattgactaagtagctctccctaatttcactaagtagctccctaggaattgcctaagtagctcggtatgattttaccctaagtagctccctctgttttctactagtttattttaaccgcttcaggtgtctatatatatatagacggttggaataatatcagaccgcaaaaataaatacactaggatattattcctagtgtattatataatttttttatagaatatttataacattgtattcaaattcatttacttcatgttgtgatttaattaaatttttaattaatccgttttgtgttttatactcttttattagtttttcattttctataattaaattattaaattcttcttttgttgtttcctcatctacataaaatttactttcatatatttcataatattttttatctgttccgccatctaaatcatctgatatttgataatttttgaatataatttcttttgtttctaattcatttactaataattgtgattttgcatattgtaatacatcttcattgtcccacattggaatatagtttattttcatttaaatcaaatccttttcttataatttttttatataatatttgtagaagcggttggggtttgtcccttgccttactacactttatatattacagtatagttattcagaagtcaatacttttgagtaactttttttaaattcttttttcttctatataatagtagtttttagccctaaaaatgtttttaaaagaatttgcattttcttattgactttattatcatatggtagtaatataaaggtacagcaagggaacagcaacaagatattagaattatataaaaaaattatttaattggagatgatttaaatggatgtaaaagaaattgcaaatactataatggagttgtggcaaatggacggctacagatgtgcagaacctccattatatgaaagcacactaaaccatacacgcacatatacggcgttaattgtttctattaatggaaactatgacacagtgcagatgttccgcaaaacgcctataatgagcatgagagggcaaagccaaccggctagcatgttagttaatgtgattgacgatgtaattataatcgtatatgaaaatgtagtgtacggagttcaaaacaaagaaataaaatttattgaagaaatttaaaaataggggttgcaatcctcaagcatctatagtaatataataggtgtaggggatagcaacacacctcaaaaaactttttaaaaaagttaaagaaaagtgttgacaccttacaagatacatgttattattaagataacaaataagacaagccacctagcaaataacgaaattaaataaaaaaattatagaataggatttgattattatgacaaacaaaaattacttatacgaagaaactcacacagtacaagggcaagacattacggctttcagaattccaaatgacgcaaacggcaacccacgttatgtagtgcatttcatggatttagatattaaactagcagactatgacaacatcaataaactatacggctttaaaaaatatactgctaaatggtttggcggtggtgtagtattccaaagttataatatagcggatactctagaatatgcttacacacaagttaaaacaaatagaattagtcaataaaattaaatatgaagcggttgaatataaccgcttctgaattaaataaaaaaattatataaaaaggatatgataatatgaaatttaaaatagaaaaaaataatagtgatataaaaactttatggaatttagcaaaaaatggatatatgagctatcaaactgtacacaatatatttaaaaatgaatcagatgaatttattatatttaacagtaaacaaacttataataaatttatgaaattaagatataatagaagtgcaatacaataatataaaaaaaattatacaattccctaggattagatttctagggatttttatttattttaatttatataaaaaaattatttaataaataagttagtgtaaaattgactgttgacaaggttgtattttttatggtataatgaagtgaagaccttttttagtataaaaaaattattatataaaaaatttatattaaatggttttaaaaccactctttcccggaacctcttctcttatatagcggacacgtaggctccttaccgctttcttactatacttatagtatactatagaaaaaagaaaaggtcaacccttttctttaatcttttttatatttttttataattcttctaacggttatttcacttatattatattcctttgctaacataggttgagtgtactttctaggcttgtatttgcttgctatggcttttctttcatcttctgttaatgcataacccctattatacctattgaaagtattgtctttatggtgccttttattgtgttctgacgggctaatacattctaggttatcaatacaattattttgtttgttaccgtctatatggtgtacatggtttgatttaataaaatcattgccaaaatattcatatacaagtctatgcaccatgtgttttttattattgacccttacggttaaatatccgtcacggtcttgatgtactttactcaacctatcatttatttttaccctacctaaattagaaacataaaaattatatttattaaagtattctttttttataggtttccaaatttcattaattttattttcaaccatttttttactcctccttttttggtatcacttccattatataataattcggtcttaatgtcaatagataaatgtaaaaaagttttttaaattaatttcattaaatctattgactttaatatcattatagtttaatataaaggtataccaaatgaaagggattgaacaaaatgataaaatttaaatggaaaaacaaaacaatcaaatcaacacaaaaaacagataacattctcttacttattatagggggtttagttgcaacagtcacacctaaacttgtaaactggtttttactactacaagataatataaatatttttttaagataactattgacaacctagaaacaacatgttaatattaagataacaaataaatcaataaaggaaatgataaaaatgaaaaaaatcacaacaactttaaacttaatcggcatgaaaaataatgaaaggtttacagaagagttaaaaaactaccgtcaagatgttactttcttgaaagcaaataaaattgtaaaatattcaaaataaggcttgacaacttaaacaatagatgttaatattaagatacaaggtaagggaagcggtcaaccgcttccaacctaaataaaaaaagtttaaaaaaaactattgacagtcacttgaaaccatgatattattaagataacaaaaaacaaacagaaaaggaattgatacaaatgaaattaatcaatagagataatgaaatcgtaattagcatagcaacacttgagagtgtaaaacaagccctaatttgggagtacatcgaccacttagataataacatcctagacaaagaaatacatgaccaggaagcggttgttattacttcagacactttgcaatcactcaaatttgcggacactatggaagaactagaagaatatgtaaacgacatcggttggaaattagtttagaaaaggtattgacatcctaacatatagatggtaatataagagtatagaaaaaataaaaaagaaaaggatttgattattatgacaaatacaatacaagcatttttacaaggacaagaagcaagcacagttaaggacgtagcaactcatggagtacaaagcggagcaattggcaaattaatctacacatcagacgtagtaaacttctttgatagttacgagcaggacattgaagcggtcatcactgaatacattgaagaggttacaggacaacaatattatgacttattgaactatgagcttatgagagacctcgagaattatgcaaatgtagaatttgaagacgaagacgaatataataacattcaatttgacctagcagaaaacattgcttctgatgaggttgaaggatttgaagacatggacgaagcagaccgggcggaagcaatctatgaggctatggatgatgttgaattagaactacaagaaactgacaaggttcaatatgttaatctagcggttgagattgtagctcaaagaatggcactatagaaagcacacagagaagcggaacaatacagatgaacaataatactacttcatatagtaatagcccatatggtagcttagaagagcttagagaagcttatgacctatcgtcattatctactggtgagattaaagaactaatacaaacatttgtttaaattattttatcaaaacgctttacaatcttttagtttgtatggtataatgaacttaacaaattaaaagaaaaggaaatgatgaacatgagaaacttattagagcaggaacaattagaaaaagatgtaaaagacattatttgggtattagatagaatgattgctaaaggagaacaatacactgaagcttacgatattttagttaacaaattagaaagacaagaaaaaagaatcgtagaaataaaaaaacaaaatggaatattttaaataacaaatagagggaataaaatccctcttttatttttatcctattatataatttttttatattatacgggggcaggggtaaaatgccactcaatgggggtgggtctatatacccctatggtctacccaggtacttattttttgggaaaaattatgaaaataaatatttaaaagtcaacacctaaaatatagaaagtaagtcaacaccctatattaaaagtcaacattataacccctccctgtaagtcaataatttatagtacaaatagacaacctctaaatataaagtcaacatacctaaaataaaaaaagagggaaattaaatccctcctcttaggtattattaacaaactctaattcatgtatagtaatcatatccattccatagaaatctcttgggtctcctttaatgaactcttgctcacctctatggtttgtttcctctttataaccttcttctttaatacgtttaattaagttctccttatctgtatatatcttatcttctctaaaatggaagttatcttcataaggttcacaattatcatgttctacttggtatagtttcatattacttattctccttgattttttttagatagttttcataataagctcttgattcttgtgttaacttttctctttcttctctagaccattgtaatgctccccaaaatttctcataattttcttttgataagtcacctagtggactgcccttaggtaagtctttagtagcttctttttgtattttttctctttcttttttaatttcatctagtttttcttgattacctttaaatgtgactcttggtttggatagtaagaattctagtatttcttcatctgtaaatacctttgcataactatctttccatgtagtatcttcataagagtgtttacctgtatcttctaattcttttaacgccaattttgagtaaccttttgcatctataagccctctagtatacatacttgcagggtctaagaattttatttggttctcataattaataatacttgtttcgagtttaagtttggcttgttctttgttcatacttaatcctccttatacatttacactcatgattaagtaaaagtcatagaatttaccttcatattgttctacattgtatatatgagacattaatctataataatattctgcatcatgtgtacctttaaacgttttttcattaaactcatttttcattagattaactaacttgtctttagttagtacgaatacaccttcacaccattcttcaaactcatcataatatgtctctgcatctcttaaaagtaagtccataaaatatcctggttttctaaagtcatcttcaaagttaaatcctttataataattcttttctaatatatgtctatacctagcgttattaataaagtgctgtaagcctttctgttccacttttctattacttaatgtcacaccgattacttcaaagtctagtcccattatttttcctcctcttcataagaccacgtaccatattctgcattatagtgtgctgtatctgcaccttcatcttcatattctacactataccatgcatcctcctctgtttctgcatctatatatcttacctcttctgtagtaatagtacgttttactttgtatcttttcaactgtttttcctcctttatgttttcctctagtatttgttttaatgtctgacagtctttttggtctagagtatcccaactctctagattttgtaattgataaagtaactcatttacaatttcatcgaaggcttctgcttttttatatacttcttctagttctgtttctgctaattttctattccttttaatttgttctgctttagttactaagatatgggctttattcatttcctctataataaagtttttatagttttccattattatttatccctcctattttctatccgttgttttatctcttctctattgcggtggtgctccttactcatttctttacgttccttatttgttaaccttattctataaacaaggtagttaatgtataagataccggctgaccatagtagcaagaatgatattaaataagtccatgagatactaatttctatcattgtgagtcctccttatattctttatagctcttaatggctattttacaaatacctctatttacagcaacaaatactataaatgataatagtgttataactgctcttacatcccctgtaaaaggtaatgattgaaagagcaaatagttttctaaaacactaatagctgtaatagtagttagatataatatagataataagtaatcctttaattttagtttaacaaatggttttttgtgctcacctgttcttacaataccataaagtattatgaaccacataacaggtactaactgtataataaaatcattgtctacatttaatgcatgtagagcgtaaataataactgcaggaatacctataatgaatgctaggaatacagaaaatataattaacattatagggagggctacaagaaaacctagcccctgttttgaatactctaatgtgtttttacctaggaacttaaaaaatgttttattcatcttcttcctccttggaattactttctgtaattgtaatttctaacatattattgtaataatcattcttttgattgatattatagttatcattgtattcattaaagtctacataaatatattcatttgcgtcattttcataaataatatctatagctgtaatatctgaatatgctgtaatcatttcataagcgttcgtattatcaggataagcaaaaccaacttgaggtatttccataggcttatcaataagaataccgaaataagtacagtgacgtgttcgacttatacttgaagtccctttatatgtaccatagtaatctataccttctgtaatacctgatatatggaacctgcttacgtctttaggctctaatcttacaacatcgcaattttctaatactaaatcaatatatttgatattcattttaactctccttttatattaataattctttccattctttatcaacctttttaagttcttttttattatagtccccgtctttagttactacagtgttccattggaacttttgtaataagctaaaattatttataatccatatattacttttactataatacatattgtcttcaaatcttatatctttttctataaaatatttatatattttatatcttctttcatctgcacctgatattttaataatttcattagtatttaattgagtggataactggaagataacatcttttactttcaataggtctttaacattacctctgcctacatggtcattatagcaatcatatttaacttttttcttttgttttctatcattaactacaatgaatatattatatacgatataagctttaaaatgggtataggtagtaggtgcttctgaatcatcacattcttttcttaggtctgtacattgtatttttaatgtaatattatttgatatgttaactacagtagagccctcatgttttttattaagatttatcttatccattttataattacctacttattgtagatacaatgtactcgaacatcttccattactttgcctaatagattctgacctttccagttactttgctctaatattttagggtcatttgctttaagacctactccccatattttatcataaggtgaagcttctacaaaatctttacgtacatctgtgtctaatattctttgctttaggtgtgtagtcataaatttatctttaactacttctaccataatattatatcttactttattccactgttcttcattaaaattacgaactttacgacctaaacttttagcatggtttgggttcttagcatttagtatttcacctgctatttgaaagtcattaaagtatcttgctttgcgccacataaaggcttgctctgagttattaaatgttcttccttggtgtttaaactttatagggtagaaattagaataaatatcctctttaccccaaaacataatatattcccttgtttctctcataatatttctcctttaattccatagtgatggtaatacaattttaaaattatctaatattttactttgtacctgttcaagctcatcatatttatccatatcaaaatcatccatttctttatgataatattttattaagcttaaaatatgttttatcatatctatttgtgttctttctttgccgtctacatctacaaaagtatggtattccatatccacatgattactactctctataaatgcatttaggtcagcgtatagctgaataaaaaaggacatgtcataattccaatacttaggttcatttctacctagttttttcttcattttcttatattttttattcttttttatcccaaaaacttctttttcaaagtcattcaatttaagacctttaaaatattttttcttcatttcttaacctccaatttaataaatggaaaatcaatgtttctaaatactgcgccaacatcacacattaacatgtctccattaatttctacttctccactgtctgtaggggtgtgaccacatacataggtaaaaccatcttttctaggttgaaagtctcttgaccatattaattggtcaattgtttgttcttctacaggtttccaactaaccccacctgaatgagagaatatatacttgtcttctttatagtactttctacaattaaccataagtattttaaattttctatagtcgtctgattctttaagtttctttagttcacttttaataaaatcataattatttcttagattttcctctacactactatattttaaagttactgtactcacaccgtaagagttaagtgtttctatacaatatcttgagagccattcaatatcatagatacttaatcggtctacgttttccataatattataaaactcatcatcatggttccctaacagagttactacattatcatcattagacattaaatcaaatatatagttaacaacatcttttgaccttttacctctatctacataatctcctaaaaatactattgtttcttcaggttttctttcattatttattttatccataattgttaataatttttggtattctccgtaaatatcgggaacaacgtatatagccatctaatctcctccttattgtatataactatcttaccatacttagtaaaaaaagtcaataaaaaaacacctattaaattaataggtgtttatcatttaatgttattttaaagtatcattaccatgtgctaattttttatcatctattgcatggtcattataaatatatttaacctctatatactggtcttcacttttcagtgcatctactatagaagcattattagttattgagcttgttctagggtaagtaaatttttgaccgtcagataaaataatagtaacatcaacttcaaagttaacaggtagtctgtatccataatcttccaaataattaataaagttattaagagaaaatggtttatacttgccatctaaggtatagtcaatatattcatttaatgcattagtaagttctgattctgttaactccattgtatcataatctttttcgttatagaatactacaacattaggttgttctatactagaatctccgtctttatacttagatataaaaaatccaatatttcctttatgctctaaataatctgctttcataattttaagtacttcttctgctataggttttgctaatagtgttacccattcacctttttctgcgtcataaacactaggtagtacgtttaccatcatttaaatctcctcttcttaatttattggtttaaaccacaatttactcttatcacttggttctgtttcactaactacgaaagagttagaatcaatgtttaaagtattaaaaacaatttcttgtttgtcttcattactttttgttgtaaattcgggaacatctgttaatatagactctttaccattaatagtccatgatattttaaaagacccttggctatacactgtattcggtgtcagtttttcaattataattttagcggatgcacctgtaattttttctgaagattttaataatttacctttggaatcatataagtttaatgttctctccacaaattttatctcctttactatattttgtacaattaatataacaaaaaaacacctattagtttaaataggtgtccgacagagctcccgtacttagattacggttaataatattttacgacaactatatgagaccctctgtcgttgaaactcttgtcactgcgttattccacaagatattttagaaggtagcttgtggaagaagattgtttttaaaggtacaattagcgtttttaagcctattcgatacccaggacactatgtccgtactaactattacgtcaataaaggttctacggtctcaattacctactctttattgttaaaactaaaattaagcttgagtgctctagaagccaaaatcaattaattaactatagatacggaatggaggggcactaccatccggagtctacggtcagatacaaagcctctgccgggcaacatacggtatctctcgtacatcaggttgactagacctttagagtttttcactccttctcttataaccagtaacttaagagaaataggttttacttagtagatatgaaacaataaatccacatacaatattaaatcatagtcaagtgattgcacatatgtctaacacctataagttttttgctagcctggtatatggactctgcaggattcgaacctacagtcaaaccgttatgagcggttggctttacctttaagctaagagtcctagaaatatcctgagagaggactcgaacctcaacgactaggtagctacatctagccaatgccattactcaggattgctagtaacgctaaatagaattataacgttaccgtagaccttttctacgcttggtagataggtaaaatataatgatttcaaagtacccatatagttaggctcttattctcattataaggttaaaaaggctaactgtgtttagcattatataagaggctttagttaactactatactaataatataccataaattatacttaatgtcaagttaatttatcaattgaatctataatttttgatgtgctacgtatatctgcttctttactatgtttaaggagatattttaatttcatttaaaaaagaatttttttctttttctataatatcttctttatcatcatattctgaaaacataatgaattctatacctatactatttctattatgtgaaaacatatttatagaaaaaggtgaatcaaaatttttatcatctttattaatactaaagtcttcagtaacctgtaagtcatttatttcagatatttcaaagtaaccattaactcttttaagttcaatataactattgtatctaaagtaacgttgttcttctattaacttctcttttgttatataaggatattcatttatgaatataggattacttgttccatagttatctctaatatattctgcatcctctagggaatcagtataacctaaaatttcataacttgttgtatacactgtatcttcttcccacaagtcatagtccatttcctctatttcttcttctaatatataaatttttttcatatattactcccaaaccccgataagatttttaagcttagctataacctcttcttctgtttgataagaaaatacccctgtaatatgttcatagttacctacaatttcataatcttgtgtaccatgtttatctactaagtatgagttattcataacatttaaactatcttctgagtaactaaaatttatgttatagtctactaaaaaattaataatatttttcatttacataacctctcctatcggatattgtcctagcattcttgttccattttcattataaaaagtatattctactacaataatattcatcatatctacatatatagcttctatatacggtgtaatattttcctcttcttgtatgtgtttacctatgatatcatataataattctgagtgtattcgtttatctctcattatagacctccgtaaggaattctacagttttgtctttcaaagatttttctactaattccatagcatctttatagtgtttgatattagattcattagacttaagtttatcttttacttcttgaattaggggctctactttattaaccaaatcttttttcttttcaatacttacattgcttctcttattgtctaatacttcttttggcatatatttaacttttgcaaagtctttatagctaacatttaagttatctaaatcatctaataaatcattatagtattctaaatgattatagaatgtataaaacttaacaaggtctttaccagttaattctcctttttttagtatattattaatattaccgataacagaatatgctataggcttaaaattagctctaacataagttaaaaatataaaatcatcataaaataaatctaaaacagttttattgaatctagtatttttagcttgctctaattgagcacataaattaagaacattatcaaacccactttttagaactaaagagataaatctttctactgcatagtatcttgatacttctgtatgcttacttgctttttcattattcctaaatatagtatctgataaaggttgaacaactaaactcatgtaatctttatctgaatgctcatctgatgttccttgataagtacttccaaattctattgttgataataagaaacttttttctaagttcattataacatcctccttttatttgttatttaaataataacatatattgataataatgtcaatacttatatatcttcttctgtatcaacttcatcttgtttatacttaaagtgttcatagactttaaatagtataatccctagtgttattaatcctaaaatatatttcatagcaatcctccttaataaccatgtttagttacccaccctgctaaagcatccatagctatatcatattcttcttcatttttaattcttataattttctctatttcttcctttgcttgcttagaactaataaaatcaatatcagtatcctctaggttagttaattctaagttttctctaataaaattcttttgacttggtgttatagaattaactcttacattttcgtgatttagaaattggtagaagtccatattactcatcctttttaacgtattctgccatatcttttaaaatacttagtacatactctaaatctctatattggtcatctaacgaacctataatagcatatggtgtcatatcccaggcatgtgcacagtcaaaccctaatactctcttaccctcatagtcataatcatcgtaagtgatacctctatgggcacgtctttctaaggagtcatattctttttcattgatatctgaaggtaaagttatatatccatttagatgaccagtttcagggtgtctcttaacagttagtttaactcctttataataaatatcaagacttaaatcttctcctagaatattgttttctttttctactttttccataatgtattgaggtgcttttttaaacataattagtcatctcctttttatttatatctttactatacactattttttatattttgtcaacaaaaaaaggctactaattaaagtagcctaaatattaattatttagcgttataagaccaacgccaataaccatttttctgtgagaactcaaagtgaaaaccatcatagtcaaattcaatattatagtctccatcttgaagtggttttgaatttagtacaggactattactctttgccaattctgctagaaactcatgatttactttttccatagggtttattcctcctaattattcttacagtactaatatatcataggtctttttctaagtcatttttaaaagtttcctcgtaagaactagcgtaagtaacctcataacccactacgttagtatatcctacatataatgacttataattagattttatcttaatatcttctgattgttctagcttatttaagacttcacctaaatcatctgaagaatagtgttcattatctattgttattgttttaccttgggtatagatatcaatttcttgtatcatcatttcatccttttgattattcattatttgattataagtttctaaatcatcaatgttatctgtatctgaaccttttactaaccattctcctctcttcttaaggaggtcatcaaacttctcatgctctttaattatcttttctacctcacttggtattaacacagccctagcatagtttatatgccacatagacatattatcaataagataattaaccattcttataatctctttttcatttgccatataccaacctccttatatctattactaatataagagaaaagcagacttattaaaagtctgcttctgtacctaattctaatcttctatttttcatatgaggaatcatttttttatctcctgttaatagagataattctctagctttttctttagataatgttagtagtccattataattatctactttactattatattgtctgactaagtactctagttcatcttctatacctgctagttctcctgatttaactccaagtaactttctatacatgtcataatcttcagaaagactttctactttgtttttagatacagaatcataaactgcttgtaaattaccttcttcaataagtttaaaattatattcaccaatgattaattctttttcagaagagtcaagggtaactaaaccacttgtattacctgtaaagtcacctttataatctacaacaattccttcagttattttatctcctaattcaatagttccatcttcattttctttaaatttatgagcatcataaacttctactttatcacctaatctcaaatcttgagttaagttatgtttaccaataattctatccattacttaacctctcctttattaatagggtcttgtgttaagaacatttctaagttctcttttgtaataggtaaccaaaaatatttactttccggaattgtaactgtatagaagtcttcatcattattaactttgatgttaacatctgtaaactcatcttgcattaaccaatgggttacagttaagttatatgacccatcactaacatatcctaaatcaatatcatgtctaaaagccaaatcttctaaatgttctaataaatcgttcttttcattatgtttttcttcttctgtattatttttaattgggttaattaattctgtacaaacaatatcatacaattcaccatctgtaacctcatagttcttttcaattaatacatcttgtattttattgattgaatttgtaactactttcccatattcttcttctgtaaacttacatttatctaaatcaacatctgtaattaattctgcaatccatttatttaaaattgatactgccattgttcgagaaataatactatcgtataccatatttatttaatctccttatttaggtgaatgtggtcttctaatgaaaaatcaaaaggcgctacaccatttcttttattatttgtttcttttttaagtataacataagttagtgaaaaagtcaagatagttactacaaccattgataaaagtttaatcaggtttttcatagttactctaactccttaagtttattttttactttctctttatcgtacttataatctttactagagttttcatttttttctttctcttcttcattaagttctctatactgagcttcttctacctcttgttctttattatcgttatcttcttctgctttttgaatttctacattcttactactaccatttaccttttttctaaaaagaaaccaaagtattaataaaatgatgagtaaaataacaatgctcaatacaacagcccaaatattattagccattacaacctacctccgaatagtttttttacagctcttaagttttcagatgaatcgttatttatatcaattcctacgctagaatcaaaaattacagcattatcaagtatatgctctgttaatttattaccataactacttttacttaccacactaccataaccatgattagttaggtcaaccatatcaggttcaacttctagtactctaaaagatattctacgtaagaatgaaggatttactaagtaaaaggaagatttaaaaacatttaatctttgataagaatgttttatattaacaacaaaccctgttaacttatcttcatatcctgaatttgataacttacctaagtaaaggtttatactatatccttttgtttctaatgtttgaatagcacttaacattatagcccctctgtaagcaagattttcagggtcttccatccaactaatactagaattataaaatacatcaataactttcttctctgctttaactctttgctgagacatcatagaattaggtaacccttttatagcattaggtacgtgaggttgatacccttccggagctacgacaggttttctttttactgacttatccattctaaataatgcatctgtcatttttttaagtttaactaccatatcatatgactctctatcacccttaaccattaagttataggcttcttgaaaactatgagtccctgtaaaatcatagctacctgtatctgatgaattatctctacctgaaactctattcttttttaaagcagaaaagaaatcaggtagaccatcatatttaattacatttaattctgagttatctattaatcgtctacccattgattttcctcctattctaatcctaatttatccataattgtatcaaagtccattgaatcttttgatgtactatcagattttctaggttcctgcttaggctcttgttgcatacctaaaagctttcttgttgcttctgtgtatctgttaccctcaggtaaagagctaataaattgattaatctcatctttcggtacagatttaaagataatactttctacaacaaactcatcttccattactccatctaatttactaccattaataattgcacgcattgagaatacataaggtaatcctttttcatcattctcatgtcttaattgttgtacaaagttaactaggtcttcattgcttgataactgatgttccaccttagtatcatagtcaaattcaacttgagcaaagcggtctaatgtagctccgtctaattgttgtctacctacataaatatggtctgctcctgttcccatagtattacctgctgacacaactctgaaatcttcatgagctgttacacgtccaatagggaagtcaaagtatttatttgcaatagctgaattaagaattaatagtacttcaggaatagatgcatccatttcatctaagaagaataacccaccttttgtaaatgctttatagaattgggtttcatgaaacttaccatttgcatcaataaatcctgttaatttaaattcttgagtaattgcattactgaaataaaaatctaaatctagagcttctgctacttgttctaatacatggttcttacctgaacctgctccaccttttaaaaatactggaatattttggttaactaactttagtatatcttggtatctataatggaagattcctgagatatctttaattgtttttccttcttgttgtaattcaattttaactggtaaattactaagttgttcttctacatattcttcaatttgttttttaacgtcagtaataataatttctctactctcagttcctgctttctcaacaattgcatctacaattgcttgttcatacggattagagtttttctctcctagtttgtctgccaagtcttttgttgtttccatttgttgttctaccaatctctctaatctttcaatagtatcttgctttgccatatttatcattctcctttgatttgttatacatttattatattacaagtatttgtatttgtcaacaactttctaaaactttttttagttgttaataaaaaaaataccttacacctataacttaacatagggtaaggtaattgtcaacacttttattaaaaatacattaatttaaaaaaatcatcaatatctttagtttcatgtgtatccatatcatacataaacatacaattatatgtatgattattcattatttctaacatgttatgcatagaagttgcattattgaattcctctaaatcaatagttaccgtaagttcttgaccttcataaagtatgtttgctatataatatttcctaacaccttccattgttccatgagaagtttcattatgattaagtacttctacacctagtgaaggtaaatattctgaaaagtaatatttacagaaatatataaaattgtctgttcttttagacacgagtactatctccgtactttatatttctttctaatcgtacataatatgttttaattttttgtacttctttatctactgcatcctttcttcctaaccttgtagtatattttacaatattaaatatcatagaatcaacaaagccatcataagaaaaatgttcttctagaaaagaaataacatccttactacctttatagtgttcaggtaaatgtgcatctacttgtatattataataatcttctaaaagacctatactttcaccaagactagataaagcgtaacctaaatcatttgaatcattagaccattctttagatactgatagtgcatcttctataattgttacttttaatttatctaaataatcttctacttgagcttgtgttttcataaattcttttgcgttcatgtaataccctcctaaattatataaaaaaaaacaaccctgcttggctacaagcaaggtgaaaaaggaaagatattatggaagtgtactatctaagtacacctcataatataacagttttccttgctagttattacttattttttaaggtcttcttctttgacaaacactccattaataagcttaccttttctgtcttttatctcatcataagccatatcaatacactcttcaatatctatatctaactgtaaacatagtactgttaatactacaaaaatatccccaacactatctcttgttacatggtcattacttttagcaatacctgaagctaattctcctgcttcttctaataactttaacatttgaccttcaggtttacctgtttgtaaatttctatcttttgcccattgtttaataagttctactttttccattattctatatctcctttaatttctgtatctttgataattaggttatcagagtcacttgttacatttaaattatcttcaactaattcatgtagattattagtaatatcttcttcatacctataacctacacgaacataagctttaactctgatatctatattaacataatcttcttggaatttttccatttctaacttcctttattatatcatattatgatactattgtcaattaatctgagtagtttcctttagcaagttgatactttttgtgtaattcttcatataattctctcataccttcatagtttctcatatcatcttccaagaaactaaggtaatctaataatacttttacatcctcaggttctaaagttataactggttttaccattaggcaacctccttaaattcttctttatttattttcttgatatctttttctaatgcttcttttaattcattaggtaatttataggcatcaattgattgttgttgacctaatacataaccattatctgtaatacgtatttccactgtaaaccatgaattatctaaatcttcttctaatcttgctaataatattaaacaactattttttagaattctgttagcatacccaccaacacaatgagataacattttaccttcatctttaagtttacttacagtatctgcaggaaggaattttacttttctaccatcttttaatttataagttttatcaattattttttctaatttattatcatatttagctttaagttctgcatcatctaattgttgttgtatagattgtttctcatctgtaactatatcatgttctagttttagtgaaaatggtgttaaactaacactctctaatgttctataaccttctcttattaatattgataaatcatgtaagtaatctaagtaatagttatctagtgcatatcctgttatacgttgtctgtcttgagcatctacatctaaatagtgcgtcatctttttgtaattagcaaaagatatagataatatttgattcacaataggtttaacttttaaagcatctgtaacatctcttgaatctctaaccattaaaaaggtatcgtcaaacaattggtgtaaattaacttcattgtgtaaatgattatagtgcttatagagaatattagcaaatcttaagtaattaccttgctcaaatttatttaaagttagtaactttttataagtctgttttgtaagattgaaggcttcatgaactttccatttaggttttttaggtatatggaatagtaatgcatttctttcaaataacccaaattcttctaagttatttattttatcaatatttttaacaatatctgttaaagttattaagtaattagaagttgaattttctcctataaaaatcctatatttatctcctctataatttatatgaccataaacatctgtattatcaggacaccaactagaaaaatcaaaattatggtgctctaatgtttgttctattatctttattataattcctctagttaagttaggttgtgcatagttttttaaaataacatttaataaaacagataaagttaattcattcttatattcacttttactaatatcatctttatataggtttaaagttatttctttattaacaagactatctgttaagaaaaccttaacttctcctgttttaacgtcaaatgaacttttattttctaaaacccatttgttacccatattatgtttatctctaatatgtttaactttaagaccaaaagatgaatagttttcagtacttggatgcatgtaccaaacacggctatataattcatctgtcatagcactatagtactcactagaacctttttctatatcttcattcataacaataatagatgactttataagaccatatttaccttggtctagtacatccataatatcattatttaaactatctactacttttttatattcttctaattgtctagattcattcctccataaatggtcattaccttctagttctttaattttttcttcaacataaccttttgattgtatacgtcttctactcttatcaccatatacaggaaaatcttttctttcttctctattagattcaatatactctttgtaacttcttcctttattatcatcaactacaccttcaactaatttttcaactgtttcataagggttaccttcaaagtttgttacttctttattaccacatagggctaaaaataaatgtatttctgtggctgtatcaaaactaaatatattatgaatatctctaaataattctttagaacctaagttaattatattatttttctttttcttaagaaatacatcttcttctcctatatagatacatcctttattaaccttaggtaaattaataatttcttgttctgttaatcctttttgtttataagttattgccatttaaaatcactccttatttgttatgtactaatcataccatagtaaataatatttgtcaacaaaaaaagaagaactttttaaagtccttctaaatgagtttcgtatataactttttgaattttatttaatggttctaaatctaaattcataataagttttttatactttcttgaatttttaaaattgatagtatttggcatagcaagagcttcatcaacatctttagtatagcttacaacatctgaatagatatctacttcttttacatatagaccttgagttaaactcctaaatactacctcattatgtgctataacttcttctttcttttctatgctcatttataaacctcctggtctactctacacaaacaagtacgtattctaaattagttaaagaaactgatttaatattgtttaattcttgtaatttcttaatttccacatcatagttcttacttatagtccataatgtctctcctgctcttactttgtgataatatttatttccctctttgataaggtcattcaatattacctaccctcttgagtaataattagcttgtagataacatataagtataagaacaaagtttacaaattcagtagctataatatgaacataggtatgtgttaaaaccatacttacaattaatgaagctaatcctaatccaataataagaaatagaaatctatttgttccttctgcacttttagttttataaaaggttgttatctgagttacatacgcaaggataatagtaatagttgctacagtttgtgttaaggctgtaaagtcacttaataaaaatagtaacagtgagaacacaataataaaaggtatagagaaatagtccttttttctatatgaagctactaataagcaaacaatacctaaggttaaattaagacctacagatactatttgaaatactgaagcatcagttaataataagttgtaaaaacttatacctactgtagctacaattaaataccaaaaatagttactaactcccttgacactttctgctttaactaatgctactaaacctggtatatatcctactgtaattaatatagcatataatatacttaagtaatgtgataaattatccatcttgttcccctaatttctctaatctattaataacttcttcccatgaaataaatccttctccgtctgttagttctaaaaccataccatacacaaattggttcgtactaaattcagctctgtcagggtcattgtatggtttaccatgtccttgtctaatatccgagcagtagattaatacgggtttatttacaatgttttcaagtttatctactattaattcttcttcagtatttaatgacgtttcaaacttatttgtaaaataattaaaatactcttctccattatcatatatatggttaattgtttcttctgcttgatgtttcatacctaataaaataccgagttctgcaattgttcctaatccttcattaaggatatcaaatacaaaaatatctgattcttgcatagccttaaagtcattagttaaaatacgttctgctagcttagtttgttctgcattagctttatcatttattgacttatctttgtgagggctataaggagttactcctacaatgccatctacttctttatgttgtttatctctgtaatctaccatagcttcatttaggatatgtccacccatataaattactttgtctttaattttattacccatctatagtatctcctttttcttctaaaatttctcttaaaatatgtggcatttttttcttaatttgtttatctactattttcagtatattttctttttcttcttccataatatcatcaacaaagttttgacctacttgtttcataattagaccgaagttttctaattctaaatcatcttcagataatctatcttcttctatagccctaaaaatcattttttccattcttgctcttgtaatggcataatctgccactgactcgttcttttttacttctgttttcattttttgacgactaaattctttaaactcattagatactaatttaaagtagtcatcatattctgatttaccatctaagtatttaattactataccttcacccttatcaggtttaactgtcatgtcagattttcctactaattcttgaatttcttcaggttttaaatcatttaagtagtgagatggtttagatactagcaaagttttaactgtttttaaccctaaatgatgtgcaattacattcatgtcttctactgataaataaacttcattttctttatcataaacatcaaatacataaaaattgttgtaaaattcttctttgtactgaatcttatgtttgactaaccattcaccaaaaataatgtatttttctaaggctgatacgtacgtatttcttacatttatattttcatgtacccaatcataaaaaccatttaaagtttcattctcatttaatttttttctacgtgaaaaacatactaattcaccattttctactgtgaagcttgcattacttccatctaatttttcttgtacaactagacctctttctttaaatttatctagtacaatacctttattttttactttagtatacgatttcattaattatcctcctttgaattatgtactatagaaaacaaaataagacttacgcttgctaaaaatgctaatactactaaaccaggtaaatttaaaactgttgataagaataatgatattgcacttataacataaactagaccgcttagaaataaagttaataatacaattgttataagtttcaccaaccaattgttattaataaataccttagctaaataattcataaaaaaactcctccttagttattatagaataattataccataactaaggggatttgtcaacatattattttaccatttaaaattatctgcatattgtgcaagcttagagcggaaattaactgtaaaattatgaaatactgctccttcataatttttaaagtattccatataatctccaaaacctgatttactttcgttctttaaatctatttgtttaaaattaccttctactattacagtagaattttttgtatgaacccttgtaagaacttttttaagttcactacgtttaaagttctgtgcttcatttataattatagtagcatctcttagatttccacctcttaggaatagatgggatatttgagatacccaacaatctcctagtttatcttctttaacattatcttccatcattaacatttcagttatttgttgttcaggattcatattaagttcaataagggcatcatgtaatcccatgaaataagccatttctttttctgtctgattacctggtctgcttcctaaatcttctgatactggtgaaattataaatactagctttctatttttattaagatagtctgcgtaagcacaggctactgagcacattgttttacctgtaccggcttgactctcattccaaagtatttcaacattatcattaaagaaatcctcacagaaatctaactgctcggttgtagctttttcaaggaattcattaaagactagatgttctcccatgttgtatcttacattaggataatcttttaacttaaagtctaactcttttagttgtattgccatattttaaagttcccctatctataaatagttttactctcttttaatatagtactaatttccgatatattctcctgttgaagagcaataattactacattcacattcagggtagttatcacaaacatcttcatcttctacatcatcataaccaatatcataattattataattaaaatctacaatacaattttcactattacctttagataatcctgtataaataatatcatccacagaatcccaatcgttatctgccaagtaatttacactatctagtactgattcattatcaggtaaataaatactaccgtctgaaaatttaattaaaatatcaccttgaggtaaggtatcattaattaaatcaatctctgtttcttcttcaatagtgaatacagttccttctaatctttccggtgtagtatgtgttaaatgttttacagtatcccctgattcttcatagaatcctactgcattcatatctttattatattttgcaataaatttaccattgtcacttaccaaatattgactagttgcattatagtcgtttgcgtcatctactgtcatgcaagggttataatctttaacataataactaattttcctaacatctgctgtttgtactttcttaccttcacctttaattactgaattaattttcttcataatattttctcctttttatatatcaattgatttttttgcaagattatcggcatagtcattccatttgtcatttgaatggctctttacttttacaaagtttatatctattactttttggtattctcgtatcatattaatatatgttttacttagaatatttcttgcagaccaagtaccttcataccaatgtattaaaccaatataatctatataaactattgcctgattgtatcctagttttatagcctcttcaataccataacaacaagccaatatttcacctgcaacattattatactttattaatcctggtttgtcaacacttttactaatttccgatattatatttccttctttacttaccaagacagcacctgagcctactttacctttattatatgaggagctaccgtctgtgtatatatttacactatcctgcatacttataatcctccataaattgagggaattcacaatctgaatagacttctctgcaaaaagatactgagatatagttaaaatcaaaacatttgaaacagtgttcttgaacttcttttttatctttagcaatcacattaaatttaaaaccatcagctattactgtaaatactccttttttcataaaacaaatacctccactaattttattttaaattaataactaactcaataaatgatttaatagttttatttttaccttcatcaatatctgaaaagaaattaattaaactgtcatcctcatcaaataaatcttcaacatcatcaaatttatttaatatgtctgtaacactgtaaccctcttctgatatatactcatgtaagtcttctccatcttctgacagtgttgcttctattttaccatttttactttcaattaaatataaagtatttaacactttaacagaatctacaactacactgtagttactaatagtaggatactctgtataaagtatttctacattagtattcatataactatcaattacagagttaactgtatctctttttagctcagatacattatgttttcgtatagtaggaaattcttcatcatattctactaattcttttctatctgtattcaataacttgtctaaagaggacaacaatactattttatattggttatcaggaagactgtctgtaatttccattattgttaaaaacgtatcttcacctagaactttgtttatatcttgtaattcaaatgaatctaccatttcaatagtatcatctatatcatctgtagtcattaaaaaattaactaaattattattctccatcgtcttcctccaattctttaaataactcttttcctggagtatttaacgctttctctaaccgcattaaattagcacttcttggtttcttttttccatactcccaataagatataagagagtaatgaacacctatctcagaagctagacttcttaacgtatgtcctttttctactctaattttttgaaggtttagaggtttactttcctttttttcatccataattatttctcctctacttttaaaaatttaaaatcctcagatgcttttgcattttttagtatatactcttgtgatttatttcttgcctctgccttacttttagcatataactctatatgaaatacatgaggtttttttaaagacggtgactcgtatctccaataaactttaaaaagtagtgtttctttttttaaaacattaattcgaaaccatcttttaaatttattcattcattatcctcctctatttatttgttaaactaattatagcatagttaacttatgaagtcaactataatatacaaaaaaagactaagaaattaatcttagtcttaatatattaataactattatgtgcgttgtggtatgcaagagctcctgatgttgaaccgtaacggtcaatcatatattgttttgcacctttagtttgttctgctatagaaccaccactccatgatttacctaatccttggaataatccttgagctcctgatgatgcattaacagcattagggttcattgtagattcacgcatagcaatttcaatcattgcctcgtctccacctgcttgtctaatctgttctgctacagagcctcctgtagaactagttgattgtgtaggttgtttagtttccttttgaactggtgctgatgttgtttgtacttcttttttagtatcttgtttattttgagtatcaaattgtgcttgttgttggtctactttttgttcaggtgtttgttcttctcctgctaatctagatactgtattatctacttgagttgagcctgaatggtattcataaccaaagttaccattataattatagaaatgataagtaaattcaccatcactaaaagagaaatcataattacctgcttgaattggttttgtatttacttctgctgaatttgatttagcttgttctgctaacttattataatcaatttcgtctgcactagcttcatttgtagcaatacctccaaaagtaatagctgtacctaatgctaatgttgcaaaaattgttttcttcataaatttaaaactccttaaataattttttagaattgtttatttgtaaaccgacataagtaatcataacatatatctttaaataacgcaagtataatatagcactaattagtgtaatattattaaggttttattacaaacattacagttatcagataattaaatacaaaaaaagagaggtaattatatttaccactctccagtttcattatatttatttattacattatctcttaattctatagcctcttctaaagattttgcactaaaatatttaactttattttttcttacaatttgtatattatatacattatttgattttttgtatatattatgtgtacttttctttcttatattagttgagttttcagacctagtagaccacttaacattaccaggttcataattaccatcattatttattctatctatttgataattttcattcggagggtctcccatatagtcgtagaattttttaaaatcattcttccattcttcacatatttctataccccttcctccatagtttttataatttatagcgttaacatcgtaacaacgctgtttcatacctaaccatctttggtacattgggttactggataatccatgggtagtattcctttctctcattaactcactatgaattttattactctcacacccacaagattttattttaccttgccttaaagtggagcttctaactattattacttctccgcattcacataaacattcatacattttacatcttgatttatccctcttactagactcttttataacttttagtttattaatagtttctccaatcataatatctcctcctataataaaactatagcataaaaaaccacctatgtcaataggtggttaaacatattatttatttaagtttgtaataacactatctgaacctatactaataggttgttttccattccatttttcaattaattgttgttgtaaaacttcctctgttaaggattcacttctaatgtcattggctttcttttcaccttttgcttcaatttctttctttttagcgttttcttctgctatttgcttatcaactttagtgcgctctagttcttggtttgcttttactctctcatcaattgctttttgagtatttttatctgcagttgggcttgataatgcaatatcatcaataataaaaccttgcttttctaaattatcattaagtttatttaaagtatcttgtttaatttctcctgtttttacaccaaaagcatcaattacagagtacttagaaattgcttgtctaacattatcttgtactctagaacgaagataccctttttcaagttcttctatgtcagcacttccgaatctattaaaaaggtttactgccttagttgcatctactttataagatacatcaatgtctaatttaatatttttaccatctgaagttgctacatttaaatctttatatttatgtgtttgtgttttagttgggtatttatttaccttatcaaaaggtgctgttaaatgccaacccggtgatttagtatcttccttaacaccatttactgagtatacaactccaacatgaccttgtggaatcttagtaatacacattaataaaataataaaccctataattgctaaaaaccctaatactcctgaaataactactgactttctcattacatttctcctttttctatttcttttattaagctatttaaagctttttcctcttggtctatttcttgtttatcggctctagttacaattgattgtctacggtcatttaagaattgttttttatactttacatattgttctaaaccgtattcatctaatgtaccttgcctaactaattccctgtattgttttcttatgttactcttcttctctttcattgaaagaaaatcaaataaataacttataccaaaacctacaaggactagaaaaacaataaaaatagcaaaatatgttaaaagtagtgccatgtaattcctcctttatttgattacatatataactatacactatgtatttaattttgtcaacacttttttgcaaaaaaaatagacggatttaaaatccgtctaaatttatattctatttgaatactccccaggcaacgccaggtatttgattaggtggaacaccttgacaagttctaacagggcaatatactctgttaccgttgtaagcattataacctatccaaatgtgacctgcttggatacaaacttcgtcatatacaattgtagcccctgccggtaagttaccgcctactggagcatttaagaatggagaacctattctagttactataggttggttaccattgacaaatgttgcattttccggtttataccaagttccgtactggttctttttccaagaacctgtaactggtctagttgccggtgtacttgcgctacttgttttaccatctttaactactgtagaacttgaagttcctttatccatgtagtttttaatttgtttaatgaaataatcttttaatttattcattattgcttgtgatggtcttccttgtgttactggattaaatcctgtatgaagaaccatagaacggtgaggacaggcagttggtacaaattccatatgcaatcttacagttttacggttaggagtaagaccccattctttaaatttctctgctgtaaattggaatactgcttgttcatttttaaggaattgagcatcactagcactcattgattgacagacttcaatacctgcaaatctaaagttacctgagtttgctcctgttccatccgattattcaataaaagacgtaactcctttaccggttctcttatgaactccttatagtttcctataagactagactatatcttcaccctaattttgttaggggttctccatttcgatttaagggattctcacccactccattaacttgagccctactcctattgacgatttatttttattggcactcgtccgagggatagtcgttgaaccttaatattgtttccaaatgtatccgtaggctgtttttctctctcctttgatacatcttcctatattaccacttcttttattagaaatacctaagaactctaaagcctcttgagctgtgtaaaagacattcaataaattaccttctaagtcatactgagctatattataatttatttcattttttaatcctgtctttattgcatgttctctgttctcggaattagaaacccattctaaatttcctacactattatcatttttaccatttaaatggttaacttgttctttattatcaggattaggtataaaagccatagcaactaaacgatgtattttaggtgaatggtatcgtaaccttacaaacaagtaacccttgttatttttttgaagttttaactttttaggctctttacctttataagatattacttctcctttatcagtaatagtgtaattttcatatatttctaatccaggtatttcatttaatttcttttccataataacatctcctttacttaagtatataggaaagttattatgttgtcaagtagttttttaaaacaatattcttggatgctgattagcatagcttgatagccttagcctcccagtcaattaaaagaactttcattatacattactgtataacagggcaatttatttacccgtgtgccaagcaatttgattcttagcatctattgcttcccatacataaccttcagagccgtagtaatgagcaataccattagcgtatctagcataacctgcattagctaatgaattctcgtattgttgtcctgaagaacgacctgcatcgttgtgtattaccattccttcaggttttttaccacgtttatccattgtatagttaatgtgattcttagaaacttttagtgttgctttctttttaggtgcaggcgttttacttgcgcttttcttagctgtttcttttttaacagtagttcctgcttttacaggtatttcaatgaagtgagttaatccgtaataattatctacacgttttgtaggttttttattagcataaccattccagttttgctctaaaatagtaaatgtagaagtattacctccatcatatacaatacctatgtgaccccactgttcataactaccggatgtaaataccgcaatccaaccttttttaggtacagtagaaggtttattttcatgtattttaaatccagtaccataactctgtttaatttggtctttagcattaccccaagttctaactttattatctgttaaccataaaacatagtctgtaataaggtcttgacactgagcgtgatagtaaccatctgcatcaatggctcctgcttccattacaccaaatgatgggtcataacttgtagcttttttaactctgtaagggctatctactgttccttttgcataagcatctaaacgtttatttatttctgcttgagtcttagccattacttaacttcctcctctgcaaatactttaccatgttcctcggtatcttcttcatcttgagaaggtgctgaaccaccatcaatttcatcttcaatagcaggtacttcatcactatcatctgtgtcaggttctgcattgttttcgtagctgtctatctcaaaagtactagtgttatttgcatttgcttgccattgaacgaattcattagggtctttactatcacgaggtttaagatagtctgtttgaacaatatcactatctttaagacctttagtattattatcaacaataatacctaaacctgctaatagtgttagtatagaacctacaatatttacaccttgctcaatttgagctgagtagtctaaaccgaaagcacctataatttggttagcaaataatgctactgctgatataattgctacccaaaatgttttgctcttagttcttgtgctaaggtttattcctccaacaactttaggttgtttagtttcattagccattaaaaaaccgacctttctattatatttatttctaacaataatataacagtaggtcggtcatgtttatctatattaatttaacacttactcattaatttggtttagttttttgataacttcagacatttgtttgttatctaaatcttctaatttagtttcaggaagtagctctaacttatcccaaacttcttctttattagatactttattattaataattgccttaccaactaaactttccgtataatataattgttttgctgatgccatttgtatctctccttttaaatatgtaaagtatatagctagtatcgtatcctaggaacaaacacttgcgctatatactcaatgaaatcctaccctcattcgaggacacagcaaaccggttcgtcaaccgcacatatgaattctcagatttcatttatgtaaaacacaccctctttgatttgcacaaagactaagggttttggagacccttgtactactaattatactaagggtgtttattatggtttctattggatttgaaccaatgacacctagagcttcaatctagtgctctaccatctgagctaagaaaccttaaaacgacccatacgagactcgaactcgtactctctgccgtgacagggcagtgtgttaaccagttacaccaatgagccaaaattataatgctataccctaaccttaccttaatgtatagcaggtttttatataagctcgaagcaacgattattaccactcataacaactatatattaagtgaaaggaggtgaaatgaacaaaacgtggtaattggtacttatataggaaatatgtataatctacaaggagtaagttattggttcataaaggagtgtgaacaataaatacatgaaagagtgaaagtttactccctgtagattctttttttaattatcaatcaaaggaggaaactgataattgttaataataaactataaagaggaaaatatttatagtcacattctgatataatgcaactaaatatccaagcataacccgtctcacgaggaacctacctataagacctgttattaagtgaatcactacgattgactctattaaggagctaccttaagtccatctcacgcaatttaaaagggacttacaaaccgtaaaacggtaataagtttattaaataatgtgatattaacatattagttaataactttcacatggtcgaagaaaagtaaatttatttgattaccaaattatttttatcaaatatagctcttttgaacctgtagatttatgctacttatactgataacctctattatctaacacatttctgtgctccaactacagttagtcgttacagcgtatctttctaggattccgctaagaccctaaaaagaaattaaaccctagccgttatcatactctacagaccttataagtaagtaccaagtataccaatcgtatttaacaatactaatgacgacccatcctaccgatatatctccgataggttttgattcgtttgattatcttgtaccttatgactaccaaatcattattcagtcactatgctcagatatttagttgtattatttatatattaattataacataatttttattacttgtcaagttaatttcaaaaaaattatagaagtagggacgtttacctacttctatttaatttacacaaggatgataacattgttattgttttatactggaaaacaatgtaataaaaacagtgatgtgtaaggtatttgttttattgttaattatattatagcatatactgatacctttgtcaagttaatttaatactttttttaaaacattagttatcttttgttagttcctcctgaatagcatcccatcttctttctgcttcactacgattatcttctatatgttttgtagttttacaacatttgatacaatatatatctttgatatgaccttcttctcttttatttgctctttttcttggtactttgaatacatttccacattctttacatattaaacttgagtaaaacattttttgtcttttcataattaatcaattccttttctcttttatttgataatttaactatatactatattgataaataagtcaacagttttctaaaaataatttaaattattttgaagaatactttaatatcaagggttacaagagaaaaagtacgtatttagaaaataaggagtactcctattatatataattatattctgatatagagtaataaataatattaaatatataattataattaataaggttgggaaaattgatataaacataactgatactgcttatagatactcagtataaaagtaaaatcccttagtatcagtacttacaagagaaaaagtacgtatttagaaaataaggagtactcctattatagttatatatatttattactattattaattactatttaaatatataattataattaacaatgttagaaagtcaacaatagtataaataaaaaagtgactacttaaagtcactcaataattagaatactattttaaaagattctattctgtttggattaatatatacttgaggtgaagttatagcactttcagtatatacttttatagaggtttcatccattcctcttaacatataatctatatcttgcctattgtaactcttttcatcagtagatactaaaaagtatttagctccacttgacattgttatttcaatatgttttgacatctacaatctctcctatgcaaatttgttaaagacaaaggataatatagctcctagaacaagtaaaagaaccttctcagttgtatcctttttctcagtatccttagtttttgtactttcagcaagttctgaaatcttttcatcaagtctttctaattggacgtaaattgctgattgtttttcactattgacagctacatctttatctatactaactatcatttttcttagttcagctacctcaacttctaaatctttgaaagttcctctatctatataattaccttcttgtatcttagacttaatagtttctacttgagaaacaaggttgtttatctccttatccaactagaatcacctctaaggtctaaccgtttcagattcagaatggatatcataattttctaagaaatcattgataatctccatataattatccgtaacgacttttccgtaagatgtttttgtatcaatttcaaacctaagcttaccaaaactttggaggtctaattcttttattacaatattagggtcatcagaaggaaggtaataatagtcgaagtatataattgagccatttattaatactctgtctattctatagacgtggaaatagcgtctgtctcttttaaaatgggctagtgcatctttaaactctaacttaaggatatccttatatttaatcaaagtggtaacctccttactattaatttttaaatttacttattttgtggtataatagttatgataaaggcagttattataattatattaagaataatgataataattattttttctgagaaaataagccaaatactaaaaacagataaagcatagatagctgatagatatactatattaagagttaccttacttttatcttttctatagatagaataacctaaagacgttgtaacaccactaagtataaaataatagaaacaaaaaagaggtatagacagaaaaaaagatacgataatcattgttaaacacctatttctttttgacctattatttctagaacttttagattacaccactaatataacattaaaagccagtcataaaagtcaattgttagattaataatataataaaaaaagacaataggaggttaaagtggttgaataataacatagctatattcatattcaaaacactggttatcattatattcttactactaattttgtctgttattaattccttgtcccttatttactcaataagaccgagtgtagttatgacatactttatctttggtggtattgtttctaatgtcgcacttactgtaacagataagttcttactgaagaaagaagaccccctacctgaatatgttcttaaaaaagtagagataaatgataaagaaataagaataatcaagaaaataatagaaagtaattatggtataacagcagaagagataaaagttagggctaaagcacaaagaagagtagaggaagatagtaaaaaggaagattacaatgaaaacaaagaaagaaattaaagaacaaaggaaagaacttaaggatggtgctacatctgtttctttagtaaaaaagggagataagagaatagctagccctagtagaatctgtagtctatgtggtcagcagttatcaggtatgaattacactaaaggaaaagcattatcaaaagttaatcattttcatttacagtattctaagtatatttattttgatatttgcgcagatatcaacaattgttataaaaatttaagaaaacgaggtgaaatggattgagtgcagaaaatattagagatataattaacaagaaaaagttagaagaagaggatacaagaaaatatatagctgatgggtttatgaatggtatcggtaaattaatgtacgaattcaataagaaagtagataacaaagaaatagaagttaaagacccgaatgatttatacaaattatttgtgatattctctcaaatgcaaaatatggtcaatgaaacttctgaaggaggagcaatacctcaactatctagacctcaacaggaattatttgatgagattacaacagaagatagtaatggagaatctacagttgatttacagaagatatcagaaatgtcagcggaagatattacagcaatgatttctgaaaaggaaaaagtaatgaatgaggaaaattcagaaacattctaaggagaaagatataaatggatggaaaagaactaattaagatagcacaagaaacatttcaaactgaaaaaataacaagagaacagatagaccatataatcaatatgctaaatccttctacctatatgcttaagtatcatacactgagagggcatcctataacttttagtattcctaatagagatagaagtaaagcacaggctcatagaccgtaaattttgttgcggtctctaaactctgttaaacgggcatagtaaaataatgtatagaaaaataatatactggtaagaaaccctaaatctgactatacacagacagtgggaaccaaccgtgctaaatctgtaatttaattattgacaaaataacaatatatgatataattatattatagtaaaagcctaacgactaaatttctagttatctatataaatggatatagtgagaactagataagaaaattaactatacttaaaaaaactaaaatattttaaaagagaggtgaaaacagttgacattagaaaagagaaaacaagaatatcttaagaagttaaagcaaattaaaaatgatgagtttgaattgctaggaggatttactaaaacaagagagaaagctttatttaaacataaagtttgtggttatgaatggtatactactccttataatttattgaagtctaaagggacaggatgtcctaaatgtcaatacagagataaatcatatactactgatgaatttaaaaagaaacttaaagataaatttggttatgaatatgagttaatagaaggacaagagtataaaaatagtagagaaaaattattgtttattcataataagtgcggtactgaatttaaaattacaagtgatagtttatttcgaagtaaagtaccttgtcataaatgttctaaagaaaatagaaaaactaaaaagaaaacaacagaacagtttaaaaatgaattgtataataaacataaagatgaatatatacttgttgaagggtcagaatataagacagctcttgaaaaggttagaataattcatacgaaatgtggatatacatgggatgttagagcctcacatattttgcatactagtaaatgtcctaattgtaatgagtctaaaggtgagagtttaattaaagacattcttgaagataataatttttcttatataagagagtatacttttgaagatttaaagaatgttaaaaaattaccttttgattttgcactattcattgataatgaattagtaggtttaattgaatatgatggttctcaacactttattccttttgaacactttggaggtaaggaaaaattaagaaaaactcaatataacgatagaaagaaaaatgagtattgtgataagaacagaattccgttaaagagaataaagtatgatttagatgaaaaagaagtaataagagaaatagaaatgtttttaaatagtatagttaaaagtaaagcagagagttattaaccaaaaatagtaactatgatatagtctaatccctaagtaaatattgagaaatcaagggtataaatgggcaagttaaaatagtaaatgatactcatcctaataaggctgtaataaaatcacgtcagttaggtcttagtgaaatgggtgtaatggaaatggttcattttgcagatatgcatagttatgctaacgcaaagtgtctgtatacattccctacaaatgaacaaatgaaaaaatttgttcagtcacgtttgaaccctgttttagagaaagaatattttagagacattgttgattgggataaagactcgttaggttttaaaaagataagaaactctagtttattctttagaacaagttctaaagcaagtaccgtagagggtgtggatattgattatttatctttagatgagtatgacagggtaaacttattagcagaatcgtctgcattagaatcaatgtcttcatcaccttttaagattgtgagaagatggagcacaccttctgtacctgggatgggtatacacaaattataccaacaatcagaccagtggtattacggtcatagatgtcaacattgtgattacttaaatgaaatgagttataatgattacaaccctgataatcttgaagaaagtggaaatatgttatgtgttaatcctgaaggtgtagatgagcaagctaaaacagtacaaaatggcagttaccaatttgtttgtcaaaaatgtggtaaaccactagatagatggtataacggtgagtggcattgtaagtaccctgagcgtacaaaaggtaataaaggggtacgagggtatctaataacacaaatgaacgctgtatggatttctgctgatgaattaaaagagaaagaaatgaatacagaatctaagcaagcattctacaactatattttaggttatccttttgaagatgttaaacttagagttaatgaagaagatgtttatggtaacaaatcacctattgcagaaacacaattaatgaaacgagatagatattctcatatagctattggtatagattggggaaatactcactggataactgttcatggtatgttacctaatggtaaggtagacttaatacgattattctctgttaaaaaaatgacaagacctgatttagttgaagcagatttagaaaaaataatttgggaaatatctaagtacgaccctgatattataattgcagataacggggactcaggtaataatgttttaaaactcattaatcattttggaaaagataaagtatttggatgtacttataaatcttctcctaaatctaccggacaattaagacctgaatttaatgagaacaataacagggttacagtggataaattaatgcagaataaaagatacgtacaagcacttaagacaaaggatataagtgtttatagtacagtagatgatgatttaaaaactttcttaaaacattggcaaaatgttgttattatggatgaagaagatgaaaaaactggagaaatgtaccaagttatcaaacgtaaaggtgacgaccactatgcacaagcaagtgtctacgcctatataggattaacaagaataaaagaacttcttaaagaaggaaatggtacaagctttggttctacatttgtttctactgattacaatcaagaaggaaataaacaattctactttgatgaatagaggtgaaatagacttgacagataaattattttatggtacaattagtaatgaagaaattaataaaagtgtattgaatttgttattgggtgaggaattatccttagattatgtttctaaaaatagtgatattttagatgttaaatatgaacatgtttataaatctctaggattcgataatttctttgattgttttttatatgctaatagagagcctgaaatagtccataaaggtggagataaaaatcttggtggactaaataaggttaaacgtactgttattcgtaatggtaaagaaatggaaatgacagtttacgaagatggtaataaagagaacgatagtaaagaaaaacaagaaggaaaagaagaagttagtagaagtgcagtaggagcaagagctatttctaatggtgaagaaggaaaggtaaaccctaaaaaagtagcaaattcattatctaatttaagtaaaaaaggtgtagatgtatcccatattaatacaaactcatcattgtataaagagtttgttgatgataacggtgatacaataggaattacatcttttaaacgaactgaaaatgatataatattagaatcttatgcaagttcacctgattcagatggtgtaggagcaagagctattatggaattattacgtttaagtattaaagaaaataaaaatgcagttgtgtatgacatagaattacctgaagcaatagagtatttaaaaactttaggatttaaacctaataaagatggatacatcttaagaaaaaaagatgtaaaacaattcttaggtgattatagtgattttatttagcactatagtcatctattctattgtatttattctatatattgtattaaaaacaatttatataaagtctaatatgagtagaatagataacacaactgaattattaaaaatattacaggaagatattgaaggtaagataaaaaaggaaggaagaaataaatgactttagaagaaaataaattaacattagaagaatcaataactccacttagcaaagaggagaaagaagatagtattaaagaatttagcagtttattatgtgaaatggtaaatagactatataagtcttataatgtatttagacaagaccctatggatgaaactcaacgtctagatggctctttaatggtctttcaaagtagattaaatgaccctttaacaggagatttacatgataagatgtataaacttgctttttcaaaacgtattgatattttcgaagctaataagcaatttagaaaagatgtagaagcaggtaaagcaattgagttaggtgatgtagctattatagatacagcattaagtaacatcctttcaggcaatgagttccaaggaagtatttcatttatgcttagaaaagactttgaagaaaaagaacgaattagaaaagaagaagaagagaaacttaataacttataaaagggaagaattatgagactatataaaatgaggtatcataattgaaaaagaaaccacaaggcaatgaggtaatcataaccataataacggttatgatagcagtatttgtagtcattatgaccatatttttttaataaatatcaagatgctaaagaagataaagatagatatcaaagattagtagagatttataaaaaagcagatgataatgatggtgagactaaaaagaaatatgttaaaagattaaataaggctgaagaagaacttaaaaaagtaaaaaaagaaacaaattataaagattataataagaagtcaagtaaagaaagacaaaaagaagataaagaaactagagagaaaatatatgatgtaactggtgatgatgacttaatattagtaaaaaataatattgagtttagtgataaagtagacaagcccgaaatacttattagtgaagatggaattggtacgataactgttcctgtagatagtgggtatgaaaaacaaacagtaggttctattattactagtgtattaggttctcctttcctatcacctggttcaaatagtatagatggtttaagtgttattaacgataatgtttatccaaatacagtagatagcatagtagaagatacaaaaccttctattaacttaccaacggataatcctattataacaaatccagttgaaccaactataccttcagatattatacctcctattgataatccttcagttccgatatctcctgagaacccaggagataataatcaaggaaatacagataatccaaatcctccccctccagggtacacagatgaagatggtggaagaggctccggtggtggaggaaattctgaaccaccatcaacggaagaaccttcggataatggtaacaccggaggaggagattgggaagaaaaacctgacccaggagaagaaccttcagataatggtaatacaggaggaaatggtggagaagttacgcctgaacctgaacctgaacctgaacctgaacctgaacctgaacctgaaccatctgaaccgtctgacaatcctgatgaaaatggaggatgggaaacggaaccaactgaacctgagtcaccttcagagccggacgataaagtggacgaagaggataaaaatgaagatactacagatgataaacagtccactgaacaaccggacgataacaacatagataatgaagataaaactgaagaggagtaattactcctctttttttgtttgctatattaaataagagctaaatataaaaaaattgaacattacggtggtgaaaactttgttaggaatgaatattataacgtcactatcagtagtatttacctgtttaagtcttttaactttaatgatttttgttcatagtaagttctctagtaaaaacgtttttgttttgtatgtaatttatgctataataggaataggtacatacatagttttaactatgtttcaaacaacatctgtacttattaagaatgatgtaatagattccatagaaaatactgaacattatattgtattcaatgaccctataattatatttattataagttttataggtgcaatacttggaggaatttggtacaagatgatgaaaattattaaaaaaagtaactttaaagataaaaaataaaaaagacggtgaataggttgatattctctaaagataaaaaatgggatgaagcaaaagatttcatcaaaggtcaaggtatgcaagataattggatagagattgtagattattatagacagataggtggaaaacacgtagctgtttttattgctttaaacaaagtaaaatacatgattctagaagcaacaaaagacaataaagtaatattagtagataaagataataatatactattagaagattatgatattgttatggaaagtaagaagatgttttattacattgaagaaccgtttgaggttaaaataaatatccctcaacatattagagatgtaacttataataatactgttgtattaactacagtaagagggagtagaggtgactagtaattggcagatttatttaagcaattcagattaggtaaagactatggtaataatagtaccattgctcaagttcctattgatgaaggattacaagctaacattaaaaaaatagaacaagacaataaagagtatcaagatttaactaagtctttatacggacagcaacaggcttatgcagagccatttatagaaatgatggatacgaatcctgaatttagagataagagaagttacatgaaaaacgaacataacttacatgatattttgaaaaagtttggtaataaccctatccttaatgctatcatacttacacgttcaaatcaagtagctatgtattgtcaacctgcaagatattcagagaaaggtttaggttttgaggtaagattaagagacctagatgcggaacccggtagaaaagaaaaagaggaaatgaaacgcatagaagattttattgttaatacaggtaaagataaagatgtagatagagattcatttcaaactttctgtaagaaaattgttagagatacttatatctatgaccaagttaactttgaaaaagtatttaataagaataataaaactaaattagaaaaattcatagcagtagacccttctactattttttatgcaacagataaaaaaggtaaaattattaagggtggtaagagatttgttcaagtagtagataaaagagtagtagctagttttacttctagagagttagctatgggtataagaaaccctagaactgaattatcttcttcaggatacggattatcagaagtagaaatagctatgaaagaatttattgcctacaataatacggaatcatttaatgatagatttttctcacatggtggtactactagaggtattttacagatacggtcagaccaacaacaatcacaacatgcattagagaactttaagcgtgaatggaaatctagtttatcaggtattaatggttcatggcaaattccagtggtaatggcagatgatattaaatttgtaaatatgacacctactgctaacgatatgcaatttgagaaatggttaaattaccttatcaatattatatctgctttatatggtattgaccctgcagaaattggtttccctaatagaggaggagctacaggttctaaaggtggttctactttaaatgaggctgacccgggtaaaaaacaacaacaatctcaaaataaaggtttacaacctttacttagatttattgaagacttagttaatagacatattatatcagaatatggagataagtatacattccaattcgtaggtggagatactaagagtgctactgataaacttaatattcttaaactagagactcaaatatttaaaacagttaatgaggctagagaagagcaaggtaagaaacctattgaaggtggagacattattctagatgcttcattcttacaaggaacagcccaattacaacaagataaacaatataatgatggtaaacaaaaagaacgtttacaaatgatgatgagtttactagaaggagacaatgatgattctgaagaagggcaatcaacagattctagtaatgatgataaagagataggaacagatgcacaaataaaaggtgacgataatgtttatcgtactcaaacatctaataaaggtcaaggaagaaaaggagaaaaatcttctgactttaaacattaattaataagcctagaataaatctaggctttgtttatttttttcgtaatttaattttgataaatgtaataactatgatatactatatgtaattgatattaatacataaaaaatattaatatttcacttacaagttattattgttatattattaacgtaaaagtaaataaaataacaagtggaggtgtagacacctttggaagaaataaaatttaatgcttttgtacctatggatttgaagaaatctgtatcaacagcttctgatactaatgagtattctatcgtttcaggatgggctagtactccaagtatggatttacagaatgatatagttaatcctaaaggaatagatatagagtattttaagtcacaagggtacattaattatgagcatcaaagtgataaagttgtaggtatacctacagagaattgctatgtggatatagaaaaaggtttatttattgaagcaaagctatggaagaatgacgaaaatgttgttaagatgcttgatttagctgagaaattagaaaaatcaggtagtggaagacgtttaggtttttctattgaaggtgcagttaaaaaacgtaatataaatgacaatcgagttattgatgaagttatgataaccggagttgcattagttaaaaaccctgctaatcctgaagcaacatgggaaagctttatgaaatcatttttaactggtcatggtacatcacctgacactcaagttgatgcaggagctttaagaaaagaagaaatagcatctagcattacaaatttagcttacgtcactaagattaaagatttaaaagagtttaatgatgtatggaatggcgttgttgaagatttgagtaaatctaatagtatgggatatgaggaatcagtccttacgttacaactagctaaaggtttatctcgtaaagatgcagaactagcagtaatggatataaacaaacaaaaactagaataggtaaggagaatacattctatgagtaaagaaatgcaaaatattttagaagagtatgataagttaaatgctcaagaggcagtttcgaaatctgtagaagatgatgaaaagaatacagtagaatctaccgaagagcaagtagcagaaacaactgaagaacctgctaaagaacctgaaaaagtatctgaggaagatgctaaagaagcacaagagcaaggtgaaaaagttgaatctgaagaggtagcagaaggcaatgaagatgaggaagttgaaaaatcagctaaagaatcaaaagaccctgtagaccaaaaagatactaagacagaaaataaagacaacgagaaacgtaaaaataaaaaagataaaaaagaagattctgattctgacgatgaagataaagatactgacgatgataaagataagaaagaagataagaaggaaaaaacttctaaatcaatttctgatgaagatatcacaacagtatttaaatctatcttaacatcttttgaaaacttaaataaagagaaagaaaactttgctactaaagaagatttaagtgaagttagtaaatctattaatgagttatcagcaaaaatttctgaaatccaagctgaagatgtttctaaatcagtagacactgatgaagaagctgtagaaaaatcagtaacatctacaaacggagagcaagaaaaagtagaaggttacgtttctaaatcagtagacactgaagaacaagctgaaactggtgaagcaaaatcagaagaagctgaagaagtacaagaagataacacatttaaaggattaagtcaagaagaacgaactaagttcatggattcttacaaagcacaagctaaagaccctagagcttctaaacatgacttacaatcagcttaccaatcttacttgaacattaacactgaccctactaatgcatcagagaaagatattaaaactgtaaaagactttgcacaaatttaattaatgcacaaagttgtgttatattatacggtgtaactaaagaatataaatagggtacattttactgtaccctacataaaataaaaagaacacaaatgaaaggtgataaatttatatgactatcgaaaagaacctgtcagacgttcaacaaaagtacgctgaccaattccaagaagacgtagtaaagtcattccaaactggttatggaatcactcctgatacacaaattgacgcaggagctttacgtagagaaattttagatgaccaaatcacaatgttaacatggactaatgaagacttaatcttctatcgtgatatctcacgccgtcctgctcaatctacagtagtaaaatacgaccaatatttacgtcatggtaacgtaggtcactctcgtttcgttaaagaaatcggagtagcaccagtatctgacccaaatatccgtcaaaaaactgtatcaatgaaatacgtttctgatactaaaaatatgtcaattgcatcaggtttagtaaataacattgctgacccatcacaaatccttacagaagatgctatcgcagttgttgcaaaaacaattgagtgggcttcattctacggtgacgcttcattaacttctgaagttgaaggtgaaggtctagagtttgatggtttagctaaattaattgacaaaaataacgtaattaacgctaaaggtaatcaattaactgagaaacacttaaatgaggcggcggtacgtatcggtaaaggtttcggtacagctacagatgcttacatgcctatcggtgtacacgcagacttcgttaactcaatcttaggtcgtcaaatgcaattaatgcaagacaacagcggtaacgttaacactggttacagcgtaaatggtttctactcatctcgtggattcattaaattacatggttctacagtaatggaaaatgaactaatcttagatgaatcattacaaccattaccaaatgctccacaacctgctaaagttacagctactgttgaaactaagcaaaaaggtgcttttgaaaatgaagaagaccgtgcaggattatcatataaagtagtagttaactcagatgacgctcaatcagctccttctgaagaagtaacagctacagtatctaacgtagacgatggtgttaaactttcaattaatgttaacgctatgtaccaacaacaaccacaattcgtttctatctaccgtcaaggtaaagaaacaggtatgtacttcctaatcaaacgtgtaccagttaaagatgcacaagaagacggaacaatcgtattcgtagataagaacgaaacattgcctgaaacagcagacgtatttgttggtgaaatgtcaccacaagtagttcacttattcgaattacttccaatgatgaaattaccattagctcaaattaatgcttctattacatttgcagtattatggtatggtgcattagcattacgtgctcctaaaaaatgggctcgtattaaaaacgttcgttatatcgcagtttaatagaataagaaaaactgaatacaagagaatagggataaacttagggtttatcccttttttattaaaataaacttgaagggatttaataaatatgttatactataagaaactattagataaaaaaatggctactgtttatggtacagtggagattgacaaagatggagtagtcaaaggattaactaaagaacaagaaaaagaatttgccaatgttccaggttttgaatttgaagaagaaaagaaaactactagaaaacaatcagcttctactagtaaagaagaagagcctaaggaagaggaaaagaaagcctctactagaaaaactacaaatactactagaaaatctacagcacgtaaaacaacagccaaaaaagatgaaaataagtaaagggtgaattaaatggttaactcaatgtttggaggggacttagacccttatgaaaaatcattaaactatgaatatccttatcatcctagtggtaatcctaaacacatagatgtaagtgagatagataatttaacattagctgattatggatggtcaccggatgcagttaaagcatatatgttcggtattgtagttcaaaatcctgatacaggacagcctatgggtgacgagttctataaccatatattggaaagagcggtaggtaaagctgaaagagcattagatatatctatactacctgacactcaacatgagatgagagattatcatgagacagagtttaatagttacatgtttgtacatgcttacagaaaacctatattacaggtagagaacttacagctacagtttaatggtagacctatatataaataccctgctaactggtggaaagtagagcatctagcaggacatgttcaattattccctacagcacttatgcaaacaggacaatcaatgtcatacgatgcagtattcaatggataccctcaattagcaggtgtatacccaccatcaggagcaacatttgcacctcaaatgatacgattagaatatgtatcaggtatgcttccacgtaaaaaagcaggaagaaataaaccttgggaaatgccccctgagttagaacagttagttataaaatatgcattgaaagaaatataccaagtatggggtaacttaattattggtgccggtattgctaataaaacattagaagtagacggtattacagagacaataggtactactcaatcagctatgtatggtggagctagtgctcagatacttcaaataaatgaagatataaaagaactattagatggtttaagagcttactttggatataatatgataggattataaggagggttagaaaatggaaaaaccgtatatgataggagctaactctaaccctaatgttattaataagtcaacaacatatactactacaacacaagcagatgaacaagataaacctaagtatactactagactagagtttgatacgattgacatgattaggtttattaatgaccgaggtataaaagtactatgggaagaagcatatttctgtccttgtcttaatcctgatacaggacatcctagagtagattgccctagatgtcatggtaaaggtattgcatatctacctcctaaagagacgataatggcaatacagtctcaagagaaaggaactaaccagttagatataggtatattagatacaggtactgcaataggtaccactcaattagaaaagagaatttcctatagagacaggtttactgttcctgaggtattgatgccccaacaaatgatttattttgtgaataaagatagaattaaaaaaggtatacctttatactacgatgtaaaagaaataacttatatagccactcaagacggtacagtctatgaagaagattatgaaatcaagaataatagattgtatttaaatgaaaaatatgagaatcatacagtaactttaaagatacttatgactttaagatatgtagtatcagatatactaaaagaaagtcgttatcaatatactaagtttaatcaacctaaatcaaaatttgaaaacttacctcaaaaattacttcttaaaagggaagatgtcattgtactacaagacccttataaagttaatgatggtatagaagaagacctagaaattcaagtagatgaccctaaggcttcggcatctaatcctagtaatttaggtggattcttcggaggtgcatttaaataatgccagttcatggaaagagacctaatttatttaaaaataaaaactataagcaggtaggtaagagaacaattgatggtatgcgttcagaagttcttgataaattacaagcaacagcacagcaagtagagaatactagtattaaacgtatgcctacttatctacaaataacagagaaaaagcttgaaaaagaaggagtagtagaccttaaaaaagcttttgctcactcatctaaaaagaaaactagtaaagatggcggatggtatttaactgtaccaatccgcatcaaaactagtagaatgaataacagtacttatcaagatatgagaactttaaaagtagataaaggaacaggttcagtttcgaagataactgattacctagaaggacgtaggaagaatgtaagccacccttcaatgaagcctgaacctatgactcataatatgactaaagttaaaagaggaaagcaatcttcttactttatatttagaactgtttctagtaagtcacctgctagttcttggatacttaacagagataaagttaatgaagataacttctctaaaacaactctaaaaactgttaagcaattaatgaactggaagatgaaaaatttaaattaagaggagggttagtattaaatggcaataacatcagttgattcatatttattatcagaaataaagcctagacttaacactgtgctagagaattgttatattatagatgaagttttaaaagactttgattatcaaactagagagagctttaaagaagctttctgtggtaagaatgcacaacatgaagtaacggtaggatttaacttcccaaaatttaaaaataactatgaagctcattacttgatacaattaggtcaaggacaagagacaaaaaactctttagggagtattcagtcatcttactttgaggcaacaggagatactttagtcgaatcttctacagcaataagagaagatgataagttagtttttactgtttctaaaccaataggagagttaataaaggtagaagatatagagtttgctaaatacgataatcttcaggttgaaggtaataaggtatcatttaagtatcaaacaaatgaagattatgagaactacaatgctaacattatatttaccgaaaagaaaaatgattctaaaggtttagtaaaaggattcacagttgaagaacaagtaacagttgtaggtctttcatttaatgtagacgttgcaagatgtttggatgctgtactgaaaatgattttaatatctatgagagatagtatagaagagcaacaaacattccaattacagaatttgtcttttggtgatattgcaccaataatagaagatggtgactcaatgatttttggtagaccaacaattattaagtacacaagttctctagatttggattatactattacacaagatattaataaactaacttttaaagaaagaaaggattggaagtaggatggctagaaaaaagacacctgaaaataacactcctaaatttaatggttatgttcatatagatacattccttgatactgcaaaaaccctttttaatatgagggattcacaagtagcaggatttaaagcttatatggaaggtagtcattatttgtttagtgagcaagaattcttaccatcattagagaagtatctaggtaggaaattagatatataataacattcagataaggagaattaaatatggcagtagaaccattcccaagaagacctattacccgtcctcatgcatctattgaagtagatacttcaggtatcggtggctcagcaggttcaagtgaaaaagtattttgcttaatcggtcaggctgaaggcggagaaccaaatacagtttatgaattacgtaactattcacaagctaaacgtttattccgttcaggagaattacttgatgcaatagaattagcatggggttctaaccctaactatacagcaggacgtattttagctatgcgtatagaagatgctaaacctgcttcagcggaaattggcggattaaaaataacatctaaaatctacggtaatgttgctaacaacattcaagtaggattagaaaagaatacactaagtgattcattacgtttaagagtaatattccaagatgaccgtttcaatgaggtttatgataatatcggtaatatcttcacaatcaagtacaaaggagaagaagctaacgcaactttctctgtagaacatgatgaagaaactcaaaaagcaagtcgtttagtattaaaagttggagaccaagaagttaagtcatatgatttaactggtggagcttatgactacactaatgctattattacagacattaatcaattacctgatttcgaagctaaattatcacctttcggagataagaacttagaatctagcaaattagataaaattgaaaatgcaaatataaaagataaagctgtatatgtaaaagcagtttttggtgacttagaaaaacaaacagcttacaatggtatcgtatctttcgagcaacttaatgcagaaggagaagtaccaagtaatgtagaggttgaagcaggagaagaatcagctacagtaactgctacttcacctattaaaactattgaaccgtttgagttaactaagttaaaaggcggtactaatggtgaaccacctgctacatgggcagacaagttagataaatttgcacatgaaggcggatactacattgttccattatcatctaaacaatcagttcatgcagaggtagcttcttttgttaaagaacgttctgatgcaggagaaccaatgagagctattgttggtggaggattcaatgaatctaaagaacaattgttcggtagacaagcatcattatctaatccacgagtatcattagtagctaactcaggtacttttgttatggatgatggacgtaaaaaccacgtacctgcttacatggtagccgtagctctaggtggtcttgcaagtggtttagaaatcggtgaatcaatcacattcaaaccactacgtgtaagttcattagaccaaatctatgagtcaatagacttagatgaattaaatgaaaatggtattattagtatagagtttgttcgtaaccgtactaatacattcttcagaatcgttgacgatgtaactacattcaacgataaatcagacccagttaaggctgaaatggctgttggggaagctaatgacttcttagtaagtgagcttaaagttcaacttgaagaacagtttattggtactcgtactattaatacaagtgcttcaatcattaaagactttatccaatcttacttgggtcgtaagaaacgtgataatgaaattcaagacttccctgctgaagacgtacaagttattgttgaaggtaacgaagcaagaatttcaatgacagtttacccaatcagaagcttcaagaaaatctctgttagcttggtttacaagcaacagacattacaagcctagtctaggtgacggagtacctggattaggtactcctattaatataatttgaatactttaggagagtgaatacagatggcatcagaagctaaacaaaccgtccatactggtaataccgtcctacttatgattaaaggtaaaccggtaggaagagcacaatcagcatcaggtcaacgtgaatacggtacaactggtgtatacgaaatcggttctatcatgcctcaagaacacgtatacttacgttatgaaggtacaattacagtagaacgtttacgtatgaaaaaagaaaactttgcagatttaggatatgcttcacttggtgaagaaattcttaagaaagacatcattgatattttagtagtagataacttaacgaaacaagttattatctcatatcatggttgctctgcaaataactacaatgaaacttggcagacaaatgaaattgtaacagaagaaatcgagtttagttacttaacagcaagtgacaaagcacgtacttaatagattagaccaactaaaaagttggtcgtttttttattgacactttaaaatttatatgttattataaatataataatttaaacaggagatgtactagatggcaaataagagaaaaacaataggaaaaatgagtaacacaagagcaacatggaatattaatccggtaactaaagttaaaaaagataaaacaaaatattctagaaaaaataaacataaaggtcttgacaattataattaactaaggtatattattagtataacaaaaaaaggagatgggtatatgagtacattttggtcagaaagaagaacaactaataaagataggcaagttaaaaaacattatactcaaatgagtatgtatgaaagaaagaaatgtgtagagttattacaagagacaattactgaaaatagaattattaattttacacgacatagtgcaaaaaaggttaaaggtaaaccaacaacaaatatacctaaattaataggttttatttttaaaaataagtttgcctacgaaaatatcatagagtacaataacacagattataatggtaatattgagaggagaattgttgttaaacatcctaaagttataactgtagaaggaaaacctagctatcagtttttgacaattagtcttgaagatgctagagttattacggtgtggtataacagtgtagatgatacacatagaacactagatttaaattattatagtaaagacttgacaattcaataaggaggtattataatgggtataacaatagtaaatagttattttattctgtctagcattttcctcatcatattaaccatattaaatggtaagggtacagttacaagggaatcattaactatgagtaaaatattagtagtaataacatcaattcaatttttagcatgtttaattattaatggtatttattggtcactaaaatttatgtagtagaactagaataaaagtattgacaaattaaaactaataaattataataaaggtataacaaattaaaggagaagatataaaatgtcacaagataaattaagagcaatttacacagaaatgaaagtagaattacacaaatttcctaaagaggtagatataacaagtaaatcaactgcaattgcaatcaatcagattttagataaattcaaaacattaacagaacaagcaggaaagattactagaaaatatttagaaggtcaagaaatattaactattgattatgagtattatgattcattacaagaatactatatttacctacttagaaatagtgaaaagattgaacaaagtttacaagaaattactaagcgtacaggtgaatatgtaaagtaattttgatttaaaaacaaaatatgatatactatgtttaaagtagtaagcctacactagtccgtgttatattaatattgaatcggataagcgtaggctttattaatatttaaaaaaggaaggtatatcatattatggcagaagaaattaaaaaggaacaagatgtacaagaaacaactaaagaagaaaaaaaagatgttagtaaaatgacaccggaagaaatagataaattaaaatatcaagacaaacaagaaaaagaacaagttattaacaaagttattaaaggcgttaatgatacttgggaaaaagaatataactttgaagaactagacttaagatttaaagttaagattaaattacctaatgcacgagaacaaggtaatatctttgcgttacgttctgcttacttaggtggtatggatatgtaccaaacagaccaagtgattagagcatatcaaatgttagctaccttacaggaagtaggtattgaagttcctaaggaattccaagaccctgacgatatttataacttatatcctttaactgttatgtatgaagattggttaggattcttaaactcctttcgttactaatagtatagaaacattagataaagatatagaacgattgggcggtatggaatcaattgttaaacaacctttatctagaaatctatgggctattatgaaagagtttaatgttttacctaccgagcaaagatttaaggacttagacgattatcagatagagtttattattgggaatatgaatagagatgtttatgaacataataaacaacttaaacaagctcaaaaaggtggaaaattcgatagtcaattcgaagatgatgatagtagttggtggaatgaatctcatgaagactttgacccagtacctgatttcttagatgctgatgatttagcacaacagatggaagctaaattatccgatagagataaggaagaaagagctaagagaaacgatgcagagttaaatgatgaaacagaaggacttactacacaacatctagctatgatggaatacatcagacagaaacaacaagaattagatgatgaagtaggaaatggtaagactagtgaagatgacgctactatatcacaagatagcgttaataaagcactagaagacctagatgatgactggtatatgtaaagggtggtaggtgatactaccatccttagtttttttaaaatggatggtgaataatgatggcaatgaatgacgattatagattggtcttgtccggtgatagttcggatttagagaatagtctaaaggcaatagaactttatatggattctttagagtctaagaatattgatgctcctttagataatttcttaaaaaaattaaaagtaattgctaaagaagttaaaaatgtacagaacgcaatggataaacaagatggtaaatctgttatatcttctaaagacatggatgaatctattaaatccactcaatctgctacaaagaatataaatgaattaaagaaagctttagatgaccttcaaaaagagaatatatctaaaggtattgcacctgaccctgaagttgaaaaagcatatgctaagatgggtaaagttgtagatgaaactcaagaaaaacttgagaaaatgtcttcacaaaaaataggttctgatgccagtattcaaaatagaattaaggaaatgaaaaccttaaatcaagtaactgaagaatacaataaaataagtaaagattctagcgcaactaaagattatacaaaacgattaagagctaatcgtaatatgactagaggttacatggagcgttcagaaggaacaggacgattaacatatgaccaaggtgcacgagttagaagtgagctaggtaaaataagttcttatgagagccaaagaaaacaaaaccaacgtaatttaggacaagcaagagagcaatatagcaactatagaaatcaacaacaagacttgactaaacgtagagctagcggtcaaattaataaggaacaatatgaacaagagttagcttctattaaacaggaaatgaaagctagagaagaacttatatctaactatgagaaattaggagcagaacttgataaaacagttcagtattataagggttcagttcaaaaggatttccaatctagagacgtagaccaacaaagaggaacatttggtagaatggttcaagaacgtttgccatctattggttctcatgctatgatgggtactacagctatggctacaggtttatacatgaagggtgcctcattaagtgaaactaatagaccgatggttacatcattaggtcaaaattccgataatatggatatagattctgtaagaaatgcatatggagacttgtcaattgataacaaattaggttataatagtactgacatgttgaaaatggctacttcatatgaagcatcagtaggacataaaagtgatgaggacacaatggcaggaactaaacagcttgctattggaggacgttctttaggcattaaagaccaagaagcttatcaagagtctatgggtcaaatcatgcataccggtggagtaaattctgataacatgaaggaaatgcaagatgcattcttaggtggtattaaacaatcaggcatggttggtcgtcaagatgaacaacttaaagcactaggttctatagcggaacaatcaggagaaggaagaactctaactaaagaccaaatgagtaaccttactgccatgcaatctacttttgcagagtcaggaagtaaaggattacaaggtgaacaaggtgccaatgctattaacagtatagaccaaggacttaaaaatggtatgaatagttcttatgctcgtatagcaatgggatggggaacgcaataccaaggtcttgaaggtggatatgatttacaaaaacgtatggatgaaggtatatctaatcctgaaaacttgacagatatggctgatatagctactcaaatgggtggcagtgaaaaagaacaaaaatacctatttaatagaagtatgaaagaaataggcgctaacctaactatggagcaatctgatgaaatatttaaagatgctcaatccggaaaactatctaaagaagagttagctaagaaagctaagaaaatggaaaaagaaggtaaaaaagaaggagaagataacgccactgattataaagaatctaaatcaggaaaaaatgaccaaaataaatcgaagactgatgataaagcagaagatacttatgatatggctcaaccactaagagatgctcatagtgctttagcaggtcttcctgcccctatatatttagctataggagctataggagcatttacagcttcactaattgcatctgcaagtcaatttggagcaggtcacttaattggtaaaggagccaaaggacttagaaataaatttggtagaaataaaggcggtagctccggtggtaaccctatggcaggtggaatgcctagtggtggtggttcacctaagggtggaggctcacctaaaggtgggggcactcgttctactggaggaaaaatacttgatagcgctaaaggtcttggaggattcctagtaggtggcgcaggatggaaaggtatgtttggcggggagtctaaaggtaaaggctttaaacaaacatctaaagaagcctggtcaggtactagaaaagtatttaatagagataatggtagaaaagccatggataaatctaaagatatagctaaaggtaccggtagtggtcttaaagatatctataatgatagtatatttggtaaagaaagaagacaaaacctaggagaaaaagctaaaggttttggtggcaaagctaagggtctctatggtaagtttgctgataagtttggtgacggaggtaaaaatggtattctttcacaatcaccaaaagcaggtggaagtggcatagggaaacttggaaaacttgcaggtggacttggaaaaggagccggagttttaggtgttgctacgtctgccttatcattaatacctgctttagcttccggagatagtaaagctatcggcggaggaataggctctatgggtggaggaatggcaggtgcatcagcaggagcttctataggagctttatttggtggtgtaggtgcaatacctggagctttaataggtggagctataggttccttcggaggaggagctgttggtgaaaaagtcggagacatggctaaaaaagctaacactaaagaaggatggaacctaggatggactaacggagataaggatggtaagaataaattccaagattctttattaggaaaacctatatctaaagcatggagcggtataacaggtctctttgataatgacgctgaagcatccgaagaagatagtaaagataagaaaaaaggtgttaaaggcgttaaaggagatactaagaagaaagaaaaaatgacagcagaacaacttagagaaaagaataaccaatctgaaactaagaatcttaaaatctatagtgatttacttgacagagctcagaaaattattgagagtgctaaaggtattaatatagatggaggaacttctgatagtggttctgatagtggaggctctgcatctgatgtaggtggagaaggcgcagagaagatgtacaagttccttaaaggaaaaggactatctgataatcaggtaggagctgttatggggaacttacaacaagaatctaatcttgaccctaatgctaagaatgcttctagtggagcatttggtattgctcagtggttaggggctagaaaaacaggattagaaaattttgctaaatctaaaggtaaaaaatctagtgacatggatgttcaattagattacctatggaaagaaatgcagtctgattatgaaagcaataatcttaaaaatgcaggttggagcaaaggtggaagcttagagcagaatacaaaagcatttgctactggatttgaacgtatgggagcaaacgaggctatgatgggtactcgtgttaacaatgctaaggaattcaagaagaaatacggaggctccggtggcggaggtggtggaggagccctatcctctacttaccaagaagctatgagtaatcctgtattaactactggttctaattataggggctctaatgatgcttctaatgcttctacaactaacagaataacagtcaatgttaatgttcaaggtggaaataatcctgaagaaactggagacattatcggaggaagaattagagaagttctagatagtaatatggatatctttgcaaatgaacataagagaagttattagtaattttgtattgacacaagagtagtatcataatatactactcttatacatataaaaaataaaaggaagtatgtgtatatgcgtagaataagaagacctaaggtaagaatagaaatcgttacagatgataatacatttacattgagatttgaagatacacgtgactataatggtgatgagtttggagctaaacttttaggattccaaactaaaaactctatggaagatgatagttcagttttccaaataaatatggcaggagatacttattgggataagctagttatggctaatgatatcataagaatatttattacacctaatgatgaccctaacgataaagaaggaaaacaagaacgacttatccaggtaggtatggtttctcaagtatcaaaagtaggtagttacggtaatgaccaaactcaatttagaataacaggtcaatcttttgtaaaaccttttatgaaatttggattaggcgttattcaggaagttcaagctgtattacctgaagtaggttggcttattgatggtgatggagataatgaagtaaaatttactggtagctcagctcatgaagtaatgactggtattatacgtagatttataccttatatgaaatataactatactgaaaaaacatataatacaattgataactatcttgattatgatgatttaagtagttgggatgagtttgaaaaacttacagaagtttcagcctttactaattttgatgggtcattaaaacagttaatggatatggtaacagctagaccttttaatgagttattcttcaaaaattcagaaaaaacacctggaaaggctcaacttgtattaagaaagaccccttttaatcctactgagtggagagctttagatatgattaaagtacctactgaggattttatagaagaggatgtaggtaaaagtgatgtagagacatattctatatttacagcaacacctgcaggtatgttgaaagagcttaacggtgatgtattttctaaaccacaattccaccctgaattaactgatagatatggttatactaaatttgaagtagaaaatatttatcttagtacaaaatcaggttcagctactgaggattcagattcttcaggtgatgataatggcacagaacgaggaacttattctaaaattatgaaagatttaagtaactatggaagagataatatatctaaaggtatagataagtatacaagtaaattatcttcaaaatataaaaacttaaaaaaagcccaagctaaaaaaattatagagaagtttgttaaagaaggaaaagtaacagaaaaagaatatgaaaaaataacaggtaataaggtagatgatgaattaacatcagataacagaccgaagttgacaaaagataaattaaagagtatactaaaagagaagtttaaaacacaagatgattttaataattctaagaaaaagaaaaaagctaagacagatgcacttaaagaattgacaactaaatatcgttttggtaataaaacacatgctacaactttattagatgaatatattaaatataaaggagagccacctaacgatgaggcttttgataaatatcttaaagctattgaaggtgttagtaatgtagctacagacacaggttcagatgcaagtgatagccctttagttatgttttctagaatgctatttaattggtatcatggtaaccctaacttctatgcaggagatattattgttttaggagaccctaagtatgacctaggtaaaagattatttattgaagataagcaacgaggagacacttgggagttctatattgaatctgtagaacataaattcgattataaacaggggtattatacaactgtaggagtaactagaggtttaaaagacgctattctagaagatggtaaaggtagtccgcatagatttgcaggattatggaatcaatcatcagacttcatgggaggtcttatgggtgaagatacttctaaagaacttaaagaaaaaggtgtagcagagaaacaaagtagtggagataaagatggtggttctgatagtggtggagctcaagatggtggctctttagattcacttaaaaaatataacggcaaacttcctaagcatgacccaagttttgttcaacctggtaaccgacattataagtatcagtgtacatggtatgcttataatagaagaggtcaattaggcatacctgtgcctttatggggggacgccgccgactggataggtggtgctaaaggagcaggttatggtgtaggtagaacacctaaacaaggtgcttgtgttatatggcaaagaggagttcaaggaggtagcccacaatatggtcacgtagcgtttgtagagaaagtattagatggaggtaaaaaaatatttatctctgaacataactatgctacccctaatggatatggtactagaacgatagatatgagttcagccataggtaagaatgcacaattcatttacgataagaaataaaggaggatagtctatggcaacagataaagaagctaaagatgttattgataaatttatagacaatgtatttaattttgatgtacttacaaaagaaagaataaaagaaaaagatgaagaaattaaaaaaataactacagatgatatgtatgaaaaggttgtgtatatacgaccttatgttggagtaatacaaagccttaaccctcagcatgttcagtatgaatcattttctaataatggttatgatatagaggcagaattaagtttcaggaaagtaagttatttagttgataaagggtctatacctacagattctttatctactttaacagttcatttagtagaacgaaatcaagaactattaatagattactttgatgagatacaagatgtgttgtatggagaatatatggaagaagaatatgtatttgatgaagatgtaccattaagtacgatactagcattagacttaaatgataatcttaaatccttatcaaatataaagtatatgttcaaaggtgctcctaaagagaatccatttggaacagataaagatgtttatatagatacttataacttattatactggttatatttaggtgaagatgaagagttagcatatcctatgaatattaactacttctttacagagggaagattctttactatattcggtaaaggacataagtataaggtagatgttagtaaatttatagttggagatatattattctttggtagaagtgatactaatataggtatttatgtaggagatggggagtttatatctatgatgggtaaattccctaaagatgaaacacctataggaaaatataaacttgatgattactggaatgaatttaacggaagagttatgagattcgatgaagaggtgtatatttaatggtagtaagattccaatcttccatggggagaagtttaaaaagagtagattcggatgatttaaatgtaaaaggattagttttagctacagttagtaaaattaattataaatatcaatcagtagaagttaaagttaacaatttaactctaggaagccgtataggtgatgatggtagcttagctgtaccttatcctaaatctttcataggaagaacacctgaaggaagcgtattcggtacaaaacctcttattactgaaggttctgtagtattaatagggtttctaaatgatgatataaatagtcctattattttaagtgtttatggtgataatgaacaaaataaaatgattaataccaatcctctagatggaggtaagtttgatacagaaagtgtttataaatatagtagttcactatatgaaattttaccatctttaaattataaatatgatgatggagaaggaacaagtattaggacttataatggtaaatcatttttctctatgacatcaggtgaagaagagaaacctcaggcaacagatttttatactggaactgagtatcaagatttatttacttcttattatggtaataagacattaattgagcctagaatacaaaaggctcctaatatgttatttaaacatcaaggagttttttatgatgatggcacgccggataatcatataactactttatttatatctgaaagaggggatataagagcctcagttttaaatacagaaacacagaaaagaactacacaggaaatgtcaagtgatgggtcttatagagttatcaaacaagatgacgatttaatgttggatgaagctcaagtttggattgagtatggtattagtgaagataataaattttatattaaaaatgacaagcataaatttgaatttactgatgagggaatctatatagatgataaacctatgttagaaaacttagatgagagtatagcagaggctatgaagaatttgaatgaaatacaaaaagaactcgatgatataaactaccttctcgagggtgtaggtaaagacaatttagaagaattaatagagtctacaaaagagtctatagaagcttctaaaaaagcaacttcagatgtcaatagacttacaactcagatagcagaagtgagtggtagaactgaaggtattataacacagttccaaaaatttagagatgagacttttaaagatttttatgaagatgcttctactgttattaatgaagtaaatcagaatttccctactatgaaaacagatgttaagaccttaaagactaaagttgataacctagagaaaactgaaataccaaatattaaaactagattaacagaactagagaacaataataacaatgctgataaaataatctcagatagaggagaacatataggtgctatgatacagttagaggaaaatgtcacagtacctatgagaaaatatatgccaataccatggagcaaagttacttataataatgcagagttttgggattctaataatcctactcgattagtagtacctaaaggaataacaaaagtaagagttgcaggtaatgttttgtgggactctaacgccacaggacaacgtatgttgagaatattgaaaaatggtacttatagtataggattaccttatacaagagatgtagctatatctacagcacctcagaatggtactagtggagttattcctgttaaagaaggagattactttgagtttgaagctttccaagactcagaaggtgacagacaattcagagcagacccttatacatggtttagtattgaagctatagaattagaaactgaaactatggagaaagactttatgcttataggacatagaggagcaaccggatacacagatgagcacacgataaaaggatatcaaatggctttagataaaggtgcagattatatagaattggatttacaattaacaaaagataataagttattgtgtatgcatgattctactatagacagaacaacaacaggaacaggtaaggtaggagatatgaccttatcttatatacaaactaactttacatctctcaatggtgagccgataccatctcttgatgatgtactaaatcattttggaacaaaagttaaatattatatagaaactaaacgtccgtttgatgctaatatggatagagaattattaactcaattaaaagcaaaaggattaataggaataggttcagagagattccaagtaattattcaatcatttgctagagaatctttaattaatattcataatcaattctctaatatacctttagcttacctaacaagtacattttctgaaagtgaaatggatgattgtttaagttatggtttttatgctattgcgcctaaatatacaactataactaaagaattagtagatttagctcatagtaaagggcttaaagtccatgcatggacggtaaacacaaaagaagaaatgcaaagcttaatacaaatgggtgtagatggattctttacaaactacctagatgaatataaaaagatttaatattaaagacctattaatttaggtctttttttagttgtaatttaaactagttcgtgatatattagtagtatgagatttatatacatactgaaaaggagaggataaaatgccacaatcagatggaataagtaatcttcatagaatagctttacgcttccctaaagaaggcggtggttatgatatgtatagatttaaagttaaccctgagaactacacaatagattcaccacaacgtacgacagcaattaaaacaaaatcagatattgtaatagaagattatggtaaagacatagaagttattaacttcacaggtacaactggttttagacctgttagagaagcagatggattaaaaacaggtaagcagaaaatggaagagttacaaagtagagttagtgaatatgctatgcaaggtggcagtggtaatgtaagtggttcttacttacaattttttaactttacagatgatagttattataaagttcatttagctcctcaggggttaaagataactaggtctaaagatgaaccattactttttagatatgaaataacattagtagttattggttcattaacagaagcagatagaagtgctgtaacaacagaagagtttggtaacgttaaacctaatgcttctcaaagagtagatgagggtataaaagaattagataaaaatgctcgtaaaacgagagatagaaacaatcaagaaatatctagaagagaaaatacaatacctaaatctacaggagataatacgaacgagggtaatagacttaagcaaagcttccctagtagttctatatataatcctagacaatctactaacggattaaaaggtaatattgacaatatggcgctgataataggttacggtgatggaggtgtatctagctaatgaataattttataccacaacctcaaggtctacttagatttttaaatgccctagatacagatttaacttcttctcatatgaatttactggatgaagaggtatcatttgtatctaaattttatacaccacagctacaattaagtgaattagcaaaaaaagtattgacaaatataaagacagatgatatacctgtattagaaagagaatttaatgataatacaattatccataaagctaacgatacattactaaaagtacaggctccaagaatgtatatgattctacagtcgattgtacttgaagcatatgctattgttaattgctttgtagaaaatccgagctctttaaaatacttaactgaagaagatgttagtataacacgggaaaatttaaattatgtagctgactacttaggtaactatgatgactataatagtgttgtcttagacttaagagatttagacttatgttttagtgctatagaattacaattacctctaatcaaaaaggaggctaacgtataatgagatttaagaagcacgtagttcaacatgaagaaacgatgcaagcaatagcacagagatactatggtgatgtgagttattggatagacctagtagagcataataatttaaagtacccctatttagtagaaactgatgaagaaaaaatgaaagaccctgaacgattggcttctacaggtgatacactgattatacctatagaatctgatttaacagatgtatcagcaaaagaaattaattctagagataaagatgtactagttgaattagctttaggaagagatttaaatattactgcagatgaaaagtattttaatgaacatggtactagtgataatatactagcattcagcacaaatggtaatggagatttagatactgtaaaaggcatagataatatgaaacagcaattacaggcacgtttattaactcctagaggttctttaatgctacatcctaattacggttcagatttgcataatttatttggtcttaatatacctgaacaagctacattaatagaaatggaagtattgagaacattaacatcagataatagagtaaaatctgctaatctaattgattggaaaattcaaggtaatgtttattcaggtcaattttcagtggaaataaaatctgttgaagaatcaataaattttgtcttaggacaagatgaggaaggaatttttgctttatttgaataggaaaggattaaattatgaaaactagaaaattaactaacatactatcaaaattaatagataagacaatggcaggtacaagcaagataacagactttactcctggttcagcttctcgttcattattagaagctgtatcattagagatagagcaattctatattctaacaaaagaaaatattgattggggtatacaagaaggtatcattgaagcttttgattttcaaaaaagacaatctaaaagagcttatggtgatgttactattcaattctaccaacccttagatatgagaatgtatatacccgcaggaacaacttttacttcaacacgacaagaataccctcagcaatttgaaacattagttgattattatgcagagcctgattctactgagattgttgttgaagtttattgtaaagaaacaggggttgcaggtaatgttcctgaaggaacaattaatactatagcatcaggttctagtttgattagaagtgttaataacgagtattcttttaatacaggaactaaagaagagagccaagaagactttaagcgcagattccactcttttgtagaatctagaggtagagcaactaataaatcagtaagatatggtgcactgcagatacctgatgtagaaggtgtttatgtttatgaagaaacaggacatattacagtatttgctcatgatagaaacggtaatttatcagataccttaaaagaagatataattgatgctttacaagactatagaccaagtggtataatgttagatgttacaggtgtagaaaaagaagaagttaatgtttctgctacagtaactatatctaataaatctagaattggtgatacattacaaaaacatatcgaaagtgttattagaagctatttaaataatttaaaaacttctgatgacctaataattacagaccttattcaagctataatgaatattgatgatgtattaatatatgatgtgtcatttgataacctagatgagaacattatagtaccgccacaaggaattattagagcaggagaaataaaagtagaactaaagtaaagagaggtgaaacttaagtcgtggctaattttttaaagaatcttcatccattattaagaagagatagaaataaaaaagataatcaagaccctaactttgctctgatagatgcactcaatgaagagatgaatcaagtagagaaagatgctatagaaagtaagttacaatcttctctaaagacatctaccagtgaatatttagataagtttggggattggttcggagtttatcgtaagaccgatgagaaagatgatgtttatagagcaagaattataaaatatttactcttgaaaagaggaactaataatgctataatagatgctataaaagattatttaggtagagatgatattgatgtaagtgtatatgaaccttttacaaatattttctatactaacaaatcacatttaaatggtgaagaccacttaatgggatactattatagatttgctgttattaatgtctctataggtgattatttccctgtagagattatagatgtaattaatgaattcaaacctgcaggtgtaactctatatgtcacttatgatggggcttctactattagaggtggagcaattattaagtggttagatgggttacctaaaatagaaacataccaagagtttgatagatttacaggttatgatgatacattctatggtcatattaatatgaatcaaagtaaagatactgataacagttcatcagatatttttaaaacaaaccatagcttaattaatagtttagatgttttaacaggttcatctagtgtagggagacagtatattaactacggatatgtaacatcatatgtttataatccaggtatgacatcttctgtaaatcaaataagcgctagtacaaaaggtagaggtcaagaagtacctactgactattatatgtatactagtactaagaataacaatacagtagaacttagtatgcaaactacttccggtgtgtcttatttatataataactttaattttagggactatatgagtaaatatagacctcaagtagatttacaatctgatgaggctagaagaattgtatctgattatataaaagaattaagtattgattactatcttagtgctgtgatacctcctgatgaaagtatagaaattaaactacaagtttatgatttttctattaatagatggcttacagtatcaattaataatttatctttctatgaaaaaaatatcgggagcaatataggatatataaaagattatctaaacagtgaattaaatatgtttactaggttagagataaatgcaggtaaaagagattcagtagatattaaagttaattacttagatttaatgttttattactatgaacgaggtatttatacaataaaaccgtataaagcattaatagaaaattatttagatatatctagagagacttatgtagaagacatttaaaatagcatca

SEQ ID NO: 4 - DNA, 141676 bp, Staphylococcus aureus
  source 1..141676: mol_type = genomic DNA
  misc_feature 1..141676: note = bacteriophage MB403
ggaaaacttttacctctcccattcgttcaatcattcactaccgacttccctaactacttattctatagttataatattcatttattatacaatacttaaactatagtattctagtcttaatctatactgaagcggtattaatctattgttattatataataatcttatctaatagtggtataatctaggttattacattagaatgattctaatctagtattttaatctttagaccctaggaaaagtggtactaaaatatagaaccctataggtacgggattcttatttttaaaattactaaaaagtattaggttttccctagggtaaagttttaatgtacttaaaatcgtaagtagctccttatcatttaggtctgtttaattgagaatattgaaagatatccgcttcaattacaattaagtgttgacaatcatgaagcggtatgttatacttagtatataaattaataggagatgaattaaatgattataccattaattatactcatgatgaccttcggtacatttgcattcagttatgttgcacatgatgcatacaaggtagatgaaaaaggtatcatgtatgctatggtagttggtattgtagttataaatgtaattggtttagaaatgataattgtagaatgtttatagaggagatgatttaatatgattgatatttatttacacagtgaatatgataaagataagttaaaatttatccttaaagcaataagggatttttctcctagagaattaacctacgattttaggaatccaaaagcggatgttcgtattgaggaactactaggagatgacatagacatatttgaatctatagcattagattaccctaatgatattaatatccttataggagatagtggatactcgatagtttatcagaatgattttcttacaattaatggattgagtatagctataaaggaggtagtaagatgaataaagtattatattttgttttatgttatgtgtggtatcttataatgggttttataggtgtacggttaggaagtttaattatagggatgaatattaacccttttacaagttggataccttatgtcactgcaatagtatttacattgtttgactatatgtttttaactaaaaaatattaagggtatgttttaatatccttttagagcttatttaagaagcggttaagtagttcatgataaattggtctagaaatatactaccacttctctatggaaggcttagagggcttagaattgaaaggagagatataatgattcatatatttgtaaaagaagattataataaagaagtgttaaagcatttacttgagtatattaatgatactgtaggtagggaattaacttatggtattaatacagactatgataaggatgtagtgattgaaaccgatgaccctatagatgaggagaatacgattgatttatcaaatcttcctcactttaaagatgacttatgtattcttatagaagaattatactgtaaagcatttgttaatggggaacctgttattatacgtaagtatgtagaggagatgttataatgattataatatttttaactgaaaaatatgattccaaggctttaaagaaagtattagaacatattgataattgtagtagtagaggtcttagctatttaatgggaaaaggagaagcggatgtatgtatagagaaaaatgtatttatagaaagagatgatgtaaggattaactcaaacattattgatgaaggtaaactctgtgtgttaataaatagacatggtttagaatgtagttactatagaggtgtatcatgtaatattggtgccttcgtaaaggagagatataatgatagagatatatctcagtgagaattatgataaggatttgctcaaagcagaattgaaatggattaaagagaccgcttcaagagaactaacttatgatgttaatagaagtccaggattagatgtacatattagtccttttaggtataccaaggatgaggttgaagaaatgagcttgcatcctcagtttgaagacgatatatgtgtatttatagctgaaacctggatacatgagtaccgtaatggtaagtcaataggtgtagatagcatggaggagtatgtaaaggagatgtagttaatggtagaaacttattatacagtatatcataagaatagtatgaaaactattaaagattacttaagtagaagtgatttaattgacttcttatataagacttggcataaggatataagtaatatatacccgatagattatactacgtatgaaacaaaattctgtctagacatagatgtagatagattaattgaagcggttcatgaggaagggatactacttattaatgaaggtaagattataggataaaaataacttaggatgcttaggattataggataaaaataccttaggatagaaggagataaatatgaatcagtttttcacgtatacagtgtatcaccaagattcaggtaaagtagtagctaaggatttgtttgaggaatcattagtgcaccttattaataagatgagggtggaagaattagttaaggatattactttaggatattacatgaatacacgtggtgtagttatagatatgagtagactactggatgatatagaatcacagggtgtatttttatttaaacgtaagatacatgggtaggataggagggataggatgatagttatatattcagatgagtctaagggttacttaagagatgagttcttaccttggcttaatgatagagatagatatttagagtattataaggataagttacctgaagacatagactcttcttatgttgtatcagttgtatactgtagaaatatggaaggtcttatgaatagaaaagatattcttattggtaatagttatagagaacctgtagctgtattaggtgttcctgagttttttggtaactattctaattactattattataaaggggagtatataagtaagcatgacctaggagagataatcaggttaaaggcttggcaacgtaattttgactaagtagctctccctaatttcactaagtagctccctaggaattgcctaagtagctcggtatgattttaccctaagtagctccctctgttttctattagtttattttaaccgcttcacgtgtctatatatagacaatcgtaataatagcagaccgcaaaaataaatacactaggatattattcccagtgtattgtataatttttttatagaatatttataacattgtattcaaattcatttacttcatgttgtgatttaattaaatttttaattaatccgttttgtgttttatagtcttttattagtttttcattttctataattaaattattaaattcctcttctgttgtatcctcatctacataaaatttactttcatatatttcataatattttttatctgttccgttatcaatatcatctgatatttgataatttttgaatacaatttcttttgtttctaattcatttactaataattgtgattttgcatattgtaatacgtcttcattgtcccacattggaatatagtttattttcatttaaatcaaatccttttctataatttttttatataatatttgtagaagcggttgggggtttgtcccttgccttactacacttaatatattacagtatagttattcagaagtcaatacttttgagtaactttttttaaattcttttttcttctatataatagtagtttttagccctaaaaaagttttttaaaagaatttacattttcttattgacttattaatcatatggtagtaatataaaggtacagcaagggaacagcaacaagatattagaattatataaaaaaattatttaattggagatgatttaaatggatgtaaaagaaattgcaaatactataatggagttgtggcaaatggacggctacagatgtacagaaccgccattatatgaaagcacattaaaccatacacgcacatacacggctttaatcgtaagcattaaaggaaactacgacactgttcaaatgttccgcaaaacgcctataatgagcatgagagggcaagcccaaccggctagcatgttagtaaatgtaattgatgatgtgattataatcgtatatgaaaatgttgtttacggggtacagaataaagaaataaaatttattgaagaaatttaaaaataagggttgcaataccccttaagatgtagtaatataatagatgtaagggatagcaacacaccttaaaaaactttttaaaaaagttaaagaaaagtgttgacaccttacaagatacatgttattattagtatagaagttaagacaagccacatagcaaataacgaaattaaataaaaaaattatagaataggatttgattattatgacaaacaaaaattacttatacgaagaaactcacacagtacaagggaacgaaattacggctttcagaattccaaacgatgtaaacggaaacccacgttatgtagtgcatttcatggatttagatattaaactagcagactatgacaacatcaataaactatacggctttaaaaaatatactgctaaatggtttggcggtggtgtagtattccaaagttataatatagcggatactctagaatatgcttacacacaagttaaaacaaatagaattagtcaataaaattaaatatgaagcggttgaatataaccgcttctgaattaaataaaaaaattatataaaaaggatatgataatatgaaatttaaaatagaaaaaaataacagtgatataaaaactttatggaatttagctaaaaatggatatatgagttatcaaactgtacacaatatatttaaaaatgaatcagatgaatttattatatttaacagtaaacaaacttataataaatttatggaattaagatataatagaagtgcaatccaatagtataaaaaaattatacaattccctgggattaaattcctagggatttttatttgttttaatttatataaaaaaattatttaataaataaattagtgtaaaattgactgttgacaaggttgtattttttatggtataatgaagtgaataccttttttagtataaaaaaattattatataaaaaatttatattaaatggttttaaaaccactctttctccccaccttgtcatatttatagtggaagggttaggcgggttaccactgttttactttctatatatagtatactatgaataatggtaattgtcaacacctttcagaaactttttttacttttttttattattatataaaaaaattatacataaaaatgggctccacttccattatataataattcggtcttaatgtcaatacttttattcaaaaaagttttttaaattaatttcaacaaatctattgacttatgtttatttctatagtaatatataggtataccaacaagggaggcaatacaaatgctaaaatttaaatggaaaaacaaaacaatcaaatcaacacaaaaaacagataacattctcttacttattatagggggtttagttgcaacagtcacacctaaacttataaactggtttttactactacaagataatataaatatttttttaagataactattgacaacttaaacaatagatgttaatattaagatacaaggtaagggaagcggttgaccgcttccaacctaaataaaaaaagtttaaaaaaactattgacagtcacttgaaaccatgatattattaagataacaaaaacaaacagaaaaggaattgattataatgaaatttatcaaaactatcgaaaacttattagaaaaagcagaaaacaaagggcaagcaattttaaacggtcgttactttgacggatatagcaaaggtgagcttgaggaaaaatatgcaatcgaaattaatggcaataaattaattatgagacattggggcacacaaacaattgagattgacttaagcgtaaatgaaattgtttcatactatggtgaaagcaattcagaccgtgacagtttaaacacacttgtatattgcttaggaattgcgccaaactttagatacttaccaagcaaagacttattcatttacgaaaattaattaaataaagggcttgacttccaagccctaccatgttattattaaattgtaaggtaatcaagcacaacgacaaaataaactgaaaagggattgatgaaaatgacaaataataaggattttaataaaaatttcttaagactactaaaagttaaaaatgtaaatgacaaaagggtgcaaggtttcataaacaagcatgatattaaacttaaggataaagaagaaagttcacaaggatatgttaacgtattatacaaaggttatgattttgtaatatcaaaagactatgcaggtcaaacggcgttatatgttaacatgcgtaatctaagggttgactacaaacattttaaacatttagattttaataatattgttaaagcattagaaagaaaaaatattaatcatgttgaatacaaggaaacaaaaacacaacaatataaagaatttaaacaccgtcaaaaaacagaaaaagaaatattaacatactatgaaaaggatatcgaaaaactaaaagaagaattggaacgaacacaaaaaatctacaatgataaagtaaagaaaatagagaattacgataaaaaaatatctagactaaaaaaatcaaaataaggcttgacactttcaagccctcatgatatacttagtataacaaatcaaagagaaaaggaattgatgaaaatgttcaaattacaaaataaagtggaaattatcgtacctaaggaagataacaacggcgttgagattgcagacaaacgtattaaagaatatgtaaacagtatcacaatggaagcgggcggttgtactattacagaaattaagggacaatggtattcagaagatgaaaaacgtatcatggaagataacaacttaaatcttgaatggtactatatgaaagacagtgcaaaatttatgacagttgaattaaaaggcattgtaagacgtttaattgaagtttacggacaagaagcaataagcattaaagttaatggcacattgtacattgtagaccaatcagatattgaagaattacacacaacattattaaatatcatgaaataaaaaatttatataaaccgcttcggattaaattcttgaagcggttattttcttttaaattagtgcttgacaaatggactaaaaaatgagataataaggtgatgactttttttagtataaaaataatattatataaaaaaattatataaatgaattaatttcaggtctttcccgtaacccctcttgggctaccctggtatctctacaattattattatactataggttatgaagattgtcaataactttcttcaaaataatttatttaaaaaagtttcataaatctattgacttctatatcattatggtgtaatatatagatataaggttaaggaagcggaacacctaaaaaaagaaattaaaaaaacttttaaaaaggtgttgacaaacttcaaaccacatgttaatattaagataacaaataaaacaaaaaggaattgataattatgaacagattagaaatagtaaaagacacggcaatggaatacatcattatgatggataacggagttatggacggacaattgacacaagaagaatacaacgaagcagttagctttgaaaaggtttacgattacactttatcagaagcaaatcaagaatgtaaattcttaggcggtaaaattttaactttcctagtacatgaagcaattgaagaatacgcataaaaaaacttaataaaaggggttgacattcaacccctaccatgttaatattaatatataccaaatgagaggaattgataattatgagatacgaaatcataacattagttaatggagaattattcatgtttgcaacatttaagaaagcagaggcagaaaataaatatcaagaatggtgtgacttgtatggtcaagaaaatgtgagcatggaaaaaaattaaaataagcggttgacaaactaaccgcttcatgataatattaaactatactaaagaaaaggaaatgatgacaatgacaaaaacaattaaacaattagaaagccaacttgaaagactagaaagaaaatcagatgaggaactagcaaacggatattatgaagcctttgaaagaacttgcgcacagattagagaattagacctacaaatcgaattaaaaaagaattcagaaactgtttaaaaaaattaaaataaggggttgacacttagccccttagatgttattattaatatatactaaagaaaaggaattgataaacatgaaattattaaaccaagaaaaccaaatcgtaattagcatagcaacacttgagagcgtaaaacaagccctaatttgggagtacatcgaccacttagataataacattctaggcaataaaatctatgacacagaagcggttgtcgttacttctgagactctacagacaatcaaatttgcagagactatagaagacctgcaggaatacattgcagatattaattggaaactagtttaaaaaagttttaaataactgttgacatcctagcaaatagatggtaatataagagtataaagaaaaacaaaaaaaaacgaaaaggatttgattataatgacaaacacaataaaaggatttttacaaacagaagaagcaagcacagttaaggacgtagcaactcacggagtgcaaagcggagcaattggcagattaatttacacatcagacgtagtaaacttttttgaccgatacgagcaggatattacagacgttatcactaactacttagaaggtgttacaggcgctcagtattatgacttgttagactatgaactaatgagtgacctcgagaactatgcaaatgtaaactttcaacatgaagatacttatagagaaattgaattcgaactagcagagggtattgcttctcgatatgcaggttttgacgaagaagatgaagacatacaacaagacttaatttttgaagcattagactatgttgaaatggatattcaagacattgacaaagtgcagtttgttaacttagcagttgagattgtagcacaggaattagcaggagaaagaggagacatctaagaccctgcaggaagcacatagagacacacagagaagcttaaccgcttctctaatacaattaatcaggagatgttgaacatgaacacaagaagggtaaacaaagcgttaaacgaagcagttagattattagacaagcaaatagcagacacacagaaaacaatgcaggagttaaataaccaacagataagagctaagcaggagctaatgacattagttgatgctatgactggtgatgatgagtaatgaacattagagaggttcataatgtcgttaagagtgcaaagagcaaactcctgcaggagcagaataatattaataatgtaataatagatgactacatcacagaggagcttcacagacgcacacagagaagcgggacaatacagatgaacaataacaccgcttcatatagtaatggctcatatggtagcttagaagagcttagagaagcttatgacctatcttcattatctactggtgagattaaagaactacttgaaacatttgtttaaattattttatcaaaacgctttacaactatttaatttgtatgatataatgaacttaacaaattaaaagaaaaggaaatgatgaacatgagagacttacaagaaagaaaaagagaattgaaaacattactatttaacttagctatagagaagaacagagcaactgacgagacactaagaagtgtattagaagaagctcatcaagaggtaggaaaccaactaagaaaagtaagaaaagaaattgaaattttagttgaagaaaaagaaagagaattttggaacgatttcgactttaatggattagactaaaagggaataaaatccctcttttatttttatcctattatataatttttttatattatacgggggcaggggtaaaatgccactcaatgggggtgggtctatatacccctacggtctacccaggtacttattttttggggaaaattatgaaaataaatatttaaaagtcaacacataaaatatagaaagtaagtcaacataccttataataagtcaacattataaccttacttggtaagtcaacaatttagggtacaaatataaagcctctaaatataaagtcaacatatctaaaataagaagagaggtattatcccctctcctaaactatataagtacgcttctattatttaattccttctcaactatattcaaatgctcccaaacctcaatccagtttactcttgaatcatcataataaagatgaaagtcttgtgggttttctaaatacgcttctagttcatcatagtataaaacatcaaaagatgtgtcgtcaagacctatttgaatttttagtatatctttatatcctaattgtttactcccgcaataagctgtatcttctaactgcttctctagtttattaaatagtgtttgtataccttgtaattcattcataatgtattacctcctaattgattatatacttagtataaaggataggtgttagaatgtcaacctttatataaaaaaagagaaagaatattattccccctctaaggtattattgataatttctaattcatggatagtaatcatatccattccatcgaatcctcttgggtctcctttaatgaactcttgttctcctctataattagtctcttccctatacccttcttctttaatacgtttaattaagttctccttatttgtatataccttatcttctctgaaatggaaattatcttcataaggttcacaattatcatgttctacttgatacagtctcatattagttatcctcctattctctatagtagtcatatacttctaagtaaggtgaaaatggtgagtcacctctagtattaataataacttcaccctcttggttacttaactctataagctcttctaagctattaatttctacgcaccaacgttctatagtaaatggattacctttttggtctacacgttctaattcctttatgtaggcaccttctataggctttttattaattgtgcttgttctatctataaaaaattccattagttatcctccttttcacaagaccattctccgtattctgcgttaaaatgggctgtatctgtaccctcatcttcatattctacactataccatgcatcctcttctgtttctgcatctatatatcttatttcttctgtagtgatggtacgttttaccttaaatctctccatatgtttttcctctcctatcttctatcctttgttttatttcttctcgattacggtggtgttctttactcatttctttacgttccttatttgttaatctgattctataaataaggtagttaatatataatgttccggatgaccatagtagtaggaatgatattaggtaagtccatgagacactgatttctatcattgtgagtcctccttatattctttataacttttaataacaatcttacagatsaacctctattaacagctaaaaatagtataaatgataataaagttataactgctctaatgtctcctgtaaaaggtaatactttaaatagtaaaacactttctaaaatacttgtagctgtgataattgtgagatataagatagataataagtaatcctttaatttcagtttaacaaaaggttttttattatcttgtgttcttgttataccgtatagaattataaaccatgcaacaggtatgagctgagttataaagtcattgtttatgcctagcgcatataaaccataaatgatacctgcaggaataccaataatgaatgctaaaaatacagataatataattagcattatagggagagctacaagtaatcctagaccttgtattgagtgctctaatgtattctttcctaaggctttaaaaaatgttttattcatctgtttcttcctcttcttctatagaattactttccgtaatagttacttctagcatattattataatactcattcttttgattaatattatagttatcattatattcattgaagtctacataaatgtattcatttgttccatcttcataaataatatctatagctgtaatatctgaatacgctgtaatcatttcataagcattcgtattatccgggtaagcaaaaccgacttgaggtatttctttaggattatcaataagcataccaaaataagtacaatgacgtgttctatttatgtgtgaagtccctttatacgtaccatagtaatctataccttctgtaataccttctatatggaacctgtttacatctttaggctctagtcttacaacatcacaattttctaatactaagtctatatattttatattcattttaactctccttttatattaataattctttccattctttatcaatctttttaagttcttttttattatagtccccatctttagttgtcacagcattccattgaaacttctgtaataaactaaagttgtttataatccatatattactcttgctataatacatgttatcttcaaatcttatatctttttctataaaatatttatatattttatatcttctttcgtctgcccctgatattttaataatttcattagtatttaattgagtggataactggaagataatatcttttacttttaacaagtctttaacattacctctgcctacatggtcattatagtaatcgtacttagcttttttcttttgttttctatcattgactacgataaatatgttataaataatataagctttaaaatgggtgtaagaagtaggtgcctctgagtcatcacattcttttcttaggtctgtacattgtatctttaacgtaatattatttgatatgttaattacggtagaaccatcatgttttttattaagatttatcttatccatcttataattgcctacttattataaatacaatgtactcgaatgtcttccattactttacctaatagattttgacctttccaattactttcatctaatattttagggtcatttgctttaagacctactccccatattttatcataaggtgatgcctctacgaaatctttacgtaaatctgtatcgagtatttcccgttttaaatgtgtagtcataaatttatctttaaccacttctaccataatgtcatatcttaccttatcccattgctcttcattaaaattacgaactttacgacctagacttttagcatggttcggattcttagaatttaatatttctcctgctatctgccaatctttaaaatattgagctttgcgccacataaaagcttgttctgagttattaaatgttcttcctttatgcttaaatgttattgggtaaaagttagaataaatatcttccttaccccaaaacataatgtattctttagtctctctcatttattcttacctccctatacatatttatctaaagtactagccattttatcgagttgtttttgtttctctatctttaattggtttgatttatcacattcttctttgtttgattctctgaactcccctttatactcgatgtactcatctcttaattgaataagggttttaaaaaaatcatcataagtatttggtatacgtttacttacatatccccttaaataacctgctaatagacagagttcaatatcatcattaccataaccttcaaaagagttgagtttaacaataattgtgaaaaagttttcaaatttaggtgttttaaaatcaaagtgtatagtatgacctttactatccttatcctgacgatatgcaggctctaaagctataaaattatctttattaagtttatttaatgtttttgtatctttgaatagttctagtaatcttcctaaacttattgtttccatatttaaaatccctcctttaattccatagtgatggtaacacaattttgaaattatctaatattttattttgtacttgttcaagctcatcatatttatccatatcaaagtcatccatttcttcatggtagtactgtattaagcttaaaatatgttttatcatatctatttgtgttctttcttcaccatctacatctacaaaagtattccattccatatctatacaagaacgttcactactttctataaaggcatttaaatcagcatataattgaacaaagaaggacatatcatagttccaatacttaggttcatttcttcctaattttttattcatttttttatatcttctatttctctttaacccaaaaacttctttctcaaaatcattaagtttaagacctttaaaatattttttcttcatatctaatcctccaatttaataagcggtaaatctatatctctaaatacagaacctacatcacatagcagtatatcattatgttcttctacttctccactactagtaggtgtatgaccacacacatatataaatccatcttttctaggttgaaagtctctagaccatattaattggtctactgtttgctcttctataggtttccaactaactccccctgagtgggagaatatatacttaccttctttataatactttctacaattaaccataagtatcttaaactttctataatcttcagattctttaagtttsactttaattcacttttaataaaatcataatgatttcttaaattaccttctacactactatactttaaagttacagtgcttacaccataagaattaagtgtttctatacaataccttgataaccattcaatatcatagatacttaaccggtctacattctccattatattgtaaaattcatcatcgtgatttcctagtaaagctactgcattgtcatcatttgatataaaatcaaacatgtagtttacaacatctttagaccttttgcccctatctacataatcacctagaaatacaatagtatcttcgggtcttctttcttcatttatcttacccattaatgttagtaatttattatactccccatgaatatcaggaaccacgtatatagccatttaatctcctccttattgtatataactatcttaccatacttatttataaaagtcaataaaaaaacacctattaatgaaaataggtgtccgacagagctcccgtacttagattacggttaataatattttacgacaattatatgagaccctctgtcgttgaaacgctcgtcactgcgttagatacggaatggagggatactaccatccggagtctacggtcagatacaaagcctctgccgggcaacatacggtatctctcgtacatcaggttgactagacctttagagattttcactccttctcttataaccagtaacttaagagaaataggttttacttagtagatatgaaacaataaatccacatataatattaaatcatagtcaagtaattgcacatatgtctaatacctataagttttttgctagcctggtatatggactctgtaggattcgaacctacagtcaaaccgttatgagcggttggctttacctttaagctaagagtcctagaaatatcctgagagaggactcgaacctcaacgactaggtagctacaactagccaatgccattactcaggattactagtaacgctaaatagagtcataacgttaccgtagaccttttctactcttggtagataggtaaaatataatgatttcaaagtacctatatagtcaggctcttactctcattataaggttaaaaaggttaactgtgtttagcattatataagaggctttagttaactactatactaatagtatatcataaatagtacttagtgtcaagataatttatcaattgaatccataatttttgatgtacttcttatatccgcttctttactgtgtttaagaagatattttaatttcattagaaaagaatttttttctttttctatagtatctttcttgttatcgtattctgaatacatcataaattctataaatatactatttctgtcagatgaaaacatatctatagaaaacggacaatcaaaacttatgtcatctttattaatactgaaatcttcattaatgtgtaaatcatttatttcgaatacttcgaaatatccgtcaactcttttaagttctatagcaatattgtacctataataacgctgttcctttattaacttttcttttgttagataaggatattcatttataaatataggattacttgttccatagttatctttaatatactcagcatcttctaaggaatcagtataacctaaaacttcataacttgttgtatacacagtatcttcttcccacaagtcatagtccatttcctctatttcttcctctagtatataaatttttttcatatattactcccaaacaccaataagatttttaagtttagctataacttcttcttctgtttgataagaaaagactcctgtaatgtgttcatagttacctacaatttcataatcctgtgtaccatgtttgtctactagatatgagttacccataacatttaaactatcctccgagtaactgaaatttatattatagtctactaaaaaattaataatttttttcatttacataacctctcctatcggatattgtcctaacattcttgttccattttcattataaaaagtatattctactacaataatattcatcatatctacatatatagcttctatataaggtgtaatattttcttcttcttgtatatacttacctataatatcatataataattcagagtgtattcttttatctctcattatagacctccgtaagaaatgctacggttttatcttttaaagacttttctactagttccatagcatctttataatgttttatattagattcattagacttcagtttatcttttacttcttgaattagaggttcacctttagtaactaaatcttttttattttctacactcacattgcttcttttattatctaatacttcttttggcatatatttaacttttgcaaagtctttatagctaacattcaagttatctaaatcatctaataaatcattataatattctaagtgattatagaatgtgtaaaacttaacaaggtctttacctgttagctcttctttttttagtatgttattgatattaccaataacagaatatgctataggcttaaaattagctctaacataagttaaaaatataaaatcatcataaaacaaatctaaaacagttttattgaatctagtatttttagcttgctctaattgagcacataaattaagaacattatcaaacccactctttaatactaaagagataaatctttccactgcatagtacctagatacttcagtatgtttgcttgctttttcattatttctaaaaatagtatctgataaaggttgaactactaaactcatataatccttatctgagtattcatctgatgttccttggtaggtacttccaaattctattgtagataataaaaaacttttttctaaattcattataacatcctccttttacttgttattataatattaacacatatgtttattaatgtcaaacattaaatatcttctgttgtgtcaacttcatcttgtctgtatttaaagtgttcataaactttaaatagtataactcctagtgttattaatcctaaaatatatttcataacaatcctccttaataaccatgtttagttacccatcctgctaaagcatccatagccatatcatactcttcttcatttttagttcttataattttctctatttctttttttgcttgtttagagttcataaaatcaatatctactgtatctaaatttgtataatcaatattttctctaataaagttcttttgacccggtgttatagaattaactcgtacgttttcgtgatttagaaattggtagaagtccttattactcatcctttttaacgtattctgccatatcttttaaaatacttagtacatactctaaatctctatattggtcatctaatgaacctataatagcatatggtgtcatatcccaggcatgcgcacagtcaaaccctaatactctcttaccctcatagtcataatcatcgtaagtaatacctctatgagcacgtctttctaaagagtcatattctttttcattgatatctgaagataaagttatatatccatttaaatgaccagtttcagggtgtctcttaacagttagtttaactcctttataataaatatcaagacttaaatcttctcctaaaatattattttctttttctactttttccataatgtattggggtgcttttttaaacataatcagtcatctcctttttatttatatctttactatacactattttttctattttgtcaacttttattttttactccgccctcaacataaattcccaaagacttaactaaattttttatagcaggggcacttatgtcatatctagaacctatacgcttatacccttccccttttagatatcttctttttacatcctcaacgtctatcttactataagttatattaaaaactaattgattttttattttttcttcattgataagtaggaaaggtaactttaattttaaatcatctataagttcttttatattagttgaaaaataattaatttctataagtaatatattattatttttacaaaattctttttttattttatcattttcaattatagttttatggtcaaatcttcctgagtaggtatcttctatatgctgtctcccttgtacctcaatacagtactctatatcattataggtaaaatagaaatcaaaccttaaattaaatcctttattactttttaaattactaaatgttttctctgttatataatttatattgttatcgagcagtatagtttcaacagctttctcacctttagacctcttatcagaacacatacgacaacctaaaccataattatataaatcagataattttatttcttttttactacaacattcagggcaagttaccttaacaattttattagattgaagtgttaaatcttttacctcttcccagttattaggaaataattttttcatatcttcatcatcagacagccttttctcaaaaagttttttaactcttacttcctttaagtttttaccacatatatttatagttcttttattacaactattataaagttctaaataatgtgttttattaaaataataatttattacatttatatcatatacttcattattataacttattttaatagtttctccaggtaaattatcccattttaaattaccattatcatatattgatgacaggtctacagaatttatttctttacctctatcacaataatatataccttctatgctatatctttttatagtcataatatatacactcctattattttaagtattctttaagttgctcttctttatagaatcttctattagtaggggttctataagctaccagtataccctctctatcccatctttgtagtgttttaacagtaacacctaataacttagctacttcttttggtttaagtcccataattaattacctcctgttttttttatctatatataatataacacaaatgaacacattttaacacctttttttaaaataaaaaaaataggcatgaataaacatgcctaataaagtattacttttggtagtacttccatttgaagtaaccatttttctgtgagaactcaaagtgaaaaccatcatagtcaaattcaatattgtagtctccatcttgaagtggttttgaatttagtacaggactattactctttgccaattctgctagaaactcatgatttactttttccatagggtttattcctcctaattattcttacagtactaatatatcacaggtctttttctaggtcgtttttaaatttctcctcataagaactagcataagttacttcataacctattatcttagcataatctatgcaaagtaatttataattggactttattttaatatcttctgattgttctattttgttgataacttcatttagctcatccgaagagtaatgttcattatctacttttattgtctttccttgggtatagatatcaatttcttgtatcatcatttcatccttttgattattcattatttgattataagtttctaagtcatcaatgttatctgtatctgaaccttttactagccattctcctctcttcttaaggaggtcatcaaacttctcatgctctttaattatcttttctacctcacttggtattaacacagccctagcatagtttatatgccacatagacatattatcaataagataattaaccattcttataatctctttttcatttgccatataccaacctccttatatctattattaatataagagaaaagcagacttattaaaagtctgcttctttacctaattctaatcttctatttttcatatgaggaatcgtttttttatttcctgttaataatgataattctctagctttttctttagataatgttagtagtccattataattatctactttactattatattgtctgactaagtactctagttcatcttctatacctgctagttctcctgatttaactccaagtaactttctatacatgtcataatcttcagaaagactttctactttgtttttagatacagaatcataaactgcttgtaaattaccttcttcaataagtttaaaattatattcaccaatgattaattctttttcagaagagtcaagggtaactaaaccacttgtattacctgtaaagtcacctttataatctacaacaattccttcagttattttatctcctaattcaatagtcccatcttcattttctttaaatttatgagcatcataaacttctactttgtcacctaatctcaaatcttgagttaagttatgtttaccgataattctatccattacttaacctctcctttattaatagggtcttgtgttaagaacatttctaagttctcttttgtaataggtaaccaaaaatatttactttccggaattgtaactgtatagaagtcttcatcattattaactttaatattaacatctgtaaactcatcctgcattaaccaatgagttacagttaagttatatgaaccatcactaacataacctaaatcaataccatgtctaaaagctaaatcttctaagtgctctaataaatcattcttttcattttgttcttcttctgtattatttttgattggattaattaactctgtacaaacgatatcatacaactcaccatctgtaacttcataattcttttctattaatacatcttgtattttattgattgaatttgtaactactgtcccgtattcttcttctgtaaaattacatttatctaaatcaacatctgtaattaattctgcaatccatttatttaaaattgataccgccattgttctagaaataatactatcatataccatatttatttaatctccttgtttaggtgaatgtggtcttctaatgaaaaatcaaaaggcgctacaccgtttcttttattgtttgtttcttttttaagtataacataagttagtgaaaaagtcaagatagttactacaaccattaataaaagtttaatcagattcttcataattactctaactccttaagtttattttttactttttctttatcatacttataatctttttctgagttttcatttttttctttctcttcttcattaagttctctgtattgagcttcttctacctcttgttccttattatctttgcctgcgtctgctttttgaatttctacatttttactactaccgccactcaccttttttctaaaaagaaaccaaagtattaataaaatgatgagtaaaataataatgcttaatacaaccgcccaaatattattagccattacaacctacctccaaatagtttttttacagctcttaagttttctgatgactcattgtttatatgaatacctgtacttgaatcgaaaataacagcattatcaagtaagtgttctttaagttcattttcataacttcctttacctactacactaccataaccatcgtgtgttaaatcagtcatatcagattcaacttctaatactctaaacgatactctacgtaaaaatgatgggtttactaagtaaaaggatgatttaaaaacatttaatctttgataagaatgttttatattaacaacaaacccagtaagtttatcgttataacttgagtttgatagcttacctagatataaatttatactatacccttttgtttctagggtttgtatagcactaagcagtatagctcctctataagcaagattctcagggtcttccatccaacttatactagaattatagaatacatcaataactttcttctctgctttaactctctgttgagacatcatagaattaggtaaaccttttatagcgttaggtacatgaggttgataaccttcaggagctacaactggttttctttttacagatttatccattctgaataatgcatctgtcatctttttaagcttaacaaccatatcataggactctctatctcctttaaccattaagttataggcttcattaaagctatgagtgcccgtgaagtcgtagctacctgtatctgatgaattgtctctacctgaaactctattcttttttaaagcagaaaagaaatcaggtagaccatcatatttaattacatttaattctgagttatctattaatcgtctacccattgttttttcctcctattctaatcctagtttatccatgatagtgtcaaagtccattgaatcactagatgaactttgagtttttcttggttcttgtttaggttcttgtttaatacctagaagattttttgttgcttctgtgtatctgttatcttcaggtaaagagctaataaattgattaatttcatctttaggtacagacttgaaaatgatactttctacaacaaactcatcgtccattactccatctaatttacttccattaataattgcacgcattgaaaagacataaggtaatcctttttcatcgttctcatatcttaatttttgtacaaagtttactaaatcctcattacttgatagttggtgttctaccttattatcataatcaaactcaacttgagcaaaacggtctaatgtagctccatccagttgctgtcttcctacataaatgtggtctgctccagttcccatagtattacctgctgatactactctaaagtcttcatgtgctgtcacacgaccaatagggaagtcaaagtatttatttgcaatagctgagttaagaattaataatacttcagggatagatgcatccatttcatctaggaagaataatcctccatttgtaaatgctttataaaactgagtttcatggaatttaccatttgcatcaataaatcctgttaatttaaattcttgagtaattgcattactgaaatagaaatctaagtctagagcttctgctacttgctctaatacatggttctttcctgaacctgctccaccttttaaaaacactggaatattttgattaactagttttaatatatcttgatatctataatggaagattcctgagatatctttaattgttttaccttcttgttgtaattcaatcttaactggtaaattattaagttgctcctctacatattcttcaatttgttttttaacatcagtaataattatttctctactctcagttcctgctttctctacaattgcatctacaattgcttgttcataagggttagagtttttctctcctagtttatctgataagtctttcgttgtctgcatttgttgttctactaatctctctaatctttcaatagtatcttgctttgccatatttatcattctcctttgatttgttatacatttattataatacaagtattttcatttgtcaacaactttctaaaacttttttagttattaataaaaaaataccttacacctataacttaacatagggtaaggtaattgtcaacacttttatctaaaaatacattaatttaaaaaagtcatcaatatcattagttccatgtgtatccatatcatacataaacatacaattataagtatgtttattcactatttctagcatattgtgcatagaagttgcattattgaattcctctaaatcaatagttattgtaagttcttgaccttcataaagtatgtttgctatatagtatgttttaacaccttccattgtcccgtgggaagtttcattatgattaagaacttctatacctagtgaaggtaaataatttgaaaaatagtatttgcagaaatatataaaattgtctgttcttttagacacgagtactatctccatattttatatttctttctaatcttacataatatgttttaatcttttgaacttctttgtctactgcatcttttcttcctaatcttgtagtatattttacaatattaaatatcatagaatcaacaaaaccgtcataagaaaagtgttcttctagaaaagaaataacatccttactacctttatagtgctcaggtaaatgtgcatctacttgtatattatagtaatcttctaaaagacctatactttcaccaagactagataaagcataacctaaatcattagagtcattagaccattctttagatactgataacgcatcttctataattgttatttttaatttatctaagtaatcttctacttgagcttgtgttttcataaattcttttgcattcatttaataccctcctaaattgtataaaaaaacaccctgcttggatacaagcaaggtgaaaaaggaaagatattatggaagtgtactatctaagtacacctcataatataacagttttccttgctagttattacttattttttaaggtcttcttctttgacaaatactccgttaataagcttacctttcctgtcttttatctcatcataagccatatcaatacactcttcaatatctatatctaattgtaaacatagtactgttaatactacaaaaatatctcctacactatctcgtgttacatggtcattacttttagcaatacctgaagctaattctcctgcttcttctaataactttaacatttgaccttcaggtttacctgtttgtaaatttctatcttttgcccattgcttaataagttctactttttccattattctatatctcctttaatttctgtatcttttataattagattatcagaatcacttattacagttaaattatcttcgactaattcatgtaggttattagtaatatcttcttcatacctataacctacacgaacataagctttcaccctgacatctatattaacataatcttcttggaatttttccatttctaacttcctttattataccatattgttatacctttgtcaattaatctgagtagtttcctttggcaagttgatactttttgtgtaattcttcatataattctctcataccttcatagtttctcatatcatcttccaagaaactaagataatctaataataatttagtttcttctacttctaaagttataactggtttgaccatttatgcaacctccttaaattcttctttatttattttcttaatatctttttctaatgcttcttttagttcattaggtaatttatatgcatcaattgattgttgttgtcctaacacataaccattatctgtaatacgtatttcaactgtaaaccatgaattatctaaatcttcttctaatcttgctaataatattaaacagctattctttagaattctattagcgtatcctccaacacaatgagataacattctaccttcatcttttaatttacttacagtatctgcaggaaggaattttacttttctaccatcttttaatttataagttttatcaattattttttctaacttattatcatacttagctttaagttctgcatcatctaattgttgttgaatggattgtttttcatctgtaactatatcatgctctagcttcagtgaaaaaggcgttaagttaacactctctaatgttctatacccttctctgattagtattgataaatcctgtaaatagtcaaggtaatatctatctagtgcatatcctgttatacgttgtctgtcttgagcatctacatctaaatagtgcgtcatctttttgtagttagcaaaagatatagacaatatttcatttacaataggttttacttttaaagcatcggtaacatctcttgaatctcgaaccattaaaaaagtatcatcaaataattggtgtaaattaacctcattgtgtaaatgattatagtacttatatagagtattagcaaatcttaagtaattaccttgctcaaatttatttaaagttagtaattttttataagtctgttttgtaagattgaaagcttcatgaactttccatttaggatttttaggtatatggaatagtaatgcatttctttcaaataacccaaattcctctaagttatttattttatcaatatttttaacaatatctgttaaagttgttaagtaattagaagttgaattttctcctataaaaatcctatatttgtctcctctataatttatatgaccataaacatctatattatccggacaccaactagaaaaatcaaaattatggtgttctaatgtttgttctattatctttattataacccctctagttaagttaggttgggagtagttttttaaaataacatttaataaaacagctaaagttaattcattcttatattcacttttactaatatcatctttatataggtttaaagttatttctttattaacaagactatctgttaagaaaactttaacttctcctgttttaacgtcaaatgagcttttattttctaaagtccatttattacccatattatgtttatctctaatatgtttaactttaagaccgaaagatgaatagttttcagtacttggatgcatgtaccaagcactgctatacaatgaatctgacatttccttataatacttactagaacttttttctgtatcttcattcatataagaagtaatagatgattttattaaaccatacttaccttggtctagaatatccataatatcatcatttaaactctctactacttccttatactcatctaattgtctagctccatacccccataatcgggtttcattttctaattctttaattttttcttctacataacctttagattttatttgtcttctgctcttactaccatataaaggaaattcttttctttcttctctactggattcaatatattctttgtaatttcttcctttattttctccaattacaccttcaactaatttttcaactgtttcataagggttacctgtaaagtttgttacttctttattaccacatagtgctaagaataaatgtatttctgtagcagtatcaaaactaaatatgttatgaatatctctaaatagttccttagagcctaagttaattatattatttttcttcttcttaaggaatacatcttcttctcctatatagatacaccctttattaaccttaggtagattaataatttcttgttctgttaatcctttttgtttatatgttattgccatttaaaatcactccttatttgttatgtactaatcatatcatagtaagtaatatttgtcaacaaaaaaagaagaacttttttaaagtccttctaaatgagtttcgtatataaccttttgaattttatttagaggttctaaatccaaattcctaataagtttttcatactttcttgaattcttgaagttaatagtattaggcatagcaagagcttcatcaacgtctttagtatagcttacaacatctgaatagatatccacttcttttacatatagaccttgagttaaacttctaaatactacctcattatgtgctataacttcttcttttttttctatgctcatttgtaaacctcctggtttattctacacaaacaagtacgtactctaagctagttaatgatactgatttaatattatttaattcttgcaatttcttaatatctacatcatagtttttacttatagtccataatgtctctcctgctcttactttatggtaatacttatttccctctttaataaggtcatccaatattacctacctccttgagtaataattagcctgtagataacatataagtataagaacaaagtttacaaattcagtagctataatgtgaacataggtatgtgataaaaccatacttactatcaacgaagctaatcctaatccaataataaggaatagaaatctgtttgtcccttctgaacttttagttttatagaatgttgttatctgagttacataagcaaggataatagtaatagttgctacagtttgtgttaaggctgtaaagtcacttaataaaaatagtaacaatgagaacacaataataaaaggtatagagaaataatccttttttctatatgaagctactaataagcaaacaatacctagagttaaattaagaccaactgagactacttgaaacattgtagcatcagttaatagtaaattgtaaaaactaatacctactgtagctacaattaaataccaaaaataactactaactcctttaacactatccgatttaactaaagctattagacctggtatataacctactgtaactaatatagcatataatatactcaagtaatgtgataaattatccatcttgttctcctaatttttctaatctttctaaaacttcttcccaagagataaatccttctccacctgttaattctaaaaccataccataaacaaattggttagtgctaaattcagctctatcagggtcattatacggcttaccatgtccttgacgaatatcagaacagtaaattaaaacaggtttatttacaatattttcaagtttgtctactattaatttctcttcattagttaatgaagtttcaaatctatttgacatataattaaagtatccttctccattatcgtatatatggttaattgtttcttctgcttgatgtttcatacctaatagaattcctaattccgcaatcgttcctaacccttcattaagaatatcaaagacaaaaatatctgattcttgcatagctttaaaatcattgtttaggatacgttcagccaaaccagtttgttctgcattagccttatcattaattgatttatctttatgggggctataaggaattactcctacaataccttccacttgtttatgttgtttatctctatattctaccattgcatcatttaaaatatgtccacccatataaattactttgtctttaattttattcaccatttatagtatctcctttttcttctaaaatttctctcaaaatatgtggcatttttttcttaatttgtttgtctactattttcagtatattttctttttcttcttccataatatcatcaacaaaattttgacctacttgtttcataattaaaccaaaattttctaattctaaatcatcttcagttaatctattctcttctatagctctaaaaatcattttttccattcttgattttgtaatagcatagtctgctaccgagtcattacttcttatttctgttttcattttctgtctactaaactctttaaattctttagatactaatttaaagtaatcatcatattctgatttaccatccaaatatttaattacaataccttctccagtattaggtttaacagtcatatctgattttcctactaaatctttaatttcttgagtatttaaatcatttaagtaaatagaaggttctacaatcattaatgtttttacagtttttaaacctaatgtttccgctaaggaaagtacttctgaatgaggtaaataggtttcactatccttatcgtatacatcgaatacataaaaatcattataacaatcttctttataatttaccttatgtttaactaaccattctccaaatataataataccttctaatatagataaatccaacttatctgtcatgttttcatgtacccaattataaaaaccatttaatgtttcgttttcatttaatttttttctacgagagaaacaaactaactcaccattctctgtagtaaagcttgcgttactcccatctaatttttcttgaactacaaaacctctatctttaaatttgtctaaagataatcctttattttttactttagtataagatttcattaattagttatcctcctttgaattatgtactactgaaaataaaataagacttacacttgctaaaaatgctaataccactaaaccaggtaaatttagaactgttgataaaaataaggatacagcacttataacataaactagaccgcttagaaataaagttaataatacaattgttataagttttaccaaccaattattattaataaatatcctagctaaataattcataaaaaatcctccttagttattataagataagtataccataactaaagaggtttgtcaacatattattttaccatttaaaattgtctgcgtactgagctaacttagaacggaaattaactgtaaaattatgaaataccgcaccatcataatttttaaaatattccatataatctccaaaacctgatttactttcattttttaaatctatttgtttaaaattaccttctactattacagtagaattttttgtatgaacccttgtaagaacttttttaagttcactacgtttaaagttctgtgcttcatttacgattatagtagcatctctcagatttccacctcttagaaatagatgtgatatctgagatacccaacaatctcctaatttatcttctttgacattatcttccatcattaacatttcagttatttgttgctcagggttcatattaagttcaataagagcgtcatgtaaccccatgaagtaagccatttctttctctgtctgattacctggtctacttcctaaatcctctgatactggtgaaattataaatactagctttctatttttattaagataatctgcataagcacaagctactgagcacattgttttacctgtaccggcttgactttcattccaaagtatttcaacattatcattaaagaaatcctcacagaaatctaactgctcagttgtagctttttcaaggaagtcattaaatactagatgctctcccatattatatcttacattaggataatcctttaacttaaagtctaactcttttagttgtattgccatattttaaagttcccctatctataaatagttttactctcttttaatatagtactaattaccgatatattcacccgttgaggaacaataattactacagttacattcagggtaattttcacaaacttcttcgtcttcatcatcaaataagtcgtcatgatagatactagaatcaaaatctacaatacaattttcccctttacctttagatatacctgtataaataacttcatcaaccatatcccaatcattacctgctaagtaatttacagtatttgatactgaatcattatcaggtaaatagatatcaccatctgtaaacttaattaaaatatccccttgaggaagtacatcattaattaaatcaatttttgtatcttcttcaatagtaaaaacagttccttctagtttttcaggagtagtatgtgttaaatgtttaacatcttcccctgattcttcataaaatcctactgcattcatttctttgttatactttgcaataaatttaccattatcactgactaaatattgactgcttgcgttataatcttctgcatcttctaaagtcatagatgggttatggtctttaacataataactaatttttctaacatctgttgtttgaattttctttccttcacctttaattactgaattaattttcttcataatattttctcctttttatatatcaattgatttttttgcaagattatcagcgtagtcattccatttatcatttgagtgactttttacttttacaaagtttatatctattactttttggtattctcgtatcatattaatatatgttttacttagaatatttcttgcagaccaagtaccttcataccaatgtattaaaccaatataatctatataaactattgcctgattgtatcctagttttatagcctcttcgataccataacaacaagctaatatttcacctgcaacattattatactttatcaatcctggtttgtcaacacttttactaatttccgatattatatttccttctttacttactaagacagcacctgagcctactttacctttattataagatgagctaccgtctgtgtatatatttacactatactgcatatttataatcctccataaattgagggaattcacaatctgagtatacttctctgcagaaagatactgaaatataattaaaatcaaaacatttgaaacagtgttcttgaacttcttttttgtctttagcaatcacattaaatttaaaaccatcagctattactgtaaatactccttttttcataaaacaaatacctccactaattttattttaaattaataactaactcaataaacgatttaatagttttatttttaccttcatcaatatctgaaaagaaattaattaaactatcatcttcatcaaataaatcttcaacatcatcaaatttatttaatatgtctgtaacactataaccttcttctgatatatactcatgcaagtcttctccatcctctgacagagttgcttctatcttaccattcttgctttcaattaaatacaaagtatttaatactttaatagaatccactactacactgtaattactaatagtaggatactctgtataaagtatttccacattagtattcatataactatcaattacagaattaactgtgtccctttttaattcagatacattatgttttcgtatagtagggaattcttcgtcatattctactaattcttttctatctgtattcaataacttatctaaagaagacaataatactattttatattgattgtccggtagactatctgtaatttccataatggttaaaaaagtatcttcacctaaaactttatttatatcctgtaattcaaatgaatctaccatttcaatagtatcatctatatcatctgtagtcattaaaaaattaactaaattattattctccatcgtcttcctccaattccttaaatagctctttacctggagtatttaacgccttctctaacctcattaaattagcacttcttggtttctttttaccatactcccaataagatataagagagtaatggactcctatctcagaagctaggcttcttaatgtatgtcctttttctactctaattttttgaagatttagaggtttactttcctttttttcatccataactatttctcctctacttttaaaaatttaaaatcctcagatgcttttgcattttttagtatatactcttgtgatttatttcttgcctctgccttgcttttagcatataactctatatgaaatacatgaggtttttttaaagacagtgattcgtatctccaataaactttaaaaagtagtgtttctttttttaaaacattaattcgaaaccatcttttaaatttattcattcattatcctcctttatttatttgttaaactaattatagcatagttaacttatgaagtcaactataatatacaaaaaagactaagaaattaatcttagtcttaatatattaataactattatgtgtgttatattctatttactacttttggtaaaccttttctttctcggtaatcatctctaagttttgtagcctcctctaaagtaaacctagtacctaaatactctttacctactttaaccatataagcatctctagctcttctatttgtgcctttatattgtatagtaatattaggcaggtctcctctttgtcgtctattaaaagcttgttctgtatggtctgaccatttaatattacccttaatgtaacctagattattgtttattctgtctatagaatgatttttacttggtttacctcctatttccttataaaactcccaagggtcatctatccaactttcttctattaatttaccttgtataaaatctttcccataagtcatttcatttttataattatattctgaatttactgttctctgtatcatactgttataattagggtaccaaggctctttagatgctccatgtgttttagagttatctgtcattctttttgaagcagattctctgtgtaggcaaccacaagaatttgtattacctctcttgacttggtttatatttagtactttcgtatttccgcattcacacttaaattcccatttagagcctcctttagatttttcgataattctaatagcagttaacctaccgaaaactttacctctttcatctaaaatttcacctttaattctgttaggtttcttagaatatacaaaattttctagcattacatttactcctttctaataactattactaatatatcataaataaagacatctgtcaagatgtctttaaatggttttaccaccaacctttagattgtctttcagagattgcgttttctactgaaccatatctttcgttggcatatttaattgctgattttgtttgctctgctactgagccagtaccattacctgattgaggtgacatttggaataatcctgagtattgaccatttgtggcattagggtttcctgtactttcaggcataacgatagaattccacatagcctcagtacctcctgcttgtaagaactgagcttttacagagcctcctgtagaactagttgattgtgtaggttgtttagtttctttttgaactggtgctgatgttggttgtacttcttttttagtatcttgtttattttgagtatcaaattgtgcttgttgttggtctactttttgttccggtgtttgttcttctcctgctaatctagatactgtattatctacttgagttgagcctgaatggtattcataaccaaagttaccattataattatagaaatgataagtaaattcaccatcactaaaagagaaatcataattacctgcttgaattggttttgtattgacttctgctgaatttgatttagcttgttctgctaacttattataatcaatttcgtctgcactagcttcatttgtagcaatacctccaaaagtaatagctgtacctaatgctaatgttgcaaaaattgttttcttcataaatttaaaactccttaaataattttttagaattgtttatttgtaaaccgacataagtaatcataacatatatctttaaataacgcaagtataatatagcactaattagtgtaatattattaaggttttattacaaacattacagttatcagataattagagacaaaaaaaagagaggtattatcccctctctttattatttacctgttacgtctacgatagttccgtctccaccaatttgaataggttgtttaccatcccatttttcaattaactgttgacgtaatatcttatcagataaagaagattctctaatctcattggcttttttatcaccattagcttctacttcttttttcttagcattttgttcagcaatttgtttatcaactttagtacgttctaactcttgattagctttaacacggctgtcaattgctttttgagtgttcttatctgctttagggctagataatgcaatatcctcaattacaaatccttgtttttctaagttatcattcaaactatctaaagtgtcttttttaatttctcctgtcttaacaccaaatgcatcaattacagaatacttagatactgcttgacggacattatcttgtacacgtgagcgtaagtatcctttttctagttcttcgatatctgcgctaccaaaacgattaaataaatctacagctttagttgcatctactttataagatacatcaatatccatttgtaaattcttaccatctgaagttgctacatttaaatctttatatttatgtgtttgtgttttagttgggtatttatttaccttatcaaaaggtgctgttaaatgccaacctggtgatttagtatcttccttaacaccatttactgagtatacaactccaacatgaccttgtggaatcttagtaatacacattaataaaataataaaccctataattgctaaaaaccctaatactcctgaaataactactgactttctcattacatttctcctttttctatttcttttattaagctatttaaagctttttcctcttggtctatttcttgtttatcggctctagttacaattgattgtctacggtcatttaagaattgttttttatactttacatattgttctaaaccgtattcctctaatgtaccttgcctaactaattccctgtattgttttcttatgttacttttcttctctttcattgaaagaaaatcaaataaataacttataccaaaacctacaaggactagaaaaacaataaaaatagcaaagtatgttaaaaatagtgccatgtaattcctcctttatttgattacatatataactatacactatgtatttaattttgtcaacacttttttgcaaaaaaatagacggatttaaaatccgtctaaatttatattctatttgaatactccccaggcaacaccaggtatttgattaggtggaacaccttgacaagttctaacagggcaatatactctgttaccattgtaagcattataacctatccaaatatgacctgcttggatacaaacttcgtcatatacaattgtagcccctgccggtaagttaccacctactggagcatttaagaatggagaacctattctagttactataggttggttaccattaacaaatgttgcattttccggtttataccaagttccgtactggttctttttccaagagcctgtaactggtctagttgccggtgtacttgcgctacttgttttaccatctttaactactgtagaacttgaagtccctttatccatgtagtttttaatttgtttaatgaaataatcttttaatttattcattattgcttgtgatggtcttccttgtgttactggattaaatcctgtatgaagaaccatagaacggtgagggcaggcagttggtacaaattccatatgcaatcttacagttttacggttaggagtaagaccccattctttaaatttctctgctgtaaattggaatactgcttgttcatttttaaggaattgagcatcactagcactcattgattgacagacttcaatacctgcaaatctaaagttacctgagtttgctcctgttccatctcctgtgtgccaagcaatttgattcttagcatctattgcttcccatacataaccttcagagccgtagtaatgagcaataccattagcgtatctagcataacctgcattagctaatgaattctcgtattgttgtcctgaagaacgacctgcatcgttgtgtattaccattccttcaggttttttaccacgtttatccattgtatagttaatgtgattcttagaaacttttagtgttgctttctttttaggtgcaggagttttacttgcgcttttcttagctgtttcttttttaacagtagttcctgcttttacaggtatttcaatgaagtgagttaatccgtaataattatctacacgttttgtaggttttttattagcataaccattccagttttgctctaaaatagtaaacgtagaagtattacctccatcatatacaatacctatgtgaccccactgttcataactaccggatgtaaataccgcaatccaaccttttttaggtatagtagaaggtttattttcatgtattttaaatccagtaccataactctgtttaatttggtctttagcattaccccaagttctaactttattatctgttaaccataaaacatagtctgtaataaggtcttgacactgagcgtgatagtaaccatctgcgtcaatggctcctgcttccattacaccaaatgatgggtcataacttgtagcttttttaactctgtaagggctatctactgttccttttgcataagcgtctaaacgtttatttatttctgcttgagtcttagccattacttaacttcctcctctgcaaatactttaccatgttcctcggtatcttcttcatcttgagaaggtgctgaaccaccatcaatttcatcttcaatagcaggtacttcatcactatcatctgtgtcaggttctgcattgttttcgtagctgtctatctcaaaagtactagcgttatttgcatttgcttgccattgaacgaattcattagggtctttactatcacgaggtttaagatagtctgtttgaacaatatcactatctttaagacctttagtattattatcaacaataatacctaaacctgctaatagtgttagtatagaacctacaatatttacaccttgctcaatttgagctgagtagtctaaaccgaaagcacctgtaatttggttagcaaataatgctactgctgatataattgctacccaaaatgttttgctcttagttcttgtgctaaggtttattcctccaacaactttaggttgtttagtttcattagccattaaaaaaccgacctttctattatatttatttctaacaataatataacagtaggtcggtcatgtttatctatattaatttaacacttactcattaatttggtttagttttttgataacttcagacatttgtttgttatctaaatcttctaatttagtttcaggaagtagctctaacttatcccaaacttcttctttattagatactttattattaataattgccttaccaactaaactttccgtataatataattgttttgctgatgccatttgtatctctccttttaaatatgtaaagtatatagctagtatcgtatcctaggaacaaacacttgcgctatatactcaatgaaatcctaccctcattcgaggacacagcaaaccggttcgtcaaccgcacatatgaattctcagatttcatttatgtaaaacacaccctctttgatttgcacaaagactaagggttttggagacccttgtactactaattatactaagggtgtttattatggtttctattggatttgaaccaatgacacctagagcttcaatctagtgctctaccatctgagctaagaaaccttgaaacgacccatacgagactcgaactcgtactctctgccgtgacagggcagtgtgttaaccagttacaccaatgagccaaaattataatgctataccctaaccttaccttaatgtatagcaggttttctcttaggctcgaagcaacgattattaccactcataacaactatatattaagtgaaaggaggtgaaatgaacaaaacgtggtaattggtacctaagaaggtaatatgtataatctacaaggagtaagttattggttcataaaggagtgtgaacaataaatacatgaaagagtgaaagtttactccctgtagattcttttttaattatcaatcaaaggaggaaactgataattgttaataataaactataaagaggaaaatatttatagtcacattctgatataatgcaactaaatatccaagcataacccgtctcacgaggaacctacctataagacctgttattaagtgaatcactacgattgactctattaaggagctaccttaagtccatctcacgcaatttaaaagggacttacaaaccgtaaaacggtcataagtttattaaatactgtgatattaacatattagttaataactttcacatggtcgaagaaaagtaaatttatttgatttccaaattatttttatcaaatatagctcttttgaacctgtagatttatgctactcatactgataacctctattatctaacacatttctgtgctccaactacagttagtcgttacagcgtatctttctaggattccgctaagaccctagaaagaaattaaaccctagccgttatcatactctacagaccttataagtaagtaccaagtataccaatcgtatttaacaatactaatgacgacccatcctaccgatatatctccgataggttttgattcgtttgattatcttgtaccttatgactaccaaatcattattcagtcactatgctcagatatttagttgtattatttatatattaattataacataatttttattacttgtcaagttaatttcaaaaaaattatagaagtagggacgtttacctacttccatttaatttacacaaggatgataacattgttattgttttatactggaaaacaatgtaataaaaacagtgatgtgtaaggtatttgttttattgttaattacattatagcatatactgatacctttgtcaagttaatttaatactttttttaaaatattagttatcttttgttaattcttcctgaatagcatcccatcttctttctgcttcactacgattatcttctatatgttttgtagttttacaacatttgatacaatatatatctttgatatgaccttcttctcttttatttgctctttttcttggtactttgaatacatttccacattctttacatattaaacttgagtaaaacattttttgtcttttcataattaatcaattccttttctcttttatttgataatttaactatatactatattgataaacaagtcaacagttttctaaaaataatttaaattattttgaagaatcctttaatatcagtacttacaagagaaaaagtacgtatttagaaaataaggagtactcctattatatataattatattctgatatacagtaattaataatattaaatatataattataattaataaggttgggaaaattgatataaacataactgatactgtttataaatactcagtataaaagtaaaatcccttagtatcagtacttacaagagaaaaagtacgtatttagaaaataaggagtactcctattatagttatatatatttattactattattaattactatttaaatatataattataattaacaatgttagaaagtcaacaatatcataaataaaaaagtgactacttaaagtcactcaataattagaatactattttaaaagattctattctgtttggattaatatatacttgaggtgaagttatagcactttcagtatatacttttatagaggtttcatccattcctcttaacatataatctatatcttgcctattgtaactcttttcatcagtagatactaaaaagtatttagctccacttgacattgttatttcaatatgttttgacatctacaatctctcctatgcsaaaatttgttaaagacaaaggataatatagctcctagaacaagtaaaagaaccttctcagttgtatcctttttcttagtatccttagtttttgtactttcagcaagttctgaaatcttttcatcaagtctttctaattggacgtaaattgctgattgtttttcactattgacagctacatctttatctatactaactatcatttttcttagttcagctacctcaacttctaaatctttgaaagttcctctatctatataattaccttcttgtatcttagacttaatagtttctacttgagaaacaaggttgtttatctccttatccaactagaatcacctctaaggtctaaccgtttcagattcagaatggatatcataattttctaagaaatcattgataatctccatataattatccgtaacgacttttccgtaagatgtttttgtatcaatttcgaacctaagcttaccaaaactttggaggtctaattcttttattacaatattagggtcatcagaaggaaggtaataatagtcgaagtatataattgagccatttattaatactctgtctattctatagacgtggaaatagcgtctgtctcttttaaaatgggctagtgcatctttaaactctaacttaaggatatccttatatttagtcaaagtggtaacctccttactattaatttttaaatttacttattttgtggtataatagttatgataaaggcagttattataattatattaagaataatgataataattattttttctgagaaaataagccaaatactaaaaacagataaagcatagatagctgatagatatactatattaagagttaccttacttttatcttttctatagatagaataacctaaagacgttgtaacaccactaagtataaaataatagaaacaaaaaagaggtatagacagaaaaaaagatacgataatcattgttaaacacctatttctttttgacctattatttctagaacttttagattacaccactaatataacattaaaagccagtcataaaagtcaattgttagattaataatataataaaaaaagacaataggaggttaaagtggttgaataataacatagctatattcatattcaaaacactggttatcattatattcttactactaattttgtctgttattaattccttgtcccttatttactcaataagaccgagtgtagttatgacatactttatctttggtggtattgtttctaatgtcgcacttactgtaacagataagttcttactgaagaaagaagaccccctacctgaatatgttcttaaaaaagtagagataaatgataaagaaataagaataatcaagaaaatcatagaaagtaattacggtataacagcagaagagataaaagttagagctaaagcacaaagaagagtagaggaagatagtaaaaaggaagattacgatgaaaacaaagaaagaaattaaagaacaaaggaaagaacttaaggatggtgctacatctgtttctttagtaaaaaaaggagataagagaatagctagccctagtagaatctgtagtctatgtggtcagcagttatcaggtatgaattacactaaaggaaaagcattatcaaaagttaatcattttcatttacagtattctaagtatatttattttgatatttgcgcagatatcaacaattgttataaaaatttaagaaaacgaggtgaaatggattgagtgcagaaaatattagagatataattaataagaaaaagttagaagaagaggatacaagaaaatatatagctgatggatttatgaatggtatcggtaaattaatgtacgaattcaataagaaagtagataataaagaaatagaagttaaagacccgaatgatttatataaattatttgtgatattctctcaaatgcaaaatatggtcaatgaaacttctgaaggaggagcaatacctcaactatctagacctcaacaggaattatttgatgagattacaacagaagatagtaatggagaatctacagttgatttacagaagatatcagaaatgtcagcggaagatattacagcaatgatttctgaaaaggaaaaagtaatgaatgaggaaaattcagaaacattctaaggagaaagatataaatggatggaaaagaactaattaagatagcacaagaaacatttcaaactgaaaaaataacaagagaacagatagaccatataatcaatatgctaaacccttctacctatatgcttaagtatcatacactgagaggtcatcctataacttttagtattcctaacagggatagaagtaaagcacaggctcatagaccttggcaaactaggattgtaaatgatactcatcctaataaggctgtaataaaatcacgtcagttaggtcttagtgaaatgggtgtaatggaaatggttcattttgcagatatgcatagttatgctaacgcaaagtgtctgtatacattccctacaaatgaacaaatgaaaaaatttgttcagtcacgtttgaaccctgttttagagaaagaatattttagagatattgttgattgggataaagactcgttaggttttaaaaagataagaaactctagtttattctttagaacaagttctaaagcaagtaccgtagagggtgtggatattgattatttatctttagatgagtatgacagggtaaacttattagcagaatcgtctgcactagaatcaatgtcttcatcaccttttaaaattgtgagaagatggagtacaccttctgtaccagggatgggtatacacaaattataccaacaatcagaccagtggtattacggtcatagatgtcaacattgtgattacttaaatgaaatgagttataatgattacaaccctgataatcttgaagaaagtggaaatatgttatgtgttaatcctgaaggtgtagatgagcaagctaaaacagtacagaatggtagttaccaatttgtttgtcaaaaatgtggtaaaccattggatagatggtataacggtgagtggcattgtaagtaccctgaacgtacaaaaggtaataaaggggtacgagggtatctaataacacaaatgaacgctgtatggatttctgctgatgaattaaaagagaaagaaatgaatacagaatctaagcaagcattctacaactatattttaggttatccttttgaagatgttaaacttagagttaatgaagaagatgtttatggtaacaaatcacctattgcagaaacacaattaatgaaacgagatagatattctcatatagctattggtatagattggggaaatactcactggataactgttcatggtatgttacctaatggtaaggtagacttaatacgattattctctgttaaaaaaatgacaagacctgatttagttgaagcagatttagaaaaaataatttgggaaatatctaagtacgaccctgatattataattgcagataacggggactcaggtaataatgttttaaaactcattaatcattttggaaaagataaagtatttggatgtacttataaatcttctcctaaatctacaggacaattaagacctgaatttaatgagaacaataacagggttacagtggataaattaatgcagaataaaagatacgtacaagcacttaagacaaaggatataagtgtttatagtacagtagatgatgatttaaaaactttcttaaaacattggcaaaatgttgttattatggatgaagaagatgaaaaaactggagaaatgtaccaagttatcaaacgtaaaggtgacgaccactatgcacaagcaagtgtctacgcctatataggattaacaagaataaaagaacttcttaaagaaggaaacggtacaagctttggttctacatttgtttctactgattacaatcaagaaggaaataaacaattctactttgatgaatagaggtgaaatagacttgacagataaattattttatggtacaattagtaatgaagaaattaataaaagtgtattgaatttgttattgggtgaggaattatccttagattatgtttctaaaaatagtgatattttagatgttaaatatgaacatgtttataaatctctaggattcgataatttctttgattgttttttatatgctaatagagagcctgaaatagtccataaaggtggagataaaaatcttggtggactaaataaggttaaacgtactgttattcgtaatggtaaagaaatggaaatgacagtttacgaagatggtaataaagagaacgatagtaaagaaaaacaagaaggaaaagaagaagttagtagaagtgcagtaggagcaagagctatttctaatggtgaagaaggaaaggtaaaccctaaaaaagtagcaaattcattatctaatttaagtaaaaaaggtgtagatgtatcccatattaatacaaactcatcattgtataaagagtttgttgatgataacggtgatacaataggaattacatcttttaaacgaactgaaaatgatataatattagaatcttatgcaagttcacctgattcagatggtgtaggagcaagagctattatggaattattacgtttaagtattaaagaaaataaaaatgcagttgtgtatgacatagaattacctgaagcaatagagtatttaaaaactttaggatttaaacctaataaagatggatacatcttaagaaaaaaagatgtaaaacaattcttaggtgattatagtgattttatttagcactataatcatctattctattgtatttattctatatattgtattaaaaacaatttatataaagtctaatatgagtagaatagataacacaactgaattattaaaaatattacaggaagatattgaaggtaagataaaaaaggaaggaagaaataaatgactttagaagaaaataaattaacattagaagaatcaataactccacttagcaaagaggagaaagaagatagtattaaagaatttagcagtttattatgtgaaatggtaaatagactatacaagtcttataatgtatttagacaagaccctatggatgaaactcaacgtctagatggctctttaatggtctttcaaagtagattaaatgaccctttaacaggagatttacatgataagatgtataaacttgctttttcaaaacgcattgatattttcgaagctaataagcaatttagaaaagatgtagaagcaggtaaagcaattgagttaggtgatgtagctattatagatacagcattaagtaacatcctttcaggcaatgagttccaaggaagtatttcatttatgcttagaaaagactttgaagaaaaagaacgaattagaaaagaagaagaagagaaacttaataacttataaaagggaagaattatgagactatataaaatgagggtatcataattgaaaaagaaaccacaaggcaatgaggtaatcataaccataataacggttatgatagcagtatttgtagtcattatgaccatattttttaataaatatcaagatgctaaagaagataaagatagatatcaaagattagtagagatttataaaaaagcagatgataatgatggtgagactaaaaagaaatatgttaaaagattaaataaggctgaagaagaacttaaaaaagtaaaaaaagaaacaaattataaagattataataagaagtcaaataaagaaagacaaaaagaagataaagagactagagagaaaatatatgatgtaactggtgatgatgacttaatattagtaaaaaataatattgagtttagtgataaagtagacaagcctgaaatacttattagtgaagatggaataggtacgataactgtccctgtagatagcgggtatgaaaaacaaacagtaggttctattattactagtgtattaggttctcctttcctatcacctgattcaaatagtatagatggtttaagtgttattaacgataatgtttatccaaatacagtagatagcaaagtagaagatacaaaaccttctattgacttaccaacggataatcctattataacaaatccggttgaaccaactataccctcagatgttatacctcctattgataatccttcagttccggtatctcctgagaatccagtagataataatcaaggaaatacagataacccaaatcctccacctccaggatatacagatgaagatggtggaagaggctccggtggtggaggaaattctgaaccaccatcaacggaagaaccttcggataatggtaacactggaggaggagattgggaagaaaaacctgacccaggagaagaaccttcagataatggtaatacaggaggcaatggtggagaagttacacctgaacctgaacaaccgaatgaaaatcctgatgaaggtaatgaagaaaaaccatctgaaccgtctgacaatcctgatgaaaatggaggatgggaaacggaaccaactgaacctgagtcaccttcagagccggacgataaagtggacgaagaggataaaaatgaagatactacagatgataaacagcccactgaacaaccggacgataacaacatagataatgaagataaaactgaagaggagtaattactcctctttttttgtttgctatattaaataagagctaaatataaaaaaattgaacattacggtggtgaaaactttgttaggaatgaatattataacgtcactatcagtagtatttacctgtttaagtcttttaactttaatgatttttgttcatagtaagttctctagtaaaaacgtttttgttttgtatgtaatttatgctataataggaataggtacatacatagttttaactatgtttcaaacaacatctgtacttattaagaatgatgtaatagattccatagaaaatactgaacattatattggattcaatgaccctataattatatttattataagttttataggtgcaatacttggaggaatttggtacaagatgatgaaaattattaaaaaaagtaactttaaagataaaaaataaaaaagacggtgaataggttgatattctctaaagataaaaaatgggatgaagcaaaagatttcatcaaaggtcaaggtatgcaagataattggatagagattgtagattattatagacagataggtggaaaacacgtagctgtttttattgctttaaacaaagtaaaatacatgattctagaagcaacaaaagacaataaagtaatattagtagataaagataataatatactattagaagattatgatattgttatggaaagtaagaagatgttttattacattgaagaaccgtttgaggttaaaataaatatccctcaacatattagagatgtaacttataataatactgttgtattaactacagtaagagggagtagaggtgactagtaattggcagatttatttaagcaattcagattaggtaaagactatggtaataatagtaccattgcccaagttcctattgatgaaggattacaagctaacattaaaaaaatagaacaagacaataaagagtatcaagatttaactaagtctttatacggacagcaacaggcttatgcagagccatttatagaaatgatggatacgaatcctgaatttagagataagagaagttacatgaaaaacgaacataacttacatgatgttttgaaaaagtttggtaataaccctatccttaatgctatcatacttacacgttcaaatcaagtagctatgtattgtcaacctgcaagatattcagagaaaggtttaggttttgaggtaagattaagagacctagatgcggaacctggtagaaaagaaaaagaggaaatgaaacgcatagaagattttattgttaatacaggtaaagataaagatgtagatagagattcatttcaaactttctgtaagaaaattgttagagatacttatatctatgaccaagttaactttgaaaaagtatttaataagaataataaaactaaattagaaaaattcatagcagtagacccttctactattttttatgcaacagataaaaaaggtaaaattattaagggtggtaagagatttgttcaagtagtagataaaagagtagtagctagttttacttctagagaattagctatgggtataagaaaccctagaactgaattatcttcttcaggatacggattatcagaagtagaaatagctatgaaagaatttattgcctacaataatacggaatcatttaatgatagatttttctcacatggtggtactactagaggtattttacagatacggtcagaccaacaacaatcacaacatgcattagagaactttaagcgtgaatggaaatctagtttatcaggtattaatggttcatggcaaattccagtggtaatggcagatgatattaaatttgtaaatatgacacctactgctaatgatatgcaatttgagaaatggttaaattaccttatcaatattatatctgctttatatggtattgaccctgcagaaattggtttccctaatagaggtggagctacaggttctaaaggtggttctactttaaatgaggctgacccaggtaaaaaacaacaacaatctcaaaataaaggtttacagcctttacttagatttattgaagacttagttaatagacatattatatcagaatatggagataagtatacattccaattcgtaggtggagatactaagagtgctactgataaacttaatatccttaaactagaaactcaaatatttaaaacagttaatgaggctagagaagagcaaggtaagaaacctattgaaggtggagacattattctagatgcttcattcttacaaggaacagcccaattacaacaagataaacaatataatgatggtaaacaaaaagaacgtttacaaatgatgatgagtttactagaaggagacaatgatgattctgaagaaggacaatcaacagattctagtaatgatgataaagagataggaacagatgcacaaataaaaggtgacgataatgtttatcgtactcaaacatctaataaaggtcaaggaagaaaaggagaaaaatcttctgactttaaacattaattaataagcctagaataaatctaggctttgtttattttttttgtaatttaattttgataaatgtaataactatgatatactatatgtaattgatattaatacataaaaaatattaatatttcacttacaagttattattgttatattattaacgtaaaagtaaataaaataacaagtggaggtgtagacacctttggaagaaataaaatttaatgcttttgtacctatggatttgaagaaatctgtatcaacagcttctgatactaatgagtattctatcgtttcaggatgggctagtactccaagtatggatttacagaatgatatagttaatcctaaaggaatagatatagagtattttaagtcacaagggtacattaattatgagcatcaaagtgataaagttgtagggatacctacagagaattgctatgtggatatagaaaaaggtttatttattgaagcaaagctatggaagaatgacgaaaatgttgttaagatgcttgatttagctgagaaattagaaaaatcaggtagtggaagacgtttaggtttttctattgaaggtgcagttaaaaaacgtaatataaatgacaatagagttattgatgaagttatgataaccggagttgcattagttaaaaaccctgctaatcctgaagcaacatgggaaagctttatgaaatcatttttaactggtcatggtacatcacctgacactcaagttgatgcaggagctttaagaaaagaagaaatagcatctagcattacaaatttagcttacgtcactaagattaaagatttaaaagagtttaatgatgtatggaatggcgttgttgaagatttgagtaaatctaatagtatgggatatgaggaatcagtccttacgttacaactagctaaaggtttatctcgtaaagatgcagaactagcagtaatggatataaacaaacaaaaactagaataggtaaggagaatacattctatgagtaaagaaatgcaaaatattttagaagagtatgataagttaaatgctcaagaggcagtttcgaaatctgtagaagatgatgaaaagaatacagtagaatctaccgaagagcaagtagcagaaacaactgaagaacctgctaaagaacctgaaaaagtatctgaggaagatactaaagaagcacaagagcaaggtgaaaaagttgaatctgaagaggtagcagaggacaatgaagatgaggaagttgaaaaatcagctaaagaatcaaaagaccctgtagaccaaaaagatactaagacagaaaataaagacaacgagaaacgtaaaaataaaaaagataaaaaagaagattctgattctgacgatgaagataaagatactgacgataataaagataagaaagaagataagaaggaaaaaacttctaaatcaatttctgatgaagatatcacaacagtatttaaatctatcttaacatcttttgaaaacttaaataaagagaaagaaaactttgctactaaagaagatttaagtgaagttagtaaatctattaatgagttatcagcaaaaatttctgaaatccaagctgaagatgtttctaaatcagtagacactgatgaagaagctgtagaaaaatcagtaacatctacaaatggagagcaagaaaaagtagaaggttatgtttctaaatcagtagatactgaagaacaagctgaaactggtgaagcaaaatcagaagaagctgaagaagtacaagaagataacacatttaaaggattaagtcaagaagaacgaactaagttcatggattcttacaaagcacaggctaaagaccctagagcttctaaacatgacttacaatcagcttaccaatcttacttaaacattaacactgaccctactaacgcatcagagaaagatattaaaactgtaaaagactttgcacaaatttaattaatgcacaaagttgtgttatattatacggtgtaactaaagaatataaatagggtacattttactgtaccctacataaaataaaaagaacacaaatgaaaggtgataaatttatatgactatcgaaaagaacctgtcagacgttcaacaaaagtacgctgaccaattccaagaagacgtagtaaagtcattccaaactggttatggaatcactcctgatacacaaattgacgcaggagctttacgtagagaaattttagatgaccaaatcacaatgttaacatggactaatgaagatttaatcttctatcgtgatatctcacgccgtcccgctcaatctacagtagtaaaatacgaccaatatttacgtcatggtaacgtaggtcactctcgtttcgttaaagaaatcggagtagcaccagtatctgacccaaatatccgtcaaaaaactgtatcaatgaaatacgtttctgatactaaaaacatgtcaattgcatcaggtttagtaaataacattgctgacccatcacaaatccttacagaagatgctatcgcagttgttgcaaaaacaattgagtgggcttcattctacggtgacgcttcattaacttctgaagttgaaggtgaaggtttagagtttgatggtttagctaaattaattgacaaaaataacgtaattaacgctaaaggtaaccaattaactgagaaacacttaaatgaggcggcggtacgtatcggtaaaggtttcggtacagctacagatgcttacatgcctatcggtgtacacgcagacttcgttaactcaatcttaggtcgtcaaatgcaattaatgcaagacaacagcggtaacgttaacactggttacagcgtaaatggtttctactcatctcgtggattcattaaattacatggttctacagtaatggaaaatgaattaatcttagatgaatcattacaaccattaccaaatgctccacaacctgctaaagttacagctactgttgaaactaagcaaaaaggtgcttttgaaaatgaagaagaccgtgcaggattatcatataaagtagtagttaactcagatgacgcacaatcagctccttctgaagaagtaacagctacagtatctaacgtagacgatggtgttaaactttcaattagtgttaacgctatgtaccaacaacaaccacaattcgtttctatctaccgtcaaggtaaagaaacaggtatgtacttcctaatcaaacgtgtaccagttaaagatgcacaagaagacggaacaatcgtattcgtagataagaacgaaacattgcctgaaacagcagacgtatttgttggtgaaatgtcaccacaagtagttcacttattcgaattacttccaatgatgaaattaccattagctcaaattaatgcttctattacatttgcagtattatggtatggtgcattagcattacgtgctcctaaaaaatgggctcgtattaaaaacgttcgttatatcgcagtttaatagaataagaaaaactgaatacaagagaatagggataaacttagggtttatcccttttttattaaaataaacttgaagggatttaataaatatgttatactataagaaactattagataaaaaaatggctactgtttatggtacagttgagattgacaaagatggagtagttaaaggattaactaaagaacaagaaaaagaatttgcaaatgttccaggttttgaatttgaagaagaaaagaaaactactagaaaacaatcagcttctactagtaaagaagaagagcctaaggaagaggaaaagaaagcctctactagaaaaactacaagtactactagaaaatctacagcacgtaaaacaacagccaaaaaagatgaaaataagtaaagggtgaattaaatggttaactcaatgtttggaggggacttagacccttatgaaaaatcattaaactatgaatatccttatcatcctagtggtaatcctaaacacatagacataagtgagatagataatttaacattagctgattatggatggtcaccggatgcagttaaagcatatatgttcggtattgtagttcaaaatcctgatacaggacaacctatgggtgatgagttttataaccatatattagaaagagcggtaggtaaagctgaaagagcattagatatatctatactacctgacactcaacatgagatgagagattatcatgagacagagtttaatagttatatgtttgtacatgcttatagaaaacctatattacaggtagagaacttacagctacagtttaatggtagaccgatatataaataccctgctaactggtggaaagtagagcatctagcaggacatgttcaattattccctacagcacttatgcaaacaggacaatcaatgtcatatgatgcggtattcaatggataccctcaattagcaggtgtatacccaccatcaggggcaacatttgcacctcaaatgatacgattagaatatgtatcaggtatgcttccacgtaaaaaagcaggaagaaataaaccttgggaaatgcctcctgagttagaacaattagttataaaatatgcattgaaagaaatataccaagtatggggtaacttaatcattggtgccggtattgctaataaaacattagaagtagacggtattacagagacaataggtactactcaatcagctatgtatggtggagctagtgctcagatacttcaaataaatgaagatataaaagaactattagatggtttaagagcttactttggatataatatgataggattataaggagggttagaaaatggaaaaaccgtatatgataggagccaactctaaccctaatgttattaataagtcaacaacatatactactacaacacaagcagatgaacaagataaacctaagtatactactagactagagtttgatacgattgacatgattaggtttattaatgaccgaggtataaaagtattatgggaagaagcatatttctgcccttgtcttaatcctgatacaggacatcctagagtcgattgtcctagatgtcatggtaaagggattgcatatctacctcctaaagagactataatggcaatacagtctcaagagaaaggaactaaccagttagatataggtatattagacacaggtactgcaataggtaccactcaattagaaaagaggatttcctatagagacaggtttactgttcctgaggtattgatgcctcaacaaatgatttattttgtgaataaagatagaattaaaaaaggtatacctctatactacgatgtaaaagaagtaacttatatagccactcaagatggtacagtctatgaagaagattatgaaattaagaataatagattgtatttaaatgaaaaatatgagaatcatacagtaactttaaagatacttatgactttaagatatgtagtatcagatatactaaaagaaagtcgttaccaatatactaagtttaatcaacctaaatcaaaatttgaaaacttacctcaaaaattacttcttaaaagggaagatgttattgtactacaagacccttataaagttaatgatggtatagaagaagacctagaaattcaagtagatgaccctaaggcttcggcatctaatcctagtaatttaggtggattcttcggaggtgcatttaaataatgccagttcacggaaagagacctaatttatttaaaaataaaaactataagcaggtaggtaagagaacaattgatggtatgcgttcagaagttcttgataaattacaagcaacagcacagcaagtagagaatactagtattaaacgtatgcctacttacctacaaataacagagaaaaagcttgaaaaagaaggagtagtagaccttaaaaaagcttttgctcactcatctaaaaagaaaactagtaaagatggcggatggtatttaactgtaccaatccgcatcaaaactagtagaatgaataacagtacttaccaagatatgagaactttaaaagtagataaaggtacaggttcagtttctaagataactgattatctagaaggacgtagaaagaatgtaagccacccttcaatgaaaccggaacctatgacccataacatgactaaagttaaaagaggaaagcaatcttcttactttatatttagaactgtttctagtaagtcacctgctagttcttggatacttaacagagataaagttaatgaagataacttctctaaaacaactctaaaaactgttaagcaattaatgaactggaagatgaaaaatttaaattaagaggagggttagtattaaatggcaataacatcagttgattcatatttattatcagaaataaagcctagacttaacactgtgctagagaattgttatattatagatgaagttttaaaagactttgattatcaaactagagagagctttaaagaagctttctgtggtaagaatgcacaacatgaagtaacggtaggatttaacttcccaaaatttaaaaataactatgaagctcattacttgatacaattaggtcaaggacaagagacaaaaaactctttagggagtattcagtcatcttactttgaggcaacaggagataccttagtcgaatcttctacagcaataagagaagatgataagttagtttttactgtttctaaaccaataggagaattaataaagatagaagatatagagtttgctaaatacgataatcttcaggttgaaggtaataaggtatcatttaagtatcaaacaaatgaagattatgagaactacaatgctaacattatatttaccgaaaagaaaaatgattctaaaggtttagtaaaaggattcacagttgaagaacaagtaacagttgtaggtctttcatttaatgtagatgttgcaagatgtttagatgctgtactgaaaatgattctaatatctatgagagatagtatagaagagcaacaaacattccaattacagaatttgtcttttggtgatattgcaccaataatagaagatggtgactcaattatttttggtagaccaacaattattaagtacacaagttctctagatttggattatactattacacaagatattaataaactaacttttaaagaaagaaaggattggaagtaggatggctagaaaaaagacacctgaaaataacactcctaaatttaatggttatgttcatatagatacattccttgatactgcaaaaaccctttttaatatgagggattcacaagtagcaggatttaaggcttatatggaaggtagtcattatttgtttagtgagcaagaattcttaccatcattagagaagtatctaggtaggaaattagatatataataacattcagataaggagaattaaatatggcagtagaaccattcccaagaagacctattacccgtcctcatgcatctattgaagtagatacttcaggtatcggtggctcagcaggttcaagtgaaaaagtattttgcttaatcggtcaggctgaaggcggagaaccaaatacagtttatgaattacgtaactatgcacaagctaaacgtttattccgttcaggagaattacttgatgcaatagaattagcatggggttctaaccctaactatacagcaggacgtattttagctatgcgtatagaagatgctaaacctgcttcagcggaaattggcggattaaaaataacatctaaaatctacggtaatgttgctaacaacattcaagtaggattagaaaagaatacattaagtgattcattacgcttaagagtaatcttccaagatgaccgtttcaatgaggtttatgataatatcggtaatatcttcacaatcaagtacaaaggagaagaagctaacgcaactttctctgtagaacatgatgaagaaactcaaaaagcaagtcgtctagtattaaaagttggagaccaagaagttaagtcatatgatttaactggtggagcttatgactacactaatgctattattacagacattaatcaattacctgattttgaagctaaattatcacctttcggagataagaacttagaatctagcaaattagataaaattgaaaatgcaaatattaaagataaagctgtatatgtaaaagcagtttttggtgacttagagaaacaaacagcttacaacggtatcgtatctttcgagcaacttaatgcagaaggtgaagtaccaagtaatgtagaggttgaagcaggagaagaatcagctacagtaactgctacttcacctattaaagctattgagccgtttgaattaactaagttaaaaggtggtactaatggtgaaccacctgctacatgggcagataagttagataaatttgcacatgaaggcggatactacattgtcccattatcatctaaacaatcagttcatgcagaggtagcttcttttgttaaagaacgttctgatgcaggagaaccaatgagagccattgttggtggaggattcaatgaatctaaagaacaattgttcggtagacaagcatcattatctaatccacgagtatcattagtagctaactcaggtacttttgttatggatgatggacgtaaaaaccacgtacctgcttacatggtagccgtagctctaggtggtcttgcaagtggtttagaaatcggtgaatcaatcacattcaaaccactacgtgtaagttcattagaccaaatttatgagtcaatagacttagatgaattaaatgaaaatggtattattagtatcgagtttgttcgtaaccgtactaatacattcttcagaatcgttgatgacgtaactacattcaacgataaatcagacccagttaaggctgaaatggctgttggggaagctaatgacttcttagtaagtgagcttaaagttcaacttgaagaccaatttattggtactcgtactatcaatacaagtgcttcaatcattaaagactttatccaatcttacttgggtcgtaagaaacgtgataacgaaattcaagacttccctgctgaagacgtacaagttattgttgaaggtaacgaagcaagaatttcaatgacagtttacccaatcagaagcttcaagaaaatctctgttagcttggtttacaagcaacagacattacaagcctagtctaggtgacggagtacctggattaggtactcctattaatataatttgaatactttaggagagtgaatacagatggcatcagaagctaaacaaaccgtccatactggtaataccgtcctacttatgattaaaggtaaaccggtaggaagagcacaatcagcatcaggtcaacgtgaatacggtacaactggtgtatacgaaatcggttctatcatgcctcaagaacacgtatacttacgttatgaaggtacaattacagtagaacgtttacgtatgaaaaaagagaactttgcagatttaggatatgcttcacttggtgaagaaattcttaagaaagatatcattgatattttagtggtagataacttaacgaaacaagttattatctcatatcatggttgttctgcaaataactacaatgaaacttggcagacaaatgaaattgtaacagaagaaatcgagtttagttacttaacagcaagtgacaaagcacgtacttaatagattagaccaactaaaaagttggtctttttttattgacattttaaaatttatatgttattataataacaggttaggagtaataagtatgaataataggcaagctaaaataaaaggatataaccaatttcattattatgattttccaacaactaaaggtaagtttaaagatataatgaaaagaaaatctagaacagaacttaaaaaagatttacaaaaagaaaggaagtattatcttgacaaataagagaaaaacgataggtaagatgagtaacacaagagcaacatggaatattaatccggtaactaaagttaaaaaagataaaacaaaatattctagaaaaaataaacataaaggtcttgacaattataattaactaaggtatattattagtataacaaaaaaggagatgggtatatgagtacattttggtcagaaagaagaacaactaataaagataggcaagttaaaaaacattatactcaaatgagtatgtatgaaagaaagaaatgtgtagagttattacaagagacaattactgaaaatagaattattaattttacacgacatagtgcaaaaaaagttaaaggtaaaccaacaacaaatatacctaaattaataggttttatttttaaaaataagtttgcctatgaaaatatcatagagtacaataacacagattataatggtaatattgagaggagaattgttgttaaacatcctaaagttataactgtagaaggaaaacctagctatcagtttttgacaattagtcttgaagatgctagagttattacagtatggtataacagtgtagatgatacacatagaacactagatttaaattattatagtaaagacttgacaattcaataaggaggtattataatgggtataacaatagtaaatagttattttattctgtctagcattttactcttcatattaaccatattaaatggcaagggtacagttacaagggaatcactaactatgagtaaaatattagtagtaataacatcaattcaatttttagcatgtttaattattaatggtatttattggtcactaaaatttatgtagtagaattagaataaaagtattgacaaattaaaactaataaattataataaaggtataacaaattaaaggagaagatataaaatgtcacaagataaattaagagcaatttacacagaaatgaaagtagaattacacaaatttcctaaagaggtagatataacaagtaaatcaactgcaattgcaatcaatcagattttagataaattcaaaacattaacagaacaagcagggaagattactagaaaatatttagaaggtcaagaaatattaactattgattatgagtattatgattcattacaagaatactatatttacctacttagaaatagtgaaaagattgaacaaagtttacaagaaattactaagcgtacaggtgaatatgtaaagtaattttgatttaaaaacaaaatatgatatactatgtttaaagtagtaagcctacactagtccgtgttatattaatattgaatcggataagcgtaggctttattaatatttaaaaaaggaaggtatatcatattatggcagaagaaattaaaaaggaacaagatgtacaagaaacaactaaagaagaaaaaaaagatgttagcaaaatgacaccggaagaaatagataaattaaaatatcaagacaaacaggaaaaagaacaagttattaacaaagttattaaaggcgttaatgatacttgggaaaaagaatataactttgaagaactagacttaagatttaaagttaagattaaattacctaatgcacgagaacaaggtaatatctttgcgttacgttctgcttacttaggtggtatggatatgtaccaaacagaccaagtaattagagcatatcaaatgttagctacattacaagaagtaggtattgaagttcctaaggaattccaagaccctgacgatatttataatttatatcctttaactgttatgtatgaagattggttaggattcttaaactcctttcgttactaatagtatagaaacattagataaagatatagaacgattgggcggtatggaatcaattgttaaacaacctttatctagaaatctatgggctattatgaaagagtttaatgttttacctactgagcaaagatttaaggacttagatgattatcagatagagtttattattgggaatatgaacagagatgtttatgaacataacaaacaacttaaacaagctcaaaaaggtggaaaattcgatagtcaattcgaagatgatgatagtagttggtggaatgaatctcatgaagactttgacccagtacctgatttcttagatgctgatgatttagcacaacagatggaagctaaattatctgatagagataaggaagaaagagctaagagaaacgatgcagagttaaatgatgaaacagaaggacttactacacaacatctagccatgatggaatacatcagacagaaacaacaagaattagatgatgaagtaggaaatggtaagactagtgaagatgacgctactatatcacaagatagcgttaataaagcactagaagacctagatgatgactggtatatgtaaagggtggtaggtgatactaccatccttatttttttaaaatggatggtgaataatgatggcaatgaatgacgattatagattggtcttgtccggtgatagttcggatttagagaatagtctgaaggcaatagaactttatatggattctttagagtctaagaatattgatgctcctttagataatttcttaaaaaaattaaaagtaattgctaaagaagttaaaaatgtacagaacgcaatggataaacaagatggtaaatctgttatatcttctaaagacatggatgaatctattaaatccactcaatctgctacaaagaatataaatgaattaaagaaagctttagatgaccttcaaaaagagaatatatctaaaggtattgcacctgaccctgaagttgaaaaagcatatgctaagatgggtaaagttgtagatgaaactcaagaaaaacttgagaaaatgtcttcacaaaaaataggttctgatgctagtattcaaaatagaattaaggaaatgaaaaccttaaatcaagtaactgaagaatacaataaaataagtaaagattctagcgcaactaaagattatacaaaacgattaagagctaatcgtaatatgactagaggttacatggagcgttcagaaggaacaggacgtttgacatatgaccaaggtgcacgagttagaagtgaactaggtaaaataagttcttatgagagccaaagaaaacaaaaccaacgtaatttaggacaagcaagagagcaatatagcaactatagaaaccaacaacaagacttgactaaacgtagagctagcggtcaaattaataaggcacaatatgaacaagaattagcttctattaaacaggaaatgaaagctagagaagaacttatatctaactacgagaaactaggagcagaacttgataaaacagtccagtattataagggttcagttcaaaaggatttccaatctagagatgtagaccaacaacgaggaacatttggtagaatggttcaagaacgtttgccatctattggttctcatgctatgatgggtactacagctatggctacaggtttatacatgaagggtgcctcattaagtgaaactaatagaccgatggttacatcattaggtcaaaattccgataatatggatatagattctgtaagaaatgcatatggagacttgtcaattgacaacaaattaggttataatagtactgacatgttaaaaatggctacttcatatgaagcatcagtaggacataaaagtgatgaggacacaatggcagggactaaacagcttgctattggaggacgctctttaggtattagagaccaagaagcttatcaagagtctatgggtcaaatcatgcatactggtggagtaaattctgataacatgaaggaaatgcaagatgcattcctaggtggtattaaacaatcaggcatggttggtcgtcaagatgaacaacttaaagcactaggttctatagcggaacaatcaggagaaggaagaactctaactaaagaccaaatgagtaatcttactgccatgcaatctacttttgcagagtcaggaagcaaaggattacaaggtgaacaaggtgccaatgctattaacagtatagaccaaggacttaaaaatggtatgaatagttcttatgctcgtatagcaatgggatggggaacacaataccaaggtcttgaaggtggatatgatttacaaaaacgtatggatgaaggtatatctaatcctgaaaacttgacagatatggctgatatggctactcaaatgggtggcagtgaaaaagaacaaaaatacctatttaatagaagtatgaaagaaataggtgctaacctaactatggagcaatctgatgaaatatttaaagatgctcaatccggaaaactatctaaagaagagttagctaagaaagctaagaaaatggaaaaagaaggtaaaaaagaaggagaagataacgccactgattataaagaatctaaatcaggaaaaaatgaccaaaataaatctaagactgatgataaagcagaagatacttatgatatggctcaaccactaagagatgctcatagtgctttagcaggtcttcctgcccctatatatttagctataggagctataggagcatttacagcttcactaattgcatctgcaagtcaatttggagcaggtcacttaattggtaaaggagccaaaggacttagaaataaatttggtagaaataaaggtggtagctccggtggtaaccctatggcaggtggaatgcctagtggcgggggttcacctaagggcggaggctcacctaaaggcggaggaactcgttctactggaggaaaaatacttgatagtgctaaaggtcttggaggattcctagtaggtggcgcaggatggaaaggtatgtttggcggggagtctaaaggtaaaggctttaaacaaacatctaaagaagcctggtcaggtactagaaaagtatttaatagagataatggtagaaaagccatggataaatctaaagacatagctaaaggtactggtagtggtcttaaagatatctataatgatagtatatttggtaaagaaagaagacaaaacctaggagaaaaagctaaaggttttggtggcaaagctaaaggtctttatggtaaatttgctgataagtttggtgacggaggtaaaaatggtatcctttcacaatcaccaaaagcaggtggaagtggcatagggaaacttggaaaacttgcaggtggacttggaaaaggagccggagttttaggtgttgctacgtctgccttatcattaatacctgctttagcttccggagatagtaaagctatcggcggtggaataggctctatgggtggaggaatggcaggtgcatcagcaggagcttctataggagctttatttggtggtgtaggtgcaatacctggagctttaataggtggagctataggttccttcggaggaggagctgttggtgaaaaagtcggagacatggctaaaaaagctaacactaaagaaggatggaacctaggatggactaatggagataaagatggtaagaataaattccaagattctttattaggaaaacctatatctaaagcatggagcggtataacaggtctctttgataatgacgctgaagcatccgaagaagatagtaaagataagaaaaaaggtgttaaaggcgttaaaggagatactaagaagaaagaaaaaatgacagcagaacaacttagagaaaagaataaccaatctgaaactaagaatcttaaaatctatagtgatttacttgacagagctcagaaaattattgagagtgctaaaggtattaatatagatggaggaacttctgatagtggttctgatagtggaggctctgcatctgatgtaggtggagaaggcgcagagaagatgtacaagttccttaaaggaaaaggactatctgataatcaggtaggagctgttatggggaacttacaacaagaatctaatcttgaccctaatgctaagaatgcttctagtggagcatttggtattgctcagtggttaggagctagaaaaacaggattagaaaactttgctaaatctaaaggtaaaaaatctagtgacatggatgttcaattagattacctatggaaagaaatgcagtctgattatgaaagcaataatcttaaaaatgcaggttggagcaaaggtggaagcttagaacagaatacaaaagcatttgctaccggatttgaacgtatgggagcaaacgaggctatgatgggtactcgtgttaacaatgctaaggaattcaagaagaaatatggaggctccggtggcggaggtggtggaggagctttatcctctacttatcaagaagctatgagtaatcctgtattaactactggttctaattatagaggctctaatgatgcttctaatgcttctacaactaacagaataacagtcaatgttaatgttcaaggtggaaataatcctgaagaaactggagacattatcggaggaagaattagagaagttctagatagtaatatggatatctttgcaaatgaacataagagaagctattagtaattttgtattgacacaagagtagtatcatagtatactactcttatacatataaaaaataaaaggaagtatgtgtatatgcgtagaataagaagacctaaggtaagaatagaaatcgttacagatgataatacatttacattgagatttgaagatacacgtgactataatggtgatgagtttggagctaaacttttaggattccaaactaaaaactctatggaagatgatagttcagttttccaaataaatatggcaggagatacttattgggataagctagttatggctaatgatatcataagaatatttattacacctaatgatgaccccaacgataaagaaggaagacaagaacgacttatccaggtaggtatggtttctcaagtatcaaaagtaggtagttacggtaatgaccaaactcaatttagaataacaggtcaatcttttgtaaaaccttttatgaaatttggattaggcgttattcaggaagttcaagctgtattgcctgaagtaggttggcttattgatggtgatggagataatgaagtaaaatttactggtagctcagctcatgaagtaatgactggcattatacgtagatttataccttatatgaaatataactatactgaaaaaacatataatacaattgataactatcttgattatgatgatttaagtagttgggatgagtttgaaaaacttacagaagtttcagcctttactaattttgacgggtcattaaaacagttaatggatatggtaacagctagaccttttaatgagttattcttcaaaaattcagaaaaaacacctggaaaggctcaacttgtattaagaaagaccccttttaatcctactgagtggagagctttagatatgattaaagtacctactgaggattttatagaagaggatgtaggtaaaagtgatgtagagacatattctatatttacagcaacacctgcaggtatgttgaaagagcttaacggtgatgtattttccaaaccacaattccatcctgaattaactgatagatatggttataccaaatttgaagtagaaaatatttatcttagtacaaaatcaggttcagccactgaggattcagattcttcaggtgatgataatggtacagaacgaggaacttactctaaaattatgaaagatttaagtaactatggaagagataatatatctaaaggtatagataagtatacaagtaaattatcttcaaaatataaaaacttaaaaaaagcccaagctaaaaaaattatagagaagtttgtcaaagaaggaaaagtaacagaaaaagaatatgaaaaaataacaggtaataaggtagatgatgaattaacatcagataacagaccgaagttgacaaaagataaattaaagagtatactaaaagagaagtttaaaacacaagatgattttaataattctaagaaaaagaaaaaagctaagacagatgcacttaaagaattgacaactaaatatcgttttggtaataaaacacatgctacaactttgttagatgaatatattaaatacaaaggagaaccacctaatgatggggcttttgataaatatcttaaagctattgaaggtgttagtaacgtagctacagacacaggttcagatgcaagtgatagccctctagttatgttttctagaatgctatttaattggtatcatggtaaccctaacttctatgcaggagatattattgttttaggagaccctaagtatgacctaggtaaaagattatttattgaggataagcaacgaggagacacatgggagttctatattgaatctgtagaacataaattcgattataaacaagggtattatacaactgtaggagtaactagaggtttaaaagacgctattctagaagatggtaaaggtagccctcatagatttgcaggattatggaatcaatcatcagacttcatgggaggtcttatgggtgaagatacttctaaagaacttaaagaaaaaggtgtagcagagaaacaaagtagtggaggtaaagatggcggttctgatagtggcggagctcaagatggtggctctttagattcacttaaaaaatataacggcaaacttcctaaacatgacccaagttttgttcaacctggtaaccgacattataagtatcagtgtacatggtatgcttataatagaagaggtcaattaggcattcctgtgcctttatggggggacgccgccgactggataggtggtgctaaaggagcaggttatggtgtaggtagaacacctaaacaaggtgcttgtgttatatggcaaagaggagttcaaggaggtagcccacaatatggtcacgtagcttttgtagagaaagtattagatggaggtaaaaaaatatttatctctgaacataactatgctacccctaatggatatggtactagaacaatagatatgagttcagccataggtaagaatgctcaattcatttacgataagaaataaaggaggatagtctatggcaacagataaagaagctaaagatgttattgataaatttatagacaatgtatttaattttgatgtacttacaaaagaaagaataaaagaaaaagatgaagaaattaaaaaaataactacagatgatatgtatgaaaaggttgtgtatatacgaccctacgttggagtaatacaaagccttaaccctcaacatgttcagtatgaatcattttctaataatggttatgatatagaggcagaattaagtttcaggaaagtaagttatttagttgataaagggtctatacctacagattctttatctactttaacagttcacttagtagaaagaaatcaagaactattaatagattactttgatgagatacaagatgtgttgtatggagaatatatggaagaagaatatgtatttgatgaagatgtaccattaagtacgatactagcattagacttaaatgataatcttaaatccttatcaaatataaagtatatgttcaaaggtgctcctaaagagaatccatttggaacagataaagatgtttatatagatacttataacttattatactggttatatttaggtgaagatgaagagttagcataccctatgaatattaattacttctttacagagggaagattctttactatattcggtaaaggacataagtataaggtagatgttagtaaatttatagttggagatatattattctttggtagaagtgatactaatataggtatttatgtaggagatggggagtttatatctatgatgggtaaattccctaaagatgaaacacctataggaaaatataaacttgatgattactggaatgaatttaacggaagagttatgagattcgatgaagaggtgtatatttaatggtagtaagattccaatcttccatggggagaagtttaaaaagagtagattcagatgatttaaatgtaaaaggattagttttagctacagttagtaaaattaattataaatatcaatcagtagaagttaaagttaacaacttaactctaggaagccgtataggtgacgatggtagcttagctgtaccttatcctaaatctttcataggaagaacacctgaaggaagcgtattcggtacaaaacctcttattactgaaggttctgtagtattaataggatttctaaatgatgatataaatagtcctattattttaagtgtttatggtgataatgaacaaaataaaatgattaataccaatcctttagatgggggtaagttttatacagaaagtgtctataaatatagtagttcactatatgaaattttaccatctttaaattataaatatgatgatggagaaggaacaagtattaggacttataatggtaaatcatttttctctatgacatcaggtgaagaagagaaacctcaggcaacagatttttatactggaactgagtatcaagatttatttacttcttattatggtaataagacattaattgagcctagaatacaaaaggctcctaatatgttatttaaacatcaaggcgttttttatgatgatggcacgccggataatcatataactactttatttatatctgaaagaggggatataagagcctcagttttaaatacagaaacacagaaaagaaccacacaggaaatgtcaagtgatgggtcttatagggttataaaacaagatgacgatttaatgttggatgaagctcaagtttggattgagtatggtattagtgaagataataaattctatattaaaaatgacaagcataaatttgaatttactgatgagggaatttatatagatgataagcctatgttagaaaacttagatgagagtatagcagaggctatgaagaatttgaatgaaatacaaaaagaactcgatgatataaacsataccttctcgagggtgtaggtaaggataacttagaagaattaatagagtctacaaaagagtctatagaagcttctaaaaaagcaacttcagatgtcaatagacttacaactcagatagcagaagttagtggtagaactgaaggtattataacacagttccaaaaatttagagatgagacttttaaagatttttatgaagatgcttctactgttattaatgaagtaaatcagaatttccctactatgaaaacagatgttaagaccttaaagactaaagttgataacctagagaaaactgaaataccaaatattaaaactagattaacagaactagagaacaataataacaatgctgataaaataatatcagatagaggagaacatataggtgctatgatacagttagaggaaaatgtcactgtacctatgagaaaatatatgccaataccatggagcaaagttacttataataatgcagagttttgggattctaataatcctactcgattagtagtacctaaaggaataacaaaagtaagagttgcaggtaatgttttgtgggactctaacgccacaggacaacgtatgttgagaatattgaaaaatggtacttatagtataggattaccttatacaagagatgtagctatatctacagcacctcagaatggtactagtggagttattcctgttaaagaaggagattactttgagtttgaagctttccaagactcagaaggtgacagacaattcagagcagacccttatacatggtttagtattgaagctatagaattagaaactgaaactatggagaaagactttatgcttataggacatagaggagcaaccggatacacagatgagcacacgataaaaggatatcaaatggctttagataaaggtgcagattatatagaattagatttacaattaacaaaagataataagttattgtgtatgcatgattctactatagacagaacaacaacaggaacaggtaaggtaggagatatgaccttatcttatatacaaactaactttacatctctcaatggtgagccgataccatctcttgatgatgtactaaatcattttggaacaaaagttaaatattatatagaaactaaacgtccgtttgatgctaatatggataaagaattattaactcaattaaaagcaaaaggattaataggaataggttcagagagattccaagtaattattcaatcatttgctagagaatcgttaattaatattcataatcaattctctaatatacctttagcttacttaacaagtacattctctgaaagtgaaatggatgattgtttaagttatggttcttatgctattgcgcctaaatatacaactataactaaagaattaatagatttagctcatagtaaaggtcttaaagtccatgcatggacggtaaacacaaaagaagaaatgcaaagcttaatacaaatgggtgtagatgggttctttacaaactacctagatgaatataaaaagatttaatattaaagacctattaatttaggtctttttttagttgtaatttaaactagttcgtgatatattagtagtatgagatttatatacatactgaaaaggagaggataaaatgccacaatcagatggaataagtaatcttcatagaatagctttacgtttccctaaagaaggcggtggttatgatatgtatagatttaaagttaaccctgagaactacacaatagattcaccacaacgtacgacagcaattaaaacaaaatcagatatcgtaatagaagattatggtaaagatatagaagttattaacttcacaggtacaactggttttagacctgttagagaagcagatgggttaaaaacaggtaagcagaaaatggaagagttacaaagtagagttagtgaatatgctatgcaaggtggcagtggtaatgtaagtggttcttacttacaattttttaactttacagatgatagttattataaagttcatttagctcctcaggggttaaagataactaggtctaaagatgaaccattactttttagatatgaaataacattagtagttattggttcattaacagaagcagatagaagtgctgtaactactgaagagtttggtaatgttaaacctaatgcttctcaaagagtagatgaaggtataaaagaattagataaaaatgctcgtaaaacgagagatagaaacaatcaagaaatatctagaagagaaaatacaatacctaaatctacaggagataatacgaatgagggtaatagacttaaacaaagcttccctagtagttctatatataatcctagacaatctactaacggattaaaaggtaatattgacaatatggctctgataataggttacggtgatggaggtgtatctagctaatgaataattttataccacaacctcaaggtctacttagatttttaaatgccctagatacagatttaacttcttctcatatgaatttactggatgaagaggtatcatttgtatctaaattttatacaccacagttacaattaagtgaattagcaaaaaaagtattgacaaatataaagacagatgatatacctgtattagaaagggaatttaatgataatacaattatccataaagctaacgatacattactaaaagtacaggctccaagaatgtatatgattctacagtctattgtacttgaagcatatgctattgttaattgctttgtagaaaatccaagttctttaaaatacttaaccgaagaagatgttagtataacacgagaaaacttaaattatgtagctgactacttaggtaactatgatgactacaatagtgttgtcttagacttaagagatttagacttatgttttagtgctatagaattacaattacctctaattaaaaaggaggctaacgtataatgagatttaagaagcacgtagttcaacatgaagaaacgatgcaagcaatatcacagagatactatggtgatgttagttattggatagacctagtagagcataataatttaaagtatccttatttagtagaaactgatgaagaaaaaatgaaagaccccgaacgattagcttctaccggtgatacactgattatacccatagaatctgatttaacagatgtatcagcaaaagaaattaattctagagataaagatgtactagttgaattagctttaggaagagatttaaacattactgcagatgaaaagtattttaatgaacatggtactagtgataatatattagcattcagcacaaatggtaatggagatttagatactgtaaaaggcatagataatatgaaacagcaattacaggcacgtttattaactcctagaggttccttaatgctacatcctaattatggttcagatttgcataatttatttggtcttaatatacctgaacaagctacacttatagaaatggaagtattgagaacattaacatcagataatagagtaaaatctgctaatctaattgattggaaaatacaaggataatttatttggtcttaatatacctgaacaagctacacttatagaaatggaagtattgagaacattaacatcagataatagagtaaaatctgctaatctaattgattggaaaatacaaggtaatgtttattcaggtcaattttcagtggaaataaaatctgttgaagaatcaataaattttgtcttaggacaagatgaggaaggaatttttgctttatttgaataggaaaggattaaattatgaaaactagaaaattaactaacatactatcaaaattaatagataagacaatggcaggtacaagcaagataacagactttactcccggttcagcttcccgttcattattagaagctgtatcattagagatagagcaattctatatcctaacaaaagaaaatattgattggggtatacaagagggtattattgaagcttttgattttcaaaaaagacaatctaaaagagcttatggtgatgttactattcaattctaccaacctttagatatgagaatgtatatacctgcaggaacaacttttacttcaacacgacaagaatatcctcagcaatttgaaacattagttgattattatgcagagcctgattctactgagattgttgttgaagtttattgtaaagaaacaggggttgcaggtaatgttcctgaaggaacgattaatactatagcatcaggttctagtttgattagaagtgttaataacgagtattcttttaatacaggaactaaagaagaaagtcaggaagactttaaacgtagattccactcttttgtagaatctagaggtagagcaactaataaatcagtaagatatggtgcactgcagatacctgatgtagaaggtgtttatgtttatgaagaaacaggacatattacagtatttgctcatgatagaaacggtaatttatcagacaccttaaaagaagatataattgatgctttacaagactatagaccaagtggtataatgttagatgttacaggtgtagaaaaagaagaagttaatgtttctgctacagtaactatatctaataaatctagaattggtgatacattacaaaaacatatcgaaagtgttattagaagctatttaaataatttaaaaacttctgatgacctaataattacagaccttattcaagctataatgaatattgatgacgtattaatatatgatgtgtcatttgataacttagatgagaacattatagtaccaccacaagggattattagagcgggagaaataaaagtagaactaaagtaaagagaggtgaaacttaagtcgtggctaattttttaaaaaatcttcatccattattaagaagagatagaaacaaaaaagataatcaagaccctaactttgctctcatagatgcactcaatgaagagatgaatcaagtagagaaagatgctatagaaagtaaattacaatcctctctaaagacatctacaagtgaatatttagataagtttggggattggtttggagtttatcgtaagaccgatgagaacgatgatgtttatagagcaagaattataaaatatttactcttgaaaagaggaactaataatgctataatagatgctataaaagattatttaggtagagatgatattgatgtaagtgtatatgagccttttacaaatattttctatactaacaaatcacatttaaatggtgaagaccacttaatgggatactattatagatttgccgttattaatgtctctataggtgattatttccctgtagagattatagatgtaattaatgaattcaaacctgcaggtgtaaccctatatgtcacttatgatggagcttctactattagaggtggagcaattattaagtggttagatgggttacctaaaatagaaacataccaagagtttgatagatttacaggttatgatgatacattctatggtcatattaatatgaatcaaagtaaagatactgataacagttcatcagatatttttaaaacaaatcatagcttaattaatagtttagatgttttaacaggttcatctagtgtagggagacagtatattaactacggatatgtaacatcatatgtttataatccaggtatgacatcctctgtaaaccaaataagtgctagtacagaaggtagagggcaagaagtacctactgactactatatgtatactagtactaagaataacaatacagtagaacttaatatgcaaactacttccggagtgtcttatctatataataactttaattttagagattatatgagtaaatatagacctcaagtagatttacaatctgatgaggctagaagaattgtgtctgattatataaaagaattaagtattgattactatcttagtgctgtgatacctcctgatgaaagtatagaaattaaattacaagtttatgatttttctattaatagatggcttacagtatcaattaataatttatctttctatgaaaaaaatatcgggagcaatataggatatataaaagattatctaaacagtgaattaaatatgtttactaggttagagataaatgcaggtaaaagagattcagtagatattaaagttaattacttagatttaatgttttattactatgaacgaggtatttatacaataaaaccttataaagcattaatagaaaattatttagatatatctagagagacttatgtagaagcatttaaaatagcatcattatctaatggagatattataactaaaacaggttttcagcctatagggtatttaaaactagttggtaattatgaaaatacaatacctagcacaataaatatagtagctaaagatacagataataaccctatagaatctaatgaattagatgtatataatacattagagaatagaaatttattacaatcttataaaggtgtaaatacgatagctagagaaataacttctacaaaagagtttactgtatcaggatgggctaaagagatatactcaactaattatctttctaaagtattaaaaccaggtaaagtgtatacgttatcttttgatatggaaataacaggtaatgacccaactcttaaatcttattctgataatcatggtatatatttatacagtaatactaagggaattgttgttaatggtgttaaatctatggaacgtactataggtaacaaagtatctgtaactcaaacttttacagcccctactattactgaccatagattattaatatatactggaagatatacatctgatggtaaagcatcaactcctccagtgttctttaatacagttaaaattacggaattaaaattgtctgagggttcctctaagctagagtactcacctgctccggaagatgaacctaatgtaatagaaaaaggaattaaatttaataatatcctaactaatatacagactttaagtattaattcggatactatcttaaaaaatgtaactttatattattcttactatggtgataattgggtagaactaaagactctaggaaatattagtactggagaaacaacagaaaccaataacttaatagatttatatggattacagacagtagattattctaatataaatccaatgtctaaagtatcattacgttccatttggaatgttaagctaggtgaattgaacaatcaagaaggttctttatctaatatgcctaatgattactttaatgctgtatggcaggatatagataaattatcagatattgagataggttctatgagaatggttaaagacactgagggtggagtattcgatggagctacaggtgaaattattaaggctactctatttaatgtaggtgcttatactgatttagacatgttatcttatactttgactaactatactgaaccgttaactttaggctctagtagattaataagtgagttaaaagaagaactactaacatcagaatcatttaatgttgataatagaattaaagtaattgactcaatatatgaggagttaccaaatacaagcattattaaaaatggatttgttgaaagagaagttacaggttctaaatatttagattatggtttatatgagcctatagaagatggtactagatataaacttattgtcgaaggagaatttaaagataatatagaatttatatctttatacaattctaaccctaattttaatgaaacatttatatatccatcagagataattaatggagttgctgaaaaagaatttattgcaaaaccatccaccgaagacaaaccaaggttaaatacagatgttagaatatatatacgaccttatgattcaactatctctaaagtaagaagagtagaattaaggaaagtttaataaataagttgacagaaagttaataatatggtatacttataaagtaatatttagtgggtataccatgttatattaataaagaaaacaacagatgaaaggaattaaaaaatatggcaattgcaacgtataattctcatgttgagttagcaaaatatctagttagtaaagctgattcagtttacttaacaattggaaagagcacaccgtggtctaatgaaacaaacccaccgcaacctgatgaaaatgcaacagtattacaggaggttattggatacaaaaaagctactaaagttactttagttagaccttctaaatcacctgaagatgataataagaatttaatttcttacggtaataaatcatgggtagaagtaacacctgaaaatgctaaagctgaaggagctaaatgggtttacttagaaagtagtattgttggtgacgaactgcctcttggaacatatagacaagtaggatttgttatggacttagtagcaaaaagtggtattagtaaatttaacttagtacctagtgaagtagaatcaactggaacattattattctttgataataaacaattccaaaatagaagtgagcagacaactgctaaagaaagatttattgtagaagtttaaagaaagggagataattctaaatggcaattaattttaaaggttcaccttatttagatagatttgacccgtctaaagatagaacaaaagtattatttaatcctgatagacctctacaacaggcagaattaaatgaaatgcagtctatagaccaatattatttaaaaaatctaggagacgctatttttaaagacggagataaacagtcaggacttggattcacattgtctgaagataatgtattgacagtaaatcctggttatgtatatatcaacggtaaaataagatattacgataatgacgattcagttaaaataactggcgtaggtaaagaaactatcggtattaagttaacagaacgtattgttacacctgatgaagatgctagtctattagaccaaactagtggagtaccaagttacttctctaaaggtgcagatagattagaagaaaagatgtcattaacagttaatgaccctacatcagcaactatttatactttcatggatggagatttatatattcaatcaactaatgctgaaatggataaaatcaataaagtattagctgaacgtacttatgatgagtcaggttcatataaagtaaatggttttgagttattctcagaaggtaatgctgaagatgatgaccacgtttctgtagttgtagatgcaggtaaagcttacgtaaaaggttttaaagtagataaacctgtatcaacaagaattagtgtacctaaatcttatgacttaggaacagcagaaaatgaaagtactatctttaataagtctaataattctattagtttagctaatagccctgtaaaagaaattagacgtgttacaggtcaagtacttattgaaaaagaacgagttacaagaggagcccaaggtgatggtcaagattttctttcaaataatacagcatttgaaattgtaaaagtttggactgaaacaagccctggtgttactacaaaagagtataaacaaggagaagacttcagattaacagatggtcaaacgattgattggtcacctcaaggtcaagaaccttcaggaggtacttcatactacgtttcttataaatataacaaacgtatggaagctggtaaagattatgaagtaacaactcaaggtgaaggtttgagtaagaaatggtacattaacttcacaccttcaaatggtgctaaacctattgaccaaacagtagtattagtagactatacttactacttggctcgtaaagattcagtgtttattaataagtatggtgatattgcaatattacctggtgaacctaatattatgagattagttacaccaccattaaacacagaccctgagaatttacaattaggtacagttacagtattacctgattcagatgaagcagtatgtatttcatttgcaatcactagattgtctatggaagacttacagaaagttaaaacaagagtagataacttagagtataaccaagcagtaaatgctctagatgatggcgctatggaaggacagaacccactaacattacgttcagtatttagtgaaggtttcattagtcttgacaaagcagatattacacatcctgactttggaattgtatttagttttgaagatgcagaagctactctagcttatacagaagcagttaaccaacctaagattattccaggagatacaacagctcatatttggggtagattaatttcagcaccatttactgaggaacgtacaatctatcaaggtcaagcatcagaaacattaaatgttaacccttataatattcctaacaaacaaggtgtgttaaaactaacacctagtgaggataactggattgatactgaaaatgttacaatcactgaacaaaaaactaaaaaagtaactatgaaacgattttggagacataatgagagttactatggtgagactgagcattacttgtattctaacttacagttagatgcaggacaaaagtggaaaggtgaaacttacgcttatgatagagagcatggacgtactggtactttattagaatcaggaggacaacgtactctagaagaaatgattgaattcattagaatcagagatgtatccttcgaagttaaaggactaaaccctaatgataataacttatatttattatttgatggggtaagatgcgctataacacctgcaactggttatagaaaaggctctgaagatggtacaataatgacagatgctaaaggaacagctaaaggtaaatttactattcctgcaggtattcgttgtggtaaccgagaagttacccttaagaatgctaactccacaagtgctacaacttacacagcccaaggacgtaaaaaaaccgttcaagatattattattagaactcgtgtaacagtaaacttagtagacccattagcacaatcattccaatatgatgagaacagaactatatcatcattaggattatactttgcttctaaaggtgataaacaatctaatgttgttatccaaattagaggtatgggtgaccaaggttatcctaataaaacaatctatgcagagacagttatgaatgctgatgatattaaagtatctaataatgctagtgctgaaactagagtatactttgatgaccctatgatggctgaaggtggtaaggaatacgctattgttattattactgagaacagtgattatacaatgtgggtaggtactaggactaagcctaagattgataaacctaatgaggttatctcaggtaacccataccttcaaggtgtattattcagctcatcaaacgcatcaacatggactcctcatcaaaactctgaccttaaatttggtatttatacttctaaatttaatgagacagcaacaattgaattcgaaccaattaaagatgtatcggcagatagaatagttcttatgtctacgtacttaactcctgagagaacaggatgtacatgggaaatgaaattaattctagatgatatggcatcttctacaacattcgaccaattgaaatgggagcctattggtaactatcaagatttagatgttttaggtctagcaagacaagttaagttaagagcaactttcgaatctaatagatatatctcaccattaatgagctctagtgatttaacattcactacattcttaacagagttaacaggttcatatgttggtagagctattgatatgacagaggctccttacaatacagtaagatttagttatgaagctttcttacctaaaggtactaaagttgttcctaagtattctgcggatgatggaaaaacttggaaaacatttactaaatcccctacaactactagagccaataatgagtttacacgctatgtcattgacgagaaagtaaaatcatcaggaacaaatactaaactacaagttagattagatttatcaactgaaaatagctttttacgtcctcgtgttcgtagacttatggttactactagggatgaataaactagaggggttgattgacccctctttatttaataaggagagatttatatgcctagagaagttagagacccttattctcaagctaaattatttatacctacagttgaggaaaaatcaattaaggaattagaaaaaacatacaaagaaaaaattgatgaagctactaagttaatcaatgaattaaagaaagagagaggagaaaaatagatggcatttaactacacgcctcttactgaaacacagaagttaaaagatatgtatcctaaagttaatgatataggtaactttttaaaaacagaagttaaccttagtgatgtaaaacaaatatcacaacctgactttaataatattttagcatctatacctgatagtggtaactattatgtaactaattcaaaaggtgctcctagtggagaagctacggcaggatttgtaagattggataaacgaaatgtaaattattataaaatttactattcaccatatagcagtaataaaatgtatatcaagacttatgctaatggtactgtatatgattggattagttttaaattagatgaaggtaacttatacaatgaaggtaatactttgaatgtaaaggaacttactgaatccacaactcaatatgcaacactagttaatcctccaaaagagaacttaaatacaggttgggttaattacaaagaaagtaaaaatggtgtttcttctttagtagaatttaacccggttaactccacttcaacttttaagatgataagaaagttaccagtacaagaacaaaagcctaacttattgaaagatagtttatttgtttatcctgaaactagctattctaatattaaaacagataactgggatacgcctccattttggggatattcttctaatagtggtcgttcaggagttagatttagaggagagaatacagtacagatagatgatgggtctaatacgtaccctttagtagtttctaataggtttaaaatgggtaaagagctttctgtaggtgatactgtaacggtatcagtatatgctaaaattaatgaccctgctttacttaaagataacttagtttactttgaattagcaggatacgatactgtagatgatactagtaaaaatccttatacaggaggacgtagagaaataacagcaagtgagataacaactgagtggaaaaaatactctttcacattcacgatacctgaaaatacaatcggagcatcaggcgttaaagttaattacgtatctttactactaagaatgaattgttcatctagtaaaggtaatggtgctgtagtatactatgccttacctaaattagaaaaatcatctaaagttacaccatttattacacatgaaaatgatgttcgtaaatatgatgagatttggtctaattggcaagaagttattagtaaagatgaattaaaaggtcactctcctgtagatattgaatataatgattattttaaatatcagtggtggaaatctgaagttaatgaaaagagtttaaaagatttagctatgacagtacctcaaggatatcatacattttattgtcaaggctctattgccgggacgcctaagggacgttctattagaggaaccattcaggtagattatgacaaaggtgacccatatagagctaataagtttgttaaattattgtttactgacacagagggtattccttacacattatattatggtggttataatcagggttggaaactcttaaagcaatcagaaacttctactttactatgggaaggtactttagattttgggtctacggaagctgttaacttaaacgactcattagataattatgatttaattgaggtaacttattggactcgttcagcaggacatttttctacaaaaagattagatataaaaaatacatcaaatttactgtatattagagattttaatatttcaaatgatagtacaggttctagtgtagacttttttgaagggtattgcacttttcctactagaacatcagtacaacctggtatggtaaaatctataactttagacgggtctacaaatacaacaaaagtagcatcatggaatgaaaaggaacgtataaaggtatacaatattatgggaattaatagaggataaagaaaggtggaataaaaaaactatggctgttaaatatgatataggtaataatgagatagtattacatttaagagaaggtaaatatataacagggtttacaacagtaggagggtatgataaggagttaggacaagtaaaagttaatagagaaatcttacctgcttacttctttgataattttgcctatgaaagatatttgtattatagtaaacctgaagaggttatagaaaataaaaactatgtaccaccacaaatcaatgatgatgaggaatcccaacaaattactgtacctaaagaacaatatgatagtttaaaagaagaactagagcttatgagaaaacaacaagaagctatgatggaaatgcttcaaaagctcttaggtcaaaaggggtaattataaatggcattaaattttactacaataacggaaaacaatgttattagagacctgactactcaggtcaataacattggagaagaattaacaaaagaaagaaatatatttgacattaccgatgatttagtttataattttaataaatcacagaaaattaaactaactgatgataaaggattaactaaatcttatggaaacataacagcccttagagatataaaagaacctggttattactatataggtgctagaacattagcaacattattagatagacctgatatggaatctcttgatgttgttttacatgtagtacctcttgatacttctagtaaggtagttcaacatttatatacgctatctactaacaataaccaaattaaaatgttatatagatttgtctcaggaaactctagttcagaatggcaatttattcaaggattacctagtaataaaaatgctgttatatcaggcactaatattctagatatagcttcaccaggtgtttactttgttatgggaatgacaggaggaatgcctagcggagtaagctccggatttttagacttaagtgtagatgctaatgataatagattagctagactaactgatgctgaaactggtaaagaatatactagcattaagaaacctacaggaacatatacagcctggaaaaaagagtttgaaccaaaagatatggagaaatatttattaagtagtattagggacgatggtagtgcatcattcccactcctagtttatactagtgataataaaacgttccaacaagctattatagaccatgtagacagaaccggacaaacaacctttactttttacgttcaaggcggtgtatcaggttcccctatgtctaatagctgtcgagggttattcatgtcagacacacctaatacttctagtttacacggtgtttataacgctataggtacagatggtagaaatgtcacaggttcagtagtaggcagtaattggacttcaccaaaaacatccccttctcataaagaattatggacaggagcacaatcattcttatctacaggaactactaagaatttatcagatgatattagtaactactcttatgtagaagtttatactaaacataagacaacagagaagactaaaggtaatgacgatacaggaactatatgtcataagttttatttagatggcagtggaacttacgtttgttcaggtacatttgtttccggggatagaaccgatacaaaaccacctatcacagagttttatagagtaggtgtatcttttaaaggttctacatggacgcttgtagatagcgcagtacaaaatagtaaaactcaatacgttacaagaattataggtattaatatgccatagactaggagaaatttcctagtcttttttttcttgacttgaaaaggattatgtggtatactataactcgtgtaaggatataaggagattaaaatgagattaagaattaagaacttatatacctatgtagaatttgaggaggatgataaatacttaaaagatatatttttaaagagagtccatacaactataggagcaaggcaagaaggattccaatatagccctgcgtacaaaagaggtagttgggatggttatgtagatttttatgtttatgaggaagataaattccctactggacttttatttaaaattgagttattattaggtgagctacaatcaaggtataatttccagtttgaaacaattgatgagcgtgatgaaagtttcttatctgaagaagatattgatgatgagataacattgcttgataataatgtcggtcaaattaccttaagagattaccaatatgaagctgtgtacaatagcttaacattttacaatggtattgctcacttagctactaatggaggtaaaactgaggttgctagtggtattatagaccaactattacctcaattagaaaaaggtgaaagagtagcattcttcacaggctctacggagatatttcatcagtctgcggatagactacaagaacgtttaaatattcctattggtaaagtaggtgcaggtaaatttgatgttaagcaggttacagttgtaatgatacctactttaaatgcaaaccttaaagacccaacacaaggggtaaaggttacacctaaacaaaatattagtaaaaagattgctcaagagatactacctaaatttgaaggtggaacaaatcaaaagaaattactaaaagtattacttgataacacaacacctaaaacaaaagtagaacaaaatgtattaagtgccttagagataatttaccaaaatagtaagacagatgcagaagttttattaaacttaagaaatcataatgcacattttcaaaaaattgttagagaaaagaacgaaaagaaatatgataaatatcaagatatgagagattttttagactcagttacagttatgatagttgatgaggcacaccattctaaatctgattcttggtacaataatctaatgacatgtgaaaaagctttatatcgaattgcattaacagggtctatagataaaaaagacgaattactttggatgagattgcaggctctatttggtaatgttattgcacgaactactaataagtttttaattgatgaaggtcattctgctagaccaacaataaatattatacctgtagctaatcctaatgacatagatagaattgatgattatagggaagcttacgataaaggtataacaaataatgattttaggaataaacttattgcaaaactaacagaaaagtggtataatcaagataaaggtacattgattattgtaaacttcattgaacatggagacacgatatcagaaatgttaaatgatttagatgtagagcactacttcttacatggagaaatagactctgaaactaggagagaaaaattaaatgatatgagaagtggtaagcttaaagtaatgatagctacatcacttattgatgagggtgtagatatatccggtattaatgcactaatattaggtgcaggaggtaagtcattaagacaaacattgcaacgtattggtcgtgctttacgtaagaaaaaagacgataatacaacacaaatatttgattttaatgatatgacaaatagatttttatatactcatgctaatgagcgtaggaaaatttatgaagaggaagattttgaaataaaagacttaggaaaataggagggtaagagatggcaacaaaaacacaaagaaagctataccaatatctagaggaaaatgctacagaaaataaatttcatatttctactaagaaagagttagcagattctctaggtgtttccatctctgctttatccaataaccttaaaaagttagaagaagaaaataaagtcgttactgtttctaaaagaggaaaaaacggtggagtaataataactttagttagagagtatgacacagaagaattgaaagaatttaataattctacagataatattattacttccgatttacagtatgctaaggcattaagagaaaagcacttcccttcttatagatatgagagaaaagaacaacgtagacgtactaaaatagaaatggcacaatacaatgccattaaggatgagaagagaagaattatagcagatatgaacttctattcagaaggtcttccttatccttctaaagatatttttaatatgtcttatgacccggaagggttttataaagcatacattctatgtaagttatatgaccaatatgctatttctcatatggatgctaaacacacaagtcatcttaaagcaatgagtaaggcaacaactaaagatgaatatgactatcatcaacatatgtctgaatactatagaaataaaatgattcaaaacttacctagaaatagcgttagtgataatttctttggtagtaaaatgtttaataccttttataatttttatttaaaaataaaagataaaaatattaatgtatttaaatatatgcaaaacgtatttaaaaatgtaacattttattatgagaacggtatgcaacctaatccaataccttctcctaacttctttagttcagataagtattttaaaaactataataattatattcaaggaataaaaaaaggtgttaacagtacgaatagacacctaggtgatacagacagcatcattaattcatcagactacgtgaaaaaccctgctgtattacatctacaccaactatatactacaggattaaattctactttacatgatattgatactatgtttgaacaagccttagaccttgaaaatgcctcctatggactatttggagatatgaaacatattattttactacagtataattctatgattgaagaagaaattaagaatttacctagagaagaaaaggatattattaataaatatgtaaaacaatgcataattaatgattattcaccaacaagtatttcaccatctgcaaggttatcaatgtttactatgcagaaagagcatatagtttacaataagcagttaaataagggaatcaagagagaggatttactaccattaagtctaggaggtatagtgaataaagattcattgagcggtatggatatacaaaacttagaacagaatggtaatgaatacctatatatgagacaacatacttcaacttattatatactaagaatgtttggtgactatctaggttatgaggtaaacttaagagaagtaaaatatattgcagagaaatataatttaattgataaaataccattgacaaaagagggtatgttggattataataaacttatacatttagtagaggaagaggttaataactatgagtaagaagataaaggagcttatccttcataaatcaatgaaggatatacattttgcaagagaagtattagataacttacctaagaatctattttcagcagagtctgaggacatgggttacttatttacagctataaagagaacagcacatatttccgataagatgtcaaatgaagcattagcaattaaagtagaacagcttatgggtaataataaggaagatgaagagaaagtaactaagacattaacttacttagaagatttatataaagtagacgttaatgaaaaagatgaatctgttaattatgaaatagaaaagtatattaaaacagaaatgtcaaaagaagttttagttaaatttattgcagaaaataaacaagaagactctgataatctacatgaacttgtagacaaactaaagcaaatagaagtaagtgacatctcaggaggtaatggagagtttattgacttcttcgaagatacagaaaagaaacaagaactattaagtaatttagctacaaataaattctctactggatttacttctattgacaaccatattgaaggtggtatagcaagaggagaagttggattaatcatagctcctaccggtagaggtaaatcattaatggcttcaaacttagctaagaattatgttaaaagtggattaagtgttttatatattgccttagaggaaaaaatggatagaatggttttgcgtgctgagcaacaaatggcaggagcagaaaagagtcaaattgtaaatcaggatatgtctttaaataataaagtttatgatgcaatacaaaatcattatcagaagaatagaaagttattaggtgacttttatatttctaaacatatgccaggtgaagttacaccaaaccaattagaacaaattattgtcaatacaacaattaagaaggataaaaatattgatgttgttattattgactatcctcacttaatgagaaatccttatgctaaatatcattcagaatcagatgcaggaggaaaattgtttgaagatattcgtagattatcacagcaatatggatttgtttgttggacgttagctcaaactaaccgtggtgcttatggttcagatgttattacaagtgagcatgtagaaggttctcgtaagattgtcaatgctgttgaggtgtctttagcagtaaaccaaaaagatgaagaattcaagagtggttttttaagattatacctagataaaatccgtaatagctctaacacaggggaacgatttgttaatcttaaagtagaaccaactaagatgattgtaagagatgaaacacctgaagaaaaacaagagcatatacaattgctatcagataatggaaaagaagacacaagtaaatttcaaaataaagataataaaatagaagctataaataacacattcggaggattaccgggagtttaattttttaaaatataccacttgacattttatatgttaggtggtataattattttataaagaataaaggagagattaataatgaaatttgtattctttacagatagtcattttcacctatttactaactacgctaaacctgataatgaatttgtgaatgatagatttaaagaacagatagaagcattacagaaagtttttgatattgctaaaaaagaagaagcaacagttatatttggtggagatttattccataaacgtaactcggtagatactagagtatataacaaagtatttagcacatttgccaaaaatgatgaggttcctgtattattacttagaggtaatcatgatgctacaactaattcattatatactgattcaagtatagatacatttgagtatctccctaatgtaagtgtaataaaatcattaaatacaattttaaaagataatgttaatattgtgtttactgcttatggggatgagacggaggaaataaagacatacattaatagtaattacgataaagatatggtcaatatactagtaggtcatttaggtgtagaaggttcattaactggaaaaggctctcatagattagaaggggcatttggataccaggatttattacctgataaatatgatttcattttactaggtcattatcaccgtagacagtatttccaaaatccgaatcatttttatggtggctcattaatgcaacaatcattttctgatgaacaagaagctaatggtgttcatttaatagatacagaaaaaatgactacagaattcatcccaattcatacacgtagatttattactattcaaggagsaaagatattcctgagaactttgaacagttaatcgaggaaggtaattttattagggttatcggtacagcaaatcatgctaaggttttagaaatggatgacagtatgaaagataagaatgttgaagttcaaattaaaaaagaatatactgtagagaaacgtattgatagtgatgtatctgatgaccctttaacaattgctagtacctatgctaaacaatactcacctgaatcagaacaagaaatacttgagtgtttgaaggaggttttataatgaaaaaatatagagaatacctaaataagacagatgcagaaaatttagcagaggattgggagaaagtaaccgaagatttatggaaagtgtttaaagatatgaaacctaaaattaatacattagatattagtaatgtagaaagtaaaaacttagataaaagtaaacctatactacaattccaagattcagatggagtaatagagaatatttgtaatgttgagggtttagaagatggtttaagtaaaatgaaaaaggtttttgatgatagtaactttgaaaagcattattatagtagagtcgtagaccatgatgagtattactggattgattatggttctcatcattgtttctttagagttacgaaaggggataagtaatggttgtatttaaacaagtagaagttaataattttttagcaattaaagaagctacgctagagttagacaatagaggattaattctaattgaaggtgagaataaatccaatgagtcatttcattcaaacggttcaggaaaatcaactttaatatctgccattacttacgctttatatggtaaaactgaaaaaggactaaaagcagacgatgtagtaaataatattgagaagaaaaatacatctgttaaacttaagtttgatattggggaagatagctatttaattgaacgttatcgtaaggacaaagagaataagaataaagtaaaattatttgttaatgaaaaagagattacaggttcaacaaatgacgttactgataaacaaatacaagacttatttggtattgagtttaatacttacgttaatgctatcatgtatggtcaaggagatatccctatgttctcccaagcaacagataaaggtaagaaagaaattcttgaatctattactaagacagatgtatataaacaagcgcaagatgtagcaaaagagaaagttaaagaagtagaagaacaacaaaataacataagacaggaaatctataaactaggttatcagttatctacaaaagatgagtacttccaaagagaaatagaacagtacaaccagtataaagaacaattggttcagatagaaaacagtaataaggaaaaagatagattaagagaacaagaggagaagcaaatagaagctcaaatagagcaattatcttcacagataccaacaatacctgaagatgaatttaagcactcagaggagtataataaagcttctcaaagcctagatttactttctaataaattaacggagctaaatcaagtatactcagaatataataccaaagaacaagtactaaaatctgaaatagctacattaagcaatagtctaaataagttagatacaaatgaccattgtcctgtttgtggctcccctatagataattctcataaattaaaagaacaggaaaatattaataatcagattgagaataagaaacaagagattactagtgtattagaaatgaaagatacgtataaagaagctattgataaagtaaaagataaatcacaagaaattaaagataaaatgtcacaggaagaccaacaagaacgggagcacaataataagattaacagtataattcaagaggcttctaggattaaatcagacattagctcattagagaataataaaacttatttaaaagtgaaataccaacatcaatctgttcaaggattagagagagaagaaccaagtaaagaaaaacatgaggaagataaaaaagaattacaagaatctattgacaaacatgaagagaatatagtacaattagaaactaagaaaggtaaatatcaacaagctgtagatgcttttagtaataaaggtatacgttcagtagtgttagactttattacaccattcttaaatgagaaagcaaatgagtaccttcaaactttatcaggttcagatattgaaatagagttccaaactcaagtgaagaatgctaaaggagaactaaaagataagtttgatgttattgttaagaatagcaagggtggaggctcatacaaatctaattcagcaggagaacaaaaacgtattgatttagcaattagttttgcaattcaggatttaattatgagtaaagatgagatatctacgaacattgcactttacgatgagtgttttgatgggttagatactatcggttgtgaaaacgtgattaaattattaaaagatagacttaatacagtaggaacgatatttgtaattactcataataccgaacttaaaccactatttgaacaaacaattaaaatagtaaaagaaaatggagtatcaaaactggaggaaaaataatgaaattaaagattttagataaagataatgcaacacttaatgtgtttcatcgtaataaggagcacaaaacgatagataatgtaccaactgctaacttagttgattggtaccctctaagtaatgcttatgaatacaagttaagtagaaatggagaatatttagaattaaaaagattacgttctactttaccttcatcttatggtttagatgataataaccaagatattattagagataataaccatagatgtaaaataggttattggtacaaccctgcagtacgcaaagataatttaaagattatagagaaagctaaacaatatggattacctgttataacagaagaatatgatgctaatactgtagagcaaggatttagagatattggagttatattccaaagtcttaaaactattgttgttactagatatctagaaggtaaaacagaggaagaattaagaatatttaacatgaaatcagaggaatcacaattgaatgaagcacttaaagagagtgatttttctgtagacttaacttatagtgatttaggacaaatttataatatgttgttattaatgaaaaaaattagtaaatagtaaggaaggatattatgaggtttgaagactttttaacccaagaattaggagaaccaaaagaaaatactataggtgagctaagatactgttgtccgttttgtggagaaaaaagttataagttctatgttaagcaagccctagactctagtaatggtcagtatcattgtaaaaaatgtgatgaatcaggtaatcctattacatttatgaagacttattataacattacaggtaagcaagcttttgatttattagagtctaagaatatagatatagagagagcccctttacttacaaccaataataaggatttaacagaatcagagaaacttatattaatgcttagaggtgtgcatcaagataagggaactactagtattaaacctcctcgattacctgaaggatataaattattaaaagataacttaaataataaagagattataccttttttaaaatacttaaaaggtagaggtataactttagaacaaatcattaataacaatataggttatgttattaatgggagcttttataaagttgacggggaatccaaagtatcattaaggaatagtattatattttttacttatgataatgatggaaactaccagtactggaatacaagaagtatagagaagaacccttatattaaatctattaatgctcctgctaaacaagatgaagtaggtagaaaagatgtcatatttaatttgaatatagcaagaaagaaaaagttcttagttataactgagggtgtatttgatgctttaacctttcatgagtatggagtagcaacattaggtaaacaagtaactgagaatcaaataaaaaaaataattgattatgttagtatagatacatcaatatatattatgttagacactgatgcattagataataatatagacttagcttataagttaaaaacacattttaacaaagtttactttgtaccacatggtgatgaagatgcaaatgatatgggaacaaggaaagcttttgagttattaaaacagaaccgggtgttagtaacacctgaaagtatacagagttacaaaatacaacaaaaacttaaactttaggcttgaccttagagaagttttatgttatactagtaattaagtaattaataaaggagaaaaaaataatgtcaaataataaaaaagatattttagaatttgtagatgaatacattacagctttaagagttggtaatgagcaacgacaacatcaattagaagaaatgggtaaagaagaaacagcaacattaacagatgtagctaaagctattactaaccttatgttaggtgttaatgagcagatgacagacttagaatataataacgagttaaacttaaatattttaattgatgctttatataaagcagagcttattaatgaagatgtattagactacattcaagaatcaattgataaatcacaagaagaacctaaaaatgaagaagaaaaaggagaacaagaataatggaaaaaaatattagcacacacacaaaaggtattagtcaagcagacatggagaaatggattgaagctgtagtacaaggaactgttgatggtaaacaagttgatgagaaaacagctaaacaattagatagaattggttcacgaagtgtttctttagaagaagcaactcgtattgctaaagtccttaatgctgtaacagctcaagaggttacaggagactttaatgatgcatttaatgcaattgacttaatgatgattatcatggaagatgagttaggagtaactcaagaaaaagtaggtaaagctaaagataaactaaatgaaaaacgagaagcttacctaaaagagaaacaagaagaattacgtcaaaaacaacaagaagaggcacaaaaagaaactgaatctgacagcaatgaaaaagtaattcagttgaagaaaaatgacgaacagtaagaaaaaaggggatacattcgaacgtaaaatagctaaggaattaactgcttggtggggatatcaattcaataggtctcctcaatcaggtggtgcctcttggggtaaagataataatgctgtcggagatatagtagtacctcaggaagctaattttcctttagtagtagaatgtaaacatagagaagaatggactatagataacgttcttttaaacaatagagaaccacatacatggtgggagcaagtcattaatgatagtagcaaggtgaataagacaccttgcttaatatttactagaaacagagctcagagttacgttgctttaccttatgatgagaaagtatatgaggatttaagaaataatgaatatcctgttatgagaacagattttattattgataatattcgaaaagataaatttttttatgatgttcttataactaccatgaatgggttgacctcatttacaccttcttatattatatcttgctacgacaaaaaagatataaaaccatacaagaaggttgagtctaatttatctgaggtaagtaagcatgaagatgaattgattaatgaccttcttaatgatatataaggaaggtaagataggtatgacaagtaaagaaagaccattaattgtatatttttcaggtacaggacaaacagaaagactagtaaacaaaattaatattaataattcatttgaaacatttagggttaaaagtggaaaagagaaagtaaataaaccttttatactaataacacctacttatatgaaaggtgcaatacctaaacaaatagaaagattcctagaaattaatgggagccctaaagaagttattggtacaggaaataaacaatggggctctaatttctgtggggcaagtaaaaagatttcagagatgtttaagattcctttaattgctaaagtagagcaatcaggacactttaacgagatacaaccaatattagaacactttagtaataaatataaagtagcgtaaaggatgagagatatatggcaacatatggaaaatggattgagttaaataatgaaataactcaattagatgacaatggaaaaaataaactctataaagaccaagaggctttagatgagtatttaaaatatattgaagataatacaagaaagtttaataatgaagtagaaagaattagagtattgacaaaagaaggaacatatgataaaatatttaacaaagttcccgatactattattgatgagatgactaagttagcttatagttttaattttaaatttcctagttttatggcaggacagaagttttatgagtcttatgcatcaaaacagtatgacgagaataaaaaacctatttttgttgaagactatgagcaacataacgttcgagtagctttatatttatttcaagatgattatgtaaaggctagagaattattagtacaacttatggagcaaacattccaaccatctacgcctacgtataataactcagggcaagctaatagaggtgaactaagttcatgttatctatttgtagtagatgattcaattgagtctctaaattttgttgaggatagtgtagctaatgctagttctaatggcggaggagttgctattgatttaactagaattagacctaaaggagctccagtacgtaatagacctaattcaagtaaaggtgttattgcttttgctaaagccattgagcataaagttagtatctatgaccaaggtggagttagacaaggtagtggtgcagtatatcttaatatattccataatgatatcttagatttattgagttctaagaaaattaatgctagtgaatcagttagactagataaattgtctattggtgttacaatacctaataagtttatggaattagttaaagaaggtaaacctttctacacgtttgacacttatgatattaacaaagtatatggtaaatatttagatgaattaaatattgatgagtggtatgaagagctactaaataatgataagattggtaaagtaaaacatgatgctagagaagtcatgacagacattgctaaaacacaattagaatcaggttacccttatgtattctatattgataatgctaatgataatcaccctcttaaaaatctaggtaaggttaaaatgagtaacttatgtacagaaatttcacaattacaagaggtatcagaaatttatccatactcttatagtaatcataatgttattaatagagatgttgtttgtacattaggttcacttaatttagttaatgtggttgaaaaaggtttattaaatgaatctgtagatattggtacaagagcactaacaaaagttactgatattatggatttaccgtatttacctagtgttcaaaaagcaaatgatgacattagagctatcggcttaggttcaatgaatttacatgggcttttagctaagaatatgattagttatggttctagggaagcattagacctcgtaaacagtttatatagcgctattaacttccagtctattaagacatctatgttaatggctaaagaaacaggaaagccatttaaaggatttgaaaaatcggattacgctacaggtgaatactttgtaagatatattagagagtctaaccaacctaaaacagaaaaagctaagaaagttttaaacaaggtttatattccgacacaagatgattgggatgaattagctaaggcagttaaagttcatggattgtataacggatatagaaaagctgaagcacctactcaatctatatcatatgtacagaatgctacaagctcaattatgcctgtacctagtgctatagagaacagacaatatggagacatggaaacatattatccaatgccttatttaagccctataactcagttcttctatgaaggagaaacagcttataagattgacaataaacgtattattaatacaagcgcagttgttcagaaacatacagaccaagcagtatctacaatactttatgtagagtcagaaatacctactaataaactagtatcattatactattatgcttgggaacaaggattaaaatcattatactatacacgttcacgtaaactttctgttattgaatgtgaaacatgttcggtttagaaaggaaatagatatggatattacacaaaaagtaaagcaacataataaaaatgcagtattaaaagcaacaaattggaatattgaagatgacgggatgtctgatatttattgggagcaaggaatctctcaattttggactcctgaagagtttgatgtatcaagagatttaagtgcttggaatagtttaactgagagtgaaaagaacacttataagaaagtacttgcagggctcacagggctcgatacgaagcaaggaggggaaggtatgaacttagtatcataccatgaaccaagacctaaataccaggctatatttgcgtttatgggtggcatggaagagatacatgctaaatcttatagtcatatctttacaacattattaagtaataaagaaacaagttatttattagatacttgggttgaagagaatgactttttaaaagtaaaagctcagtttattggatattactatgaccaattattaaaacctaatcctactgtatttgatagatatatggctaaagtagctagtgcctttttagaaagtgcattattttactcaggattttattaccctttacttcttgcaggaagaggtcagatgacacaatcaggagctattatttataaaattacccaagatgaatcttatcacggttcagcagtaggattaacagctcaatatgattacaatcttctaacagaagaagaaaaaaaattagcagatagagaaacttatgaattattaaatattctttatactaatgaagtagcgtatacacatagtctatatgacccactagagttaagtgaagatgtaattaactatgttcagtataactttaatagagctcttcaaaatcttggaagagaagattattttaatcctgaaccgtataaccctattgtagaaaatcaaactaatgtagacagattacgaaatgttgatttctttagcggtaaagcagactatgaaaaatctacaaacattaaagatattaaagatgaagatttctcattcttagatagtaaagagtatagtactgctaaggaattcctataaaaaggagaaaagatattatggatagaaaagaagcaatggatttattaagtaaaacagaaatattatttaaaaaacatgatgagttttcatgtgtgagtgatatcaatgaccctatgaagttatttagtaactctaaagatgtcaaagctgatgatacgtctaagtcttttcagttagagtttatgcatgatatgactatgtatactttatcttatggctcaggacagctaaaactaattgatttatcagaaggttatgaagcacaaaaagctacagtagttaactcatttcccgaaattattaaaacattagaaaaggatgattcagaagatggaaaaaatgaatagtttagtagatttaaatacagcaattagacaaaagaaagatgttattgtcatgattacacaagataattgtggtaagtgtgaaatcttaaaaagtgtaatccctatgttccaggattcaggggatattaagaaacctattttaacacttaatttagatgcagaagatgtagatagagaaaaggctgttaagttatttgatattatgagtacaccagtattaattgggtataaagatggtcagttagttaaaaaatatgaagaccaagttacaccaatgcaattacaagaattagagtcactttaatttggaatttcctactatctgtgctatactataatagtacaaggtagtaggattttttaatggaaggaagatgacatatcgcaaagaataaaacattaacgatatataatagtgatagatattttaatatacacacaaaagataaagataaaattaatgaggctattaaagtcacacacggtaatgaagaagaaattgaaaagaatatggatgaactaatatctaaatcgagacgatatattatgagagatgaaaatcattatatgctatttaatgagaaatacaataatgataggctcatagagaaagtatgtaaacatggtggtaaagttacatactatactgattcagtattaccttattatgttttaaaagacttatctagtcatcctgattcagaagttgtttatcgtatgcgtaatggttttactgcaaaagaagtagataatatagctttatcatttatgggaacaaaagttattattgatatttctgtggtatttccttatgtaaacccttatgatattattagaagtttacatgatattaaaacaaatgtagatgaagttcatttatcattccctcgtatactagaagtagatgagaagcaagaaaaattttatttctttgatggtgaagcttatgatttaaaacctgaatataaagttgattttgcagataaaattagagtgtccctatcagtatggaaaatgtatatctatatcttaacaagtagtcgtgattttgaagatgtagacaatgtaattacgaaactaaaacaacaacgaaagattaagatataaggtgattatatgagtacagcaaatagaagagatatagcaaggaagatatcagagaacacgggttattatatacaagatgtagaagagatactaagtgcagagacagatgctatttctgacttactagaagaagggtatactaaagtaaagaatcataaatttatgcaaatagaagttattgaaagaaaaggtaaaaaagcttgggatgggctgaataaagaatatttccatttacctaatagaaaagctataaaatttaaaccacttaaagaattagaagaggttattgatagacttaatgaagaagagaaataattctcttctttttttattgacaagatttaaaatatatggtatagtattattaagttaaaaaaggagaggaattaaatgaaagtattaatcttatttgaccacattagagaagaacatttttctgtaagtaaagatgggagtgtgaaatctaatgtattaaatacacctaatggaaaaacacttaagaaattacttgagaagtgttctaacttaaagagagataaaacaaacagagactatgatattgattttctctataacgcagtacctacacctattagaaatgattatggtaaaattattaaataccaagatgttaaacaagcagaagtaaaaccatattatgaaagaatgaataacattattattgataactcctatgatatgataattcctgttggtaaactaggtgttaaatacttattaaatgttacagctatcggtaaagtaagaggagtaccgagtaaagtaacaattgaaaatgaaacatcttctcatgatgtgtgggtattacctacgtatagtattgaatacactaatgtaaataaaaatagtgaacgtcatgttgtatcagatttacaaacagtaggtaagtttgtagaacaaggtgaggaagcatttaaacctaaggaagtatcttacgaattagtagataacattgaaagagtaagggaaatatttaataaggaagtaaagaatgataattatgatggtgtagatatcaccgcatgggacttagagactaactcattaaagcctgataaggaaggaagtaaacctttagtattatctctatcatggagaaatggtcaaggtgtgactatacctttatacaaatcggactttaattgggaaaatggtcaagaagatattgatgaagtactagaattacttaagaattggttagctagtaaagaagatattaaggtagcacacaacggtaaatatgatattaaattcttaatgagtactgagaactttaaagattttgagagtattcaagatactaaagtaggttggtatttagctgtcacacaagaagttaaagaatctttgagattatctgacttagcttacgaggtaacagatgtcggaggttacgataaaccattagaagattttaaattatggtttgttacgaagttattaagatttttctctgataaaattaaagaaatacaaaaagaaaataaaaaaattgctaagaaagagtatgatgttaaagctcctgaatacaaagagtggctagatactaaattaaatgaaacagtagtagaactagatgatactgaaaagaaatttagagttagtgaattagagaaaaaatacattcagttaggtctttcacctgaaattgtaaacatgaatttagttatgaacaacgatgaatttataagtattgcagagcaatcacctgaatacatgggattatctgattacgctaaatcttacacactgaatactgcaattgatttaattaacaagtatagagatgtaaaagatgtagttaatgatattgatggaggtaactttaactatgactggttccctattgaactaatgcatccatatgcatcaggagacacagatgtatgtagaagaattcattgtgatgttgttaaaaaacttaaagagcaagatagacctaagtcaatgcacttattagaagttaactacccaagacttactaaatcattagctagaattgaatcaaatggtttatactgtgacttagattacatgaaagaaaatgatgagtcatacgaatctgagatggctaagaaccacgctactatgagagaacactgggctgttaaagaatttgaagagtatcagtataacctttaccaaatggcgttagaagagcatgagaaaaagccaaaagatagagacaaagagattcatcagtatagagataagtttaaagatggtaaatggatgttttctcctagttcaggagaccataaaggtagagtaatttatgatattctaggaattcagttaccttatgataaagagtatgttaaagaaaaaccatttaacgctaatgttaaagaagccgaccttacttggcaggattataaaacagataagaaagctattggttatgcattagataacttagaattaaaagatgatgttaaagaacttcttgaattacttaaatatcatgctagtatgcagacaaaacgtaactcatttactaagaaattacctaatatgattaataaacaaaaacgaacattacatggttctttctctgagacaggtacagagacatcaagattaagtagtagtaatcctaacttacaaaacttacctgctcatacatcagatgtaaacaagtttgattataaacatccaattaaacgttcatttgtttctagatttgaaaacggagtactactaggagccgactatagtgccctagagatgcgtattattggattatttactaaagaccctgatatgctacaatcattcttaaatggtgaggatattcataaggctacggcaagtatcgtttataataaaccagtagaagaagtaactaaagaagagagacaagcaactaaagcagttaacttcggattagcctttggtgaatcaccattctcatttgcaggtaaaaataatatggaagtaagtgaagcagaagaaatatttgaaaaatatttccaaacaaaaccaagtgtaaaaacttctattgacaatgtgcatgaatttgtgcaacaatatggttatgtcgatacaatgcatggacatagaagatttatccgctcagctcaatcaacagataaaaaaataaaaaatgaaggtttaagacaatcatttaataccattatccaaggctcaggtagtttcttgacaaatatgtctttaacttacttagatgactttattcagtctcgtaacttgaaatcaaaagttattgctacagtacacgatagtatcttaattgattgccctcctgaagaggctaagattatggctaaagtgacaattcatattatggagaatttaccgtttgatttcttaaaagcagaaattgacgggaaagaagtacaatacccaatagaagctgatatggaaattggtttaaattacaatgatatggttgaatacgatgaagaagaaattgatacatttaattcttaccagggttatattaagtacatgatgaatttacagactttagaagattataaagagtcaggtaaattaactgatgaacaatttgaaaaagctactaatgttgttaaaagtgaaaaacatatctatcaagaaatttaataaaagtattgacaatatgtttcacttatgttatactatataagtaataagtataaggaggaaaaaaagagtgaatacaggagagattagatttaatcgttctatggatgaatggattataacaagtatgtaccaagatgaattaggtgaaatgaatattgttgttacattctataatagagaagaaaataaacatggctctaccgttttaccgacagaatcatctactggagaagtagcagaagaattagcaagtcttgaagaagaatatcctttagctttacctttaagtagtatttcagttaatatttaaaaggaggaactgataaatggaaatacacattgattccctagattttacaaattttactattaaagatagaaacgggaactcacaagagtttgatattacagatgaattaaagattacagagtatacgatacaagaggactttatgcaacaatcagctaaatatgctttttgggcttctatattagagaaggtcagagcatactctgaaatggagcaaagaaacctagaaacaattggtagtaagcttaatctcacaattagacaagagtatgaacaacaaggtaaaaagcctactaaagatatgattgagtctagtgtgtatattcatgattcctatcaacaacaacttaaagttgttgaggcttggaattataaagttaaacaacttcaatatgttgtaaaagcttttgaaacaagaagagatatgatgattcaattaggtgcagagttacgacaaacaaataaaaatggtggaattactaacccattttcacattaaaaaataaagtaaagaatataattgacaaatataaaaaactatgttataataaataagtaaattaattaaaaggagaaaagataattatggatttcaatcaatttattaataatgaggcaagcaaattagaaagtaataacagttcttttaacaacaatgtagagagctataaacctaaaaaccctgtattacgattaggtaatattaaagatgcaaatggaaataaagttactaaagaaaatgcttttgtacgagtattacctcctgcacaaggaacaaatgttttctttaaagaatttagaacaacaggtattaattactctaagaaagatggttctcaaggattcacaggactaacattacctgcagaaccaggttcatctgtacttgacccttacattcaggactggataacaaatggcgttcaatttagtagattccctaataaacctggagtacgttattacattcacgttattgaatactttaataataacggacaaattcaacctaaaacagatgagcaaggaaatgtaatgattcaacctatggagttatcaaacacaggatacaaagaattagtagcaaatcttaaggatactatgttaaaaccatcacctaatgcacctcatagttttatttcagcaaatgaagcattcttagttaatattgctaaagctaaaaaaggtgaaatgtcatggaaagttagtgtttatcctaatgctcctttaggtgcattaccccaaggatgggaacaacaattatcagatttagaacaattagcaaaaccaacagaagaacaaaatcctaactttgttaacttcttaatcaataatgttaataacacagagttaagccacgataactttaagtttaaccgtgagtctaatgtattaggtgaagaaccttcagaacctaaacaagctcctactcaacaagatattgataatcagatgccaagtaatatgggaggtcaacctaaccaaccacaacaaggtcaagtaggtcagtatgctcaacaaggacaaaataatggacaaggacagcagttacaaggtaaccaacaaccaattagtaatactcaatttggtcaaggaacaccttcaggacagcaacctaataatacaggttcagttgattgggataatttagctcaacaacaatcacaacctgattcaaatccttttaatgactttgatgtaaacagtgttgatgattcacaagtaccttttgagacacaaccccaaaacacacaacaagcacctgaaccacaacaaactactcaggagcctccaaaacaaaaacaaacgcaaagtattgacgatgtattaggtggtctagacttagataacctataagatatagagtgccttagagcactcttttatttgagatataattactaggaggatattaaatggcaagagcaaaaaaaggtaaagaagtagatttaacagatttaaatacaattgatttaggtaaagaattaggattaacattactatcagatacaaacagagcagatattaagaatgttatacctacaatggtacctcagtatgattatattttaggtggaggtattccattaggtagattaacagaggtttatggtttaactggtagtggtaaatcaacatttgcagttcatttgtctaggattgcaacacaattaggtgttattactatttggattgatattgaaggaacagcagacaacaatcgtatggaacaacttggagtagatgtttcaaaattattctctattcaatcaggagaaggtagacttaaaaatacagtagaattatctgtagaggctgtaggtaaagaattagagtactggattgacacctttaatgaaaagatacctggagtacctattgtgtttatttgggactcactaggagctacacgaactcagaaagagattgacggcggtattgatgagaaacaaatgggtcttaaagcatcagctactcaaaaagtaattaatgcggtaacacctaaattaaatgatacaaacacagggttaattgttattaaccaagcacgtgacgatatgaatgcaggtatgtatggtgaccctattaagtctacgggcggtagagcatttgaacatagtgctagtttacgtattaaggttcataaagcatctcaactaaaacagaaaagtgaattaactggtaaagatgaataccatggtcatatcatgcgtattgaaactaagaaatctaaattatcgagaccaggacaaaaagcagaagcagacttactgtctgattatatggtaggtaaagaagatgaccctatcttattaaatggtattgacttagaacatactgtatataaagaagcagttgaaagaggtttaattactaaaggtgcatggagaaattatgttacattgaatggtgaagaaattaaacttagagatgctgaatgggtacctgtattaaaagataacaaagagttatatttagagttattcagtagagtatatggagagcatttccctaatggatactcaccattatttaataacaaagtaattgtaactcagttagaagaatatcaagcattagagaattattataaagaatgggctacagataataaacaagaagaacaagaggaagaactaaaaggagaatctcaagaaaaggattctgaataatagatggataatttaatagataaaaacatgaatcaggtaaaagaatctttggggaatgcaaattcctcagatgttcttcctttaccttataaagatatagcaaagaaatttgaagaagtaaaagaaaaaggtgaatcaattatcattgaagaaggtggattcccttacacagattctacagtgatgtatatagaacatgtaacagatagatgggcaggaggatactccttaattagacatgaaggtgaagaggttaaagtacctaaaactatccatttctctgatatatatgttaaggataagtcacataaagtaagaataatcttcgagggggctaatccttatgaagaaagctaacaatggtaatagatatgtaatagatatagacggtattcctgttgattttgaaagggacttagatagtttacttaataggtataaaaatcttagatggtcattgtatcatagatacgcaggtattttatctaatgattttgaaagacaagaattaagagaatacattgatgaacaatttattaaactggttaaagaatataatattagaagtaaagtagatttcccagggtatattaaagctaaattaactttgagagttcaaaatagttatgttaagaagaatgaaaagtacaagcgtactgaaattataggtaaaaaagattatacagtagagtctctaacagaagacttaaatgaagactttgaggataatcaaattatgagttatgtatttgatgatatagaatttacagaagttcaaagtgagttacttaaagaattacttattaaccctgaaagggaagatgatgcttttatagtttctcaagtagcggagaagtttgatatgaaaagaaaagaagtagcaagtgaattgacagaactaagagactatgttagatttaaaataaatgcataccatgagtactatgcaaagaaagaattaaataaccatagagttaatactgaaaatcatatttgggaaaactagttacagtgccttccttgtgttatattattatcgagaattcaataataaagcatagggaaggctttttctatgtcttatagaatgctttaaaatagattactaaaataaagattggagattaagcttatggctaaaaagaatgttaatgatgtattacaacaagaatctgttacagtagcagataagtatttacaagttaaagttaaccgtgacggttatactcgtacacatgaaggacaatatgcgtacaaagtagtttcagagggagaagagttattcttataccctgtacaaacagatggtaaaggtacattaaatgtaatgaagaaatcacctattgcttatactgatggagacaacatccattttgtagtaaacacagtagtagacccttataatcattcatttattcgtactgaagatattaaaggattagataaaggtaaacaacttattcaagctttcttagctttcgttgaagaccgtttcaaatttggtgtttataacgtatttgttgcaaataacaaagaagatgtattatctatcgtagaccctgcagataatgatgcagatgaagttaaagatagtttagagcatgcacatgaagatgtaattgcggatttccctgctagccctgctcgtaaggacgttaaaggcgtagattcaggagaaggtcaaggagacacttcagaaccatcagcacctaagaacgttcaagttactcctaaggaagacggagcagacgtatcagcagaataatatatagataaaggatggtaaatttggctaagttaaatttatacaaaggtaatgagttactaaacagcgtagagaaaacagaaggaaaatcaacaatcacgattgagaatttagatgctaacacagattaccctaaaggtacttttaaagtatcattctcaaatgattcaggagagtcagagaaggtcgatgtccctcagtttaagacaaaagcaattaaagttatttcagttacccttgacgttgatagtttagaccttacagttggagatactcaccaactatcaacaactatcacgcctagtgaagcatctaacaaaaatgtgtcatttgaatcagacaaatcaggtgttgctagtgtaacatcagaagggttaattgaagcagttagtgcaggaacagctaacattactgtaactactgaggatggtagccatactgatattgttgctgtaacagttaaggaacctattcctgaagcacctgcagacgtaacagttgaacctggtgaaaatagcgcagatattactgcataggaggacaataaagaatggaaaagacattaaaagtttatagtaatggtgaagttgtaggctctcaagtagctaataacgatggagctactacagtatctattacaggcttagaagccggaaaaacttatgctaagggagcttttaaagtagcatttgctaatgattcaggtgaatcagaaaaagtagatgttcctgaatttacaactaaaactcctgctgaagaaccttcagaagaagcataataattaagaccaactaaaaagttggtctttttttattgacaatttataatatctatgatacactatataagaattaagaaaaggagggaaagtaatggatattccaacaatattatttagaaatccatatgattatacaaaagtaaaaaaattaatggagaacaaggaacagtacattgtagtaaagtttgattcagtttctgttcataatttaaatgttcaaggtatgatgaatgttattcaagactatctgcacatctatggttacagagtcaaagagtacggacaagaaaatgcttctaaagatgatgaaagagacgttaaaggttacttatacgaaagagtaggtgagtaagatatgggtgttataataaattctaaccatattcagtcagacactttatatgagtatgatagcttttttgatattgagaaagtagatacgtttgaagaaggattactatcaatacaggatgaaccaactgttttagcaggatttatctatgatgatattacatttaataaggttattaactctaattcaaatattgataacaatattaagaataacgatatttattatgtttctgatatagggctacttccggatacttttatcactgttgattctaataaaaaatactattcattattacagcagatagttgagttaagtaaagaccttttccctgaatgggtagaaacggacgcaaaagatttaactaggtattataactttcaagattttgaagatgtatttgatttaaatagtttttataaaaaagaagttgacatggtaagagaaaagtgctataataatggtaatgtatatttattatatgaagtcttgcctgattataaactacctctagcttatagtttactttcaaataaagagcatggtattgttattatcggttcacagacacgttctaataatgatatactgactttttacgttaaaggtatggatgctaagtcaatagctagcatgtttaatgtagaacatgattacgattctaatatttttcatacatttgtaaatagtcacattaatattttaggaaatcaaataactaagtttataagagagaaaggaagcagttatgagtaactataaaacaatagaagaagtacaagcagttattatcggagtattatttaaagatgagggtaaaattgtaacatctaagtttaataaaattactaaagagtttggtttagatagaatcggtaaagatgaccttaaagaaattgttgaagatattagacaagatgcttatctaaatgaacttaaaaacaaagcaattaaaggtaaggtaacgttaggtgatttaaaagatgttgcagataataaagtatttgaaggtaataactaccatgaagaagtctctacttatgtagtagctaaagaaaaagaattatctcacttaagagaacaacgtaagcacaacagacatactgcatatccacaaattatgtttgatgaacttaaagaacatatggttaaagaattacaaggggaaacattagtagagcatcatggaagtaaagttaatattaatgacacagagttaattgtattactatcagatttccatattggaagtattgtatctgatatgactaatggtaaatatgattttgaagtattgaagtcaagactaaatcactttattgatacgacagttaaagagattgaggatagagagatttctaatgtaactgtttactttgttggagacttagtagaacatattaatatgagagatgttaaccaggcatttgaaacagagtttactctagcagaacaaatctctaaaggtactcgattacttattgatatcttaaatgtactatctaatgtagttacaggagaattaagatttggtattattggtggtaatcatgaccgtatgcaaggtaacaaaaatcagaagatttataatgataatattgcttatgtagtactagactctttattgctattccaagagcagggattattaaacggtatagatattattgataatcgtgaagatatttatactattagggataatttcggaggtaaatctattatcgttaaccatggtgatggattaaaaggtaaaggcaatcatattaataaatttatcttagatagtcatattgatttactaattacaggtcacgtacaccatttttcagtaaaacaagaagattttaatagaatgcatattgtagcttcatctccgatgggatataataattatgctaaagagctacatttatcaaaaactaaaccttcacagcaattattatttgtaaataaagaaaataaagatattgatattaaaacagtatttttagattaaggatggttaataaatggatacaatttttattataggtgtagcgtttataacttttgcaacatttaacatagtctttagattatttgatttatggactacagagaagaaaatggtaagtcaaggacaacctccactaagtaattttgagtactatcatgtgatagtaccctacttagttggtgttattgttattatactgagtattatgtttagagattccttgtattctgcacaatcaggattcggtgttattattacaagctttatttatatgttagtttatgtgataattggtcttgtagggtcatttgtactcacaatattccaagctagaaaagctagacagtatcaaacacaggaggataataatgaagttcaatgatatttatgagcaattaattaaaaatgatacaatacaaaacattcatgaatctcaagatgataaaggaaatatttatacaatacagtttgataaaggtaatgataagtatttatttaatgttatcaatgatggattcttgaaagaaatgacaaatggtatgatagaccatcctgaaggtcagccttattcagtaagtctaatcaataaagaaacacctagtatgtcagtgaaacaatatttaacagatgtagaagatattgtacctactattagaaaaatggaaaaggatttcttatagagtcaagtctttacttgactctttttactatatatggtatattaatatagaggtgacttaaaaatggattttaattttagtgcttttgataatagctcattagcaatgagaatcagtgagggtgtatactatttcaatgataccccttattactttattgagcatgtagaagaagaaatgtctgagtatgttattgtgtatgacatacatgacagagaggaaaaagaaaatcctcagaagaaatatagagtagagccttatcaacgaacaatacctggaggtacacctcttagtaatttgattaagagtatgatgcctcaacgtaaatatcctaagaaggttatagaagaccctatatttgtagctaatgttattcctttaggaacagatacagttacaggtaaaactggaaaagggttttttgaaagagataaggatagaactatttactcacagaaagaaccaactaaggtagttcatggtcaatacacaggtgtttttataggtctaacaagtgttaaatggaatagaacatatactcctttagaaagtgttgtcgagtattgcaagagagtaaaaggagataggttaaatgtctaatgatgtagttaagttttatgaaaaagatattaaagaccttataagaactaaaaaacacatgtttaaagatgatgagataactagtgatataaatgatatacgtatctttaatgagaaagtaatttgtcaaggtaagtgtagaacagattgcttagtactagaccgtaatggtactgtcatgggtatagaaataaaaacggaaagagactctacacagagattaaataaccaattaaaatattatagtctagtatgtaagtatgtatatgtaatgtgtcatgataaacatgtacctaaagtagaacagatacttaaaagatataaacataatcatgtaggtataatgagctacattagttttaaaggtaaacctgttgtaggtaaatataaagatgctacaccatcaccgcataggagcccttaccatacaatgaatatactatggaagacaaatttaatgacaatacttagattgattagagaccctcatacgtatagaacagggtatagttataatgctagcggtaggtacagcggtggagaaggtaacttctctcaaacaactcaaagtaaaagaatgaaaaaacctgctattattaaccaaataattcattatgtaggggtagataatacttataaactctttacaagaggtgttatttatggttataataataggtgggaagttatagaagaagatttctttaatactatgaagaatggggtaagggtaatcaatgagcaaagacaaaccaaatagacgtaaagaaatacagcatcaacctgttaactttgcccctacaaatactttaacaggagctaataacagtttctttgctaaaaagccttcagaacctaaggatacaacatctgttattgaatatagaatactttttattaaaagatttgataatgtaacaagtacagatgttaagttacaaaaaaaatatgcattaaaccttattagtgaggcacttgatgttaaagaaacttatctatctcttaaacaaaaaggaaagaaaacagaatctattttacacacggatagagtttattatgtgcatagaggtaaaaaacttattggaaaatgtagtattagagaacaaagaacatttaaaggtaaacatttaatatttatatttaagacaagacatagagttaaagcagaaaggaaagataaataatgttaaaaggattttcagaacatgtggataaacctacaactattaaaaccttatataagaccctaacaagtggtaaagtagaattactgggtgtatcttatggtagtgattacttcccttcaggtgttacagtacaagcttatattgaagatataggtaatgaagatgaaggtctgcagtttgttaataaagtaaacgtagtggaatcaatgaaacaggctgtagtaggtatgaacaatcaattaggttcttcaggtcttggatatgttagaaccgaacaacttaaaaaagagctagaagaaactggattaatgacagatttacttgctagaggtactaacttaacctctactaagaaagtagatattgtaagtacttttattgagcctgaagtaacatatcaagatattactatagctaaagatattaagttacgtctatacaaactagaagaagagtcaccattaaatggttatacccatactgtatacttacttactacagagaaattatatgatggtcaaacactatttggtatgttatctaaaaaagataaattatctaaaggagatactgataaattattagcattcttcagaaataatagtttaataagtaaaagtgtattttgtgttaagttattaagtaaagactactactttaatttatataatacacatgaaacaggtatattctttttagaagatacggatattattactattgcttgtggtcagtcatatgttaaagttaatactaaagatattaaatctagtcatgttaaaattgaagatgagactcataaattaactgagctagaaattaacttaaaaggtgacgacacattaactattttattctaatagaatgttataaatatgtgataattaagtataaatatacgttatataggaagttttcataatgtttttaatacagaaactagttaagttttttacacttgctctagtttctgtgaaattatatttatgaaaagttaaaatatcttttaggtaaaggctttgtaaatagttaaaaaatatattaaaattttatacaaagtagttaataaaattatattacatttatatattatgaaataataacagaaattgtgatatattatatagtgtaaccttgaaacagttgatgttgtagggtttgtttatgttcgttaaactggtttcagaacaccagttaccataaataaatgacagttaaggagagctatataatggctagaaaaaagaatttaagaaataaaaacagtgatataaaagttgttcctgataaagaaaaagaaagtatattatctaagttatatcataataagttactacgttcaaaggtagataatgcattagatgaagatatgagttatgatgatattatagaattatgtaaagaatatgatttagaattgtctaagtcagctattacaagatataaaagtaaaagaaaagaagctattgaaaatggttgggatttaggagaattaattgataaacgtaaaaaaacaagcgtaaaagatattaaggaaaaagaaactcctatattagaagaggagcaactttctccatttgaacaatcaaaacatcacacacaaacaatttatgatgatattcaagtactagatatgattatttctaaaggtgcaaaaggattagaatttgtggaaactttagaccctgctttaatgatacgtgcaatggaaactaaagataagattaccggaaaccaattaaaaggtatgtcatttattggacttagagaactacagttaaaacaaacagctcaagatacagctatgagtgaagtattattagaatttatacctgaagagaaacatgaagaggtattacaacgattagaagaactacaaaatgaattctacaaaaatctagatttagatgaggaaagtagaaaattaaaagaagctcttgatagagtaggctatacaatttagatagtgaggttagagtaatggcagatgagattagtttaaatccaatacaagatgctaagccaattgacgatatagtagatatcatgacatacttaaaaaacgggaaagtactgagagttaaacaagacaaccaaggagatatccttgttagaatgagtccagggaaacacaaatttactgaagtatctagagacttagataaagaatcattctactataaaagacattgggttctctataatgtatctgttaactctcttataacatttgatgtttatctagatgaagaatattcagaaacaactaaggttaagtatcctaaagatactattgtagaatatacaagagaagaccaagaaaaagatgttgctatgattaaagaaatacttacagataataatggtaattatttctatgcacttacaggagaaacaatgctctttgatgaaaataaattaaataaagttaaagattagggttgacagcttctatagtttatgatatagtatatgtatactaaaaataaaggagctaacaattatgtttatttcattaaatcaagaagagaaagaattattaactaaagaggaaagtaaatacacaccattagaaacatcaagagagtttaacacacctaaagaagaattcattgtaacaagttataacgaaggtaaacctttagattacattgcaaaagaagctaaggtaagtatgggattaatttacacagtcctaaactactataaagtaggtaaacgtaataagaaatcacctgtagaagaaagaattgcacatatcttaaaagataaaaacttagtcaaagagattattaaggattatcagtatatgaatttacaggacatttatagtaaatataatcttcataagaatggtttatattacatcttggatttataccatgtagaaagaaaatctgaacttaaggacaaagcattggaagaggataatattgtcgttgagtaagtaaagaggttataatatgagaaataaaaaatcatttcaagagcagttaaatgacatgcgtaataaagagaaatgggtatctgaagaggagttcactgaagaagtgactccttctaaagaacctgaagtagaagaagagaaattatatactttaaatgagttaaaagagaacttactagatgctcagggattaaaagatgttgtcgataattttcctgcatctaagccagtatatgaatctaataagctttatatttgtacaataccaaaaggatatcgttctacagaagtacaaccaggtcaatatattggtatcagtacagggttattatcagaatcagaagattttagtcatttaagaggtcaaatgcctagaaatctttatgaaacttctcatgttttaaaacctttagtacgtattaataatacaaatctcgaatatcaacagcatgagttacttgaagatattaaagatgacaagaagatatatgatgttgaattagaagacctgagattagtaacaggagaagaaatatcccatttagaaattgtcgatagtaagttttttgaaagtcgtattaatgaaattctagaccgctatactgaattaacggattccgatgatttacttaagtactatagtaaattacgagaattagttggtagtgacaaaatgatttattgctcacttttagataaatgtgttaaaattatagattaatagttagtctcctcttatattataactgtaagaggagacatttttgtatagaggtgttaattatgtcaagaaaagcaagtatattctatatactagtggttattgttttggctttttctatttcatcttattatatatcttctttcatgtatcacgacaaagcaaagaatgaagtctctactgagttatcaaacacgggaaagattaaagaagaaaagaacgtagaatttgtcggtgactatacacttaaaaaagtggaaaataataaagcttattttatggaaacattacctacttacctacccggtagaacaggagataacagcatagatatgaggtactacaaaacaagtagatttaaagaaggggtaaatttcaagcttattagggtatatactgaagatggggaagataatccaattcataagtataggtttgaagcagtaccaaccaaaaagtaataaggaggtgacttaaatgacaacattaattgtcgtcatctttattgctatcatttattacttatggaatagtgattgagtcaagttaattcttgactctctttttgttttatggtatattaatatatagaaaggagagattaattatggaaatggcagatttagaaagattcgatacgtttgtaagattagtttcagatgatgagctttcggaggagagagtattagaattaagtgtagacttattaaatccgatactagaaggaggtacagcttaccaagctaaaaaacgcattagaagtaagttcggtaaaatagaagcaaaaaactttaaaagaaattataaattcttactcaagtcgatagctcaaatagaccaaaggagataggacaatgacagaaagggaaaagttagttaaagagattgaagatgctaatagggacatacaattaaggttaaaagaagtagatgattataaggatagtatacgttctaaaggaacaagaaattatgtgtctactaaggtattagattcagttatggtagggctaattatatgtttctttattcttgtaatgttacgtgtacttgaatattttgtaacaggtaatgctgtttattcacctttagcacctgcagttattattatgtttgttttasagccttaggtacatggaaagtaagtaaaatgaataaaatagtatcctatcggggaactattaagatgtactgggaattaagtaatgctgaacagaaccaagctaagatatttaagtatcctaatgatgaagtagatatcgtatcaaaacataacttaagacaaataacttttagtgaaattaatatacttcatcttaaatatatgagatataataaggcagtagaacagcatactaagttatctaaagaactttttaaaaaagataaagaaactgttgacaagaacaaataagtgtagtatagtattactaaaggaggagagatattatggttatacctagtattaaagcacaaaacaaattcaagaatgaattagagtattataagcaaggtcacattagtgaaagtaaaatgttagaattagcttttgattacattcaagaattagaacaaaataatgaatacgttactaatttgctagaagaggagagatatggtgagtaaatttatcggagtgtacttatttaatttattagtcgtagcactaatttatacaataggatttttattcttttatggtgtagctagtttagttattattctaactcatgccactattgacccatttgtattagctactttcttaggaataggattcttagttattagaactgcacacagaatcatggcacgagtaattaatgatgcagtagctaaagctattaaggataaagaaaatgaataaaggggaatttattatggataaaacattaccaaagtttagtgtatatgaagttattgtaaagactgtaattatgacaccaacagaaggaagttctgaccgagaatcattttacttttcaactcaagagttagcagaaagatttgttaaagaaaaccaagtagagacaaaagatgggaaacgtgtatctttttctattaaagaacgtaaagtaaatcaaccaggctaacattaatttgttagcttttttttattgacaaatcattttatatagtgtatagtaatattatacagaaaaggaggagtaattatgaaagtttcagaagaagtaaaacagagctacttagagaataaagctaatactagaatggataagataagttggtctgagttaaggtctagtcctgcaggtagaacattaggagacttaatatactttagtgtagtcataataaataatatcatagctatagcattaacattaattctaggagacattatcaaagatttaacggatagtactctagtaaaaataataagtatattacttattattatcgttgtatatggtatactatcggctttaatacctatactaatatttaaatctgtatttcctggatgggaatatactgaatggaataattcttactatttgagattacctggagaaaaagattataaatactatagctactggtatttaaatttattaggtgttactgagttttatcggacaaatgatgagggtaaagaaattaaagaaactagtttaagttgggttttttatactaaggttgatagacctgaggatattgaccattggaaaaatcaattaataactaatagacctttaacaatttctgagtacaaaaaattaaagaaactagataaagaaagtgaaattagaaaacaagaagatttagaggaatataaacaatacaatagtaattaaagaggtgaaaaaccaatgataagttcatttgatagtgtattacttgtaatatacattattatatcttttgcagtagctatggcaatactttacttagtatttaaaggtatgacccttttactagataaattaatgatgatgttattaagtaaaactactttagatgtagaagcttgctctatgataatggcagttatcagtacaattgtgtttggaattattgtacttttaatatggttagcagtaaacaatattttattgtaaggagttttactatggattttaatgactttataaacagtgaatcagatagggtaggtaaacttaagcaaaaaaagaaggtagaaaataaactaccttcttctactcctattgaagagagacaaaagaagttagaagaattaagaaagaaatcattatacattgatttaaggagaaaaagaaatgactaaagaaacaaatgtactttacaaagataagtatagagattatactatagttgtaagactagcaggtaacattattgttactgaagtagataagaagcataaaacaaaattttcacctatcatatttgataatggtcgagaaggtgtagagcttataatgcgtgtaggttctgaagaacttgatatgaaagaacttagagagtttactaaagagatatcaacagctcagaaagctttagaatattttaataaaaaactttacattaaaggcttgacagatgaagcattttaatatatactaagattataaataaaacaaggaaaagaggaatgattattatgttattaggaattttatggtttatatggggatttgtatcttattttgtagtgatgtttggaattgaattttggaaagatagatggatgcctggagttataggtgcaggagccttactactattcttattttggattatgaagtctattcataatgctatgacagtagtttacttgtattaggaggtaatatagatgaatatattagttatccattatgaagaaacaaataaacgagttctaaaagaaacaataaaaacaatacaaaatcatttaaatgatgaacatggtttggttaaaatgacagcaacaagacttagtagaaaagccatagaagaagagtttaatgattacaatatagttattgcagaaaatgaccctgatagtttttacagctattcagatgcagtagacgatgtagattttgttatagatataccaatttcatatttagatatacatgcaggtgtggagtgggatgttgataatcctgtagatatgttagataggaaccctgattttatagaagctatgggtcaattaaacgaggacttaatgttataaggaggaaataaaatgttaaatgaaaaactaaaaaatttagaagatacgaaagtgtatatgattaatagtattgcaagtttactaagtgcaagtacaggtaaatcaagtaaggtattttttgatgaaggaactattaaaattgtaagtggtgatacaaaagcagtagaagttattgataacttagttcaccctcattcaggacgtttacctattaaaacaacagaacgtattgctcttggtagactaacagattctttacagtttgttatttcagaaatagaagcagttaaagaccaaattatagatgaagaaaatgaagcttacattgattttgtaatggaagactggaattgggattaacgcctatggacttaatgacaatagcttctgttgcctttatagctgtcgttattattgatttaattaatgatgatatgagctatatgttaactggtactgcaatattaataaacatttgggcaggtttttatggatggttctttttattacaggcaggtatgttacttttcttactcttagctagaaaagttaaagatgataaggagtcgattctatactccagtgcttcattaatatgtgcactaggaatgataataaatcttcttatattttcttaaaaataagtgttgacacttctaaacttttgtattatactataagtataacaagtacaggagatgattaatatgagtaaggaaacaattagaagacaattttcaaatgcaattgagattatggcaacaactaaagaatggtggaatttcccaaaaagttttaatacaaataaagagtttaaaattaaaactttcaagaatgacacacttgtatttgaagttagagaaggtaataaaaacttaggaagctttgtagttttcacatgtattgactttgattatgataaacttgaagggacttcaacacaatatatgattaattactttgctaagaaattaactaaagatatgtttaactatcataagttacaactatagtaggaggtggaaagatgagagaggaattaaaaccttttaatagaaaacaagtcaatgttaaagggtacttagatgatgttaagtactcaaagcgtagaagacataaaggtaatcaacatggatgtgtaaaaataacagttactgatgtaaagattaatgggatatctattgaccatgttaatattgaagttggaatttctttttatgagaaattaaaggaattacagggaaaaagaattcaatttgtaggtactgtttataaatatgttaaacatgctcgaggacataaaggtagaattaaaggattttataaagaggattatagtgtaactttagataagaagttacaaaaggaggaaaaataatgattaaaagaagaaaacatttagaccatacagtacaacctgaaaaagggtggagaacagtaccttttaatggttactatgaagctcacccttcaggtttaattagaaataaagtaacaagaaaattaattaaaggtacacagacaagaaaaaatcaccctaagtggactgctcatgagattgtatatctaattaatcctaagaaaacaagttattctagaggtgttgttattgcacatacattcccggagatgattagccaatcacgaggagaccttaagaacggtcacgtatgttttaaagatggtgaccgaagtaattgtcatgtaaataatatgtttattggtaaaggtaatgttaataaaaatatctataaattaaatgattcttatttgactagaaaagatattgaagaagatatcaacaacttagttaatgagagactattctctcagttagagttacttattaagaaaaatgaacctgaaagaattacacctagtaatcactttattaaaagagataataatgtattcagcatcacggatttatctaaaaactctttagtagaatttgaattagaaatcaagaatattaaataaggtggctatataaatgaatgagtggtatgctttatgttactatgataaagtaggtaaaaagaaaatacctaggcaagttagagcacatagagatatttcagtattagaagaattaaaagaaagattagaagaaagaaatcctaacacagaatactctataaaaacaacaaaagaatttgatgaggagagataaggatgttaacaccacaacaaaaagattcattaaaagaacaacaaaagaaattaagtaaaaagaagaaataactgttgacaaatgagtgtgcataggttatacttaagttaacaaataaagaggaggtatgacctatgttattcataatttttattctagcagtattctttgtactagggtttattaacggatggaactcagaagactaaaaaaggagtggttatagtgaagttagaagataaagtattagaaagaattgattctcttggaggtaagttaggtgatattagccaacatgcttgggaagctttagtaaagtaccaaattatatatggtattatagaccttatagtaggtattgtagttatagcattaactttatttttatggaaggtatttattaatcaacacaagaaggtaaatgatatggatagagatgatgactatagtttactatttgaagattgtgaggatttatcaggcataggtttgttttatgtaatagttacatcattaatatcactatttgcatttatatacttaatctatggaatacctatggatattataaagatattaaaccctgaagtatttgcagtaaaagatttaatcgaacaagctaaaggaggaaattaaaatgaaacagagagattttgagtttgaagaagattttgtactaacttatgaatgtgaggattgcaaacatttcgaggattggggtcatgatgaagagcctgaagaatgcagtgaatgtggtagtagtgacttaattaacaatacaagtcatgaagacactgagtgtgatatgtgtaaaggatacattgatatgtggcaagatggatacagatatatgggagataataaagagtatcttgaaaaagaggaatcaggtttaatttgtgaagattgctatgagaaactagatatttaataaggaggaatttatatgccagtaacagtacctcatgttcctgttgtaagtaatccaacaactactaatcctaccactacaagtggtaatacagaaactagagaagagacgatagctcacaattgggaacctgacttaatatcagatactgaagctatgtatattactataactattgttttaatggctactatagcatgtgttggtttatggatttatgccaaaaaattcttaaataagtaggaggaattctatgactaaagaagagttagaacaaagagtaaaggaacttgaagcagagaataaagaacttaaaaaacaaatagaacgttttgaagacgagggaggaaaaacaaaagatgaatagtagacaaaagaaaattctaacattaacaataaataattttttaatgttagccttagatactgtagcactaattagatataaaaaaggtaaaattaaacaagagaactataatacaggacagattacaagaacaatagttaccacagctaattcattaggtattctttacttagaagagcaagagcgtaaagaagttaaagatattaaagtaggtacttttgaaactggagaccttaaaagatttatgaatacaaataataaataaggggttaagaaaaaatgagtattgatttaaaagaatgggaaaaaattattaataaacaagaagagattggtgggtgccctaaaaaggctaagatatcctttacaggtaataaagactgccatccgtttacagataacattaccggagaagaaggaattatattttatgtagaaaatgaaaaactagaagtacctagctcaattgttgtattagataatggtgaagctgttgttacaacagatagtaaaataactcatggtaatatacctgatgattttgaaacttcggaggagtctattatagattttctaaataacttatatatccaagctacagaagagtattataattggttacaattacaattaattggaaatcaggataagaacaaattaccaggaatcattgcagaattatcagaaggtcttaaagtaggtagttttaccattaatgataaaaatgttttactttacagagagaaagtgaatatacatttacctgtaggttatttaatcattattgaatattctatagatgaaggtaaacatatcatagacactaagttacaagattctgaaggtacggttattaatcgtatcattagaacagatattacgtataatggttcatttcatttaattaatacgttgagcagtattgtaaaaccaatttcattataaaaaagtttaagaaacctattgacattaggtttcttttattatatactgatattataagaaataaggaggttaacttatgaaaggtattattatattttacaaagaagagaataaacatattctaaaggagctcttagaattcattaacacaacagcaaaagggttatcttatactatggagggtaccttagttgataacgatatcgtgttacttaaagataatagcatgagtgaatataacctaagacagtttagtaagacaaatagtagtttagttgtaggattactaagtaaatcatatactgatgttcactactatgaaaaaggggatgcctacggtggattaaaactagatgaatatttagaggggatgagacaataatggtaattgcattttttattttaggtctattattagtattatgtttaattggtcttgctattattaaagtcttagaagcaagactaaagaaaaaagagctaagaaaagaacgagaatcttttggtattcgggaaccagggagacagttaggacacataggatatgttgaaagtaagtatgctttaattcataaagaatcaagtaaaataataataagcgcagagaaaagtaaagttatagaaacattacaaaaaatgtatagcttagaattaacatctgtagatgtttctagtgtaacaggattctctcccttaggtaccgggcgtatggaaaatatggtattgctttcgtacaaattagaaagagaaggattttataaattaattgaacttaataaggagtgatacaatggaatttatagataaaaataatgtgattaaagcttacgatataccaaatgtttatttaaaaggttatgtattacaggcatgtgataaaaatggagatacaacagcttatgatggttatgaccaaatacactataaagaaggtagagtattaacattcccttttgataaaccgttaagaaagataaatgtactatcagggtattacaaactatttaaaaaggaggatataatatgatttattttgttagtgatttacattttggtcatgataacattagagagtttgaagcacctacaagaagtcactggaactcagtagaagaaatgaatgaaggtttaattgatttgtggaataatacaattacaaataacgatattgtttataatattggagacttctttttcaatattaaaccttctaaagtagaagatatacttaatagactaaattacaaagagatgatactgattgcaggtaaccatgaccataagaaacttataaaactatatgaacgtaatggtattacagtaaagtacgcagacatgattaaaaaggacggtaagagattttatctaagccattatcctacactaataggcagaaaaaacatgtttaatattcatggtcatatacattcacagttaatggatacagagtatcatattaatgtaggttatgatgtagagggtaagattgcctatagttttgatgatattataagtagagcaggtgaatataatggagaaattcaaaggtaaagatttatataaaactagaattagaaaacaaacaattaaaaatttagttataaaaacagagaagctacataataaacacggaaagtatagacctattggtcatgtttattattatccaaaaacaaaagagtttactttatctaaacctgagcagaaaatatttatagagtatatgaaggcattaggttttagcgttaaacataagagacgtaagaaaataattatagtatacaagaatgtgttagatgaatatcttaatatgtatcaggaagcaattgaaagtacgtgttgacaattaaggtatactatgttatagtatagaaaaggaggttaactgatgaagcattttattttgattttaggaattgtaattctagttattgcattaggtattgttttacctgcatggattttacaattagtgttatctgcattcggtgttaaagtaagtatttgggtatgtatcgggatatttatcttaatcagtgcagtaggaagtatatttagtagaaattaaaggaggaactataaatgtcaaaatacgaatcaaatattaatggagaaaattatattgcaacaccatcacaagctttaagagaggcactagctaagctaatttctgaagaaaagaattttgcagagtatcaaactaagagtgaggaacaatatgaatcacagttacaactaagacactttgattcaatgatttctcaatatgaagaggctattcgagtactagaggataggtactcacctcagatttttattccaaaagataataaggaggaaaagtaattatgaaagcagaatcaatagtaagattttttcaagataaggtgctacaaatagaagggtataaagtaagattcactcaagctagttcatcttatattttagatatagatactatggatgaatcagtattgtttttagatactgtagttttcaccctatcaggtaagtacttattagatacacatattacaattaataaacctgagacactaagttctaatgaattatacacagagattagtaataaactacaagagattgtaggagaccaaactaaaacagatatagagttatcaaaatactttaaggaggtaaaataaatgagttcagaagctattacaaatcatttattaaatttaaatcaaataaaaattaaagaatataatattcatgcttacattaaaaaatctgtttgttccggtattgaaaatgcagattttgaagtaagaataaactatatagcagacaaagaccctaactatattagaactattaattctattatttttgttgattacagtaaccgtaatccaaaagaaattttactacagtttaaagaaaaaattctttctattgtaaaagaacaggtagagattgataatgattttattgaggctattaaagatattaatacaaatcatgaactagagaaattagaacctttgattaataaagaatactattctatgtttaagtcatctattgaaaaagaggtaccagtagctttatcatctgaagtacttaatagatgtacaggtaaaacaagcacactagcttatttagctattgaaaaggatttacctttaattgtgtctaataattctatgatgaaaatgcttaaaaaagattacccttctgttaaagtttcgtctgttgaagatttctcaaactataatattaaaggtgaaattgtacttatagatgaagtagatgtagaccagttatatagtgcagatagagtttctgttgatgcactactagtaggtatcataaaaaattaaataaatttgtaaatacctgttgacagcaggtattttttatagtatactttagatgtaaagaaaaaggaggtagtaatatggttggtattataactttaattgtcggtttaatattatttttagctagtggatataaattagttttaggtaaatattatgatgacatagatttaaagatgttatttacaatctttggtattggtgctatactattacttacaggatttatattataaaggaggaaattacaaatgaactataaagaagtactagaagttattaaaaagaataagccatgtaaggttagatttactggaagtattttagcaattgttaataaggaatttaacgcagatactgataaaggtatactacaaattgatgtatcaaatattaataaaaatgactacattaagttacaacagtattgtttagaaagagatgattatactgtagcaggagctattttattttaaggaggagtaattatgaattatagagattttattacggattgtattagttgtggttataaagtccacattagtgttactgagaaaagagttcacattatttcagaaatgacatcagcatcttatccaaagaaagaaattaatttggatgaattacaagcttatgtttattatatgaataattttggaagtcagattacaacggagggattataaatggaattagttattaatattatagcagtattaattggtatgtatggtatttacttttatgttacaaaatttagtactggtctatcaggtatcttaattgtactaggtatggctgtaggtctttacttttacttagattacttaaatgttagagagaatgttattcgattagtatctgtaatgtttggtgctttcttatttagtatcgagatgatttataataagattatgtttgaaattaaaaaatctaagtatgataagactgttagaacgtacagaggagaccaataagaattttactataaagagtacttaaaataggttaagtgctctatatggtaccttaaaatggcttagaattgaaattaaggagatgaaaagttatgtatcctgatataggtgagttagcttataaattaaaaagtactagagaaagactagaaggtattatgatggaggaagtttgggaaatacatcatagagaaacaggaaaactagtttttaaaggtggatacttagaagtaaaagaaatattaagaaaaatgtataaggagaatttaacattagtagatgtagacacaatgttaaatatcggtcaagggttttttgatgttgctaaaagtatatcagcagaaaatgtatttcaaataaactataaaaaggagttactacgatgattaaagtattttcagaaatggatacaaaatataaaacaattgtgcctgaaaagttccctaatggtgagattaattttaaatatgatgacttaaaatatttagtagaaggggaactatggtttgatgttttatttaaatgggaaaatgatgcagacttaatgcacttgtatatgtttactaagtatctagaacaattaggtattaaaaataatgtttccttcttggaaattgcatacttaccctatagcagaatggatagagtagaagaaggtcacagtaatatgtttagtcttaagtatattgcagaatttgttaacaatcttaattataaatctgtattgatagcagaagctcatagccctataacaggaaaactaattaaaaattctgtttctatagatgtcacacttaaattattagaccaatatattgaaatgtctgaagaacctgtaacaattgtactaccggataaaggagcatatgacagatacttatctgatgtggaagacatcttagaggattccaatattgaaaattattcaattgtatacagtgaaaagaaacgtgattttgaaacaggtaaaattaaaggtattaaaattgttaaagataaaaatactctatatgatagctgtattatactagatgatttaacaagttatggtggtacatttgtaggttgtaaaaaagcccttgacaatcttaaggtaaatagtgtatcattaatattgactcatgcagaaaaagtatttcaagatggtaaattatttgagtcagggtttaaagatattattgtaacagactcaatgttacctgataaccagtgggagaaagctattgctaaacatagagcacaagaaaacggaacagaattacaaataaagagtatcgaaagatatctataaaaggagaatactaaattatgctaaacccaacattaatgtgtgatttctataaattaagtcacagagaacaatacccggaaggtacagaatttgtatatagtacactagtacctagaagtaacaaatattatgaacatagtgataacatcgtagtatttggaattcaatcgttagttaagaagtaccttattgatatgtttaataaagaattctttaatagacctaaagaagaagttattaatgaatataaacgtacggttaagtttacattaggtcacgagaatcctgacgctaaacatttagaacaattacatgacctaggttacttacctattgatgtaagagccttaaaggaaggtacagtagttcatccgaatacaccagttatgacaattgaaaatacacatgcagatttcttttggttaactaattacctagaaacaatcattagtacacagacatggcaatcaatgacaagtgctacactagcatatgatatacgtaaaatgctagataagtatgcattagaaacagtaggtaatattgaagcagtagatttccaaggacatgactttagtatgcgtggaatgagttcattagaaacagctcaattaagctcagcaggtcatgcaattagttttaaaggtagtgacactgtacctgtagtagactttttagaatcttactacaatgcagatgtagaaaaagaaatggttgttgcttctatccctgctactgagcattcggttatgtgtgcaaatggtgattatgaaacaatggatgaatatgaaacatacaaacgtatgttaacagaaatttatccaacgggagtattctctattgtatctgatacttgggatttttggggtaatatgactaaaactttacctagattaaaagatattattatggaacgtgacggtaaagtagtaatcagacctgatagcggagaccctgttaagattatttgcggagaccctgatgcagatactgagtatgaacgtaaaggtgctgtagaagtactttgggatacttttaggggtactgaaactgaaaaaggatataaagtactagatgaacatgtaggattaatctatggtgattcgattaactatgagcgtgctcaacaaatttgtgaaggattaaaagaaaaaggttttgcaagtattaacgttgtattaggtgtaggtagcttctcttaccaatttaatactagagatactcacggttttgcaatcaaagcaacatatgctaagattaaaaatgaagagaaacttatctataagaatcctaaaacagatagcggtaaacgttcacataaaggtagagtagctgtttatgaagatggttcatgggaagataacctaaatctccatcagtggctaaataaacaaaatgttacccaattagaacgagtatttgaagatggtaagttatatagagaccagtcactaagtgaaattagagaaataattaaaaataattaataaatatttaaactccctattgacaaagggagttttttattatatagtataagtatagtaaataaaggagtgaaagaaatgatttataaaatatcaaaacacaattattatagtagatttgagccttctacttaccctcctgatgaaggatttgcgtatgtagattatgtagatgtgatttttattggtgtagataaccttaggaaaagaaaggttattaccttaaaagtagatgagttcaatcctgatgactacaaagtaggtcataagtataatattataaaattattatggtttgaaaaatgggaatggttaaagccataagtaaaaggagagaaataaaatgattatagataaattaaatggagttaaactagaaataggtggtcatatcgtatcatttagtgtaataaagtttgatacaattaatggtgagagacaattaatagactaccatcatattaaaagaggaagacagaagtactttagaactactgaagaattttataatgaatataaagaaattaagcctgacaaaaatgaaatagatgaaatgtttgaatctctaggttatgtagatactgagttagatgatgtagtaagaaacctggaaaaggttactgaaatattaggggttagtgaacaatatttaaatcagttatcttataaagctatagaggagtatgtagataaagtagttacacttgaaattaaagaattgaaaggagaaaaatagcatgaataataactgggaaaagaaggggttaactattgggaaaacgaagactgtcctagggaatacttagagaaagcatttattgacctagtagaatatgtcgaaggagttacagtaccacctaaagatgttaagcaattaagagaagataaacttagagaagatattgggttttatgagtacgtagctgataaataaattagtatctacctattgacttaggtagatatctagtatataatagtatacaaggagatgaaagtatgaaaaagttaatagtattacttacaattactatttctctattactagggggttgctctcctgataaccatgaaggtaaagtagtaggagtaggtgaatacagagaaccaactacttatataaaatcaggtagcgttactgtaccagtcattggtgaaatgaaatactatgtagatttagagacagataaaggagaagaccgtgtatatcttaataaagaggtctatcacaagtttgataaaggtgatgatttctctaatgtaggtgagaaagtgtataagaatgatgaattaatatataaaggagactaactatgtatttaaatgattatgtaggtaaatttataaaggaagataactattatggataccaatctacagacttagtatctaattatgttcaaagactaactctagataggtacaaaactaagttaaatgctaataaaatgaaatatgagagattacctagttcttggaaaataattaaagccaaagatttgttaagaacagatgattatagagaaggagatatatttgtatcagaaagaatctctgtatttggttttaatggaatcattgtacgtaatcatgattttaacaatgtaactgttattactcaaaatagagatggtaaagctactaaacctgtagaggagcatttatatccaaagaaagatattgattatattattagacctatcgaaagggactacagggaatactttaaacaatcggattcaaaagaaaaagtaactctttcgaaacaagaatataaaaaattattagaggcttataataaaatgaaggaagtgtttaagtaatatgaatagtacaaaattagtagattattttacaaataaacaaggtaaatatctaatattacctgatgaaactaaagttaagttatctaatgtagatttaatgtcttacattatgagactcactttcacttataacacagaagttgtgtctatagatattgataagttatactcagattctatagaaatgcatatacctcaaggtctttatatgacaactgttgttaaaattcctaatacacagagtgttagttcagttcttcataaggtattagaggaatgggtaagacaagtacaaaatgatggaatatttggattcgtatgggaggaattttaatgtatagatttttattgtttagttatcctgattattacccttgcggtgggatggatgatgtagagcttaagtttaatggtttttctgagttaataagtaacagagatgatttgtatttaagtgaaaatattgaaatatacgatactaagtatgacaatatactatatattagtaacaaagaaacgatattaagtgaatttagagactatttaggataatggtaaaaataattaaaaaatagtagaagataatgaatctagcttacaacacactttaattgaattattaaaactttactcagattataggaggagatagtatgaaaaagggagaaaaaatagtaaaagaatactatgcagtaaaggaaaatgaaacaaggtatttcaataattactgggggtatgattatgatatcacagaactacaagatatggatactctaatggaagatatagagactgctctagatagaaaggaatcagaaggtgcagggacaatacagaagattagagtgacttacgaggtagttgaggaggaattataatgataagtagggaatataactacacagtaaggactgttgataatagaaaatacacctattataatagttacaaggcattaggttctgtatatagagatgttatgggcaacgactatatagaagtaactaaatatgggaaagataaaaaggttcttattgatactagacatattgtatctattgaacgatggtaaataataaggaggaataaactattatgataaatgcaggacatgctaagtacctatcagaaatttatgaagatgatatacactatgaaactacagatagtattgtagaagatatactagataatattaatgatggtattattgaagaagctatgaaaggtaatacaagttatcaatatgttcttagagatttaagagtagataatgaagtagaagatagagttatagaagaacttactaacctagagtatcatgtaaaccacattagtaatgatacagagtatccttctgtatctacaaataatttagcaggattagattacttaaatattaaatggtaagaggaggaatcaagtatggattttgaaaaaatggtaactaatattttcaaccgtaacactataactagagaagatattaaatttgattatgatagcttaggaaataagtgtatttactttatagagtataattatataaaatgtttatcattaaataatattataagaatattaaagaacatgtataaagataaggaattaacagaaagtgatagtgtgatagcattgtctttattactagcaaaagtaagtagagaagtaagagaaacaagtaaacgaatagaattattcagttacttagtataaggagggagagtaaatatgaataagtttcagtatcaactgtctactataataagagataactatgatggtatatatttagatgttttaaatatctacagagaatctcataacaaaaatattccaatacccgatgaggatattcaagtacttatagatgaatttaaaaggaaaaataatgttgattaataggaaagtaaatattaattagaccaaggaaaattaaacataagcaaaacaaactaagcacctg

SEQ ID NO: 5 - DNA, 18192 bp, Staphylococcus aureus
  source 1..18192: mol_type = genomic DNA
  misc_feature 1..18192: note = bacteriophage MBSA2
aaatataatagggaaaaagttagtataaatttacacctccccaccgtttaaaataaacgattatacaaatcaaaacttataaattcacttatcatttctttactaaacttataaatactgttcaccaactttaacaacttatcaaacctattaaatattcatcaaataaaaaatttatgtatctattgacttttattcaaagttatgatttgaaattaaaatcttatttcttctattaaatagtatttataaaattatttaaactttttaagaaaaactgttgacaaaacttttaaatgtttgctatactatgtatgtaatcaaacaaggaggtaacaaaatggaaaatgaaacaaaaaacattgagttgaagcatgtttttcgttttaagaatggaaatttatgtatagcgttatttgatagaacagaaaatgaaatttcattttatgatgttgacattgatgaaattgaagatttaaatcataattctgttttacgtgtaatttcaactttattaggatgtgataataatgattaatgttgataatgcaccagaagaaaaaggacaagcctacactgaaatgttgcaactattcaataaaatgattcaatggaatccagcttatacatttgacaatgcaattaacttagtagcagcttgccaacaactattattaaactataacagttctgttgttcaattcttaaaagatgaactaaacaatgaaattaaaccagaatcaatattatcttacattgctggtgatgacccaatagaacaatggaatatgcacaaaggattttatgaaacgtataacgtttacgttttttagaaaggagtgatataataatgaaacctgatgatattgttacattacgaattaaaggttatattcttcactatttagatgataaaaatgaatatgttgaagaatttataccacttcatgagtatcatttatcaaaaacacaagcaaaagacttattacctaattcacacaaacttttatccactacacgcacaacgaaaacaattcaagtttattacaatgatttaataaaaatcgcaattccagaaagcaaataatttaaataagaggagacataaaaatgacaaacgtaaaagatattttatcaagacaccaaaacacattagcgagattcgaatttgaggaaaaagaaagagaatttatcaaactttcagaacttgtagaaaaatatggaataaaaaaagaatacatcgttagagcattattcacaaacaaagaatcaaaattcggtgtacaaggtgttatcgtcactgacgactataatgtaaacttaccgaaccacttaacagagttaattaaaaaaatgagagcagatgaggacgttgttaacattatcaatgctggtgaagtgcaatttacaatttatgaatatgaaaacaaaaaaggtcaaaaaggttactcaatcaactttggtcaagtatcattttaatacaatttcataggggatatttatcccctatttttatgaggtgctaaacaatggaaaaaatatacactgccgtattattatacaatgtatcaattaatgaaacatatgaacatgaaattgaacaattcgaaaaaataaataaagttaaggtaatatatagttattttgacgcaaacttttacaaaaaaggtgcatataatctttgtgtaaaatacattaaggagatataaaatgaaaattaaaaaaatttttaaattattagatttactttgttacattgatgataatagagaaaaatttaaagacaaaaaaatacaatttatttctgatagtggttttaattttgttggtattgattctgataatgatattactatcaatttttctgaagctcataataacgatttaacaatgaataatgatttattcactttagacgctgacattaatattcatactcatcgttttgattttcttaaagttatttatggaaaattttcaaataaaaattatagaaaagaaatatttacaggttgcactattgatgatgtattaaaatatttcgagaatctattatatgatgatattataataatttaccaaaacaaagttatatacgataacgggaaagtgatagaccatgaataatatactggatattattattgttttccttttagcatttttaattacacttgtaatacttatgacaatgcatatacgtgtgtcatttggtgttttatttactacattgattatattctacattatctttttagtggttatatatggtttatatggaggtcgttagcaatggttagacatacgtccgaaatggataaatggaaaaaagaaagagacgcaagaaaagagcaggaaaaagaattgtttttaaatgattttagtactgttaattttaaatttgatgataaagatttacaagaggcgtacatagacgcatggaaacatttcgcacatttgccctattttccaaaagaaagacacgtgtcacatgcaaatgctgtatcattggtaagaggtaaaagacatgaacaattaaactatatacttgaaatatataaccgtaaagatgaatctaataataaaaacgctaaaaaacataaatatgctttatatgaattacaagcaaaaaataataattcttctatgtataaatatataaaggaaattgacactttatacaaagaaattggtaaatcagatagaccagtgactactattgatgatgaagatgtgaggtataactttttatattatgcaacatttgaagactaattttaatactgtaaacgacataataaactattacaaggagcaaaaacatggtgaaacaaaatcgtttagacatggtaagagattatcaaaatgcggtcaatcatgtaaggaaaaaaataccagaaaactataatcaaatagaattagttgatgaactcatgaatgatgatatagactattatatatctatttcaaaccgttctgacggaaaatcgttcaactatgtttcattctttatttatttagctataaaacttgatattaaatttactttattatcacgtcattatacattacgtgacgcttatcgtgattttattgaggaaatcatagacaaaaacccactattcaaatctaagcgtgtcacattcagaagcgctagggactatttagctattatttatcaagataaagaaatcggtgtgattacagatttgaatagcgctactgatttaaaatatcattctaactttttaaaacactaccctattattatatatgatgaattcttagcgcttgaagatgactatttaattgatgagtgggataagttaaaaacaatttatgaatcaatcgaccgtaaccatggtaatgttgattatattggttttcctaaaatgtttttactaggtaatgctgtcaacttttcaagtcctatattatccaatttaaatatttataatttattacaaaaacataaaatgaatacatcaagactttacaaaaacatttttttagaaatgcgtcgaaacgattacgtcaatgaaaagcgtaatacacgtgcgtttaattcaaatgatgacgctatgacaactggcgagtttgaatttaacgaatataatttggcagatgataatttaagaaatcatattaaccaaaacggtgattttttctatattaaaactgacgataaatatataaaaattatgtataatgttgatacatttaatgctaacatcattgttataccttatacaaaacaatatgagttttgtactaaaatcaaagatatagacgacaatgttatttatctaagagaagatatgttttataaagaaaacatggaacgttattattacaatccaagcaatttacattttgacaatgcatattcgaaaaattacgtggttgataatgatagatatttatatttagacatgaataaaattataaaatttcatataaaaaatgaaatgaagaaaaacatcaacgaatttgaaagaaaagaaaaaatatatgaagataactatatagaaaatacaaagaagtatttaatgaaacaatacggtttataaaaggtgtgtaagattatgggattacttgagtgtatgaaatatcataaaaatcaaagcaaaatgatattgtattgggatattgaaacattatcatacaataaaataaacggacgtaataaacctacgctatataaaaacgtaacgtattctgttgcgattggttggtttaatggttacgaaattgatgttgaagtattccctagttttgaagccttttatgatgatttttacaagtatgttaaccgtcgtgatacaatcacaaaatcaaaaacagatattattatgattgcacataactgtaataagtacgacaatcattttttacttaaagacactatgcgttattttgataatatcacacgtgaaaatatatatttaaaatctgcagaagaaaatgaacacacactaaaaatgcaagaggctactattttagcaaaaaatcaaaatgtgattttagaaaaacgtgtaaaatcctctattaatttagatttaacaatgtttttaaacggatttaaatttaatattatcgataattttatgaaaaccaatacatcaattgcgacattaggtaagaaattacttgatggtggttatttaacagaaaaccaacttaaaacagattttaattatacgatttttgataaagataatgatatgaatgatagtgaagcctatgactatgctgtaaagtgtttcgctaaactcacacctgatcaacttacatacattcataatgacgtgattatattaggtatgtgtcatattcattatagtgatatattcccaaattttgactataacaaattaacattctcattgaatattatggaatcttatttgaataatgatatgacacgttttcagttacttaatcaatatcaagatattaaaatatcttatacacattatcatttccatgatatgaatttttatgactatattaaatcattctatcgtggtggtttaaatatgtataacaccaaatacatcaataaacttattgatgagccatgtttttctatagacatcaattcgagttatccttatgtgatgtatcatgagaaaattccgacgtggttatacttttatgaacactattcagaaccaacgttaatccctacttttttagatgatgacaattatttttcattatataagattgataaagatgtatttaacgatgatttattaattaaaattaaatcacgtgtattacgtcaaatgattgtaaaatactataataacgataatgattacgttaatatcaatacaaatacattaagaatgattcaagacattacgggtattgattgcacacatatacgtgtcaattcgtttgtaatatatgaatgtgaatactttcatgcacgtgatattatttttcaaaattattttattaaaacacaaggtaagttaaaaaataaaatcaatatgacatcaccttacgactatcatatcactgatgacatcaacgaacacccatactcaaatgaggaggttatgttgtcaaaagtggtactcaatggtttatatggtatacctgctttacgttcacattttaacttatttcgtttagatgataacaatgcactatacaatatcattaacggttataaaaacactgaacgtaatattttattctctacatttgttacatcacgttcattgtataacttattggtacctttccaatacttaacggaaaatgaaattgacgacaattttatttattgtgacactgatagtttgtatatgaaatccgttgttaaacccttattgaaccccagtttattcgacccgatagccttaggtaaatgggatattgaaaacgaacagatagataagatgtttgtactgaatcataagaaatatgcatatgaagtgaatggaaagattaaaattgcttctgctggtataccgaaaaacgcctttgatacaagcgtcgattttgaaacctttgtacgtgaacaattctttgacggtgccattatagaaaacaataaaagtatctataatgaacagggtacaatatcgatatatccgtctaaaactgaaattgtatgtggtaatgtatatgatgaatattttactgatgaacttaatatgaaacgtgaatttatattaaaagacgctagagaaaatttcgatcatagtcaatttgatgatattctttatattgaaagtgacatcggttcattttcacttaacgacttatttccagttgaacgttcagtacataataaatcaaatttaaatatattaaaaaatcaacatgatgacatcaaaaaaggcaactgttaaataacagtcgcctttttcttttgagataacatgaaaaatgtgtacgaaaattgattatgttttgtattttatttactagcattactagcatgtgttcattatagcataattctttacgcaataccactgaagaatacaatattatcaccagcgttttctggtacaccattaatgagtgtatacaacaccacacgtgacggcgcaacgtaaggtggtacattatagtttgcaactaaaaacgaaccatcgtcaaatacagcaacaacaacaccagtgtgaccaataccatatgcagttgcttgtaagtatggtggtttactagaaaaaccataccctactgttggattatgtgttgtttttgcacctaactttttatagacgtaccatacacgttgaccgtttgtgatttggccgtcgtcggtaggttgtctttttccatgtagttgtgacatatacgcccatgttaattctgtacactgaccagcattaccagtttgaggaaatatgttacctggtttgtataaatattcttttttgaataaaggtacaccaattgcttttttatatttttctggtaactgtgcgtacgtccagttaccaccaatcacacgaccactttttccatttggtttgactgatttaccactaattggtttatgatctccgtcatcatcagtagggttagaactgctacccccgctatccacttgcacgctatcaatcaatttttttaatgaatcaagtaagccaattgtcattttaatatgatatgtgttgttgaatgttttttgtaatgtaaaataatcattactaaaaaatttatcactaccaatactgtgaacgtcccattgtaatgcgtcttgtactttttttaataattcttgcatggcttgtttagctaaagcgagtagtgaactaccactgtcaccactactaccactgtcagacgtgtcactaggtgaaccacctttaccgtctaatttaccaccccatgctaaaatagtatttgcaccgtctaaaaaaggattaccatagttttgtactttattatatgacgctttcaaacctaggggataatatgcagcccaagtagccgctgctgttaatggaatatatgcacgtccaattgtacctgctttcatatttttagcaaaatctgcattaccctttctttgtacgtcttgaggtacaaagtggacaatgttacctgcgtcataccaagacggttgtccagcttgttttgattgtgatactaactttcttgcaataaatttagcgtctgttaaataatcaccacgtgcagacgtatgatttaaccaacctaaaccggcactatacccctcatttttttcatatacagcaaaaagagtaggtgacacacctatctcttttactgcttttaatacttgtctgattttactttcattaccacctagccatacattaaagcgtccatacccttttactttaggcactaactggtctatcgttaaaccgaaatcatcattaatatacgaatgtgtaaatttatctatcttctcttggtcgttcattattatcactcttttcagaatcgtttttaattactcttaatttatctttaatttgttcaggcactaatacgtccatttctgcacaattttctacgatagataagccttcattagcgatataatagaaaatcgtaatcatgagtaagccaccttttaattgtaaaatctggtcaatgatgttcgctaaaatgataatacagaatatcaataattttttagcaaaacctttcattgatttttttgaccataagttattatttttaatggctttagcaaaaccagtaattacatcaacgatcattaaaacaaataaaaaatatagtaattttaaatcacctgcatatatgaacatatgaaacgcttctgtatcggtgaatcttaattttacctcattcattttatacaccctctctaaatttattatttaatggattttgtaacattggattacctgaaccgtcattatgccaaaatctcacaccggactctaaaatcgcttttaattgctccattaacatggggtcaatgtcacgtatagtatacgtacctgtacattttaaataattgcaaacagtcatactgttaattggttcaataaatgaattatagtcatttacttcaaaaccaaacaacatataatatttttgtaaaaatgtaatttcttttggtgacggtacactaattttcatcgttaaaccgttaatgctattcgcaatttggaatgcgttgcccatttctgattcagttacagacggtggttgtaaggctaaatctttatattcagcttgttgttgtttgtagaaattatattcttcattaaacttaccaaataaagcagttggacttaaattacttgctacactcacagcgtcataaaatcgtgattttgggtcactaccatttaatacattatcaatacgatttgtaattaattgactttctgcattcttttgtctattcgcttgttgtgattgtcctaagataccattattgattaatattggtacttgtgcaaaactattaaatgttatatttgtatttaagaatgaacccgtatcaatcaatatttctttatttttagcaagtatgggtctgtcgttttcagcactgttataatctactggatatactctaacttcattatgataaccaatgatcgattttgtacgtaacttaacacctgttttttgtgaaattttaccagcgtcgagtaacatcgtatttccattccaatcataaaattcaattgtcatatactcattacgtatcatatgtttaaattcatcttttttagataacatcatttcttgaagttttgtaaaacttaatgataaatcttttagactccattcttttgatttaccaccctgttttaatgtttttaatcctgtaattttttcactggtttttacgtcctctaaatcttttgtattaataaagtctttaggtaacatttgaaccttttgaaagttttgtgtaatccatggatatgcactcattttatccataaagttaataaagtcaccatattccataacgtataagttaactggtgatgtgatattgtcatatatcgtacccttagacgtgtctaagtttggctcttttttcgtaccaaatttctttgataaatcagcacttgactggaataatactatattttccaaatattgttgcatttggttatacacatagtttttattcgatacttttaacacatcgtcattattacgcaacattggtaacatatagttataggtgcgttttgataaatgttgacgttcaatattgacgtttgagagttgctctaatacattaccttgcgtgtacgtcataatagtatcaatcacaaaatatattttaaccacaacatcattcacatattcgatttgattcacaaacgcataataacgtctgtcctcaaaatctgataaaaacgtcatgtagttaatcccttgtgcgtcatgccactgcatatcaacattgatttccattctatcacgtataaaattatacggttgtttggaatagtctaacgatttaaaatgacgtccatttaaaaaataatcatcacgctctttattactattaaaatgaatcgtattttgataatcagtaaacggtgtgttatagaaaaatttaaaatttgttaattttctcatttttacctccataaaaaatagtcgtataaataatttatacgactattataacatttttattcaatgatttgtgtatctattgcaaaacgtttatcaccatttgttaagtcactatcgctataatttgatgtaacaaaatgtaattcattattaaagtttaaatataatcttgtattcatcattttattatcaatcgcacattgtgtgtagtgatgtgttgattttaagtttgcgttaatcgtacctaatttaatatcaccgtttttcttaatcccttttaatactccttttaattgtatcgttttaacaccattaattgtgacaatacgatattgcggtgcgggatatccaccattgctatcacttccaactataccactttcgagcgatatatcttgccaacctgtatcattaaattgtaattttgcactgttaattttttgattaaaattttgttcaatttctgatttagattgagcaattttattatctatatttaagttattcacttttgttgataaactgttaatacttgattcgtttgttgatgttttttgttttaaatcactaatatcattatcatatttagataaatcaacgttaccgttactgttaacaactggtctatcttttaattcttgaatatctttttcaatttttgatattttatcattatttgtttttaaagtagcgatgtcagatttaattgaattaatatcctgtgtataatctttttggttaatactgtttaattttgtttctaactcttttatcttatcaagattagcatttgttttaatatcattaatggattgttctaacgattcaagtttacctgcacttgaattttgttttaacttattaatatcctcttgtatttcttgaattgtgttgtcattgttttgtttagattttaataattcaatttgtctttcaatgtcttttattttatcaatattaacactttttttaacttcagttaattctctttttaatgcgtcaatgttttcagcaccagtatttgctttaattaaattaatttcatctgtgttacgtcctacactatctttagtagcgtttaatgattgtgtaagggtttcatatttatcatcatattttaatattgatttttcaaattcatttaattgtgtttgatgttgttctaaactttcatttaatatgacatctttttcttttaatgaatcaatctcttttttattactatcaatcattggttggaatttttttatcttctctaaaaatgttgatattttttctttgttatcattaattgcaatatcatgactttctatttgaccactgtatttattaatcaatgtttttaaatcattataaaatgttaacttataataaccagtatcagtacggacataaatatgcccgtctgatgtactgattaagtcattttcttctgttaaaaaatctgctaacctgtctattgtttcaacttgtcttaaacttcttacgattctatcagccattgttcacacctcttatttgtatcgtttccaactaaattcaaagaaaaagcctaaaatacccattatgagaacaccccccacggtacaccaacactgtaactattacctgttttaccattccattgccttactggtaaataataacgtgtaccttgccaattataaccaatccatacgtaaccatctgataaacaaatttcgtcatatggtgtataaccatttggttggaaccaatagccattaggttctgataattttggactgccgacacgtgcaaatattggtaaaaatccacatgtaaatgttccattctcatttctgtaatacgtaccatattggttttgtttccaatttccaaatgtattgaccattgatgtttctaattttttactattactacctgaaaattttggacgaataaagtgtgttacaccgtcataataatgtgttctgatggttgctttttcccaaccgtcaaaaccaccatttaaccagttttgctctaaacatgtataataatctaaatttccacttatcacacattgaatatgaccatattgagaattagtataaacagcaacgtctcctaattgtggtttaaagctcggtgtatttttatacaccgttgctaaacctttaaagtcattattaatagcgtctttggcattgccccacatacgaactttaccgtctgtaatgtaatatacataagcaactgctaagtccatacattgaaatccatatgcaccatcaaaatcaacacctacaccctcatgattatatatccattcttttgcttgttgttgtgatttcatttatatcactcctatttttgatgttttgctacccaaccatattcacgatgtttcgtttcagcattaacattactaaagaattctttatgcactttaacgttagcctccaacgtttgtcttacttctccaacagcgttaccacttgaaacacgtaaccatgcacccacttttttaatttcttctggtgcgtctctaaacaattccatttggttgccagtaatataatattcaccaggtgtagtaacgtaagctatccaattattatattgactcgcttctaaatattcttgatatggtgcatctgttttgattgtagtccataaaccataatcccattttaatttaaatacgtcgagtgtcataccacgcacaattttaaccatttttcgaccagtagaaaaacgtgttaattcttgtactgtgcctaatgtttgtgttgtagggtatacattgataaaacaaccagcgtctataatttttttactaccattcattggcatatttttaagtttgctagctgtaccaccgtcaatataataaaatcctggttgtgttaagtcgtttaagtcgtcaatattgtctggaatagataatgcacgtccatcgtcttttgttaatttatagttttgtgaacctctcgcacgtaatgcttcaaagtgttcatactcaccaagttgaaagaacccatataaattattgaatcgttttccaccaccgccattagtcatagcaagtaataacgatttacgttttgtttttggattggtataaatacaaataccctccggctctttaaagttatcacgtggaaaattaataccgtcttgatatgataatttaaacggataatcataaatcttttgaccagtggttaatgaatatttaccaatttgaacgtgtgaattaactgaactgttaccacttaaccagtataaatcatcaccatcaacggcaatcccttgcatccatcgattatcattgttttctgaattatcaatcgtcatttctttttctacattatcaatgtgattttttacgtcagctcttgaacgtacctgtattgtcccgtcaccgaatcttaaaacgattttgtcatttgcttcatcgattaaaggtgtaaacgtatgtttgtttaaaagtgactgtggtgtgtaatctgttaaacctttagcctcttctaaatctaatacataattatctttatatgcgacttgtaacagtttcgcgacaccgtcatgatgtaaccaaattttcatttctccattggattgacgttctaatccgattgttgtaccgtgaccaccttgtacaatacgcatactagaaattaagtcaccgctaggcgttaatttattaatccaaaaaccttctgggttttgtgaatctgattgtgtagagtacatttgattcgtttctttatcgatattaattgattggtttactgcgttacgaataccaccgaagcccataacaaactttggttcaagctcatttaattcaaaaccattaactaaacggtcaatgtctttaattaggtctttcacttctgctttaaaatcattcatttgtttcatttctgcgactttaaataatgcaaatgcagatgttaaaccagcactatatttagtaaactcatcgtgaatgattttatcaatcgtaccgtcgtttaaccaacctctaaataagtcttttgcttggtctgggaatgctttcattaaatcgtcccagtttttgaaacgtttttttaactcaccgtcatagtcccaaatacgatatgctaatacttcaatgagttttgataatcttgaaatataatcataatatgattttgaattggtattataatctgctctatcatcgtaaaacggtgtgtaacgttctctatttttatatatttcgtctaaaaatggacgaatgtcgtcaaaatatttaaattcgttttcattatatgccataattttccacctttaccaaatttgtaaaaaacattttttatcaaattcatttaaaattttctttcttaaatcgtatactttatcaatattatcaattaaatactgttttgaaaattgtgtacctttcgcattacctttttgattttgattacgttttgcgttttgattactttcgttacttgatttattcacagttttaccgttatcaattgtattattgtctgcaaattgtaacgttgttttatcaacatctatgttaacctcgctttgtggtaatgacgcataagcatttctgttcgctgtcataccagttgaattgtctaaagatgtagcattttgatttgaggtttcatctgtgttgtttgttgtatcttcattatgttctgtaaaaccttgtgattgtagatatttttcaacttcgcttgatgaataaaccacattcaaataatcttcatgtgtgatacatacagtaatcacttgcatgccaaacgcttcaactgtttgtctgttgatttctctatctaaaaagtgtattgtaaatgattttttgaaaagtaagtcagataagtcatctttaagtttaaaacctttaaacactttttcattaacgatggctaaaacgtctttgtcaaacttcaacattttttgcatgaattgaaattcatcatcataaaacgttaatttatcattatttacaaattcattaaaaccttttttaattaattctgatttaataaaatcaaataaagtcattgtatatctagccattgtattcactactttcatcattagacaatgtatctatcattgtgatttctgaagtaacttcatcgtcgtaatacggtttaatatctaaaccataacgttttgataagaatgtaatcggttcacgaccttttaaataaatattactatttgatgtggtaaaacctcggttgctttttgcttcttcgtccgaaacaccgctttccttatccactgctagtgaattaatacccaaatagttacttaattcactaattttattttggtattctcttttcatctcagttagtgctggaatcacactattactggttaaatctatgatatcatcatcggcgttaaacataggtgacattttaacaaatggtgcaccgttatatatttccgatacaagttgattaactgactcatcattaatgtctgatttaaatattttgctaaattttgcttgcataattaatgaaaatcgagataaaacaacctcagataattcatcagtataatgttctataatttctatatcactattatattgaattggtttattttgcatgacaacaaagttgccactcatacagttatcatatattttatgaatctgtaagcattcatctggtattaaatagtctggaacaatgaaataaatatcatcttttgttaatcgtttttgaaattgaaaattgaaatttgatgaaaagtttggggcttgattaaaataagtgttgttaacatagcccaaaatcatgatttgttcatttcttgctttaccaacaacaacattaatgttttgccttaatgcagattctaattgaataaaatctataccaaccgaatctcgattggtatagttaattaatagtggtaaaaattccaaataacgattaaacataagacgtttaaatctgttgcgatgttctacaacacgtttattgatttctttagataattcaacctgtacgcctttattatgtttagtcatttatagcacctctataattctgttctcggtgtaacatcttggtcagtaattaaaattttattaaagaatgggctaatggctttaaatgaataataatgaatccagtgtgtgacttcatcaaattcaccattatagaatggttgttttaacataccttttgtgtaacgtttatatttaattgaattaatatccaaaataaatgcgtataaatctgattttggtttaatttcttcaacgttagacttaaattcagataaatttgaaacatcataagtgaatactgaaccaactggaattgtatcacctttatgagtttgataatcaccgtaagcacgtaagaaattaactgattcgtcacttgatacgacaatatctttagtaactttaaaaactccacctaaatcatcaaaactaataacgtggtctgtaaagtcaatccctgctacttggaacgtgttagcaatctttgtatctaaaagataagattttaagctatcagtcgttaaaataacaatatcttttaattttgaaactgttgtatattgtccaatagcaccacctgaagcacgatgaacttcattatatttagcactgttgttttgtaagttaagaatagcttcaaaaactttacttgctaaatcttcttttgatgttgctttacgtacatttgattcagataattgattcaatgaataatcaactaacattgcacgcatttctttttcttctaatacgttaatatcagaaattttctttttatagacacctaatgcataattagttgcgtctgctaatgtttggaaattgaaacgtgtatcattgttatttaatgtaaatttttgtttcttcacaataccactaccatataacttagtagccatacgtgggtagttacgtttcaacattagttcttcatttttagataagtccatgttaataggtactgtatccataattacatattcttcactgtattgaccaataaagtcctgctctttagctaaccaattaaaacggttacctaatgcaatatcgattaataacgtttcattaatcttagggaataaaaatttatttacaaatgtttcaaacattgtattagaattatcccacttatcaccaaacgtccatgattttgaataagtatgattaaagtcttgtaatgcagatttagctgactttgcaactaatagtgctgtttcgttttttgtactcttttctgccatgatttattattcctcctctacatcgccagtaaatgactgttttgaaagtgaatgaatttgtacaccataactatcttcacttttatttgtatcaattgacatattttcatttaattctgttcgtttatttaatcttgaatcttcatatgatgtacccatcatagaacgcatgttgttaccctcgtacatgtttaaattcctcctaatctaaatctaatttatcgacaagttcttcatctgaatagtctttatcattgttatctgtttcatttgattctggttgtggttgtggttgtggttgttgcatttgtgatgtttcaaaattagtaaatcgttgttctaatgaagcgatacgttgttctaaaacaactgggtctaattttgcactatcttcatctgttatagtaggttctaatttgttttcattttcttcttcgattgtttctactgttttatcttcagttgattcttcagttgattcttcagttgattcttcagtttcttctgttgattcttcagttgattcttcagttaattctttgtcgtctgattttacgatttcctcaaattctgtcatttagacacctccaaatattttataactaattatatcatagaatatttaaataagtaaattaaatttattttaatttcaaatcataactttgaataaaagtcaatagatacataaatttttatttgatgaatatttaataggtttgataagttgttaaagttggtgaacagtatttataagtttagtaaagaaatgataagtgaatttataagttttgatttgtataatcgtttattttaaacggtggggaggtgtaaatttatactaactttttccctattatattt

SEQ ID NO: 6 - DNA, 29043 bp, Propionibacterium acnes
  source 1..29043: mol_type = genomic DNA
  misc_feature 1..29043: note = bacteriophage MBPAC2
cttcgttcagttcggatgggtgtttaaccgttttcgcatttatcgtgaaacgctttcgcgtttttcgtgcgccgcttcagcttcctgatagtcgtgattggcgtggggagacgcgtcgttggtggcgcgtgtggggtgaggatccgcgtgccgggtttgtgtctgatgaggagtggctgtttctcatggatgctgcggtgattcatgattgtgtgtggcgtgagggtcgcgcggatttggtggcttcgttgcgtgcccatgtgaaggcttttatgggcatgttggatcggtattcggttgatgtggtgtctggtgtccccggtgggggttctgcggtggcgatgattgaccggtataggaagcgcaagggggcctgattaggtgtctggtgttgttgggtctcaggttcctcgtcaccgtgtggctgtggcgtattcggtgtctgctggcggtgatgctggggagttgggtcgtgcgtatgggttgacgcctgatccgtggcagcagcaggtgttggatgattggctggctgtcggtagcaatggcaggcttgcttcgggtgtgtgtggggtgtttgtgcctcgccagaatggcaagaatgctattttggagattgtggagttgtttaaggcgactattcagggtcgccgtattttgcatacggctcacgagttgaagtcggctcgtaaggcgtttatgcggttgaggtcgttttttgagaatgagcggcagtttcctgacttgtatcgtatggtgaagtcgattcgtgcgacgaatggtcaggaggctattgtgttgcatcatccggattgtgccacgtttgagcgtaagtgtggttgtccgggttggggttcggttgagtttgtggcccgttctcggggttctgctcgcgggtttacggttgatgatttggtgtgtgatgaggctcaggagttgtcggatgagcagttggaggctttgcttcctacggtaagtgctgccccgtctggtgatccgcagcagattttccttggcacgccgcctggcccgttggcggacggtagcgtggtgttgcgtcttcgtgggcaggctttgtcgggtggtaaaaggtttgcgtggacggagttttcgattcctgacgagtctgatccggatgatctatcgaggttgtggcggaagttggcgggggatacgaatcctgcgctgggtcgccgcctgaatttcgggacggtgagcgatgagcatgagtcgatgtctgctgccgggtttgctcgggagcggcttggctggtgggatcgtggccagtctgctacgtctgtgattcctgcggataagtgggctcagtctgcggtggatgaggcgagtctggttggcgggaaagtgtttggtgtctcgttttctcgttctggggatcgggttgctttggcgggtgctggccggactgatgctggggttcatgttgaggttattgatgggctgtcgggaacgattgttgatggtgtgggccggttggctgactggttggcgattcgttggggtgatactgaccggattatggttgccgggtctggtgcggtgttgttgcagaaggcgttgacggatcgtggtattccgggccgtggcgtggtggttgccgatactggggtgtatgtggaggcgtgccaagcttttttggaggctattcgttccgggaatgtttctcatcctcgtgttgatgctcgccgtgacatgttggagattgctgtgaggtcggcggttcagaagcgtaagggttcggcttggggctggggttccacgtttaaggatggttctgaggttcctttggaggctgtgtcgctggcgtatcttggtgcgaaaacagttaaagtgaagcggcgtgaacggtctggtaggaagcgggtgtctgtggtatgaactcggatgagttggctctgattgagggcatgtttgatcgtatccaaaggttgtcttcgtggcattgtcgcattgagggctactatgagggctcgagccgggtgcgtgatttgggggttgctattcctccggagttgcagcgggtgcagacggtggtgtcgtggcctggtatagctgtggatgctttggaggagcgtcttgattggcttggctggactaatggtgacggctacggtctggatggtgtgtatgctgcgaatcgtctatcaaccgcgtcatgcgacgtccaccttgatgcactaatttttgggttgtcgtttgttgcgatcattcctcatggtgatggtacggtgtcggttcgtccgcagtcaccaaagaattgtacgggcaagttttcggctgacgggtctcgtctggatgcgggtttggtggttcagcagacgtgtgatcctgaggtggttgaggctgagcttttgcttcctgatgtgattgttcaggtggagcggcggggttcgcgtgaatgggttgaggtggatcgtataccgaatgtgttgggtgcggttccgttggtgcctattgtgaatcgtcgccgtacttctaggattgatggccgttcggagattacgaggtctattagggcttacacggatgaggctgtgcgcacactgttggggcagtctgtgaatcgtgatttttatgcgtatcctcagcgttgggtgactggcgtgagtgcggacgagttttcgcagcctggctgggtcctgtcgatggcttctgtgtgggctgtggataaggatgatgacggtgataccccgaatgtggggtcgtttcctgtcaatagtcctacaccgtattcggatcagatgcggctgttggcgcagttgacggctggggaggctgcggttccggaacgctatttcgggtttatcacgtctaacccacctagtggggaggctttggctgccgaggaatctcggcttgtgaagcgtgctgagcggcgtcaaacgtcgtttggtcagggctggctgtcggttggttttttggctgccaaggcgttggattctcgtgttgatgaggccgatttttttggtgatgttggtttgcgttggcgtgatgcttcgacgcctacccgggcggctacggctgatgctgtgacgaagcttgttggtgccggtattttgcctgctgattctcgtacggtgttggagatgttggggcttgatgatgtgcaggttgaggctgtgatgcggcatcgtgccgaatctgcggatccgttggctgcgcttgctggggctatatcgcgtcagacgaacgaggtttgataggcgatggcttcgggtgttgcgtcgaggttggctgctaccgggtatcagcgtgaggcggtcaggtttgctgggaagtatgcgggctattatgccgagttgggtcgtttgtggcattccgggaagatgagtgacacacagtatgtgcgtttgtgtgtggagttggagcgtgccggccatgacggttcagcagctatggcagccaaattcgtgcaagattttcgccggttgaacggtgtcgatcctggtttgatcgtgtatgacgagtttgatgctgctgcggctttggctaggtcgttttcgactatgaagattcttaagagtgacccggatagggcgaatgacacgattgatgcgatggctgcgggttttgatcgggctgttatgaatgctggtcgtgacacggttgagtggtcggcgggtgcgcagggtaggtcgtggcgtcgggtgactgacggggatccgtgtgctttttgtgccatgttggctacgaggtcggattatacgaccaaagaaagggcactcacttccggtcatacgcggcgtcataagcgtggtggtaagcgtccgtttggttcgaagtatcatgatcattgtggttgtacggtggttgaggttgttggcccttgggaaccaaatagggctgatgccgagtatcagaggacgtatgagaaggcccgtgagtgggttgatgatcatgggttgcagcagtcgcctggcaatattttgaaggctatgcgtactgttggtggcatgagataatttgatgtggtttccggttgtgcgccgccggttattggtgcacggggttgtctcccgcacgggggtcaacaatgttgtgttgttttccgcaaggagtgtagggttaggctatggccgatcagagtgttgaggaacagaatgttgacaatgatgttgtggagtccggaaaggataacggcattgttgatacagtaaaagacgatggcgggcaggaggtagccgacaatcagttgaagaatgaaggcgagggtaaatcgccggggactgattggaaggcggaggcccgtaagtgggagtctcgtgctaaaagtaatttcgccgagttggagaagcttcgtacatcgagtgacgattctggatctactattgatgagcttcgccgcaagaatgaggaactcgaagacaggatcaacgggtttgttcttgagggtgtgaagcgcgaggtggcttcagagtatggtttgtctagtgatgcgatcgctttcttgtcgggtggcgataaggagtcacttgccgagtctgctaaggctttgaagggtttgatcgaccatagtagtggtggcgcgggtgtgcgccgtcttgcggggagtgcccccgttgatgatgttaaacgacgtgagggtgtcgcgtttgtggatgctcttgtcaataattctaggagatgatttgtgatggctgacgattttctttctgcagggaagcttgagcttcctggttctatgattggtgcggttcgtgaccgtgctatcgattctggtgttttggcgaagctttcgccggagcagccgactatttttggccctgttaagggtgccgtgtttagtggtgttcctcgcgctaagattgttggtgagggcgaggttaagccgtctgcttcggttgatgtttcggcgtttactgcgcagcctatcaaggttgtgactcagcagcgtgtctcggacgagtttatgtgggctgatgctgattaccgtctgggtgttttgcaggatctgatttccccggctcttggtgcttcgattggtcgcgctgttgatctgattgctttccacggtattgatccagctacgggtaagcctgctgcggctgtgaagacttcgctggataagacgaagaatactgttgatgcaaccgatagcgctacggctgatctgattaaagcggttgggctgattgcgggtgccggtttgcaggttcctaacggtgttgctttggatccggcgttctcgtttgctttgtcgactgaggtgtatcctaagggttcgcctcttgctggccagccgatgtatcctgccgccgggtttgccggtttggataattggcgcgggctgaatgttggtgcttcttcgactgtttcgggtgtcccggagatgtcgcctgcctctggtgttaaggctattgtgggtgatttctctcgtgttcattggggtttccagcgtaacttcccgatcgagcttatcgagtatggtgatccggatcagactgggcgtgacctgaagggccataatgaggttatggttcgtgccgaggctgtgctgtatgtggctatcgagtcgcttgattcgtttgctgttgtgaaggagaaggctgccccgaagcctaatccgccggccgagaactgattcatttgttgcggtgatgtgtcaatgtgcagggggtggtgttgatgggtatcattttgaagcctgaggatattgagcctttcgccgatattcctagagagaagcttgaggcgatgattgccgatgtggaggctgtggctgtcagtgtcgccccctgtatcgctaaaccggatttcaaatacaaggatgccgctaaggctattctgcgtagggctttgttgcgctggaatgatactggcgtgtcggggcaggtgcagtatgagtctgcgggtccttttgctcagactacacggtcgaatactcccactaatttgttgtggccttctgagattgccgcgttgaagaagctgtgtgagggtgatggtggggctggtaaagcgttcactatcacccccactattaatagtagatatgcacattctgaggtgtgttccacggtgtggggtgagggttgctcgtgcgggtcgaatattaacggctacgctggccctttgtgggagatatgatatgaccagttttccttatggtgaaacggttgtgatgcttcaaccgactgttcgtgtcgatgatctaggcgacaaggttgaggattgggggcatcctgtagaaactgtgtaccataacgtggccatctattcttcgttgtcgcaggaggatgaggctgccggccgtgactctgactatgagcattggtcgatgcttttcaagtcccctgttgtgggtgctgattatcgttgtaggtggcgtattcggggtgttgtgtgggaggctgacgggtctcctatcgtgtggcatcaccccatgtccggttgggatgctggtactcaggttaatgtgaaacgcaagaagggctgataggtagtggctcaggatgtgaatgtgaagctgaacttgccgggtattcgtgaggtgttgaagtcttctggggtgcagtcgatgttggctgagcgtggcgagcgtgtcaagcgtgctgcctcggcgaatgtgggcggtaacgctttcgataaggcccaataccgtagcggattatcatcggaggtgcaggttcaccgtgttgaggctgtggcgaggattggcaccacatataagggtgggaagcgtattgaggcgaagcatggcacgctggctaggtcgattggggctgcgtcgtgatcgtctacggtgaccccaggaagtgggctaaacgtgttttggcggatgatggctggctgtctgatgttccttgtactgggacggtgcctgaccggtttgagggtgacctgatttggcttgcgttggatggtggcccgcagttgcatgttcgtgagcgtgtttttttgcgggtgaatgtgttttcggatacgccggatcgtgctatgtcgttggcgcgtcgtgttgaggctgtgctggcggatggggttgacggtgaccctgtggtgtactgtaaacggtctactggccctgatttgctggttgatggtgcacgttttgatgtgtattcgctttttgagctgatatgtaggcctgcggggtctgaataagcttattgtttttgttttaatgtaattgtttgatatttaatgggggttgtgatggctgcaacacgtaaagcgtctaatgttcgttcagcggttactggcgatgtttatattggtgacgcgcacgcgggtgatactattaagggtgtggaggcggttccttccgggcttacagctttagggtatttgtcggatgacgggtttaagattaagcctgagcgtaaaacggatgatttgaaggcttggcagaatgcggatgttgttcgcactgtggctacggagtcgtctatcgagatttctttccagctgatcgaatccaagaaagaggttattgaactgttttggcagtcgaaggttactgccggatccgattcgggttcgttcgatatttcaccaggcgccaccactggcgtgcacgctttactgatggatattgttgatggggatcaggttattcgctactatttccctgaggttgagcttatcgatcgtgacgagattaagggcaagaatggcgaggtgtatgggtatggtgtgacgttgaaggcttaccctgctcagattaataagactggtaatgcggtatctggtcgggggtggatgacggctttaaaagctgatactcctccggttcctccgaagcctcagccggatccgaatccgccgtccgatcattgatacacatagtttgagggattgttgatagatgagtgacacaggttacacattaaagattggtgaccgtagctgggtgttggcggatgcggaggagacggctcaggctgttcctgcccgcgtttttcgccgtgcagctaagattgcccagtcgggggagtctgcggatttcgcccaggttgaggtgatgttttctatgctagaagcggccgccccggctgacgccgtggaggccctggaggggcttcctatggttcgtgttgccgagattttccgccagtggatggaatacaagcctgacggtaagggtgcctcgctgggggaataatttggctccacggcctgattgatgattatcgtggggccatcgaatatgattggaggacccggttcggttgctcggtttatgatgttggtggcccggtgatgtgttggggtgaggctgtccggctggctggcgtgttgtgtaccgatacgtcgagccagttggcggcccacctgaatggttggcagcgcccgtttgagtggtgcgagtgggctgtgttggacatgctggatcattacaggtctgctaatagtgaggggcagccggagcctgtggcgaggcctacggatgagcgtagggcccggtttacgtcggggcaggtggacgatattttggcgcgtgttcgtgccggtggcggggtgtctcgcgagattaatattatggggtgaatagtgtatgtctggtgagattgcttccgcatatgtgtcgttgtatacgaagatgcctggtttgaaggcggatgttggtaaacagctttccggggttatgcctgctgagggtcagcgttcgggtagtctttttgctaagggcatgaaattggcgcttggtggtgccgcaatggtgggcgctatcaatgttgccaagaagggcctcaagtctatctatgatgtgactattggtggcggtatagctcgcgctatggctatcgatgaggcgcaggctaaactgactggtttgggtcatacgtcttctgacacgtcttcgattatgaattcggctattgaggctgtgactggtacgtcgtatgcgttgggtgatgcggcttctactgcggcggcgttgtctgcttcgggtgtgaagtctggcgggcagatgacggatgtgttgaagactgtcgccgatgtgtcttatatttcgggtaagtcgtttcaggatacgggcgctatttttacgtctgtgatggctcgcggtaagttgcagggcgatgacatgttgcagcttacgatggcgggtgtccctgtgttgtctttgcttgccaggcagactggtaagacgtctgctgaggtgtcgcagatggtgtcgaaggggcagattgattttgccacgtttgcggctgcgatgaagcttggcatgggtggtgctgcgcaggcgtctggtaagacgtttgagggcgctatgaagaatgttaagggcgctttgggctatcttggtgccacggctatggcgccgtttcttaacgggttgcggcagatttttgttgcgttgaatccggttatcaagtctatcacggattctgtgaagccgatgtttgctgccgtcgatgctggtattcagcgtatgatgccgtctattttggcgtggattaatcgtatgccgggcatgatcactcgaatgaatgcacagatgcgcgccaaggttgagcagttgaagggcatttttgcgagaatgcatttgcctgtccctaaagtgaatttgggtgccatgtttgctggcggcacggctgtgtttggtattgttgctgcgggtgttgggaagcttgttgcagggtttgccccgttggcggttgcgttgaagaatctactgccgtcgtttggtgctttgaagggtgccgctggcgggcttggcggcgtgtttcgtgccctgggtggccctgtcgggattgtgattggcttgtttgcggccatgtttgctacgaatgcccagttccgtgccgcggtgatgcagcttgtgggtgttgttggccaggctttggggcagatcatggccgctattcagccactgtttggtttggttgccggggttgtggcacagttggcgccagtgttcggccagattatcggtttggttgccggtttggctgcccagattgtgcctttgattagtatgcttgtcgcccggctggttcctgttatcacgcagattattggtgcggtgacacaggttgctgcaatgttgttgccggcgttgatgccggtgttgcaggctgttgttgctgtgatacggcaggttgttggcgtgatcatgcagttggtgcctgttttgatgcctgtgattcaacagattttgggtgctgtcatgtctgtgctgccgcctatcatcggtctgatccggtcgctgataccagtcatcatgtcgattatgcgtgtggtggtgcaggttgtttcggttgtgttgcaggtggtggcccgcattattccggttgtgatgccgattgtgacagctgtgatcgggtttgttgcacgtattcttggcgctattgtgtctgctgaagcccgcattattgggactgtcacccgtgtcatctcatgggttgtgaatcatttagtgtctggcgtgaggtctatgggcacggccatcttgaatggctggaatcatattagggcgtttacgtcagcgtttattaacggtttcaagtcggtgatttctggcggtgtgaacgctgttgtggggttttttgcccggcttggttcttctgttgcctcccatgtgaggtctggttttaacgcggctcgtggtgctgtttcttctgcgatgaatgctatccggggtgttgtgtcttcggtggcgtctgctgttggcgggtttttcggttcgatggcgtctagggttcgtagtggtgctgtgcgcgggtttaatggggccaggagtgcggcttcttctgctatgcatgctatggggtccgcggtgtctaatggtgtgcatagtgtgctagggtttttccggaatctgcctggcaatattaggcatgctctcggtaacatggggtccttgttggtgtcggctggccgtgatgtggtgtctggtttgggtaatggtatccggaatgctatgagtggcctactggatacggtgcgtaatatgggttctcaggttgctaatgcggcgaagtcggtgttgggtattcattccccgtcgagggtgtttcgtgatcaggttggtcgccaggttgttgccggtttggctgaggggatcaccgggaatgcgggtttggcgttggatgcgatgtctggtgtggctggacggcttccggatgctgttgatgcccggtttggtgtgcgatcgtctgtgggctcgtttaccccgtatggcaggtatcagcgtatgaatgataagagtgttgtggtgaatgtgaatggacccacgtatggtgatcctaacgagtttgcgaagcggattgagcggcagcagcgtgacgcgttgaacgcgttggcttacatgtgatagggggtgttgtgcatgtttattcctgacccgtctgatcgttctggtttgactgtgacgtggtttatggatccgctgtttggcgacgagcgtgtgcttcatttgacggattatacggggtcgtctcctgtcatgttgttgaatgattcgttgcgcggtttgggtgttcctgaggtggagcatttttctcaaacacatgttggggtgcacggctcggagtggcgcgggtttaatgtgaagcctcgcgaggtgacgctgcctgtcctggtgtcgggtgtggatccggatccggttggcgggtttcgtgacggttttttgaaagcgtatgacgcattgtggtctgcgtttcctccaggggaggagggggagttgtcggtgaagactcctgtcggtcgtgagcgtgtgctaaaatgccgttttgattcggctgatgacacgtttacggttgatccggtgaatcgcggctatgcgcgttatgtgcttcatttgacagcctatgacccgttttggtatggggatgagcaaaagtttcgttttagtaacgcgaagttgcaggattggttgggtggcggccctgtcggtaaggatggtacggcgtttcctgtggtgttgacgcctggtgttggttctggctgggataatctgtctaataagggtgatgtgcctgcgtggcctgtgattcgtgttgaggggcctttggagtcgtggtctgtgcagattgatggtttgcgtgtgtcttcggattatcctgttgaggagtttgattggattactattgatacggatcctcgccagcagtctgcgttgttgaatgggtttgaggatgtgatggatcgtttgaaggagtgggagtttgcgcctattcctcctggcggttcgaagagtgtgaatattgagatggttggtttgggtgccattgttgtgtcggtgcagtacaggtttttgagggcttggtgaatagttgatggctggtcttgttccgcatgtaacattgtttacgccggattatcgtcgtgtggcgcctatcaatttttttgagtcgttgaagttgtcgttgaagtggaatggtttgtctacgctggagttggtggtgtcgggtgatcattccaggcttgacgggttgacgaggccgggtgcacggctggttgttgattatggtggtggccggattttttctgggcctgtgcgtaaggttcatggtgtgggtccttggcgttcttcgagggtgactatcacgtgtgaggatgatatccgcctgttgtggcgtatgttgatgtggcctgtgaattatcgtcctggtatggttggtatggagtggcgtgccgacagggattatgcccgctattcgggtgcggctgagtcggtggctaaacaggtgttgggggataatgcttggcgttttccgcctggtttgtttatggaggaggatgagagtcgtggccgctatattaaggattttcaggtgcggtttcacgtgtttgcagacaagttgttgccggtgttgtcgtgggctcggatgactgtcacggtgaaccagtttgagaataaggttacggatcagcggggtttagtgtttgattgtgtgcctgctgtgacccgtgagcatgtgttgactgccgagtcgggttcgattgtgtcgtgggagtatgtgcgtgacgccccgaaggctacttcggtggtggttggtggccgcggcgagggcaaggatcggctgttttgcgaggatgttgattcgatggccgaggatgactggtttgatcgtgtagaggtgtttaaggatgcccgtaacacggattctgaacatgtgcatctcatcgatgaggctgagcaggtgctgtccgagtcgggggccacgtcggggtttaagatcgagttggctgagtcggatgtgttgcggtttgggccaggcaatctgatgccgggtgatcttatctatgtggatgtgggttctggccctatcgcggagattgtgcggcagattgatgtggagtgtgtatcgcctggtgatggttggacgaaggtgacacctgttgctggggattatgaggataatccgtcggcgttgctggcacggcgtgtggctggtttggctgcgggtgtgcgggatttgcaaaagttttagtaagtgattggggtttgttgtgggtattgtgtgtaaagggtttgatggtgtgttgaccgagtatgattgggctcaaatgtctggtctgatgggtaatatgccgtctgtgaaagggccggacgattttcgtgtcggcacgacgattcagggtgccacagtgttgtgtgaggttttgccggggcaggcttgggctcacggggtgatgtgcacgtcgaatagtgttgagacggtgacggggcagcttccgggccctggcgagacccgatacgactatgtggtgttgtctcgggattgggaggctaatacggccaagttggagattgttcctggggggcgtgcggagcgtgctagggatgtgttgcgtgccgagcctggcgtgtatcatcagcaattgttggctactttggtgttgtcgtctaacgggttgcagcagcagctggataggcgtgctatagcggctagggttgcgtttggcgagtctgctgcgtgtgatcctacccctgtggagggtgatcgtgtgatggttccttctggggctgtgtgggctaatcatgccggcgagtggatgttgttgtctccgcggatcgagacgggttctaagtcgatcatgtttggcgggtctgctgtgtatgcttacactatcccgtttgcccgccagtttgctagtccgcctgttgtggtggcgtctatggctacggcggctgggggcacgcagcagattgatgtgaaagcctacaatactactaatcagggttttcagttggcgtttatcacgaatgacgggtctaagccgaatggtgtgcctgcggttgctaactggattgctgttggcgtgtgactgtacaggtgttgtggcggatggtgtgatgttggggggctgtggtgtcgtggtttactcctgcactggtggcctctatttgtaccgcgttggccacggttttgggttctgttcaggcggtcacgtctaaatctcggaggcgtttgcggcgcctgtcggctcaggtggatgcgatggaagagtatacgtggggtgtgcggcgcgaggtgcgaaggtttaacgccgggcttcctgatgatgtggagcctatgcatctccctgatttgcccgagtttttgaaggatactgttgatggtggaggtgagtagggttgagggagttggaggaggagaagcggcagcgccgcaattttgagaaggtttcactggtgttgctgtttttgtcgcttgtactactggaggtggttgctgcgggtgctttacgtttcggggctgtatcctctgagcgggattcggagcaggctagggcccagtcgaatggtacagcggctcggggtttagccagccgtgtgaagcaggtgtgtgcctctggtgggcaggagtcggtgcggcttcaccggtctggtttgtgtgtggatgctgtgcgtactgagcgtagcctgcagggtgcgcctggcccggcaggtgagcgcggcccgcaaggccctgcaggcgctgacggccgggatggtgttaatagttcggctgggctggttggccctgttggtccgcagggttctcccggtttgaatggtgtgaaaggtcctgacgggttgcctggcgcgaatggatcggatggccatgatggtgttccaggtcgtgcaggtgctgacggtgtgaacggggttgacggcgctgatggtaaagatggttcggccggtgaacgcggtgatgtgggcccttcaggtcctgccggcccgcaaggtgcacagggtgaacggggtgagcgcggccccgccggtgcgaacggatccgatggcaaggatggtaaagatgggcgctcggtggtgtctgtgtactgttccgggggccgcctggttgtgaaatatagtgacggtgtggcttctaccatatcgggttcggtggcctgccagggtgtgaaaccgtcgcctatagtgactatatcatcccacaaatagaaaggagtggctgtgatagtaatgtttggtggtggtgtgtggtgagatacattcctgcggcgcatcactcggccggttcgaatagtccggtgaatagggttgtgattcatgcgacgtgcccggatgtggggtttccgtccgcctcgcgtaaaggacgggctgtgtctacagcaaactatttcgcgtccccatcagcgggtggttcggcgcattatgtttgcgatattagtgagacggtgcaatgtttgagtgagtctactatagggtggcatgccccgccgaatccgcatagtttgggtatagagatttgcgcggatgggggttcgcacgcctcgttccgtgtgccagggcatgcttacactcgtgagcagtggctggatcctagggtgtggcctgccgtggagcgtgccgccatcctgtgtagacgtttgtgtgacaagcatggtgttccgaaaaggaagcttagtgcagccgatttgaaggctggtaggcggggtgtgtgcggccacgtggatgttacggatgcgtggcaccagtcggatcacgatgatccggggccgtggtttccgtgggacaggtttatggccgtagtcaacggcggtagtggtagtgaggagttaactgtggctgatgtgaaagccttgcatgatcagattaaacaattgtctgcccagcttactggttcggtgaataagctgcaccatgatgttggtgtggttcaattacagaatggtgatttaggtaaacgtgttgatgccctgtcgtgggtgaagaatccggtgacggggaagctgtggcgcacaaaagacgctttgtggagcgtctggtattacgtgctggagtgtcgtagccgtattgacaggcttgagtcgactgtcaatggtttgaaaaagtgatggtggtttgttgtgggtaaacagttttggttgggcttgtttgagcgtgccctgaaaacttttattcaaacgtttgttgctgtgttgggtgtgacggcgggtgtcacgtatacggcggagtcgtttcgtggtttgccgtgggagtctgccctgattacggccggggttgctgcaatactgtcggttgctacctcgtttggtagtccgtcgtttgtggccggcaaacctaaaaccacggttgtggatgctgggcttgttccaccggatgatgggggcatggttgagccgcactcggtggatgtgtcggatcctggcatgattgagccgattgatgatgcggatgttgccggctatgtgccgaggcgtgcagccgagtctgaggttggcacggtagagtctactgttgcataattgaatatgtgtgtgtgccccagcggtgctgccacgatcgtgtggtggtggccgctggggcactatttttgtgtctatagtattctatgattcgttgttgtctatattttcttcgagcatcttatacaggtggaggcaggcggagatagtttcgttggcctggtctatagcaagattgtcgaggctgacttctttgacggtaaggttcacgttgtcgagtgagattggtacacggtactggttttcgacaccgccaacaatgttttctagctgttgcatgttggtgggctgttgttggatgattcggcgtaccgctgttttgagggcagtgtaggggatattggttgtgttgttcatggttttatcccatccctgtgctgtcgtcgctatcgtctggatagtatctactgtttgcgtagcctgtgagggtgatgagtgtttggtctgcccactgtttcacggtttgtcttgtcaccccgagtcgttgggctgccaccgaataagtttgatcatacccgtatacttccctaaatgctgccaaccgtgccaaatgtttcctctgtttggatggctgacaggtgagggtgtagtcgtcgatggctagctgcaaatcgatcatggtaacaatgttgttgccgtggtgttgtggcgcggttggtgggggtggcattcctggttcgacgctcggtttccatgggcctccgttccagatccattgggcggcttggataatatctgcggtggtgtaagtccggttcatgtgtcatcccctaaacaggttgtcgaggttgtctgggttgctggtggtggtgtcgaatcgtccgacgcagtggcagtagtcgtacatgagtttgataatgtgttggtggtcgccgaggtaggtgtttccgctgatactgtaggtggctgtgccgtctttactgatggtgtatttggcggtgatggtttcgggtgtttcggtgttggtgatgatggcggtggtggtggtgcctacggtttggagtatggtggtttgggttccgtcgtcgaggatggttttaaccatggggggttctccttttagttgcttgtttggttgtcggctagatgaataatatcggataaaggtttcggttggtcgaggtgttgtatggttttgttggctagccgtttggctaccctgtagcacattttggtatagtgtttgttgtctaggttgtggtattgttcccgcaccgcaatatatagtagggagtcttggtacaggtcgtctgcactgattgcggggtagtgtgtggctactttggtgcatgcccggttgagtgtgcgaagatgatggtctgtgacccatccccacgatgcggtggtggccaggtctgcttttgttggtcgtctgctcatggcactatttcatctcgctatctgatagttgtttggtgttttgttgtggatagtgtagcacactagtccggggttgccggtggtgcctgtgcggtgccggtaccagacggattctccttccatggatgggcattggatgaaggtgcgttgtccttgctcggatatttctaggtggtgtcggtggcctgccatgaggatgtgggatgtggtgccgttgtggaattcttggccgcgccaccattcgtagtgttggttgttgcgccattggtggccgtgggcgtgcaggatttgtgtgcctgccacgtctacggtggtggtcatttcgtctcggctggggaagtggaagtgaaggttgggatattggttgttgagctggtaggcttctgcgatggcgcggcagcagtctacatcgaaggagtcgtcgtaggtggtgacgcctttaccgaaacgcacggcttcaccgtggttgccggggatggatgtgatggtgacgttggcgcagtggtcgaacatgtggacgagttgcatcatggccatgcgggtgagcctgatttgttccgtcaagggtgtttgtgtgcgccaggcgttgttgcctccttgtgacacgtatccttcgatcatgtcgccgaggaatgcgatgtggactcgttcgggtttgcctgcctgttgccagtagtgttttgcgactatgagggagtgcaaatagtcgtcggcgaagtgtgatgtttccccgccggggatgcctttgccgatttggaagtctcccgcccctaccacgaacgcaacattgctatagtcggtgtgggtgttgtcggctggttttgggggtgtccattcggctagtttatcgacgagttcgtccacgggataggggtttgttgcgggttggtggtcgatgattttttgtatggatcggccggtttctccgttggggagtgtccattcggagatgcgtgtgcggcgtacggtgccgtttgcgagatcatcatcgatagtgtcgattgcgttgtcgtggttggctagctgtgtgaggagccggtcaatattgtctatcatcggatatcctcttctgtttgcggggtggtgttggcttgtttgcggcgatagtctttaataacggtggcggagatggggtatcctgcctgggtgagcattcgggctagctgtgtggcggggatggttttgtcggcgagcacgtcggcggctttgttgccgtagcgttggatgagtatttcagttttggttgccatgatgtcctatcggttgtgtggtgggccgccatcctgtgtggcagtcgccgtcgtgtcctggttttcgggtgcaccacgatatggttccgtctgtgtggtggagtgttttaccgcacatgacttcttgtagatgctccggcagctggtcggtgttgttgctggtttgtgtgtcgaagagtgttttttgtgtggtgaaatgctcggatacggtgccattatgcacgggtagtatccatgttttccattgctgctgtcgccgggtgttccagtggaattgtttggccgcattttcggcctgtttggcggttttgtagtagccgactagtatgcgttgatgctgctggtcgggagggtttgggcctcgccagtattgtgccgctacagcgtagcggttgctggctgtgaaggcaccccagcagtattcgataatgtgctggagcacactgtctggcagactgtcagggttgatggtggtgttttggacgatgatgccgcggatggcttgccggtttttggtggtgggtttgaacgagatactcacgatagtaccggctggtcgtcttgcatgaactggttgaaggtgttgttcccggcgtgttgggcttgtgtgatttgctggtcggtccagtctgggtgttgctgtttcagatagtgccagtggcacgcattgtaggtttcgtcttgtagccgtgtgagatggttttcggtgatgatttgtttccacatggcccatgacacgtcgagccggttgaggatttcgagggcgggaatgttgaattggttgaggaagaggatttcgtgggtgtagtagtttttctcgtattggtcccatccgcttcggtgcctgttgggctggtttttggggtaggcttcccggcatactttgtgtaactgtttggccatgtcgtcgggtagtttaatgtcggggttggcgcggatcatggatcgcatcccatcataggtggtgccccaggtgtgcatgatgtaggtggggtcttcaccatcagcccatttttctgcacagatggcgaggcggatacgcctcctggctgcttggctaatgttgcgccggttggggattgggcacgtgtcgaggggatccatgatgctttagtgtacctttcttgaggtgatgtttgtttgctttgtgtggttttattgtagcactgtgttgagggcttgtgtcaaccctgttttgccggcctgaaggtaggtgtctgtgacatcccccagggtgagggtcacatgggtggcttgggggagcgcggtttggagggtttgggccatctggtcgcctgcagggtcggggtcggaccatatgtagatgtggtcgtagccttcaaaaaatttggtccaaaaagtttgccacgaggtggccccggggagtgctacggctggccatccgcattgttcgaggatcatggagtcgaattcgccttcgcaaatgtgcatttcggctgccgggttggccatggcggccatgttgtagatggagcccgtgtctcctgccggggttaagtatttggggtggttgtgggttttgcagtcgtgcgggagtgagcagcggaaacgcatttttcttatttcggctgggccgccccaaacggggtacaggtatgggatggtgatgcactggttgtagttttcgtggcctgggatggggtcattgtcgatgtatccaaggtggtggtagcgggctgtttcttcgctgatgcctcttgctgagagcaggtcgagtatgttttcgaggtgggtttcgtagcgggctgaggctttctggattcggcggcgttccgcaatgttgtatgggcgtatgctgtcgtacattcgggttttcttcttctaatcgttgtttcagttgggcgagtccgcctccgacaccgcatgtgtggcagtaccagacgcccttgtcgaggttgatgctcatggagggctggtggtcgtcgtggaacgggcagaggatgtgttgctcgttcctggacagattgtaccgtatctggtaggtgtcgaggaggcggcaggtgtcagaggtgtgggaggagctcgttgagggttgataccacataggcttcgctccatggcttgttgcgctgtttcatcactacgagtccgatggtggaattgttttgtttgtttcggtgtgtttcgtagttgcgtgcctcccggctggcttgttttacgaattgggctaggtgtggttgcccagctttcgcctcgataatgtaggttttatggccggttgtgaggatgaggtcgccttcgtcttcgcggccgttgaggtggaggcgttcgatattgtgtccggtgtcgcgtagctggtgcaataatcgtgtttcccattcggctccggcccgccggttgcgtgcctgctgtgtggccatagttttttaaagtcctttgtgtgttgtggtcatgttccagggctgtttttcggcgagtggcccgaagaatgtgtattcggggtaggctcgcagtcgttcgtatcgggtgccgtcggggctggatttgcctgtgcgctgtttgagtacggcgatgcgtgcctctgccggtatcgataggccgttgccgttatcctcaccgccataaagtgatactccgaggatgagttgtggtttttcggagaggccgtttttgatttctcgccgtgctggcgggtgttctatgtcggtgccggttttgtcggtggcgtgatgggtgacaataatggtggagccagtatccctgcccaatgctgtgatccattgcatggcttcttgctgtgcctggtagtcggattcgcagtcttgaatgtccatcaggttgtcgataacaatgagtggtgggaaggtgttccacatttccatgtaggcttgtaattccatggtgatgtcggtccaggtgatgggtgactggaatgagaatgtgatgtgttggccgtggtggatgctatctcgatagtattctggcccgtagtcgtcgatgttttgttgtatctgggcggtggtgtgttgggtgttgagtgagatgattcgtgtggaggcctcccagggtgtcatgtcccctgatatgtagagggcgggctggttgagcatcgctgtgatgaacatggctagccctgatttttggctgccggagcgccccgcaatcatgacgagatcccctttgtggacgtgcatgtcctggttgcggtagaggggttctagctggggtatgcggggcagctcggctgcggtttgggaggctctctcgaaggatctttggagagagagcatcggagcctttatctatctgtttgttgggtgtgttttggtggtcagatggagtcgatatcgatgtcagtagaggctgtggtgtcgtctagctggccgttatcgcgcttgtctacgtattcggcaaccttatcatagatggcgtcgtctaatggtttgagcacgaccgcgttgaacccgtttttggtgcgtacggtggctagtttgaaggcctgctcctcgccaaggtatgcctctaggtcgcggatcatggagtgtgggcggtcgttgttgccgcgtgctttctcaataatggcgttggggatggtttctggggtgccgttgttgaggtcgtctagggtgtggaagatggtgacatcggcgtagatgcggtctgcggtctgtccgccgtagccttcagtgttgtgttctacgtcgtggactttgaaggcgatggcggtggcatcctggtttcgggacgggttgaagaaggtgctgttgctgttgttgcggtagttggcgagtgccatgattgtgttatcctttactgttgtgtctgttattgttgtcttatattggtttatcgggtgaggctgtttcgtttgctgcggaaagcctcggaaacgtcactgttactggtgatgatcttcttgtactgtttcagaaggtcggctagctgtgccttgcttgttgcattgttgattttgttgatgacgatggtgttttctttggatgcgattttgttgacgtagtctttggctgcctggttgtatcggtcttgaaggatgattgatgcgctcgctacgagtgttgctagatcccagtctttggacacgtcatcgtttttgagtccgcctagcaggtcgatgatggcctgttttgtctgctctgctgtgtctcctcggatgaccgcccatggtgcagcatagtctccaccatatttgagtgtgatcgtgagtcgatcattgtcgatcttgtctttatcggtcatttggtgtccttttccttattgtctgtttctggtgtctgtacggtggattctaccgggtatctgtacgagtttttgccgtctacggcccagcaggcgtctcgtacggggcatcctttacagagtgttgtgacgtgtgggacgaagatgccttcgctgattcctttcattgcttgactgtacatggatgatacatgccggtaggtgttgttgtcaaggtcgtagagttcggtggatgtgccttgtgtcggggacttgtcgtcgttgcggctggtggctggcgtccaaaacatgccttttgtcacatcgttgccgtgttggttgagcatgtaccggtatgtgtgcagctgcatactgtcggcgggtagacggccggttttgaggtcgaggatgaaggtttcgccggtgtcggtgtcggtgaagatacggtctatgtagccaacgatctgggtgccgtcggggagggtggtttcgactgggtattcgatgcccggctcgccgtcgataacagcgatagcatattctggatagtggtttcgccagtgtttccacctgtccacaaagattgggccgtaaaccatccaccagtcatagtctttcttgtgtggcccgcccgactcgcacatgtttttgcatattctgccggagggtttgatttctgtgccttcggattcggcgagggctacttgtgtggcgaaaacgtttttaaaggatgcgagttcgcctggcagtgcagggtattcggcgggattgtacaggtgtaggtcgtattgttcggtgatgtggtgtatggcgcttccggcgatggtggcataccaggtatggtgttgggcgtggtagccgtgggataggcgccatttttcaccgcattcagcccactgagagagtgaactgtaggagatgtggcctgggtggctgatggttttcgggtattgtgctagaggcattactggtcgcctttgtgggtgttccatgggttgcgggtgtcttggccggcgtggtgttgctggtatgcgaggagtgcgaggcagtgccaggctgcgtgtgccagatgtggcaaatgtgattcgtggtcgaggttgtttccttgctgccatgatagtaggtgccggtagagggcgtcgacactgtggctccacgggtatcctccgatccagttgttgtcgccgtatttggtggcaccgtatccggctacttcgccgagggcgtggagggatgcggggtcgatgagggatagcctgcattgtttgagttcttttcgggctccggtgttggggtcggtgtacatgcgggttggctcatccatggggtgtgtgctccttaagcgtgggttactggttgttgttgtgggctagggcgacggcgaggatgatgatggcgagggtttcagcgatcagtatgggtgttgtgatcatttgtggtctttgggctggtaggtgagggttgaggcgcccaggagggtggtgagggcgcatgcggcaataatggcgagggcggctttgtgtggggtgccggtggcgtacatccatgtgatgatgccgccttggatccaggctaggctggtgaagaacgtttcgtaactgtgcaactcgatactgttgttgctggtgatgtcattcatggtagttttctgctttgtgtgcgatggttgtgtaaatgtcgttgagtgtggtttcgatagtgatgagagtattgatttcttggctgaggtcgatattgtctttgagggtgtcgatgcgggcggcgatatcggtggcggtgcgtaggcttactgctgcaccgtggacgatgtggcacatgtcggtgaggccgaccttggcgatgtagtgtgacatgagaggcatgggaggtgtgctgtctttcgggttagcgtgacgggttgatggacatgtcttctacctgtggtttgtcttcggtgccggagacttggcagaagactttcacgtgtgtcttggatgctccgggttgtttggcggtggcaccgtaggcgatagtaaaagtgtctttgtgggcgccgatgactttgtgtaggaagaggtcgatgtcggggtttccgttccatgtgacaccgttttctgcggctgcctgggtggctttgcggttgcaggcgtgtgcggcggtgagcatggtcaggcccttgctggtttcttcaccccttgcttgggcttgccggtgggtgcgctgctgttctgcttgtagggagcggactgctgcggcctggcgggctttcttttcggctttgcgctgttggacggtttttggtgtccattcggtgttggctgtggtggcttgtggtgcgggttgtgatgcgagtggcgggttatcgtctggggctggcaggaaggaggctgcggcgatgatggcgatcgtggcgccggcgatggtgtagccttttttcttgttcatggctgttgtcccctttccggggtgttgttcgttgctgacatgatcaatgatggtgtggatggtggcccatgtcaaggtgtcgctcaaccggtgtgagcgatccttgtgtggctaggggttttatcgggcacacaaggtgagtagatggcctacgttgatgcggctcacattccagtagagttgcgtggcttcaccgccggtgagtggcttccactcgttgtggctgaatacggtgccgtcggtggcgatgaacgtattggggcgtagcttgtggagttcggcttccacgctctgccggtaggcttcggcgaggccctcaaaatccatgtggtcgcaggagaggttttcgaggcgtgtcaggtcgaagggtgtggggcagtcgtagctggtgggggtgtagagctgggtgaagtggttggcgatcttctgcatgacgggttcctttctgttgtgtggatggtttttatcgtgtggcttcttggaggattgtggtgatgtcgatggtgtcgatctcccaggcgagttcctcggcctcatccgccgtgagctgttgccagtcttgtgggccggtgactgatccgtcgagggcgaggatccagtcggggcggaagaggtttaaggcttcttgcacagcatcccggtaaacgtgtttggcggtgtcaaggttgatgtgtttggcgtgtcggccggctagctgggtgaggtcgagggggtcgatttctgtctgcccgtagagggaggtgatggatggggtgatgagtgtggtggccatttttggtgtgtcctttcggtggtgtatgggttgttgtggtttctagagtgtgtaggttgcgaccccaccgtcaaggctgcgctcaaacccagtgagcgtttcatggggtgtgtcggatgtgacagatgtcacttaagcctttattacctctctcggcgcctgaaatatgtccggggtaggattatatagggttggccctgctggtcgattttaggcccctttctgtggctctgaggggtatgtctgggtatggaatctacaccctcataccgtgtgagatgtatcccactcacctagtatggtgtgcaccctcaagactactctgccgatctagcgtgaagggtgtagcccagaaataccgtttaaagccttcacacggcgcctaggagcgccttgcagggtggggctaggtgtttatacccccagcatattctgatcgattctagacgcctacaggagcctgatacgcgatccgctatccagacgcaaatcatcagcccctatcctggttagctaagcctcaactatgtggacagtgtgggatactgtgagggaagaaggacacggtaaaagaaagaagggggagcatcaaccttcacgcctgaggtacttaagttcacctgagggtcttagcactgagcccctcaagggctcggcatcagtccgagcaggctcagctcatcaggcacagcactgaaaggggtacacgccatcagggaaggcttgagagtacgaggagccctagcgacgagtactagaaagcctgagggaacaccctcagcactgatgggcctagcatgttcggaaaggacacaggagtacagtgtgacagtcttaccgggagtgaaacccgttctgactaggggtttcagccttaaccacctgtaaaggttacaagactctaagaaaatttaaggaaaagtttaggtttaatttttggacctttactaccaaaaacacccctttacacccctcaaacccgcctatagagccaaatccaccagtttgactcatcccaggtggggtatgataggctggacaggtagccagctggacgcaaggccgaaatccgctgacgcggctttcacccttacatccatcagtctaccaaacactttaaagcttcagtgctaagcgctaagccactcaacacttagcactgagcccttcaaggctcagcatcagtataaagaccttaacacttaatttaatatttaaaccttaaaggctaagcacttaaggatataaacttaacatcagtgtttaagactttaatactttaagtaactataaaacattaaagaccttaagtacttaaagttaatcatcagtcttaaactttaatattatacctataagctttaagacttataggtataataatataatataagtattaaagcttataagttataaaagttttagaagagctaaggggttaacttctttacttctctactctctttggtactttctctcttctcttcttttcttcatcaggggagaagaggaaccttttaccatcagcgccgatgtactttcaccgtgtgactcgtgtaccaccggtcgcacgctcccggttgcacactccccacactcttacacccgtgtccctttcaggcttggcgtgttcggctgaaggcgtacggcgtgtcacgctaacacccttaacaccaggtaagacttaaagtacatattatatgtagaagactttaaaacctataaggtgtttctgctgagcccgcgtcctacaccgctaagcgctaagccgtgaaacgcgaacacccacccccatttttcttccgtgtccttcttcttttgacacagctggggggcgatgtgatctttctcacactcatgggggtagtggagaaaacaaacacccccttcaaacgaacaaaacagggcctaggatcgactagcagggcaaggtattcatacccccaacggttcccaagccgctagagaggcaatgagaggctcacaggggccataggtgatcgggggacgcgatggcacacactaaccgcaccgcatccgcctcacaccgacgctggcggcaacgactcatcacccaagccaaacagcaaggccaaaccgaatgcccactctgcggagccaccatcacctgggacacacaccagctaccaaccagccccgaagccgaccacatcacacccgtcagcggggaggactcaacaccctcgacaacgggcaaatcatctgcagaacatgcaacagaagcaaaggcaatcgcagcgaaccaaacatcaaattccaacaacaaaccacaaaaaaccttgttccatggtaaaaacccgccaacccccaccggggacaccccctgcacacccgtgcaag

SEQ ID NO: 7 - protein, 29168 aa, Propionibacterium acnes
  REGION 1..29168: note = bacteriophage MBPAC3
GAAATACCTCCTTTTGTGGATTTGTCTGTTTGTCGAATTTTTGTGTTGGTGGTGAGTGTTGTGCAGCCTGAGCTTCCTGATAGTCGTGAGTGGTGTGGGGAGACGCGTCGTTGGTGGCGTGTGGGGTGAGGATAGTCGCGCGCAGTACGTGTCTGATGAGGAGTGGCTGTTTCTTCTTGATGCGGCGGTGATTCATGATTGTGTGTGGCGTGAGGGTCGCGCGGATTTGGTGGCTTCGCTTCGTGCTCATGTGAAGGCTTTTATGGCCATGTTGGATCGTTATTCGGTTGATGTGGTGTCTGGTGGCCGTGGTGGGGGTTCTGCGGTGGCGATGATTGACCGGTATAGGAAGCGTAAAGGGGCCTAATGTCGAGCGTTGTTGGTTCTCAGGTTCCTCGTCATCGTGTGGCTGCGGCGTATTCGGTGTCTGCTGGCGGTGATGCTGGTGAGCTTGGTAGGGCTTACGGGTTGACGCCTGATCCGTGGCAGCAGCAGGTGTTGGATGATTGGCTGGCTGTCGGTAGCAATGGCCGGCTTGCTTCGGGTGTGTGTGGGTGTTTGTGCCTCGCCAGAATGGCAAGAATGCTATTTTGGAGATTGTGGAGTTGTTTAAGGCGACTATTCAGGGTCGCCGTATTTTGCATACGGCTCACGAGTTGAAGTCGGCTCGTAAGGCGTTTATGCGGTTGAGGTCGTTTTTTGAGAATGAGCGGCAGTTTCCTGACTTGTATCGTATGGTTAAGTCGATTCGTGCGACGAATGGTCAGGAGGCTATTGTGTTGCATCATCCGGATTGTCCGACTTTTGAGAAGAAGTGTGGCTGCAGCGGTTGGGGTTCGGTTGAGTTTGTGGCTCGTTCTCGGGGTTCTGCTCGCGGGTTTACGGTTGATGATTTGGTGTGTGATGAGGCTCAGGAGTTGTCGGATGAGCAGTTGGAGGCTTTGCTTCCTACGGTGAGTGCTGCCCCGTCTGGTGATCCGCAGCAGATTTTTCTTGGTACGCCGCCTGGGCCGTTGGCGGACGGTAGCGTGGTGTTGCGTTTGCGTGGGCAGGCGCTTGGTGGTGGTAAAAGGTTTGCGTGGACGGAGTTTTCGATTCCTGACGAGTCTGATCCGGATGATGTGTCGCGGCAGTGGCGGAAGTTGGCGGGGGATACGAATCCTGCGTTGGGTCGTCGCCTGAATTTTGGGACAGTCTCGGATGAGCATGAGTCGATGTCTGCTGCCGGGTTTGCTCGGGAGCGGCTTGGCTGGTGGGATCGTGGCCAGTCTGCTGCGTCGGTGATTCCTGCGGATAAGTGGGTCCAGTCGGCGGTGGATGAGGCGAGTCTGGTTGGCGGGAAAGTGTTTGGTGTCTCGTTTTCTCGCTCGGGGATCGTGTCGCGTTGGCGGGTGCCGGCCGGACTGATGCTGGTGTGCATGTTGAGGTGATTGATGGCCTGTCGGGCACGATTGTTGATGGTGTGGGCCGGTTGGCTGACTGGTTGGCGTTGCGTTGGGGTGACACTAACCGGATCATGGTTGCCGGGTCTGGTGCGGTGTTGTTGCAGAAGGCGTTGACGGATCGTGGTGTTCCGGGTCGTGGCGTGGTGGTTGCTGATACTGGTGTGTATGTGGAGGCGTGTCAGGCGTTTTTGGAGGGTGTAAGGTCTGGGAATGTTTCTCATCCTCGTGCTGATTCTCGCCGTGACATGTTGGATATTGCTGTGAGGTCGGCTGTGCAGAAGAAGAAGGGTTCTGCGTGGGGTTGGGGCTCGACGTTTAAGGACGGCAGTGAGGTGCCGTTGGAGGCTGTGTCGTTGGCGTTTTTGGGGGCTAAACGTGTTCGTCGTGGCCGTCGGGAGCGTAGTGGTAGGAAGCGGGTGTCTGTGGTATGAACTCGGATGAGTTGGCTCTGATTGAGGGCATGTACGATCGTATCCAAAGGTTGTCTTCGTGGCATTGTCGCATTGAGGGCTACTATGAGGGCTCTAGCCGGGTGCGTGACCTTGGTGTGGCTATTCCGCCGGAGTTGCAGCGGGTGCAGACGGTGGTGTCGTGGCCTGGTATAGCTGTGGATGCTTTGGAGGAGCGTCTGGATTGGCTTGGCTGGACGAATGGTGACGGCTACGGCCTAGATGGTGTGTATGCTGCGAATCGGCTTGCTACGGCTTCGTGTGATGTGCATTTGGATGCGCTGATTTTTGGGTTGTCGTTTGTGGCTGTTATTCCCCAGGGCGATGGGTCGGTGTTGGTTCGTCCGCAGTCACCAAAGAATTGTACTGGCCGGTTTTCGGCTGACGGGTCTCGTTTGGATGCGGGTTTGGTGGTGCAGCAGACGTGTGATCCTGAGGTTGTTGAGGCGGAGTTGTTGCTGCCTGATGTGATTGTTCAGGTGGAGCGGCGGGGTTCGCGTGAATGGGTTGAGGTGGATCGTATACCGAATGTGTTGGGTGCTGTTCCGCTTGTGCCTATTGTGAATCGTCGCCGTACTTCTAGGATTGATGGCCGTTCGGAGATTACGAGGTCTATTAGGGCTTACACGGATGAGGCTGTTCGCACACTGTTGGGGCAGTCTGTGAATCGTGACTTCTACGCCTACCCGCAAAGGTGGGTGACTGGCGTGAGTGCGGATGAGTTTTCGCAGCCTGGCTGGGTCCTGTCGATGGCTTCTGTGTGGGCTGTGGATAAGGATGATGATGGTGACACTCCGAATGTGGGGTCGTTTCCTGTCAATTCGCCTACACCGTATTCGGATCAGATGAGACTGTTGGCGCAGTTGACTGCGGGTGAGGCGGCTGTTCCGGAACGCTATTTCGGGTTTATCACGTCTAACCCACCTAGTGGGGAGGCTTTGGCTGCCGAGGAGTCTCGGCTTGTGAAGCGTGCTGAACGCAGGCAGACGTCGTTTGGTCAGGGTTGGCTGTCGGTTGGTTTCCTGGCTGCCAAGGCGCTTGATTCGAGTGTTGATGAGGCCGATTTTTTTGGTGATGTTGGTTTGCGTTGGCGTGATGCTTCGACGCCTACCCGGGCGGCTACGGCTGATGCTGTGACGAAGCTTGTTGGTGCCGGTATTTTGCCTGCTGATTCTCGTACGGTGTTGGAGATGTTGGGGCTTGATGATGTGCAGGTTGAGGCTGTGATGCGTCATCGTGCTGAGTCGTCTGACCCGTTGGCGGCACTGGCTGGAGCTATATCGCGTCAAACTAACGAGGTATGATAGGCGATGGCTTCGGGTGCTATGTCGAGGCTTGCTGCGACTGAGTATCAGCGTGAGGCGGTCAGGTTTGCTGGGAAGTATGCGGGCTATTATGCCGAGTTGGGTCGTTTGTGGCATTCCGGGAAGATGACAGATGCGCAGTATGTGCGTTTGTGTGTGGAGTTGGAGCGTGCCGGCCATGATGGTTCGGCATCGATGGCAGCCCGATTCGTGCAAGATTTTCGCCGGTTGAATGGTGTGGATCCGGGTTTGATTGTGTATGACGAGTTTGATGCTGCGGCGGCTTTGGCTAGGTCGTTTTCGACTATGAAGATTCTTGAGAGTGACCCGGATAGGGCGAATGACACGATTGATGCGATGGCTGCGGGTGTTAATCGGGCTGTCATGAATGCTGGCCGTGACACGGTTGAGTGGTCTGCGGGTGCGCAGGGCAGGTCGTGGCGCAGGGTGACTGATGGCGATCCGTGTGCTTTTTGTGCCATGTTGGCTACGAGGTCGGATTATACGACCAAAGAAAGGGCACTTACTACTGGTCATACTCGGCGTCATAAGCGTGGTGGTAAGCGTCCGTTTGGTTCGAAGTATCATGATCATTGTGGTTGTACGGTGGTTGAGGTTGTTGGCCCTTGGGAACCAAATAGGGCTGATGCCGAGTATCAGAGGACGTATGAGAAGGCCCGTGAGTGGGTTGATGATCATGGGTTGCAGCAGTCGCCTGGCAATATTTTGAAGGCTATGCGTACTGTTGGCGACATGAGATGATGGTTTCCGGTTGTGCGCCGCCGGTTATCGGTGCACAAGGTTGTCTCCCGCACGGGGGTCAACAATGTTGTGTTGTTTTCCGCAAGGAGTGTAGGGTTAGGCTATGGCCGATCAGAGTGTTGAGGAACAGAATGTGGACAATGATGTTGTGGAGTCCGGAAAGGATAACGGCATTGTTGATACAGTAAAAGACGATGGCGGGCAGGAGGTAGCCGACAATCAGTTGAAGAATGAAGGCGAGGGTAAATCGCCGGGGACTGATTGGAAGGCGGAGGCCCGTAAGTGGGAGTCTCGTGCTAAAAGTAATTTTGCCGAGTTGGAGAAGCTTCGTGCATCGAGTGACGATTCTGGATCTACTATTGATGAGCTTCGCCGCAAGAATGAGGAACTCGAAGACAGGATTAACGGGTTTGTTCTTGAGGGTGTGAAGCGCGAGGTTGCTTCAGAGTATGGTTTGTCCAGTGATGCGATCGCTTTCTTGTCGGGTGGCGATAAGGAGTCGCTTGCCGAGTCTGCTAAGGCTTTGAAGGGTTTGATCGACCATAGTAGTGGTGGCGCGGGTGTGCGCCGTCTTGCGGGGAGTGCCCCCGTTGATGATGTTAAACGACGTGAGGGTGTCGCGTTTGTGGATGCTCTTGTCAATAATTCTAGGAGATGATTTGTGATGGCTGACGATTTTCTTTCTGCAGGGAAGCTTGAGCTTCCTGGTTCTATGATTGGTGCGGTTCGTGACCGTGCTATCGATTCTGGTGTTTTGGCGAAGCTTTCGCCGGAGCAGCCGACTATTTTCGGCCCGGTGAAGGGTGCCGTGTTTAGTGGTGTTCCTCGCGCTAAGATTGTTGGTGAGGGTGAGGTTAAGCCTTCCGCGTCTGTTGATGTTTCGGCGTTTACTGCGCAGCCTATCAAGGTTGTGACTCAGCAGCGTGTGTCGGACGAGTTTATGTGGGCTGATGCTGATTACCGTCTGGGTGTGCTTCAGGATCTGATTTCGCCTGCTCTTGGTGCTTCGATTGGTCGCGCTGTTGATCTGATTGCCTTCCACGGTATTGATCCGGCTACGGGTAAGCCTGCTGCGGCTGTCAAGTCTTCGCTGGATAAGACGAAGCATATTGTTGATGCAACCGATAGCGCTACGGCTGATCTGATTAAGGCGGTTGGGCTGATTGCTGGTGCTGGTTTGCAGGTTCCTAACGGGGTTGCTTTGGATCCGGCGTTCTCGTTTGCACTGTCTACTGAGGTGTATCCGAAGGGGTCTCCACTTGCCGGCCAGCCTATGTATCCTGCCGCCGGGTTTGCTGGTTTGGATAATTGGCGTGGCTTGAATGTTGGTTCTTCTTCGACTGTTTCTGGCGCCCCGGAGATGTCGCCCGCCTCTGGTGTTAAGGCTATTGTGGGTGATTTCTCTCGTGTTCATTGGGGTTTCCAGCGTAACTTCCCGATCGAGCTTATCGAGTATGGTGACCCGGATCAGACTGGGCGTGACCTGAAGGGCCATAATGAGGTTATGGTTCGTGCCGAGGCTGTGCTGTATGTGGCTATCGAGTCGCTTGATTCGTTTGCTGTTGTGAAGGAGAAGGCTGCCCCGAAGCCTAATCCGCCGGCCGAGAACTGATCTATTTGTTGCGATAATGTTCATGCTGTGTGCAGGGGTGGTGTTGATGGGTATCATTTTGAAGCCTGAGGATATTGAGCCTTTCGCTGATATTCCTAGAGAGAAGCTTGAGGCGATGATTGCTGATGTGGAGGCTGTGGCTGTCAGTGTCGCCCCCTGTATCGCTAAACCGGATTTCAAATACAAGGATGCCGCTAAGGCTATTCTGCGCAGGGCTTTGTTGCGCTGGAATGATACTGGCGTGTCTGGTCAGGTGCAGTATGAGTCTGCGGGTCCTTTCGCTCAGACTACACGGTCTAGTACTCCCACGAATTTGTTGTGGCCTTCTGAGATTGCTGCGTTGAAGAAGTTGTGTGAGGGTGATGGTGGGGCTGGTAAAGCGTTCACTATTACACCGACCATGAGGAGTAGTGTGAATCATTCTGAGGTGTGTTCCACGGTGTGGGGTGAGGGTTGCTCGTGCGGGTCGAATATTAACGGCTACGCTGGTCCTTTGTGGGAGATATGATATGACCAGTTTTCCTTATGGTGAAACGGTTGTGATGCTTCAGCCGACTGTTCGTGTCGATGATCTTGGCGACAAGGTGGAAGACTGGGGGCATCCTGTCGAAACCGTGTACCATAACGTGGCCATCTATTCTTCGTTGTCGCAGGAGGATGAGGCTGCGGGGCGTGACTCGGATTATGAGCATTGGTCGATGCTTTTCAAGCAGCCTGTCAAGGGTGCCGGTTATCGTTGCCGGTGGCGTATTCGGGGTGTTGTGTGGGAGGCTGACGGGTCTCCTATGGTGTGGCATCATCCGATGTCTGGTTGGGATGCGGGCACGCAGGTTAAGGTGAAGCGTAAGAAGGGCTGATAGATTGTGGCTCAGGATGTGAATGTGAAGCTGAACTTGCCGGGTATTCGTGAGGTGTTGAAGTCTCCTGGGGTGCAGGCTATGTTGGCTGAGCGTGGCGAGCGTGTGAGGCGTGCGGCCTCGGCGAATGTGGGCGGTAACGCTTTCGATAAGGCCCAGTATCGTGCAGGGTTGTCGTCGGAGGTGCAGGTTCACCGGGTTGAGGCTGTGGCCCGTATTGGCACCACCTATAAGGGTGGGAAGCGTATTGAGGCGAAGCATGGCACGTTGGCTCGTTCGATTGGGGCTGCGTCGTGATCGTTTATGGTGATCCGCGTGTGTGGGCTAAACGTGTGCTCAAGGATGATGGCTGGCTGTCCGATATACCGTGTACTGGGACGGTGCCTGACCAGTTTGAGGGTGACCTTATTTGGTTGGCTCTTGATGGTGGCCCGCAGTTGCATGTGCGTGAGCGTGTTTTTTTGCGGGTGAATGTGTTTTCTGATATGCCTGATCGGGCTATGTCGTTGGCGCGTCGTGTTGAGGCTGTGCTGGCTGATGGTGTGGACGGTGATCCTGTGGTGTACTGTAAACGGTCTACTGGCCCTGATTTGCTGGTTGATGGTGCACGTTTTGATGTGTATTCGCTTTTTGAGCTGATATGTAGGCCGGCGGAGTCTGAATAAGCTTATTGTTTTTGTTTTAATGTAATTGTTTGATATTTAATGGGGGTTGTGATGGCTGCAACACGTAAAGCGTCTAATGTTCGCTCAGCGGTTACTGGCGACGTTTATATTGGTAAAGCTCATGCTGGTGACACTATTGATGGTGTGAAGACGGTTCCTGACGGTCTTACCGCTTTAGGGTACCTGTCTGATGACGGGTTTAAGATTAAGCCTGAGCGTAAAACGGATGATTTGAAGGCTTGGCAGAATGCGGATGTTGTTCGCACTGTGGCTACGGAGTCTTCTATCGAGATTTCTTTCCAGCTGATCGAGTCTAAGAAGGAGGTTATCGAACTGTTTTGGCAGTCGAAGGTTACTGCCGGAGCCGATTCGGGTTCGTTCGATATTTCTCCTGGTGCCACGACGGGTGTTCACGCCCTGTTGATGGATATTATTGATGGCGATCAGGTTATTCGCTACTATTTCCCTGAGGTTGAGTTGATCGATCGTGACGAGATCAAGGGCAAGAATGGCGAGGTGTACGGGTATGGTGTGACGTTGAAGGCGTATCCTGCCCAGATTAATAAGAAGGGTGATGCGGTGTCTGGTCGGGGGTGGATGACGGCTTTAAAAGCTGATACTCCCCGGTTCCTCCTTCTCCGAAGCCTGAGCCGGATCCTAATCCGCCGTCTAATAACTGATACACATAGTTTGAGGGATTGTTGATAGATGAGTGACACAGGTTACACGTTGAAGATTGGTGACCGTAGCTGGGTGTTGGCGGATGCGGAGGAGACGGCTCAGGCTGTTCCTGCCCGCGTGTTTCGCCGTGCCGCCAGGATTGCCCAGTCTGGGGAGTCTGCTGATTTCGCCCAGGTTGAGGTGATGTTTTCCATGTTGGAGGCTGCCGCCCCAGAGGATGCTGTGGAGGCCTTGGAGGGGCTTCCTATGGTTCGTGTGGCCGAGATTTTCCGCCAGTGGATGGAATATAAGCCTGAAGGTAAGGGTGCCTCTTTGGGGGAATAGTTTGGCTCCACGGCCTGATTGATGATTATCGTGGGGCCATCGAATATGATTGGAGGACCCGGTTCGGTTGCTCGGTTTATGATGTTGGTGGCCCGCAGATGTGTTGGGGTGAGGCTGTCCGGCTGGCTGGCGTGTTGTGTACCGATACGTCTAGCCAGTTGGCGGCCCACCTGAATGGTTGGCAGCGCCCGTTTGAGTGGTGCGAGTGGGCTGTGCTGGACATGTTGGATCATTACAGGTCTGCTAATAGTGAGGGGCAGCCGGAGCCTGTGGCGAGGCCTACGGATGAGCGTAGGGCCCGGTTTACGTCTGGGCAGGTGGACGATATTTTGGCGCGTGTTCGTGCCGGTGGCGGGGTGTCTCGCGAGATTAATATTATGGGGTGAATAGTGTATGTCTGGTGAGATTGCTTCCGCATATGTGTCGTTGTATACGAAGATGCCTGGCCTTAAAAGTGATGTTGGTAAACAGCTTTCTGGGGTTATGCCTGCTGAGGGTCAGCGTTCGGGTAGTCTGTTTGCTAAGGGCATGAAGTTGGCGCTTGGTGGTGCCGCAATGGTGGGTGCCATCAATGTTGCTAAGAAGGGCCTCAAGTCGATTTATGATGTGACTATTGGTGGCGGTATTGCTAGGGCGATGGCTATTGATGAGGCTCAGGCTAAGTTGACTGGTTTGGGTCACACGTCTTCTGACACGTCTTCGATTATGAATTCGGCTATTGAGGCTGTGACTGGTACGTCGTATGCGTTGGGTGATGCGGCGTCTACGGCTGCGGCGTTGTCTGCTTCTGGTGTGAAGTCTGGCGGGCAGATGACGGATGTGTTGAAGACTGTCGCCGATGTGTCTTATATTTCGGGTAAGTCGTTTCAGGATACGGGCGCTATTTTTACGTCTGTGATGGCTCGCGGTAAGTTGCAGGGCGATGACATGTTGCAGCTTACTATGGCGGGTGTTCCTGTGCTGTCTTTGCTTGCCAGGCAGACGGGTAAAACGTCGGCTGAGGTGTCGCAGATGGTGTCGAAGGGGCAGATTGATTTTGCCACGTTTGCGGCTGCGATGAAGCTTGGCATGGGTGGTGCTGCGCAGGCGTCTGGGAAGACGTTTGAGGGCGCTATGAAGAATGTTAAGGGTGCCCTGGGTTATCTTGGTGCCACGGCTATGGCGCCGTTTCTTAACGGCCTGCGGCAGATTTTTGTTGCGTTGAATCCGGTTATCAAGTCGGTTACGGATTCTGTGAAGCCGATGTTTGCTGCCGTCGATGCTGGTATTCAGCGTATGATGCCGTCTATTTTGGCGTGGATTAATCGTATGCCGGCTATGATCACTCGAATGAATGCACAGATGCGCGCCAAGGTGGAGCAGTTGAAGGGCATTTTTGCGAGAATGCATTTGCCTGTCCCTAAAGTGAATTTGGGTGCCATGTTTGCTGGCGGCACCGCAGTGTTTGGTATTGTTGCTGCCGGTGTGGGGAAGCTTGTCGCGGGGTTTGCCCCGTTGGCGGTGTCGGTGAAGAATCTGTTGCCGTCGTTTGGTGCTTTGAAGGGTGCCGCTGGCGGGCTTGGCGGCGTGTTTCGCGCCCTGGGTGGCCCTGTTGGTATTGTGATCGGCTTGTTTGCGGCAATGTTTGCTACGAACGCCCAGTTCCGTGCCGCTGTTATGCAGCTTGTTGGTGTGGTTGGTCAAGCCCTGGGGCAGATCATGGCCGCTATTCAGCCCCTATTTGGTTTGGTTGCCGGGGTTGTGGCACAGTTGGCGCCAGTGTTCGGCCAGATTATCGGTATGGTTGCCGGTTTGGCTGCCCAGCTGGTGCCTTTGATTAGTATGCTTGTCGCCCGGCTGGTTCCTGTGATCACGCAGATTATTGGTGCGGTGACACAGGTTGCTGCAATGTTGTTGCCGGCGTTGATGCCGGTTATTCAGGCTGTTGTGGCTGTGATACGGCAGGTTGTTGGCGTGATCATGCAGTTGGTGCCTGTTTTGATGCCTGTGATTCAGCAGATTTTGGGTGCTGTCATGTCTGTTCTGCCGCCTATCATCGGCCTGATTCGGTCGCTGATACCAGTCATCATGTCGATTATGCGTGTGGTGATGCAGGTTGTTGGTGTCGTGCTACAGGTGGTGGCCCGCATTATTCCGGTTGTGATGCCAATTGTGACAGCTGTGATCGGGTTTGTTGCACGTATTCTTGGCGCTATTGTGTCTGCTGCAGCCCGCATTATTGGGACTGTCACCCGTGTCATCTCATGGGTTGTGAATCATCTGGTGTCTGGCGTGAGGTCTATGGGCACGGCCATCTTGAATGGCTGGAATCATATTAGAGCGTTTACGTCTGCGTTTATTAACGGTTTCAAGTCGGTGATTTCTGGCGGTGTGAACGCGGTTGTGGGGTTTTTGCCCGGCTTGGTTCTTCGGTTGCCTCCCATGTGAGGTCTGGTTTTAACGCGGCCCGTGGCGCTGTTTCTTCTGCGATGAATGCTATCCGGAGTGTTGTGTCTTCGGTGGCGTCTGCTGTTGGCGGGTTTTTCGGGTCGATGGCGTCTAGGGTTCGTAGTGGTGCTGTGCGCGGGTTTAATGGTGCCCGGAGTGCGGCTTCTTCTGCTATGCATGCTATGGGCTCGGCTGTGTCTAGCGGCGTGCATGGTGTGCTAGGGTTTTTCCGGAATTTGCCTGGTAATATTCGGCGTGCGCTTGGTAATATGGGGTCCTTGTTGGTGTCGGCTGGCCGTGATGTGGTGTCTGGTTTGGGTAATGGTATTCGGAATGCTATGAGTGGCTTGTTGGATACTGTGCGTAATATGGGTTCTCAGGTTGCTAATGCGGCGAAGTCGGTGTTGGGTATTCATTCTCCGTCTCGGGTGTTTCGTGACGAGGTTGGTCGGCAGGTTGTTGCTGGTTTGGCTGAGGGGATCACCGGTAATGCCGGTTTGGCGTTGGATGCGATGTCTGGTGTGGCTGGTCGGCTTCCGGATGCGGTTGATGCCCGGTTTGGTGTGCGATCGTCTGTGGGCTCGTTTACCCCGTATGACCGGTATCGGCGTGCGAGCGAGAAGAGTGTTGTGGTGAATGTGAATGGCCCGACGTATGGTGATCCTGCCGAGTTTGCGAAGCGGATTGAGCGGCAGCAGCGTGACGCTTTGAACGCGTTGGCTTATATGTGAGTAAAGGGGTATGCATGTTTATTCCTGACCCGTCTGATCGTTCTGGTTTGACTGTGACCTGGTCTATGGATCCGCGGTTTGGCGATGAGCGTGTGCTTCATTTGACGGATTATACGGGGTCGTCTCCGGTGATGTTGTTGAATGATTCGTTGCGCGGTTTGGGTGTTCCTGAGGTGGAGCATTTTTCTCAAACTCATGTTGGGGTGCACGGCTCGGAGTGGCGCGGGTTTAATGTGAAGCCTCGCGAGGTGACATTACCGGTGTTGGTGTCGGGTGTTGACCCGGATCCGGATGGCGGGTTTCGTGACGGTTTTTTGAAGGCGTATGACGAGTTGTGGTCTGCTTTTCCTCCCGGGGAGGAGGGGGAGTTGTCTGTGAAGACTCCTGCCGGTCGTGAGCGTGTGTTGAAGTGTCGGTTTGATTCGGTGGATGACACGTTTACGGTTGATCCGGTGAATCGTGGCTATGCGCGTTATGTGATTCATTTGACAGCGTATGACCCGTTTTGGTATGGGGATGGGCAGAAGTTTCGTTTTAGTAATGCGAAGTTGCAGGATTGGTTGGGTGGCGGCCCTGTCGGCAAGGATGGCACGGCGTTTCCTGTGGTGTTGACGCCTGGTGTTGGTTCGGGCTGGGATAACCTGTCTAATAAGGGTGATGTGCCTGCGTGGCCTGTGATTCGTGTTGAGGGCCCGTTGGAGTCGTGGTCTGTGCAGATTGATGGTTTGCGTGTGTCTTCGGATTATTCTGTCGAGGAGTTTGATTGGATCACTATTGATACGGATCCTCGTAAACAGTCTGCGTTGTTGAATGGGTTTGAGGATGTGATGGATCGTTTGACAGAGTGGGAGTTTGCGCCTATCCCGCCTGGCGGTTCGAAGAGTGTGAATATTGAGATGGTTGGTTTGGGTGCCATTGTTGTGTCGGTGCAGTACAGGTTTTTGAGGGCTTGGTGAATAGTTGATGGCTGGTCTTGTTCCGCATGTAACATTGTTTACGCCGGATTATCGCCGTGTGGCGCCTATCAATTTTTTGAGTCGTTGAAGTTGTCGTTGAAGTGGAATGGTTTGTCCACTTTGGAGTTGGTGGTGTCGGGTGATCATTCTAGGCTTGACGGGTTGACTAGGCCGGGTGCACGGCTGGTTGTTGATTATGGTGGTGGCCAGATTTTTCTGGGCCTGTGCGTCGGGTTCATGGTGTGGGTCCGTGGCGTTCTTCGCGGGTGACTATCACGTGTGAGGATGATATCCGCCTGTTGTGGCGTATGCTGATGTGGCCTGTTAATTATCGTCCTGGTATGGTTGGTATGGAGTGGCGTGCCGACAGGGATTATGCCCACTATTCGGGTGCGGCGGAGTCGGTTGCTAAGCAGGTGTTGGGGGATAATGCTTGGCGTTTTCCGCCTGGTTTGTTTATGACCGATGATGAGAGTCGTGGCCGCTATATTAAGGATTTTCAGGTGCGGTTTCACGTGTTTGCCGATAGGTTGTTGCCGGTGTTGTCGTGGGCTCGGATGACTGTCACGGTGAACCAGTTTGAGAATGCGAAGTTTGATCAGCGGGGTTTGCTGTTTGATTGCGTGCCTGCTGTGACCCGTAGTCACGTGTTGACTGCCGAGTCTGGTTCGATTGTGTCGTGGGAGTATATGCGTGACGCCCCGAAGGCGACATCTGTGGTGGTTGGTGGCCGAGGCGAGGGCAAGGATCGGCTGTTTTGTGAGGATGTTGACGCGTTGGCCGAGGATGAGTGGTTTGATCGTGTCGAGGTGTTTAAGGATGCCCGTAACACGGATTCTGAACATGTGCATCTCATCGATGAGGCTGAGCAGGTGCTGTCCGAGTCGGGGGCCACATCTGGGTTTAAGATCGAGTTGGCTGAGTCGGATGTGTTGCGGTTTGGGCCAGGCAATCTGATGCCGGGTGATTTGATCTATGTGGATGTGGGCTCGGGGCCTATTGCGGAGATTGTTCGGCAGATTGATGTGGAGTGTGTATCGCCGGGTGATGGTTGGACGAAGGTGACTCCTATTGCGGGGGATTATGAGGATAATCCGTCGGCGCTGTTGGCGCGGCGTGTGGCTGATTTGGCTGCGGGTGTGCGGGATTTGCAAAAATTTTAGAAAAGAATTGGGGGTTTGTTGTGGGTATTGTGTGCAAGGGTTTTGATGGTGTGTTGACTGAGTATGATTGGGCTCAAATGTCTGGTCTGATGGGTAATATGCCGTCTGTGAAGGGCCCGGACGATTTTCGTGTCGGCACTACTGTTCAGGGTGCCACAGTGTTGTGTAGTGTTTTGCCGGGGCAGGCTTGGGCTCACGGGGTGATGTGCACGTCGAATAGTGTTGAGACGGTGACAGAGCCGCTTCCGGGCCCGGGTGAGACCCGATACGACTATGTGGTGTTGTCTCGGGATTGGGAGGCGAATACGGCCAGGTTGGATATTGTTCCTGGGGGCCGTGCGGAGCGTGCCAGGGATGTGTTGCGCGCCGAGCCTGGCGTGTATCATCAGCAGCTACTGGCTACTTTGGTGGTGTCGTCTAACGGGTTGCAGCAGCAGCTGGATAGGCGTGCTATAGCGGCCCGTGTGGCGTTTGGGGAGTCTGCCGCGTGTGATCCTACCCCTGTGGAGGGCGACCGTGTGATGGTGCCTTCGGGTGCTGTGTGGGCTAACCATGCTAACGAGTGGATGTTGTTGTCCCCCAGGATTGAGACGGGTTCTAAGTCGATCATGTTTGGCGGATCTGCTGTGTATGCTTACACGATCCCGTTTAATCGTCCCTTCGGTAGTGCGCCGGTTGTGGTGGCGTCTATGGCTACGGCGGCTGGGGGTACGGCACAGATTGATGTGAAAGCCTACAATATTACTAGCAAGGATTTTCAGTTGGCGTTTATCACGAATGATGGTTCGAAGCCTTCTAATGTGCCTGCGATAGCTAACTGGATTGCTGTCGGCGTGTAATGCGCGGCTTGTGTATATGTGACGTGTTGTGGTGGTTGTAGTGGTAGGGGGCTGTAGTGTCATGGTTTACACCCACACTTGTAGCCTCTATTTGTACCGCTATCGCTACGGTCCTTGGTTCGATTCAGGCGGTTACTTACAGGTCGAAGAAGAGGCTTAGGCAGTTGTCTGCGCAGGTTGATGCGATGGAAGAATACACATGGAATATTCGCCATATTGTTCATCGCTATAACGCGAATTTGCCGGAAAATGTTGAGCCGGTAAAGATGCCTGATTTGCCCGAGTTTTTGAAGGATACTGTTGATGGTGGTGGGGGTGAATTGTGAGGGAGTTGGAGGAAGAGAAACGGCAGCGCCGCAATTTTGAGAAGGCTTCCCTGGTGTTGCTGTTTTTGTCGCTTGTGTTGTTGATGGTGGTTGCTGGGGGTGCTTTACGGTATGGTTCTGTGGCTTCCCAAAGGGATTCGGAGCAGGCTAGGGCCCAGTCGAATGGTACAGCCGCTAAAGGGTTGGCTGCCCGTGTGCGGCAGGCGTGTGCCTCTGGTGGGCAGGAGTCGGTGCGGCTTCACCAGTCTGGTTTGTGTGTGGATGCTCAGCGTGTTGAGCGTAGCGTGCAGGGTGTGCCTGGCCCTGCCGGTGTGCGCGGCCCGCAAGGGCCCGCTGGTGTTGACGGCCGGGATGGTGTTAATGGTTCGTCGGGTGTTGTTGGGCCTGTTGGTCCGCGGGGTTCCCCGGGTTTGAATGGTGTGAAAGGTCCTGACGGGTTGCCTGGTGCGAACGGATCGGATGGCCGTGATGGTGTTCCAGGTCGTGCAGGTGCTGACGGTGTGAACGGCGCTGATGGTCGGGATGGTTCGGCCGGTGAACGCGGTGATGTGGGCCCTTCAGGTCCTGCCGGCCCGCAAGGTGCACAGGGTGAACGGGGTCCTATTGGGCCTCAGGGTCCGCAGGGTTCTGCCGGTGCCGATGGCACGAATGGTCGGGACGGTAAAGATGGGCGCTCGGTGGTGTCTGTGTACTGTTCCGGGGGCCGCCTGGTTGTGAAATATAGTGACGGTGTGGCTTCTACAATATCGGGTTCGGTGGCCTGCCAGGGTGTGAAACCGTCGCCTATAGTGACTATATCATCCCACAAATAGAAAGGAGTGGCTGTGATTGTAGTGTTTGGTGGTGATGTGTGGTGAGGTTTATTCCAGCAGCACACCATTCTGCCGGCTCGAATAGTCCGGTGAATAGGGTTGTGATTCATGCAACATGCCCGGATGTGGGGTTTCCGTCTGCCTCGCGTAAGGGGAGGGCGGTGTCCACGGCAAACTATTTCGCGTCCCCATCGTCTGGTGGTTCGGCGCATTACGTGTGTGATATTGGGGAGACGGTGCAGTGCCTGTCAGAGGGGACTATTGGTTGGCATGCCCCGCCGAATCCGCATAGTTTGGGTATAGAGATTTGCGCGGATGGGGGTTCGCACGCCTCGTTCCGTGTGCCGGGGCATGCTTACACTCGTGAGCAGTGGCTTGATCCTCGCGTGTGGTCTGCGGTTGAGCGTGCCGCGGTGCTGTGCCGGCAGTTGTGTGACAAGCATGGTGTTCCGAAAAGGAAACTGTCTGTGGCCGATTTAAAGGCCGGTAAACGGGGTGTGTGTGGCCACGCGGATGTGACGGATGCGTGGCATCAGTCGGATCATGATGATCCGGGGCCGTGGTTTCCGTGGGACAGGTTTATGGCCGTAGTCAACGGCAAAGATGAGAGTGGGGAGTTAACTGTGGCTGATGTGAAAGCCTTGCATGATCAGATTAAACAATTGTCTGCTCAGCTTACTGGTTCGGTGAATAAGCTGCACCATGATGTTGGTGTGGTTCAGGTGCAGAATGGTGACCTGGCGCGCCGTGTGGAGGCTTTGTCGTGGGTGAAGAATCCGGTGACGGGGAAGCTGTGGCGCACTAAGGATGCTTTGTGGAGTGTCTGGTATTACGTGCTGGAGTGTCGTAGCCGTATTGACAGGCTTGAGTCGACTGTCAACGGTTTGAAAAAGTGATGGTGGTTTGTTGTGGGTAAACAGTTTTGGTTGGGCTTGTTTGAGCGTGCCCTGAAAACTTTTATTCAAACGTTTGTTGCTGTGCTTGGGGTTACTGCGGGTGTCACGTATACGGCGGAGTCGTTTCGCGGTTTGCCGTGGGAATCGGCGTTGATTACGGCCGGGGTTGCTGCGGTGCTGTCGGTGGCTACCTCGTTTGGTAACCCTGCGTTTGTGGCCGGCAAGCCTAAAACCACGGTTGTGGATGCGGGTTTGGTTCCACCCGATGATGGGGGCATTGTTGAGCCGCACATGGTTGATGTGTCGGATCCTGGCATGATCGAGCCGACAGATGATGCGGATCTTGGTGTAGGCTATGTGCCGAAACACGCTGCCGAGTCTGAGGTTGGCACGGTAGAGCCGATCGAATGATAAGTGAATATGTGTGTGTGCCCCAGCGGTGCTGCCACGATTGTGTGGTGGTTGCCGCTGGGGCACTATTTCTGTTTATGCGGTGTGGCTATGATTCGTTGCGGTCGATGGTGTCTTCGAGCACCTGATACAGGTGGAGGCAGGTAGAGATCGTATCGCTTGCCTGGTCTAGAACGTTCCGGCCGATAACGTTTTTGTGGTTGTCGCGGTGGCAGATGATAGCCCACATGATCTCGTCGGCTGCCGCCTGCAATAGTTTTGCCTGGTATGCGATTCCGGCGAGCCAGTCTAGTGCTTCCTGGCTTGCATAGGGGCTCTGGTCCTCGCTGTTGTCACGGGTGTTGCTGTTGTTTGTGGGGTGTCCTGCACTGTCGCATAACCACAGGATTTCGCTGCACTCGTCTAGCGTGTCCTGGTCGATAGCGAGATCGTCGAGGCTGACTTCGTTGACGGTAAGGTTCACGTTGTCGAGGGAGATGGGTACACCGTACTGGTTTTCGACACTGTCAACAATGTTTTCCAGCTGTTGCATGTTGGTGGGCTGTTGTTGGACGATACGGTGTATCGCTGTGTTGAGGGTGGTGTAGGTGATATTGTGTGTGTTGTTCATGGTTTTATTCCATCCCTGCGCTGTCGTCTTGGTAGTATCTACTGTTTGCGTAGCCTGTGAGGGTGATGAGTGTTTGGTCTGCCCACTGTTTCACAGTCTGCCGGGTGACTCCGAGTCGTTGGGCGGCTGTGGCGTAGGTTTGGTCATACCCGTAGACTTCCCGGAATGCTGCCAACCGTGCCAAATGTTTTCGCTGTTTGGATGGCTGGCAGGTGAGGGTGTAGTCGTCGATGGCTAGCTGCAAATCGATCATGGCGACAAGGTTGTTGCCGTGGTGTTGTGGCGCGGTTGGTGGGGGTGGCATTCCTGGTTCAACACTGGGTTTCCATGGGCCTCCGTTCCAGATCCATTGGGCGGCTTGGATGATGTCGGCGGTGGTGTAGGTTCGGTTCACTGGTCATCCCCTGAATAGGTTGTCGAGGTTGTCTGGGTTGCTGGTGTTGGTGGTGTCGAATCGTCCCACACAGTGGCAGTAGTCGTACATGAGTTTAATAATGTGTTGGTGGTCTCCCAAATAGGTGTTTCCGCTGATGCTGTAGGTGGCTGTGCCGTCTTTACTGATGGTGTATTTGGCGGTGATGGTTTCGGGTGTTTCTGTGTTGGTGATGATGGCGGTGGTGGTGGTGCCTACTGTTTGTAGCCTGGTGGTTTGGGTTCCGTCGTCGAGGATGGTGGTAACCATGAGGGGTTCTCCTTTTAAATGCTGGTTTGGTTGTCGGCTAGATGAATAATATCGGGTAAAGGTTTCGGTTGGTCTAGGTGTTGTATGGTTTTGTTGGCTAGCCGTTTGGCTACCCTGTAGCACATTTTGGTGTAGTGTTTGTTGTCTAGGTTGTGGTATTGTTCCCGCACCGCAATATATAGTAGGGAGTCTTGGTAGAGGTCGTCTGCGCTGATTGCGGGGTAGTGTGCGGCTGTTTTGGTGCATGCCCGGTTGAGTGTGCGTAGATGATGGTCTGTGGCCCACACCCATGATGCGGTGGTGGCGAGGTCTGCTTTTGTTGGTCGTCTGCTCATGGCATCTCTTTCATCGGGCTATCTGGTAGTTGTTTGGTGTTTTGTTGTTGATAGTGTAGCACACGAGTCCGGGGTTTCCGGTGGTGCCCGTCTTGTGCCGGTACCATGTGGATTCGCCTTCCATGGATGGGCATTGGATGAAGGTGCGTTGTCCTTGTTCGGAGATTTCGAGGTGGTGCCGGTGCCCGGCCATGAGAATATTAGATACGGTGCCGTTGTGGAATTCTTGTCCGCGCCACCAATCATAGTGTTTGCCGGTGCGCCATTGGTGTCCGTGGGCGTGCAGGATTTGTGTGCCGGCCACATCAACGGTGGTGGTCATTTCGTCTCGGCTGGGGAAGTGGAAGTGTAGGTTGGGGTATTGGTTGTTGAGCTGGTAGGCTTCTGCGATGGCGCGGCAGCAGTCCACGTCGAAGGAGTCGTCGTAGGTGGTGACGCCTTTGCCGAAGCGTACGGCTTCTCCGTGGTTGCCGGGGATGGATGTGATGGTGACGTTGGCGCAGTGGTCGAATTGGTGGATGAGTTGCATCATGGCCATGCGGGTGAGCCTGATTTGTTCGGTGAGGGGTGTTTGTGTGCGCCAGGCGTTGTTGCCGCCTTGTGACACGTATCCTTCGATCATGTCGCCGAGGAATGCGATGTGGACTCGTTGCGGTTTGCCTGCTTGCTGCCAGTAGTGTTTTGCGACTATGAGGGAGTGCAAATAGTCGTCGGCGAAGTGTGCTGTTTCTCCTCCGGGGATGCCTTTGCCGATTTGGAAGTCTCCTGCCCCGATGACGAAGGCCGCAGTGCTGTAGTCGGTGTGGGTGTCTTGTTCGGGTTTTGGGGGTGTCCATTCGGCTAGTTTGTTGACGAGTTCGTCTACGGGGTAGGGGTCGGTTGCGGGTTGGTGGTCGATGATTTTTTGTATGGATCGGCCGGTTTCTCCGTTCGGTAAGGTCCATTCGGAGATGCGTGTGCGGCGTACGGTGCCGTTGGCGAGATTGTCGTCGATGGTGTCGATGGCGTTGTCGTGGTTGGCTAGCTGTGTGAGTAGCCGGTCTATATTGTCTATCATCGGATATCCTCCTCTTGCGGGGTGGTGTTGGCTTGTTTGCGGCGGTAGTCTTTGATGACGGTGGCGGAGATGGGGTATCCGGCTTGGGTGAGCTGTTTTGCTAGCCATGAGGCGGGGATGGTTTTGTCGGCGAGCACGTCTGCAGCCTTGTTGCCGTAGCGTTGGATGAGGGTTTCAGTTTTGGTTGCCATGGTGTCCTAGGGGTTGTGTGGTGGGCTGCCATCCTGTGCGGCAGTCGCCGTCGTGTCCTGGTTTGCGTGTGCACCATGAGACTTCGCCGGCATTGTGGATGATGGCACGGCCGCATATGACGTCATGTAGGTGTTCGGGAAACTTATCGTTGTTGTTGTCCCCGTACATGTCGATCAAGTGTTGGGTTTTAGTAGCCATCATGTCTCCTATGTGTGAAAGAGTGTGCAAATACTATGCTGGTGTCATGGATGTTTATGCGGGTATGGTTTTCATCACCTTGCTGAACGTTACTTGGTTACTGTACATCATCTGGGTGATTTCCTGATCCGTTTTGTCGGGGTGCTGTTTTCGCAGGTTTGCCCATTGGCAGGCGTTGTCGGTTTCCTGCTGTAAACGTGTCAGGTGTTGTTCGTTGATGATGTGTTTCCACATGGTCCATGACACGTCGAGCCGGTTAAGGATTTCGAGGGCTGGCACGTTGAACTGGTCGAGGAAGAGTATTTCCTCCGTGTAGTAGTCTTTTTCGTATTGGTCCCATCCGCTTCGGTGCCTGTTGGGCTGGTTTTTGGGGTAGGCTTCCCGGCAGATTTTGTGTAACCGTTTTGCCATGTCGTCGGGTAGCCTAATGTCGGGGTTGGCGCGGATCATGGATCGCATCCCATCATAGGTGGTGCCCCAGGTGTGCATGATGTGGAGTGGGTCCACGCCGTCGGCCCATTTTTCTGCACAGATGGCGAGGCGGATACGCCTCCTGGCGGCCTTACTCGTGTCGCGGCGGCCGGGGATGGGGCATGTGTCGAGGGGGTCCATGATGTTTTCTATGCCTTTCTTTGTTGGGGTTGCTTGTGTGGTTTTATTGTAGCACTGTGTCTAGTGCTTGTGTCAACCCTGTTTTTCCGGCCTGCAGGTAGGTGTCTGTGACATCCCCCAGGGTGAGGGGTACGTGGGTGGCTTGGGGAGTGCTTGGGTTAGGGTTTGGGCCATCTTGTCTCCTGCTTTGTCTGGGTCGGACCAGATGTAGATGCGGTCGTAGCCTTCAAAAATTTGGTCCAGAAAGTTTGCCACGAGGTTGCGCCGGGTAGGGCTACGGATGGCCATCCGCATTGTTCGAGGATCATGGAGTCGAATTCGCCTTCGCAAATGTGCATTTCGGCTGCCGGGTTGGCCATGGCGGCCATGTTGTAGATGGAGCCTGTGTCCCCTGCCGGGGTTAGATATTTGGGGTGGTTGTGGGTTTTGCAATCATGCTGGAGTGAGCAGCGGAAACGCATTTTTCGTATTTCGGCTGGGCCGCCCCAGACGGGGTACATGTAGGGGATGGTGATGCACTGGTTGTAGTCTTCGTGGCCTGGGATGGGGTCATTGTCGATGTATCCAAGGTGGTGGTTGCGGGCTGTTTCTTCGCTGATGCCTCTTGCCGAGAGCAGGTCGAGTATGTTTTCGAAGTGGGTTTCGTAGAGGGCCGAGGCTTTCTGGATTCGGCGGCGTTCCGCAATGTTGTAGGGTTGTAGTGAGTCGTACATTAGGGTTTTCTTTCTCTAGTCGTTGTTTCAGTTTGGCGAGGCCTCCTCCGACACCGCATGTGTGGCAGTACCAGACGCCCTTGTCGAGGTTGATGCTCATGGAGGGCTGGTGGTCGTCGTGGAACGGGCAGAGGATGTGTTGCTCGTTCCTGGACGGGTTGTACCGTATCTGGTAGGTGTCGAGGAGGCGGCAGGTGTCAGAGGTGTGGGAGGAGCTCGTTGAGGGTTGATACCACATAGGCTTCGCTCCAGGGTTTGTTGCGCTGTTTCATGATGACGAGTCCGATGGTGGAATTGTTTTGTTTGTTTCGGTGTGTTTCGTAGTTGCGTGCCTCCCGGCTGGCTTGTTTCACGAATTCGGCGAGGTGTGGTTGCCCGGCTTTGGCTTCGATCACATAGGTTTTGTGGCTGGTTGTGAGGATGAGGTCGCCTTCGTCTTCTTTACCGTTGAGGTGGAGGCGTTCTATATCATGGCCGGTGTCGCGTAGTTGGTGGAGGAGTCGTGTTTCCCATTCTGCGCCGGACCGGCGGTTGCGTGACTGTTGTGTCGACATGATAGTCCTTTGTGGTGTTCGGTCATGTTCCAGGGCTGTTTTTCGGCGAGTGGCCCGAAGAATGTGTATTCGGGGTAGGCTCTGAGTCTTTCGTATCGGGTTCCGTCTGGGCTGGATTTGCCTGTGCGCTGTTTGAGTACGGCGATGCGTGCCTCGGCGGGGATGGTGAGCCCGTTGCCGTTATCCTCGCCACCATACAGGGAGACTCCCAATATGAGTTGTGGTTTTTCGGAGAGGCCGTTTTTGATTTCCCGCCTAGCCGGGGGTGTTCGATGTCGGAGCCGGTTTTGTCGGTTGCGTGGTGGGTGACAATAATGGTGGAGCCAGTATCCCTGCCCAATGCTGTGATCCATTGCATGGCTTCCTGCTGTGCCTGATAGTCGGATTCGCAGTCTTGGATGTCCATCAGGTTGTCGATGACGATGATGGGTGGGAAGGTGTTCCACATTTCCATGTAGGCTTGCAGTTCCATGGTGATGTCTGTCCATGTGATGGGTGACTGGAATGAGAATGTGATGTGTTGGCCGTGGTGGATGCTGTCTCGATAGTATTCTGGCCCGTAGTCGTCGATGTTTTGTTGTATCTGGGCGGTGGTGTGTTGGGTGTTGAGTGAGATGATTCGTGTGGAGGCCTCCCAGGGTGTCATGTCCCCTGATATGTAGAGGGCTGGCTGGTTGAGCATCGCTGTGATGAACATGGCTAGCCCGGATTTTTGGCTGCCGGACCGCCCCGCGATCATGACGAGATCCCCTTTGTGAATGTGCATGTCCAGGTTGCGGTAGAGGGGTTCTAGCTGGGGGATGCGGGGCAGTTCGGCTGCGGTTTGGGAGGCTCTCTCGAAGGATCGTTGGAGAGAGAGCATCGGAGCCTTAATCTATCTGTCTATCGGTTGGATGATGTTTTGGTGGTCAGATGGAGTCGATATCGATGTCAGCATCAGTTGAGGCTGTGGTGTCGTCTAGCTGGCCGTTATCGCGTTTGTCTACGTATTCGGCAACCTTATCGTAGATGGCGTCGTCTAATGGTTTGAGCACGACCGCGTTGAACCCGTTTTTGGTGCGTACGGTGGCGAGTTTGAAGGCCTGCTCCTCGCCAAGGTAGGCTTCTAGGTCGCGGATCATGGAGTGTGGGCGGTCGTTGTTGCCGCGCGCTTTCTCGATAATGGCGTTGGGGATGGTTTCTGGGGTGCCGTTGTTGAGATCGTCTAGGGTGTGGAAGATTGTGACATCAGCGTAGATGCGGTCTGCGGTCTGTCCGCCGTAGCCTTCGGTGTTGTGTTCTACGTCGCGGATTTTGAAGGCGATGGCGGTGGCGTCCTGGTTTCGGGAGGGGTTGAAGAAGGTGCTGTTGTTGTTGCGGTAGTTGGCGAGTCCCATTGTTGTTTCCTTTACTGTTGTGTCTGTTATTATTGGTTTATAGCGGTTTTTATCGGGTGAGGCTGTTTCGTTTGCTGCGGAAAGCCTCGGATACGTCACTGTTGCTGGTGATGGTCTTTTTGTATTGTTTTAGAAGGTCGGCTAGCTGTTGTTTGCTGGTGGCATTGTTGATTTTGTCGATGATGGTGTTGTTTCCTTCTGATGCAATGTTGTCTACGTAGTCTTTGGCTGCCTGGTTGTATCGGTCTTGTAGGATGATGGATGCTGTGGCGATCAGTGTTGCCAGGTCCCAGTTCCTTGCCGCGGAGCTGTTTTTGAGTCCGCCGAGGAGGTCGATGATAGTCTTCTTTACCTGGTCGGCGGTGTCTCCGCGGATGACGGTCCATGGGGCGGCGTAGTCTCCGCCGTATTTGAGGGTGACGGTGAATCGGTCGTCGTCTGTGTTGTCGGTCACTGGTGCTCCTTGCTTTCTTTTGTTGGGGCTGTGATGGTGGTTTCTACCGGGTATCTGTACGAGTTTTTCCCGTTGACGGCCCAGCAGGCGTCCTTGACGGGGCATCCTTTACAGAGTGTTGTGACGTGGGGTACGAAGATGCCTTGGCTGATTCCTTTCATTGCTTGACTGTACATGGATGATACATGCCGGTAGGTGTTGTTGTCAAGGTCGTAGAGTTCGGTGGATGTGCCCTGTTCGACCGATTGCTCGTCTCCCTTGGTGGTGGCGGGTGTCCAAAACATGCCTTTTGTCACATGGATGCCGTGTTGGTTGAGCATGTACCGGTATGTGTGCAGCTGCATGCTGTCGGCTGGGAGGCGTCCGGTTTTGAGGTCCAAAATGAAGGTTTCGCCAGTGTCGGTGTCGGTGAAAACGCGGTCGATGTAGCCAACAATCTGGGTGCCGTCCTGGAGGGTGGTTTCGACTGGGTATTCGATGCCTGGCTGGCCGTCAATAACAGCGGTGATGTATTCTGGGTGGTTGCGCCTCCATGTTTTCCACCGGTCCACAAAGGTGGGGCCGTATATCATCCACCAATTGTAGTCTTTTTTGTGGGGCCCGCCCGACTCGCACATGTTTTTGTACACCCTGCCGGAGGGTTTGATTTCGGTGCCTTCTGATTCGGCGAGGGCGACTTGGGTGTCGAAAATGTTTTTGAAGGATGAGAGTTTGTCTGGCAGTGCAGGGTATTCGGCGGGATTGTACAGGTGTAGGTCGTATTGTTCGGTGATGTGGTGTATGGCGCTTCCGGCGATGGTGGCGTACCAGGTGTGGTGTTGGGCGTGGTAGCCGTGTGAGAGGCGCCATTTTTCTCCGCATTCGGCCCACTGGGTGAGTGAACTGTAGGAGATGTGGCCTGGATGGTGGATGGTTTTCGGGTATTGTGCTAGAGGCATTACTTGTCGCCTTTGTGGGTGTTCCAGGGGTTGCGGGTGTCTACCCCGGCATCGTGTTGCTGGTAGGCGAGGAGTGCGAGGCAGTGCCAGGCAGCATGGGCTAGATGGGGTAGCCCGGATTCATCATCGAGGTTGCGTCCTTGCTGCCATGATAGTAGGTGCCTGTAGAGGGCGTCGACACTGTGGCTCCACGGATAGCCGCCGGTCCAGTTGTTGTCGCCGTATTTGGTGGCACCGTATCCTGCCACGGAGCCTAGATCGTGCAAGGCTGCGGGGTCGATGAGAGATAGCCTGCAAAGTTTGAGTTCTTTTTTGGCACCGGTGTTGGGGTCGGTGTACATGCGGGTGGGCTCATCCATGAGATGTGTGCTCCTTGAGTGTGGGTTACTGGTTGGGGTTGTGGGCGAGTGCTACGGCGAGAATAATGATGGCGAGGGTTTCAGCAATAAGTATGGGTGTTGTGATCATTTAGTGTCTCGGGGATTGTTGGTGAGGGTTGAGGCACCCAGGAGGGTGGCGAGGGCGCATGCGGCAATAATGGCGAGGGCTGCCTTGTGTGTGGTGCCGGTGGCGTACATCCATGTGATGATGCCGCCTTGGATCCATGCCAGTGTGGTGAAGAACGTTTCGTAGCTGTGCAGCTCAATGTTGTTGGGTGTGTTCATGCTTGCTCCTGAAGAATGGTGTTGATGGTTTTATAAATGTTGTACAGGTCGGCTTCGATGGTTTGTAGCTGTTTGATTTGGTGGTCGAGATCAATGTCTGGGTTGAGGGTGTTGATGCGGGAGGCGATGTCGGTGGCTGTGCGTAGTGTGCCGCCGGTGTGGTGAATGATGTGTGCCGTGTCGGCGAGTCCGGTGGTGACAGCGTAGTGGGAGAGGAGAGGCATAGCTGGGGGTGCTCCTTGGCGGGTTACTGTTGCGGGTTGATGTTGAGGTCGGTGACGTGCGGGTGGTCTTCTGTTCCGGTGACGAGGCAGTGGACGGTGACGGGTAGTTTGGATGCTCCCGGCTGGCGGACGGTGGCGCCGTAGACGATGGAGAAGGTGTCTTTGCCAATAATTTTGTGGAGTTGGAGGTCGATGTCGGGGTTGCCGTTCCATTTGACACCGTGTGCTGCGGCCTGTTGTTCGGCTTTGCGGTTGCAGGTGTGGGCTGCCGTGATCATGGTGAGACCTTGTGAGGTTTCTTCACCCCTTGTTTGGGCTTGCTTGTGGGCTTTCCGCTGTTCGGCTTGTAGTGACTGTTCTGCGGCTGCCTGCCGTGCTTTCTTGTCGGCTTTGCGCTGTTGGACGGTTTTGGGGGTCCATTCGGTGTTGGCTGTGGTGGCTTGCGGTGCGGGTTGTGAGGTGAGTGGCGGATTGTCGTCTGGGGCTGGCATGAATGAGGCGGCGGCTATGATGGCGGCTGTGGCGCCGGCGATGGTGTAGCCTGTTTTCTTGTTCATGGCTTTGTGTTCCCCTTTCCGGGGTGTTGTTCGTTGCTGACATGATTAATCATGGTGTGGGCGGTTCCCTATGTCAAGGCTGCGCTCAACGATTGTGAGCGATTCGTGTGTGGCTAGGGGTTTTATCGGCTGTACAGGGTGAGGAGGTGGCCAACATTGATGCGGCTCACATTCCAGTAGAGTTGTGTGGCTTCTCCGCCGGTGAGTGGCTTCCACTCGTTGTGGCTGAACACGGTGCCGTCGGAGGCGATGAATGTGTTGGGGCGTAGCTTGTGGAGTTCAGTCTCTACACGCTGCCGGTAGGTTTCGGCGAGGCCCTCAAAATCCATGTGGTCGCAGGAGAGGTTTTCGAGGCGTGTCAGGTCGAAGGGTGTGGGGCAGTCCGGGGTGGGGGTGTAGAGCTGGGTGAAGTGGTTGGCGATCTTCTGCATGACGGGTTCCTTTCTGGTGTGTGGTTGGGGTTTTATCGTGTGGCTTCGGCAATGATGGCGTCGACGTCGATCGTGTCGATCATGTCGTGGAGTTCCTCAGCCTCATCCGCGGTGAGTGGCTGCCAGTCCTGGGGTCCGTATATGGCACCGTCGAGGGTGACAGTCCACAGGGGTCGGATGAGCCGGACGGCTTCCTCGACTTTGGCACGGTGCAGGCGGCAGATGATAGACGTGTGGGTGTTGCCTATGTCACATCCTGCCAGGTGTGCGGGGTGGAGTGGGTTGATTTCTGTCTGCCCATAGAGGCTAGTGAAGGATGGTGTGATGAGTGTGCCATCCATGAGGGTGTGCTCCTTTCGGTGGTTGTGGGGGTTGTTGTGGTTTCTAGAGTGTGTAGGTTGCAACCCCATAGTCAAGGCTGCGCTCAAACCCAGTGAGCGTTTCATGGGATGTGGCAGGAGATGTGGCGTATCTCACTTAAGCCTTTATTGCCTCTCTCAGCGTCTCAAATCTTCTAGGGGTAGGATTATATAGGGTTGACCCTGCAGATCGATCCTAGGGCCCTTCTAGGGCGCCTCAGGGGTGTGTCTGGATGATAGCGGGTGTGGCAAGTGACGCAAACTGGAAGAGGGTGTTCAGTCGGGAGCACTAGATGGTCTGGCTGCACGGGTGCCTGAAGGACTCTTCGTTTATATGAGATATGTCACATCGCCTAGAGTCTAGGGACACCACAGACATCCACCAGCCTACCCTGCAGATGGCACAGGGGACAGGAATGCCTCCCTAAGGCGCATAAAGGCCCCTCTGAGGCTCTTGCACCCTCAACCCTAGGTATTTGTACCCCAATGATATTCTGATCGATTCTAGGGGCTGTTTTGGAGCTTACACGAGAACAGCACCCAAGAACTAGTCCATCAACCCTTACCCTGGTTAGCTAAGCCTCAACTATGTGGACAGTGTCGGATACTGTGGGGGAAGAAGGACACGGTAAAAGAAAGAAGGGGGATCATCAGCCTTCACACCTTCAAGCCTTAAGGTCTTAGCACTGAGCACCGAGCCCCCTCAAGGGCTCGGCATCAGCCCGAACAGGCACAGTCCTGAAAGGAGTACACGCCATCAGGGAAGGCTTGAGAGTACGAGGAGCCTTAGCGACGAGTACTCGAAAGCCTGATGGAAGACCATCAGCACTGATGGGCCTAGCGTGTTCGGAAAGGACACAAGGGTACAGTGTGAGAGCTGTTCGGGAGTGAAACCTGTTCTGACTAGGGGTTTCGGTCTTAACCACCCTCAAAGGTTACAAGACTCTAAGAAAATTTAAGGAAAAGTTTAGGTTTAATTTTTGGACCTTTACTACCAAAAACACCCGTTTACACCCCTCAAACCCGCCTATAGAGCCAAATCCACCAGTTTGACTCATCCCAGGTGGGGTATGATAGGCTGGACAGGTAGCCAGCTGGACGCAAGGCCGAAATCCGCTGACGCGGCTTTCACCCTTACATCCATCAGTCTACCAAAGACTTAAAGACCTAAGGGCTTAGCGCTAAGGTGCTGATAACTTAGCACCGAGCCCCTCAAGGGCTCGGCATCAGTCTTAAAGCTTTAAACACTTTAAGTAAACTTAAGAGCTTAGTACTTAAAGTTAATTAATAACCTTAAAGGCTTACACACTTAGCACTGAGCCCTTCAAGGCTCAGCATCAGCATAAAGACCTTAACACCTAAGTTAAGTATAAAACCTTAAAGGCTTAGCACTTAAGGATATAAACTTAACATCAGTGTTTAAGACTTTAAAACTTAAAATAACTATTAAGACTTATAAGCTTTAAACACTTAAAGTAACTATAAGACTTTAAAGACCTTAAGTACTTAAAGTTAACCATCAGTCTTAAACTTTAATATTATAACCTATAAGTCTTAAAGCTTATAGGTTATAAAAGTTTTAGAAGAGTTAAAGGGTTAACTTCTTTACTTCTCTACTCTCTTTGGTTCTTTCTCTCTTCTCTTCTTTTCTTCATCAGGGAAGAAGAGGAACCTTTGCCATCAGCGCTGATGGGCTTTCACTGTGTGGATCGTGTGCTTCTGGTCGCACGCTCCCGGTTGCACACTCCCCACACTCTGACACCCGTGTCCCTTTCAGGCTTAGCGTGTTCGGCTGAAGGCGTACGGCGTGTCACGCCAACACCCTTAACACCGGGTGAGACTTAAAGTGTATATTATATGTAGAAGACTTTAAAAACCTGTAAGGTGTTCCCGTTTAGCCTGTGTCCTTCACCGCTAGGCGCTAGGCGCTAAGCTGTGAAACGCGAACACCCACCCCCTTTTTTCTTTCGTGTCCTTCTTCTTTTGACACAGCTGGGGGGCGATGTGATCTTTCTCACATGTCAGGGGGTAGGGGAGAAAACAACCACCCCGGCATAAACAGAACACCCCCTCAAACAAACAAAACAGCCCCCAGAATCGAACAGCAGGGCAAGGGTAGAGCATTCATACCCCCAACGGTTTCCAGGCCGCTACAGGAGCAATGAGAGGCTCACAGGGGCAAGGGGAGATCAGGGGACGTGATGGCACATACCAACCGCACCGCATCGTCAGCCCATCGGCGCTGGCGGCAACGACTCATCACCCAAGCCCGACAGCAAGGCCAAACCGAATGCCCACTCTGCGGAGCAACCATCACCTGGAACACCCATGACCTGCCAACCAGCCCCGAAGCCGACCACATCACACCCGTCAGCCGGGGAGGACTCAACACCCTCGACAACGGTCAAATCATCTGCAGAACATGCAACAGAAGCAAAGGCAATCGCAGCGAACCAAACATCAAATTCCAACAACAAACCACAAAAACCTTGATTCCGTGGTGAAAAACCTGCCAACCCCCACCGGGGACACCCCTGCACACCCGTGCAAG